(19) United States
(12) Patent Application Publication
(10) Pub. No.: US 2025/0195667 A1
(43) Pub. Date: Jun. 19, 2025

(54) PHOSPHORODIAMIDATE MORPHOLINO OLIGOMER CONJUGATES

(71) Applicant: Sarepta Therapeutics, Inc., Cambridge, MA (US)

(72) Inventors: Gunnar J. HANSON, Cambridge, MA (US); Ming ZHOU, Cambridge, MA (US)

(21) Appl. No.: 18/842,988

(22) PCT Filed: Mar. 16, 2023
(86) PCT No.: PCT/US2023/064513

§ 371 Date: Aug. 30, 2024

Related U.S. Application Data
(60) Provisional application No. 63/320,773, filed on Mar. 17, 2022.

(51) Int. Cl. A61K 47/54; C12N 15/113

(52) U.S. Cl. CPC A61K 47/549; C12N 15/113; C12N 2310/14; C12N 2310/3233; C12N 2310/3519; C12N 2320/33

(57) ABSTRACT

Novel antisense oligonucleotide conjugates that cause skipping of an exon in the human dystrophin gene and their use in a method of treating muscular dystrophy in a patient suffering from Duchenne muscular dystrophy (DMD) are described.

### RELATED APPLICATION

This application claims the benefit of U.S. Provisional Application No. 63/320,773, filed on Mar. 17, 2022. The entire teachings of the above-referenced application are incorporated by reference in their entirety.

### SEQUENCE LISTING

The instant application contains a Sequence Listing which has been submitted electronically in XML format and is hereby incorporated by reference in its entirety. Said XML copy, created on Mar. 16, 2023, is named 4140_055PC01_SequenceListing_ST26 and is 572,344 bytes in size.

### FIELD OF THE DISCLOSURE

The present disclosure relates to certain phosphorodiamidate morpholino oligomersantisense oligonucleotide conjugates. The disclosure also relates to method of treating muscular dystrophy in a patient suffering from Duchenne muscular dystrophy (DMD) with an antisense oligonucleotide conjugate that causes skipping of an exon in the human dystrophin gene.

### BACKGROUND OF THE DISCLOSURE

Dystrophin is a critical structural protein that protects muscle from repeated strain-induced injury, affecting skeletal, diaphragmatic, and cardiac muscles. Duchenne muscular dystrophy is a rare, serious, life-threatening, X-linked recessive degenerative neuromuscular disease caused by mutations in the dystrophin gene. These mutations disrupt the reading frame of dystrophin messenger ribonucleic acid (mRNA), preventing the translation of functional dystrophin protein. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD. The absence of dystrophin protein is the direct cause of the disease and patients follow a predictable disease course with a relentlessly progressive deterioration of skeletal muscle function from early childhood leading to premature death, usually before 30 years of age.

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity, resulting in a BMD phenotype.

For many years it has been known that deletions of an exon or exons which do not alter the reading frame of a dystrophin protein would give rise to a BMD phenotype, whereas an exon deletion that causes a frame-shift will give rise to DMD (Monaco, Bertelson et al. 1988). In general, dystrophin mutations including point mutations and exon deletions that change the reading frame and thus interrupt proper protein translation result in DMD. It should also be noted that some BMD and DMD patients have exon deletions covering multiple exons.

Antisense oligonucleotides, e.g., splice switching oligonucleotides (SSOs), have been successfully used for the treatment of DMD to induce alternative splicing of pre-mRNAs by steric blockade of the spliceosome. SSOs have been specifically designed to target specific regions of the pre-mRNA, typically exons to induce the skipping of a mutation of the DMD gene thereby restoring these out-of-frame mutations in-frame to enable the production of internally shortened, yet functional dystrophin protein. Such antisense oligomers have been known to target completely within the exon (so called exon internal sequences) or at a splice donor or splice acceptor junction that crosses from the exon into a portion of the intron.

For example, eteplirsen is a phosphorodiamidate morpholino oligomer (PMO) designed to skip exon 51 of the human dystrophin gene in patients with DMD who are amenable to exon 51 skipping to restore the reading frame and produce a functional shorter form of the dystrophin protein. The United States Food and Drug Administration (FDA) approved in 2016 Exondys 51® (eteplirsen) for the treatment of DMD in patients who have a confirmed mutation of the DMD gene that is amenable to exon 51 skipping. For another example, golodirsen (Vyondys 53®), also an antisense oligonucleotide of the PMO subclass, has been approved for the treatment of DMD in patients with a confirmed mutation of the DMD gene that is amenable to exon 53 skipping. In addition, casimersen (Amondys 45™), also an antisense oligonucleotide of the PMO subclass, has been recently approved in the USA, for the treatment of DMD in patients who have a confirmed mutation of the DMD gene that is amenable to exon 45 skipping.

The discovery and development of antisense oligomers conjugated to cell-penetrating peptides (e.g., PPMOs) for DMD has also been an area of research (see, e.g., U.S. Pat. No. 10,888,578; U.S. application Ser. No. 16/469,104; U.S. Pat. No. 11,000,600). Cell-penetrating peptides (CPP), for example, an arginine-rich peptide transport moiety, have been shown to be effective in enhancing penetration of antisense oligomers into a cell and to cause exon skipping in different muscle groups in animal models.

Thus, despite the successes achieved in pre-clinical models with antisense oligomers conjugated to cell-penetrating peptides, the need remains for a safe and effective method for treating DMD and BMD with such conjugates in human patients.

### SUMMARY OF THE DISCLOSURE

It has been found that antisense oligomer conjugates according to Formula (I) are pharmacologically active. Certain antisense oligomer conjugates according to Formula (I) have also been found to distribute in different tissues, such as, e.g., muscle and kidney tissues.

In some aspects, the disclosure relates to antisense oligomer conjugates of Formula (I):

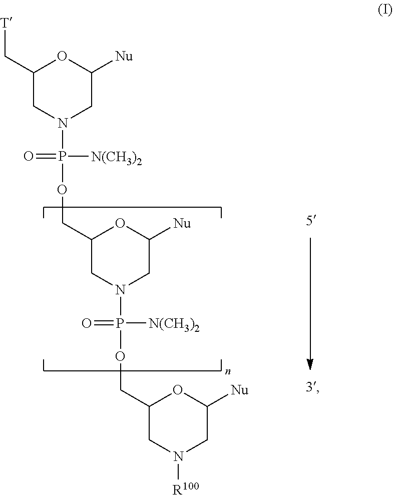

and pharmaceutically acceptable salts thereof,
wherein:
n is 1-40;
each Nu is a nucleobase, which, taken together, form a targeting sequence complementary to an exon annealing site in the dystrophin pre-mRNA;
T′ is a moiety selected from:

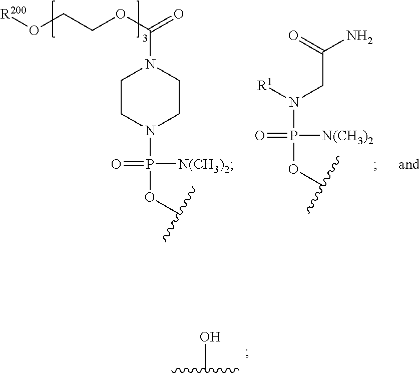

wherein
R¹⁰⁰ is selected from the group consisting of RRRRRG-, RRRRG-, RRRG-, RRG-, RG-, and G-, wherein R is arginine and G is glycine,
R²⁰⁰ is hydrogen, and
R¹ is C₁-C₆ alkyl.

In some aspects, the targeting sequence of the antisense oligomer conjugate of Formyla (I), or a pharmaceutically acceptable salt thereof, is complementary to an exon 51 annealing site in the dystrophin pre-mRNA designated as H51A(+66+95).

In some aspects, the targeting sequence of the antisense oligomer conjugate of Formula (I), or a pharmaceutically acceptable salt thereof, is complementary to an exon 45 annealing site in the dystrophin pre-mRNA designated as H45A(−03+19).

In some aspects, the targeting sequence of the antisense oligomer conjugate of Formula (I), or a pharmaceutically acceptable salt thereof, is complementary to an exon 53 annealing site in the dystrophin pre-mRNA designated as H53A(+36+60).

In some aspects, each Nu in the antisense oligomer conjugate of Formula (I), or a pharmaceutically acceptable salt thereof, is independently selected from cytosine (C), guanine (G), thymine (T), adenine (A), 5-methylcytosine (5mC), uracil (U), and hypoxanthine (I).

In some aspects, in the antisense oligomer conjugate of Formula (I), or a pharmaceutically acceptable salt thereof, T′ is a moiety:

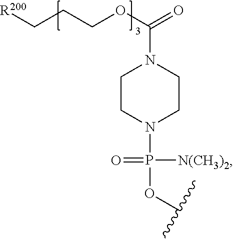

wherein R²⁰⁰ is hydrogen.

In some aspects, the present disclosure provides antisense oligomer conjugates of Formula (I), and pharmaceutically acceptable salts thereof, wherein R¹⁰⁰ is RRRRRG-.

In some aspects, the present disclosure provides antisense oligomer conjugates of Formula (I), and pharmaceutically acceptable salts thereof, wherein R¹⁰⁰ is RRRRG-.

In some aspects, the present disclosure provides antisense oligomer conjugates of Formula (I), and pharmaceutically acceptable salts thereof, wherein R¹⁰⁰ is RRRG-.

In some aspects, the present disclosure provides antisense oligomer conjugates of Formula (I), and pharmaceutically acceptable salts thereof, wherein R¹⁰⁰ is RRG-.

In some aspects, the present disclosure provides antisense oligomer conjugates of Formula (I), and pharmaceutically acceptable salts thereof, wherein R¹⁰⁰ is RG-.

In some aspects, the present disclosure provides antisense oligomer conjugates of Formula (I), and pharmaceutically acceptable salts thereof, wherein R¹⁰⁰ is G-.

In certain aspects, the present disclosure provides an antisense oligomer conjugate having the Formula (V):

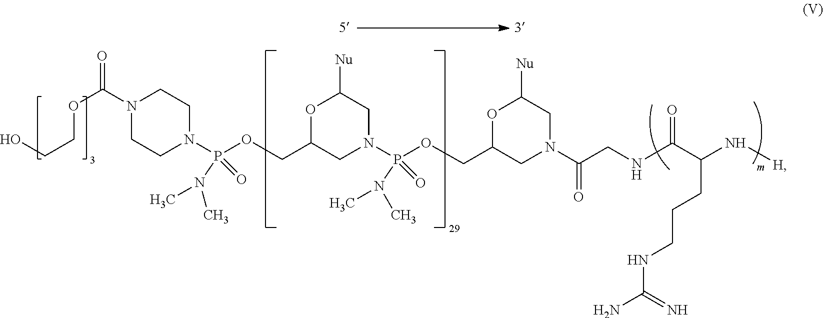

or a pharmaceutically acceptable salt thereof, wherein
each Nu is a nucleobase, which, taken together, form a targeting sequence that is complementary to an exon annealing site in the dystrophin pre-mRNA, and
m is 0, 1, 2, 3, 4, or 5.

In some embodiments, the antisense oligomer conjugate of Formula (V) is according to Formula (VA):

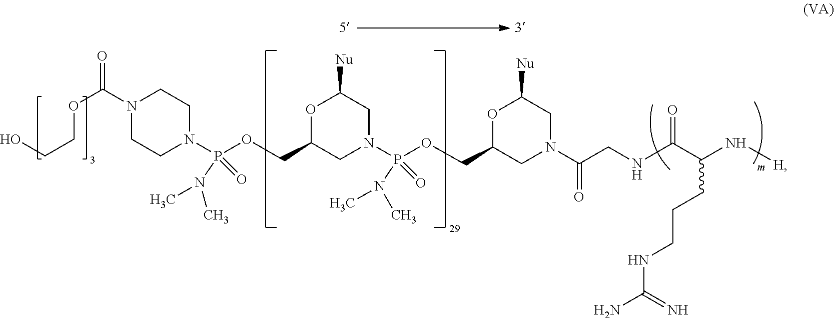

or a pharmaceutically acceptable salt thereof, wherein m is 0, 1, 2, 3, 4, or 5, and
each Nu from 1 to 30 and 5′ to 3′ is:

| Position No. | |
|---|---|
| 5′ to 3′ | Nu |
| 1 | C |
| 2 | T |
| 3 | C |
| 4 | C |
| 5 | A |
| 6 | A |
| 7 | C |
| 8 | A |
| 9 | T |
| 10 | C |
| 11 | A |
| 12 | A |
| 13 | G |
| 14 | G |
| 15 | A |
| 16 | A |
| 17 | G |
| 18 | A |
| 19 | T |
| 20 | G |
| 21 | G |
| 22 | C |
| 23 | A |
| 24 | T |
| 25 | T |
| 26 | T |
| 27 | C |
| 28 | T |
| 29 | A |
| 30 | G |

wherein A is

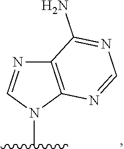 [SMILES: *n1cnc2c(N)ncnc21]

### C is

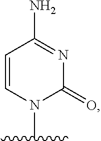 [SMILES: *n1ccc(N)nc1=O]

### G is

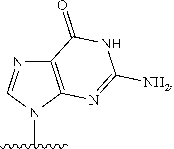 [SMILES: *n1cnc2c(=O)[nH]c(N)nc21]

### and T is

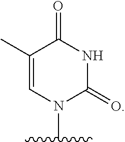 [SMILES: *n1cc(C)c(=O)[nH]c1=O]

In certain aspects, the present disclosure provides an antisense oligomer conjugate having the Formula (VII):

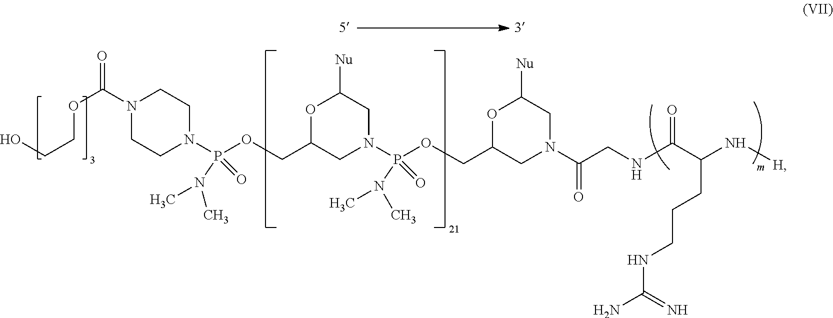

or a pharmaceutically acceptable salt thereof, wherein
each Nu is a nucleobase, which, taken together, form a targeting sequence that is
complementary to an exon annealing site in the dystrophin pre-mRNA, and
m is 0, 1, 2, 3, 4, or 5.

In some embodiments, the antisense oligomer conjugate of Formula (VII) is according to Formula (VIIA):

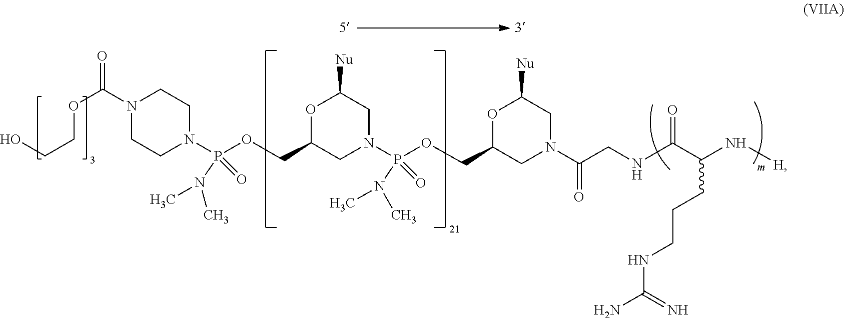

or a pharmaceutically acceptable salt thereof, wherein m is 0, 1, 2, 3, 4, or 5, and
each Nu from 1 to 22 and 5′ to 3′ is:

| Position No. | |
|---|---|
| 5′ to 3′ | Nu |
| 1 | C |
| 2 | A |
| 3 | A |
| 4 | T |
| 5 | G |
| 6 | C |
| 7 | C |
| 8 | A |
| 9 | T |
| 10 | C |
| 11 | C |
| 12 | T |
| 13 | G |
| 14 | G |
| 15 | A |
| 16 | G |
| 17 | T |
| 18 | T |
| 19 | C |
| 20 | C |
| 21 | T |
| 22 | G |

wherein A is

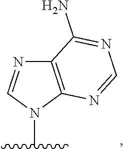 [SMILES: *n1cnc2c(N)ncnc21]

### C is

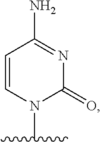 [SMILES: *n1ccc(N)nc1=O]

### G is

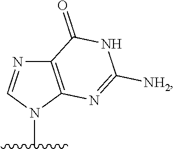 [SMILES: *n1cnc2c(=O)[nH]c(N)nc21]

### and T is

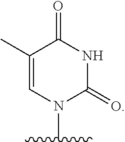 [SMILES: *n1cc(C)c(=O)[nH]c1=O]

In certain aspects, the present disclosure provides an antisense oligomer conjugate having the Formula (IX):

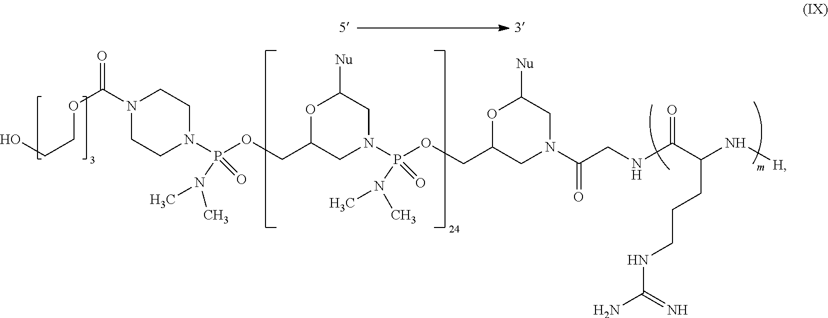

or a pharmaceutically acceptable salt thereof, wherein
each Nu is a nucleobase, which, taken together, form a targeting sequence that is
complementary to an exon annealing site in the dystrophin pre-mRNA, and
m is 0, 1, 2, 3, 4, or 5.

In some embodiments, the antisense oligomer conjugate of Formula (IX) is according to Formula (IXA):

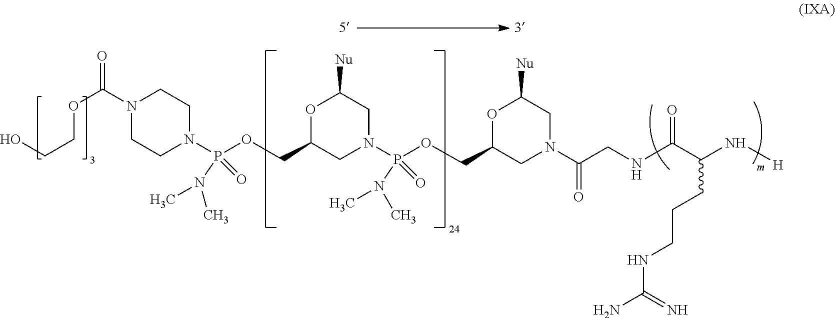

or a pharmaceutically acceptable salt thereof, wherein m is 0, 1, 2, 3, 4, or 5, and
each Nu from 1 to 25 and 5′ to 3′ is:

| Position No. | |
|---|---|
| 5′ to 3′ | Nu |
| 1 | G |
| 2 | T |
| 3 | T |
| 4 | G |
| 5 | C |
| 6 | C |
| 7 | T |
| 8 | C |
| 9 | C |
| 10 | G |
| 11 | G |
| 12 | T |
| 13 | T |
| 14 | C |
| 15 | T |
| 16 | G |
| 17 | A |
| 18 | A |
| 19 | G |
| 20 | G |
| 21 | T |
| 22 | G |
| 23 | T |
| 24 | T |
| 25 | C |

wherein A is

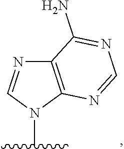 [SMILES: *n1cnc2c(N)ncnc21]

### C is

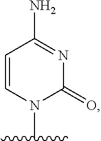 [SMILES: *n1ccc(N)nc1=O]

### G is

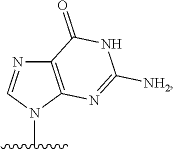 [SMILES: *n1cnc2c(=O)[nH]c(N)nc21]

### and T is

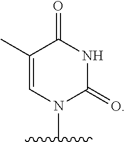 [SMILES: *n1cc(C)c(=O)[nH]c1=O]

In certain aspects, the present disclosure provides an antisense oligomer conjugate of any of Formulae (V), (VA), (VII), (VIIA), (IX), or (IXA), or a pharmaceutically acceptable salt thereof, wherein m is 0.

In certain aspects, the present disclosure provides an antisense oligomer conjugate of any of Formulae (V), (VA), (VII), VIIA), (IX), or (IXA), or a pharmaceutically acceptable salt thereof, wherein m is 1.

In certain aspects, the present disclosure provides an antisense oligomer conjugate of any of Formulae (V), (VA), (VII), VIIA), (IX), or (IXA), or a pharmaceutically acceptable salt thereof, wherein m is 2.

In certain aspects, the present disclosure provides an antisense oligomer conjugate of any of Formulae (V), (VA), (VII), VIIA), (IX), or (IXA), or a pharmaceutically acceptable salt thereof, wherein m is 3.

In certain aspects, the present disclosure provides an antisense oligomer conjugate of any of Formulae (V), (VA), (VII), VIIA), (IX), or (IXA), or a pharmaceutically acceptable salt thereof, wherein m is 4.

In certain aspects, the present disclosure provides an antisense oligomer conjugate of any of Formulae (V), (VA), (VII), VIIA), (IX), or (IXA), or a pharmaceutically acceptable salt thereof, wherein m is 5.

In certain embodiments, the antisense oligomer conjugates of the disclosure are provided in a free base form. In certain embodiments, the antisense oligomer conjugates of the disclosure provided herein are pharmaceutically acceptable salts, such as a hydrochloride salt.

The present disclosure also provides a pharmaceutical composition, comprising an antisense oligomer conjugate described herein, such as an antisense oligomer conjugate of Formula (I), or a pharmaceutically acceptable salt thereof, and a pharmaceutically acceptable carrier. In some embodiments, the pharmaceutical composition is formulated for parenteral use.

The present disclosure also provides a method of treating a patient with Duchenne muscular dystrophy (DMD) in need thereof who has a mutation that is amenable to exon skipping, comprising administering to the patient an antisense oligomer conjugate described herein, such as an antisense oligomer conjugate of Formula (I), or a pharmaceutically acceptable salt thereof. In some embodiments, the antisense oligomer conjugate causes skipping of an exon in the human dystrophin gene. In some embodiments, the exon is chosen from exon 44, 45, 50, 51, 52, or 53. In some embodiments, the exon is chosen from exon 45, 51, or 53.

The present disclosure provides a method of treating a patient with Duchenne muscular dystrophy (DMD) with an antisense oligomer conjugate, comprising administering to the patient an antisense oligomer conjugate described herein, such as an antisense oligomer conjugate of Formula (I), or a pharmaceutically acceptable salt thereof.

In certain aspects, the present disclosure provides a method of treating a patient with DMD in need thereof who has a mutation that is amenable to exon 51 skipping, comprising administering to the patient an antisense oligomer conjugate having Formula (VI):

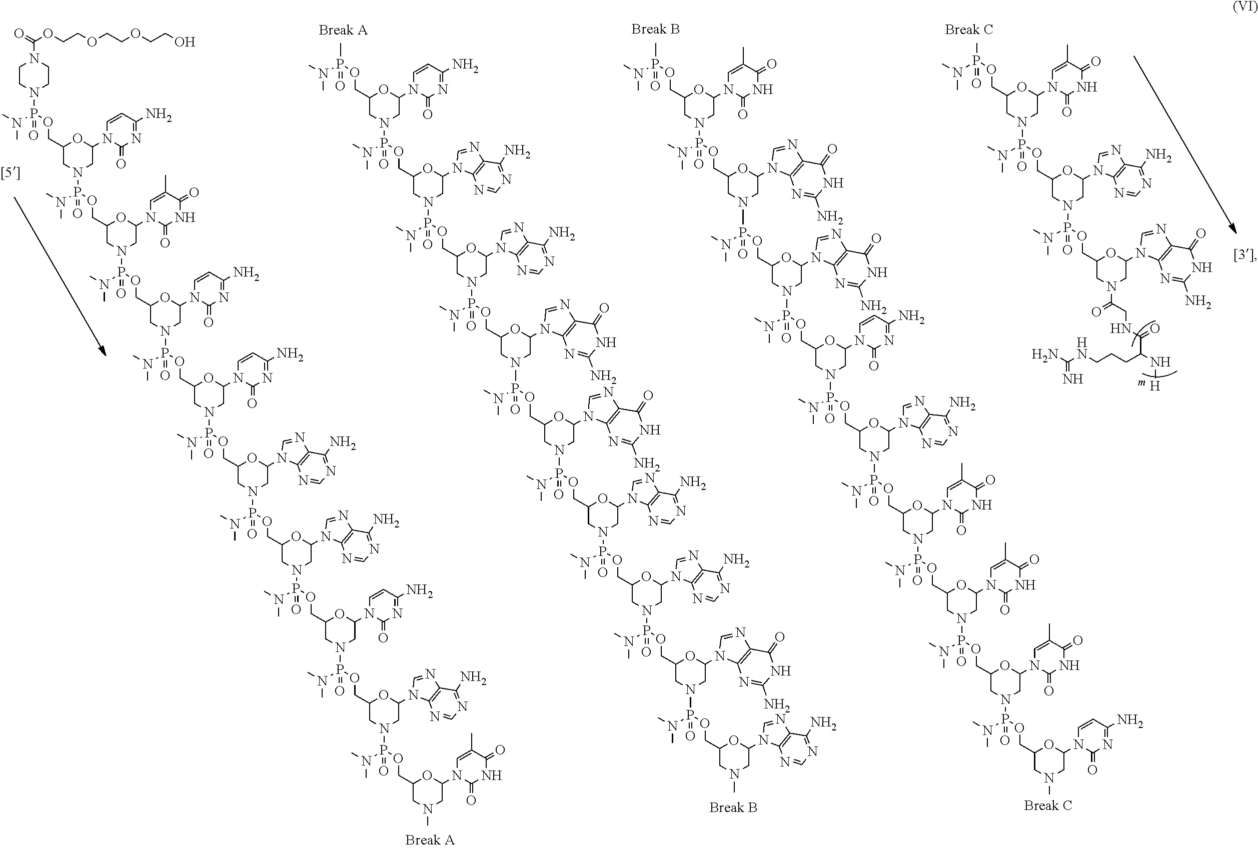

or a pharmaceutically acceptable salt thereof, wherein m is 0, 1, 2, 3, 4, or 5.

In certain aspects, the present disclosure provides a method of treating a patient with DAM in need thereof who has a mutation that is amenable to exon 45 skipping, comprising administering to the patient an antisense oligomer conjugate having Formula (VIII):

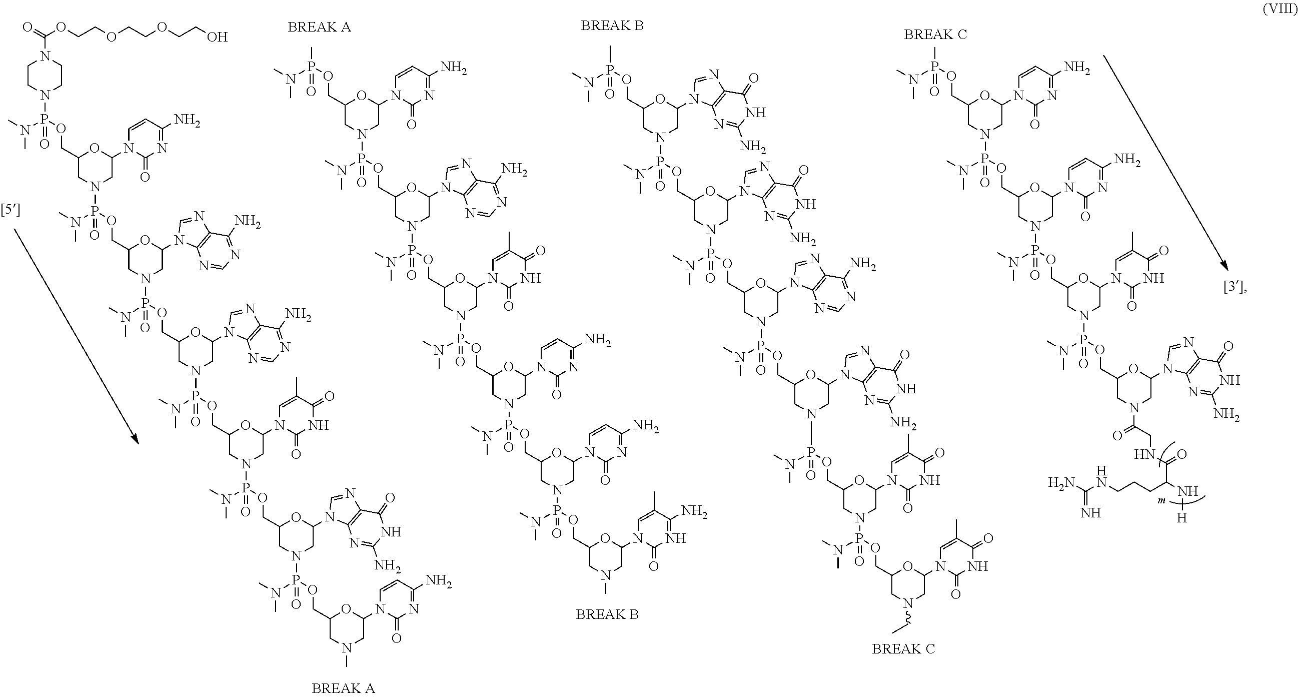

or a pharmaceutically acceptable salt thereof, wherein m is 0, 1, 2, 3, 4, or 5.

In certain aspects, the present disclosure provides a method of treating a patient with DAM in need thereof who has a mutation that is amenable to exon 53 skipping, comprising administering to the patient an antisense oligomer conjugate having Formula (X):

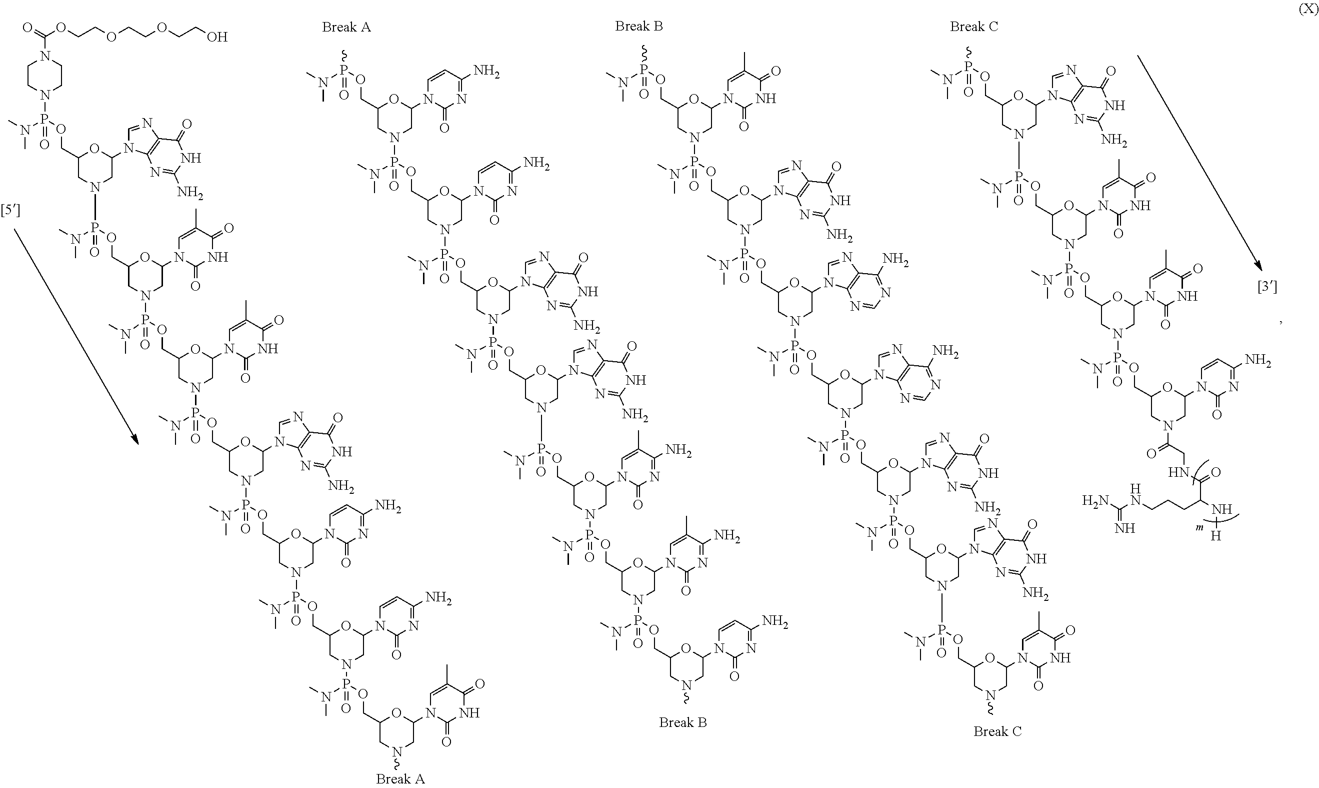

or a pharmaceutically acceptable salt thereof, wherein m is 0, 1, 2, 3, 4, or 5.

### DETAILED DESCRIPTION

The present disclosure relates to antisense oligonucleotide conjugates and their use in methods for treating muscular dystrophy, such as DMD and BMD, in a patient. The method comprises administering an antisense oligomer conjugate described herein, or a pharmaceutically acceptable salt thereof, to induce exon skipping in the human dystrophin gene.

### Definitions

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group includes one to six carbon atoms, i.e., C₁ to C₆ alkyl. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo, or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

“Amenable to exon skipping” as used herein with regard to a subject or patient is intended to include subjects and patients having one or more mutations in the dystrophin gene which, absent the skipping of the particular exon of the dystrophin pre-mRNA, causes the reading frame to be out-of-frame thereby disrupting translation of the pre-mRNA leading to an inability of the subject or patient to produce functional or semi-functional dystrophin. Determining whether a patient has a mutation in the dystrophin gene that is amenable to exon skipping is well within the purview of one of skill in the art (see, e.g., Aartsma-Rus et al. (2009) Hum Mutat. 30:293-299; Gurvich et al., Hum Mutat. 2009; 30(4) 633-640; and Fletcher et al. (2010) Molecular Therapy 18(6) 1218-1223.).

The terms “oligomer” and “oligonucleotide” are used interchangeably and refer to a sequence of subunits connected by intersubunit linkages. In certain instances, the term “oligomer” is used in reference to an “antisense oligomer.” For “antisense oligomers,” each subunit consists of: (i) a ribose sugar or a derivative thereof, and (ii) a nucleobase bound thereto, such that the order of the base-pairing moieties forms a base sequence that is complementary to a target sequence in a nucleic acid (typically an RNA) by Watson-Crick base pairing, to form a nucleic acid:oligomer heteroduplex within the target sequence with the proviso that either the subunit, the intersubunit linkage, or both are not naturally occurring. In certain embodiments, the antisense oligomer is a PMO.

The terms “complementary” and “complementarity” refer to two or more oligomers (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some embodiments, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The terms “effective amount” and “therapeutically effective amount” are used interchangeably herein and refer to an amount of therapeutic compound, such as an antisense oligomer conjugate, administered to a mammalian subject, either as a single dose or as part of a series of doses, which is effective to produce a desired therapeutic effect. For an antisense oligomer conjugate, this effect is typically brought about by inhibiting translation or natural splice-processing of a selected target sequence, or producing a clinically meaningful amount of dystrophin (statistical significance).

By “enhance” or “enhancing,” or “increase” or “increasing,” or “stimulate” or “stimulating,” refers generally to the ability of one or more antisense oligomer conjugates or pharmaceutical compositions to produce or cause a greater physiological response (i.e., downstream effects) in a cell or a subject, as compared to the response caused by either no antisense oligomer conjugate or a control compound. A greater physiological response may include increased expression of a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or 100%. The percentage of muscle fibers that express a functional dystrophin can also be measured, including increased dystrophin expression in about 1%, 2%, 5%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibers. For instance, it has been shown that around 40% of muscle function improvement can occur if 25-30% of fibers express dystrophin (see, e.g., DelloRusso et al, Proc Natl Acad Sci USA 99: 12979-12984, 2002). An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times, including all integers and decimal points in between and above 1), e.g., 1.5, 1.6, 1.7, 1.8, etc.) the amount produced by no antisense oligomer conjugate (the absence of an agent) or a control compound.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vivo biological activity of wild-type dystrophin, as measured according to routine techniques in the art. Included are truncated forms of dystrophin, such as those forms that are produced following the administration of certain of the exon-skipping antisense oligomer conjugates of the present disclosure.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some embodiments can include one or more but preferably 6, 5, 4, 3, 2, or 1 mismatches with respect to the target pre-mRNA. Variations at any location within the oligomer are included. In certain embodiments, antisense oligomer conjugates of the disclosure include variations in nucleobase sequence near the termini variations in the interior, and if present are typically within about 6, 5, 4, 3, 2, or 1 subunits of the 5′ and/or 3′ terminus.

The terms “morpholino,” “morpholino oligomer,” and “PMO” refer to a phosphorodiamidate morpholino oligomer of the following general structure:

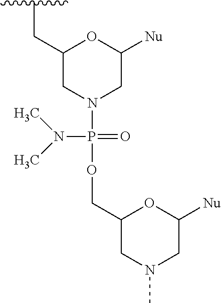

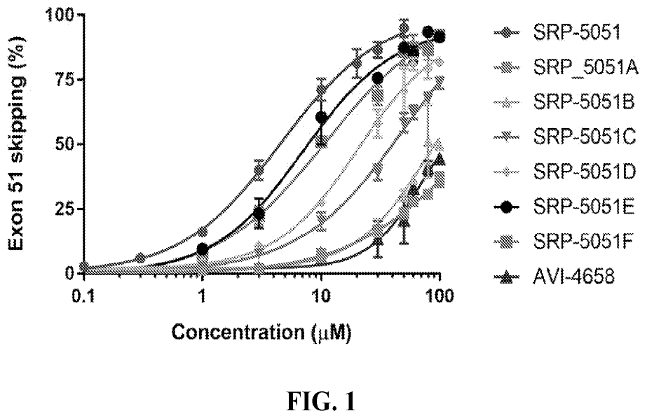

and as described in FIG. 2 of Summerton, J., et al., Antisense & Nucleic Acid Drug Development, 7: 187-195 (1997). Morpholinos as described herein include all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206; all of which are incorporated herein by reference.

In some aspects, a morpholino oligo (PMO) is conjugated at the 5′ or 3′ end of the oligomer with a “tail” moiety to increase its stability and/or solubility. Exemplary tails include:

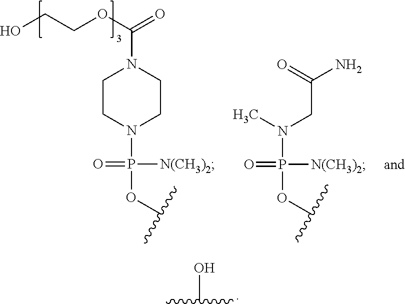

Of the above exemplary tail moieties, “TEG” or “EG3” refers to the following tail moiety:

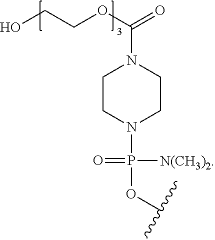

Of the above exemplary tail moieties, “GT” refers to the following tail moiety:

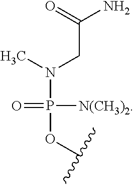 [SMILES: *OP(=O)(N(C)C)N(C)CC(N)=O]

The term “RRRRRG-” as used herein refers to the structure:

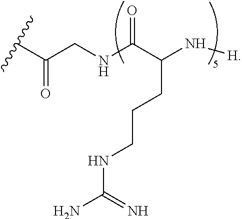

The term “RRRRG-” as used herein refers to the structure:

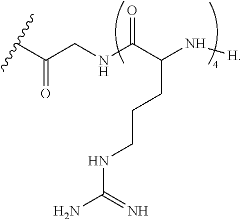

The term “RRRG-” as used herein refers to the structure:

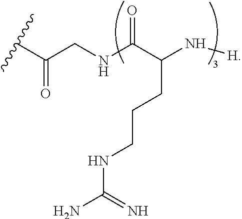

The term “RRG-” as used herein refers to the structure:

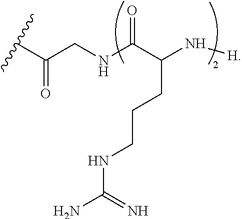

The term “RG-” as used herein refers to the structure:

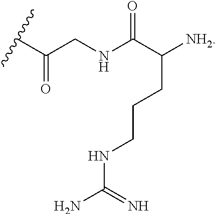 [SMILES: *C(=O)CNC(=O)C(N)CCCNC(=N)N]

The term “G-” as used herein refers to the structure:

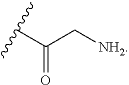 [SMILES: *C(=O)CN]

Synthesis of arginine peptides and methods of conjugating to an oligomer are described in U.S. Pat. Nos. 9,161,948, 10,888,578, and 11,000,600, U.S. Application Publication No. 2012/0289457, and International Patent Application Publication Nos. WO 2004/097017, WO 2009/005793, and WO 2012/150960, the disclosures of which are incorporated herein by reference in their entirety.

The terms “nucleobase” (Nu), “base pairing moiety” or “base” are used interchangeably to refer to a purine or pyrimidine base found in naturally occurring, or “native” DNA or RNA (e.g., uracil, thymine, adenine, cytosine, and guanine), as well as analogs of these naturally occurring purines and pyrimidines. These analogs may confer improved properties, such as binding affinity, to the oligomer. Exemplary analogs include hypoxanthine (the base component of inosine); 2,6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine (inosine) having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in: Chiu and Rana, R N A, 2003, 9, 1034-1048; Limbach et al. Nucleic Acids Research, 1994, 22, 2183-2196; and Revankar and Rao, Comprehensive Natural Products Chemistry, vol. 7, 313; are also contemplated, the contents of which are incorporated herein by reference.

Further examples of base pairing moieties include, but are not limited to, expanded-size nucleobases in which one or more benzene rings has been added. Nucleic acid base replacements described in: the Glen Research catalog (www.glenresearch.com); Krueger A T et al., Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F E., et al., Curr. Opin. Chem. Biol., 2003, 7, 723-733; and Hirao, I., Curr. Opin. Chem. Biol., 2006, 10, 622-627; the contents of which are incorporated herein by reference, are contemplated as useful in the antisense oligomer conjugates described herein. Examples of expanded-size nucleobases include those shown below, as well as tautomeric forms thereof.

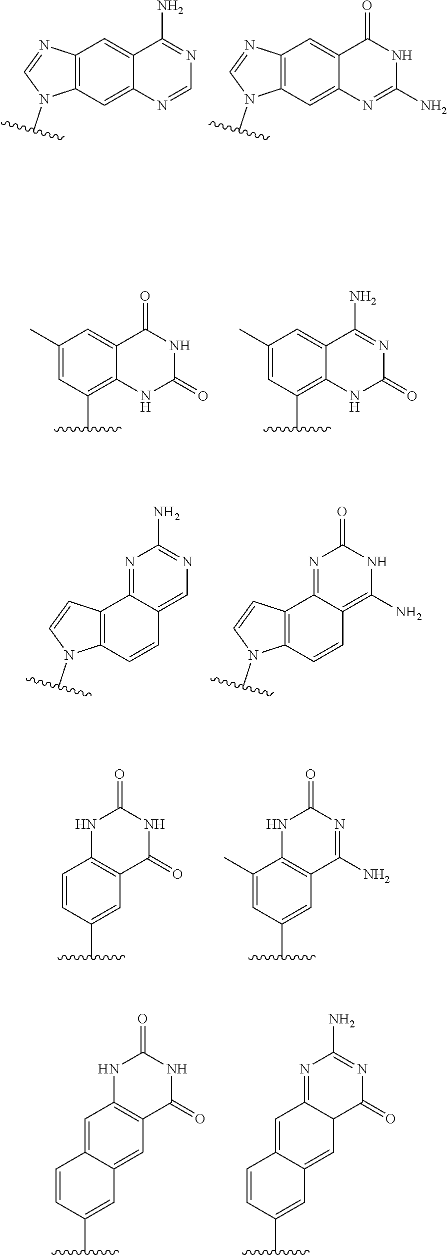

The term “exposure” refers to dose (PPMO input to the body) and various measures of acute or integrated PPMO concentrations in plasma and other biological fluid (e.g., Cmax, Cmin, Css, AUC). The term “response” refers to a direct measure of the pharmacologic effect of the drug. Response includes a broad range of endpoints or biomarkers ranging from a potential or accepted surrogate (e.g., effects on blood pressure, magnesium levels, or cardiac output) to the full range of short-term or longterm clinical effects related to efficacy and safety.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

For clarity, structures of the disclosure are continuous from 5′ to 3′, and, for the convenience of depicting the entire structure in a compact form, various illustration breaks labeled “BREAK A,” “BREAK B,” and “BREAK C” have been included. As would be understood by the skilled artisan, for example, each indication of “BREAK A” shows a continuation of the illustration of the structure at these points. The skilled artisan understands that the same is true for each instance of “BREAK B” and for “BREAK C” in the structures above. None of the illustration breaks, however, are intended to indicate, nor would the skilled artisan understand them to mean, an actual discontinuation of the structure above.

As used herein, a set of brackets used within a structural formula indicate that the structural feature between the brackets is repeated. In some embodiments, the brackets used can be “[” and “],” and in certain embodiments, brackets used to indicate repeating structural features can be “(“and”).” In some embodiments, the number of repeat iterations of the structural feature between the brackets is the number indicated outside the brackets such as 2, 3, 4, 5, 6, 7, and so forth. In various embodiments, the number of repeat iterations of the structural feature between the brackets is indicated by a variable indicated outside the brackets such as “n”.

As used herein, a straight bond or a squiggly bond drawn to a chiral carbon or phosphorous atom within a structural formula indicates that the stereochemistry of the chiral carbon or phosphorous is undefined and is intended to include all forms of the chiral center. Examples of such illustrations are depicted below.

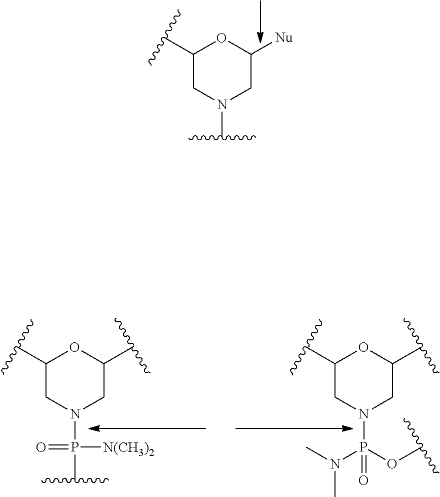

### Antisense Oligomer Conjugates of the Disclosure

In various aspects, the disclosure provides antisense oligomer conjugates according to Formula (I):

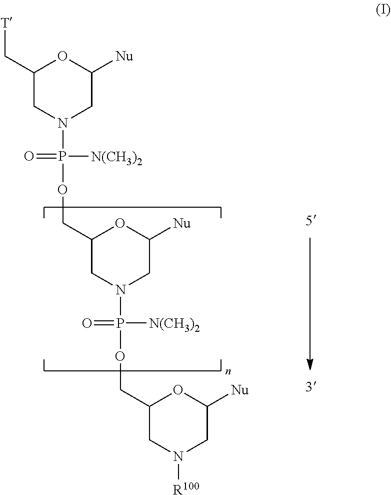

or a pharmaceutically acceptable salt thereof, wherein:
n is 1-40;
each Nu is a nucleobase, which, taken together form a targeting sequence that is
complementary to an exon annealing site in the dystrophin pre-mRNA;
T′ is a moiety selected from:

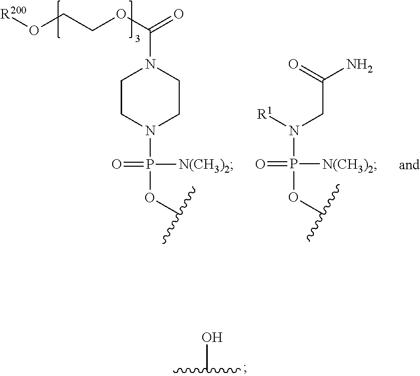

wherein
R¹⁰⁰ is selected from the group consisting of RRRRRG-, RRRRG-, RRRG-, RRG-, RG-, and G-, wherein R is arginine and G is glycine,
R²⁰⁰ is hydrogen, and
R¹ is C₁-C₆ alkyl.

In some embodiments, T′ is a moiety:

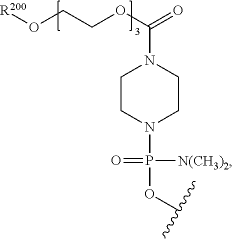

wherein R²⁰⁰ is hydrogen.

In some embodiments, the antisense oligomer conjugate is according to Formula (I), or a pharmaceutically acceptable salt thereof, wherein R¹⁰⁰ is RRRRRG-.

In some embodiments, the antisense oligomer conjugate is according to Formula (I), or a pharmaceutically acceptable salt thereof, wherein R¹⁰⁰ is RRRRG-.

In some embodiments, the antisense oligomer conjugate is according to Formula (I), or a pharmaceutically acceptable salt thereof, wherein R¹⁰⁰ is RRRG-.

In some embodiments, the antisense oligomer conjugate is according to Formula (I), or a pharmaceutically acceptable salt thereof, wherein R¹⁰⁰ is RRG-.

In some embodiments, the antisense oligomer conjugate is according to Formula (I), or a pharmaceutically acceptable salt thereof, wherein R¹⁰⁰ is RG-.

In some embodiments, the antisense oligomer conjugate is according to Formula (I), or a pharmaceutically acceptable salt thereof, wherein R¹⁰⁰ is G-.

In some aspects, the antisense oligomer of the antisense oligomer conjugate has n+2 base pairs, where n in Formula (I) is 1 to 40, optionally 13-38, optionally 13-28, optionally 13-23 or optionally 13-18. In other words, the oligomer is 15-40, 15-35, 15-30, 15-25, or 15-20 nucleotides in length.

In some aspects, the antisense oligomer conjugate of Firmula (I) causes skipping of an exon in the human dystrophin gene. In some aspects, the exon is chosen from exon 44, 45, 50, 51, 52, or 53. In certain aspects, the exon is chosen from exon 45, 51, or 53.

In various aspects, an antisense oligomer conjugate is according to Formula (II):

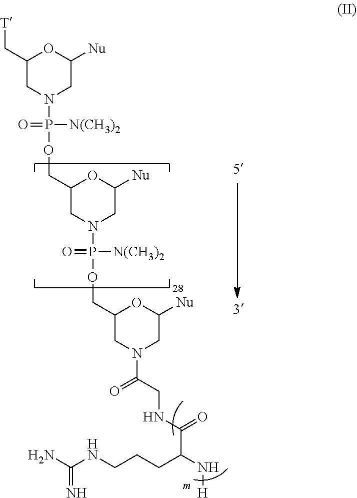

or a pharmaceutically acceptable salt thereof, wherein:
each Nu is a nucleobase which taken together form a targeting sequence;
T′ is a moiety selected from:

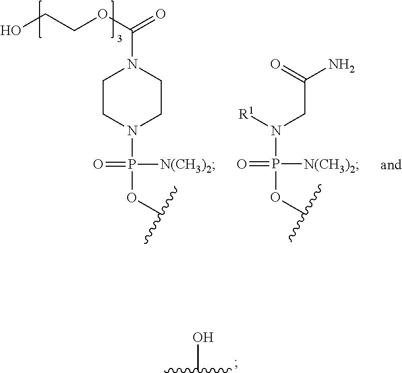

R¹ is C₁-C₆ alkyl; and
m is 0, 1, 2, 3, 4, or 5;
wherein the targeting sequence is complementary to an annealing site in the dystrophin pre-RNA.

In various aspects, an antisense oligomer conjugate is according to Formula (III):

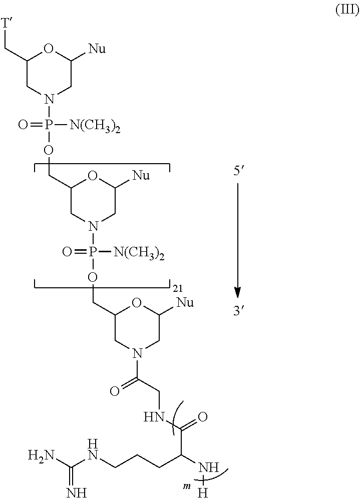

or a pharmaceutically acceptable salt thereof, wherein:
each Nu is a nucleobase which taken together form a targeting sequence;
T′ is a moiety selected from:

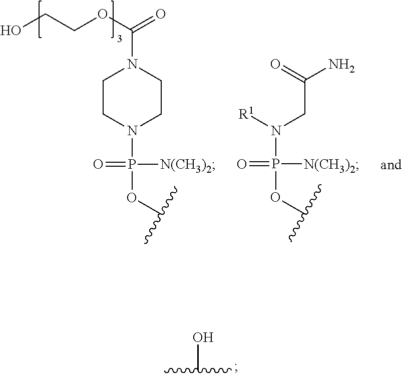

R¹ is C₁-C₆ alkyl; and
m is 0, 1, 2, 3, 4, or 5;
wherein the targeting sequence is complementary to an annealing site in the dystrophin pre-RNA.

In various aspects, an antisense oligomer conjugate is according to Formula (IV):

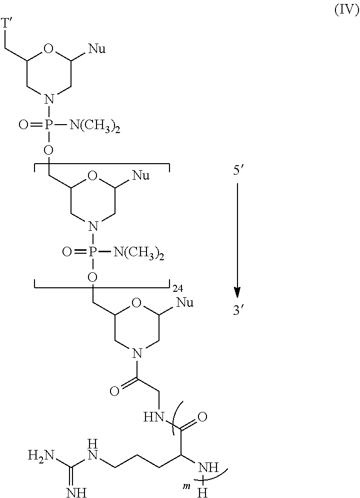

or a pharmaceutically acceptable salt thereof, wherein:
each Nu is a nucleobase which taken together form a targeting sequence;
T′ is a moiety selected from:

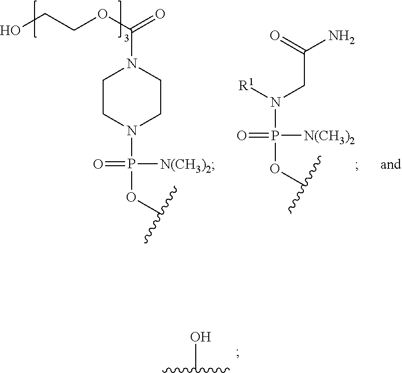

R¹ is C₁-C₆ alkyl; and
m is 0, 1, 2, 3, 4, or 5;
wherein the targeting sequence is complementary to an annealing site in the dystrophin pre-RNA.

In some aspects, the antisense oligonucleotide conjugate in the composition comprises a sequence that is complementary to 15 to 35 nucleobases of an exon 44, exon 45, exon 50, exon 51, exon 52, or exon 53 target region of the dystrophin pre-mRNA. Oligonucleotide sequences designed to target and skip these dystrophin exons have been described in the art. See for example, the following PCT published applications and issued US patents: WO2018/129384, WO2019/060775, WO2020/219820 WO2018/007475, WO2018/091544, WO2020/089325, WO2004/048570, WO2020/028832, WO2017/062862, U.S. Pat. Nos. 10,683,322, 8,969,551, 10,781,448, 9,988,629, 9,840,706, 10,851,373, WO2020/004675, and WO2020/0158792, the sequence disclosure of which is incorporated herein.

A number of exemplary targeting sequences are described below. These sequences can be provided as morpholino targeting sequences and incorporated into the antisense oligonucleotide conjugates of Formula (I).

In some aspects, the targeting sequence is complementary to an exon 51 annealing site in the dystrophin pre-mRNA. In some aspects, the site is designated as H51A(+66+95). In some aspects, the targeting sequence is complementary to an exon 45 annealing site in the dystrophin pre-mRNA. In some aspects, the site is designated as H45A(−03+19). In some aspects, the targeting sequence is complementary to an exon 53 annealing site in the dystrophin pre-mRNA. In some aspects, the site is designated as H53A(+36+60).

In various embodiments, T′ is

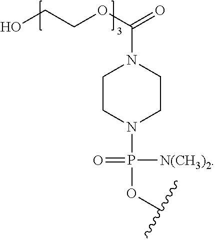

In various embodiments, R¹ is methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, or 2,3-dimethylbutyl.

In some embodiments, an antisense oligomer conjugate of Formula (I) is an HCl (hydrochloric acid) salt thereof. In certain embodiments, the HCl salt is a ·5HCl salt. In certain embodiments, the HCl salt is a ·4HCl salt. In certain embodiments, the HCl salt is a ·3HCl salt. In certain embodiments, the HCl salt is a ·2HCl salt. In certain embodiments, the HCl salt is a ·1HCl salt.

In some embodiments, each Nu is independently selected from cytosine (C), guanine (G), thymine (T), adenine (A), 5-methylcytosine (5mC), uracil (U), and hypoxanthine (I).

In some embodiments, the targeting sequence is 5′-CTCCAACATCAAGGAAGATGGCATTTCTAG-3′ (SEQ ID NO: 1), wherein each thymine (T) is optionally uracil (U).

In various embodiments, T′ is

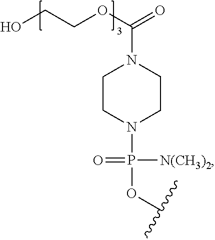

and the targeting sequence is 5′-CTCCAACATCAAGGAAGATGGCATTTCTAG-3′ (SEQ ID NO: 1), wherein each thymine (T) is optionally uracil (U).

In various embodiments, T′ is

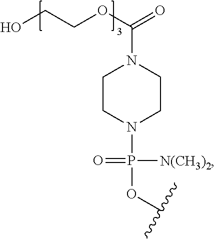

and the targeting sequence is 5′-CTCCAACATCAAGGAAGATGGCATTTCTAG-3′ (SEQ ID NO: 1).

In some embodiments, the targeting sequence is 5′-CAATGCCATCCTGGAGTTCCTG-3′ (SEQ ID NO: 2), wherein each thymine (T) is optionally uracil (U).

In various embodiments, T′ is

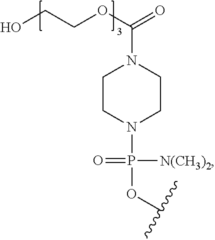

and the targeting sequence is 5′-CAATGCCATCCTGGAGTTCCTG-3′ (SEQ ID NO: 2), wherein each thymine (T) is optionally uracil (U).

In various embodiments, T′ is

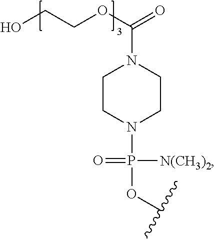

and the targeting sequence is 5′-CAATGCCATCCTGGAGTTCCTG-3′ (SEQ ID NO: 2).

In some embodiments, the targeting sequence is 5′-GTTGCCTCCGGTTCTGAAGGTGTTC-3′ (SEQ ID NO: 3), wherein each thymine (T) is optionally uracil (U).

In various embodiments, T′ is

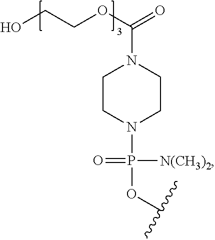

and the targeting sequence is 5′-GTTGCCTCCGGTTCTGAAGGTGTTC-3′ (SEQ ID NO: 3), wherein each thymine (T) is optionally uracil (U).

In various embodiments, T′ is

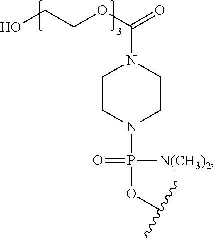

and the targeting sequence is 5′-GTTGCCTCCGGTTCTGAAGGTGTTC-3′ (SEQ ID NO: 3).

In some embodiments, including, for example, some embodiments of Formula (I) and Formula (II), an antisense oligomer conjugate of the disclosure is according to Formula (V):

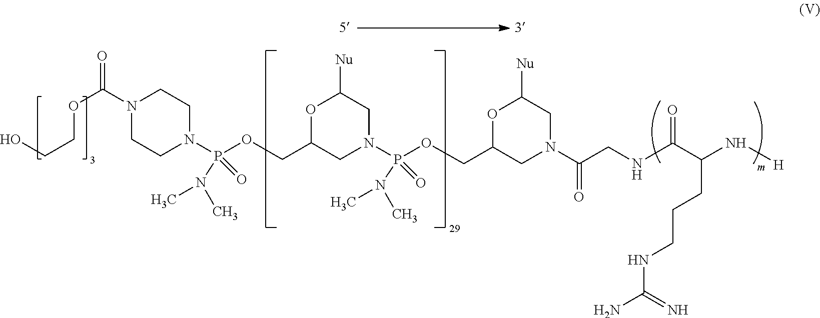

or a pharmaceutically acceptable salt thereof, wherein:
each Nu is a nucleobase which taken together to form a targeting sequence that is complementary to an exon 51 annealing site in the dystrophin pre-mRNA designated as H51A(+66+95); and
m is 0, 1, 2, 3, 4, or 5.

In some embodiments, including, for example, some embodiments of Formula (V), an antisense oligomer conjugate of the disclosure is according to Formula (VA):

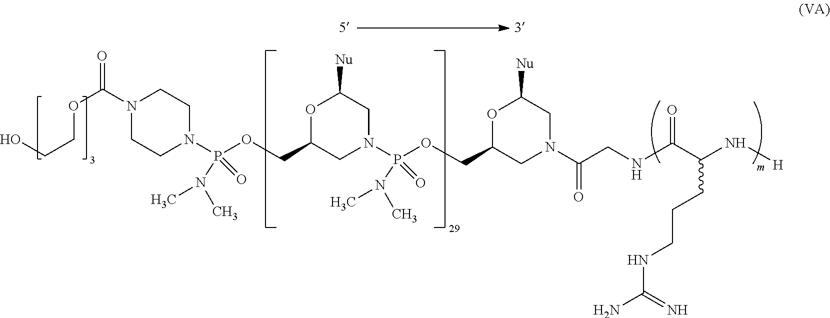

or a pharmaceutically acceptable salt thereof, wherein:
each Nu is a nucleobase which taken together to form a targeting sequence that is complementary to an exon 51 annealing site in the dystrophin pre-mRNA designated as H51A(+66+95); and
m is 0, 1, 2, 3, 4, or 5.

In some embodiments, each Nu in Formula (V) or Formula (VA) is independently selected from cytosine (C), guanine (G), thymine (T), adenine (A), 5-methylcytosine (5mC), uracil (U), and hypoxanthine (I).

In various embodiments, each Nu from 1 to 30 and 5′ to 3′ is:

| Position | |
|---|---|
| No. 5′ | |
| to 3′ | Nu |
| 1 | C |
| 2 | X |
| 3 | C |
| 4 | C |
| 5 | A |
| 6 | A |
| 7 | C |
| 8 | A |
| 9 | X |
| 10 | C |
| 11 | A |
| 12 | A |
| 13 | G |
| 14 | G |
| 15 | A |
| 16 | A |
| 17 | G |
| 18 | A |
| 19 | X |
| 20 | G |
| 21 | G |
| 22 | C |
| 23 | A |
| 24 | X |
| 25 | X |
| 26 | X |
| 27 | C |
| 28 | X |
| 29 | A |
| 30 | G |

wherein A is

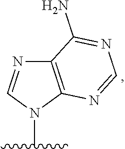 [SMILES: *n1cnc2c(N)ncnc21]

### C is

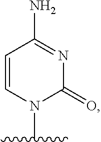 [SMILES: *n1ccc(N)nc1=O]

### G is

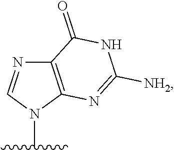 [SMILES: *n1cnc2c(=O)[nH]c(N)nc21]

### and X is

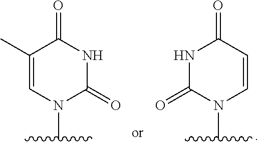

In certain embodiments, each X is independently

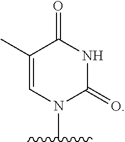 [SMILES: *n1cc(C)c(=O)[nH]c1=O]

In some embodiments, an antisense oligomer conjugate of Formula (V) or Formula (VA) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, some embodiments of Formula (VA), an antisense oligomer conjugate of the disclosure is according to Formula (VB) or Formula (VC):

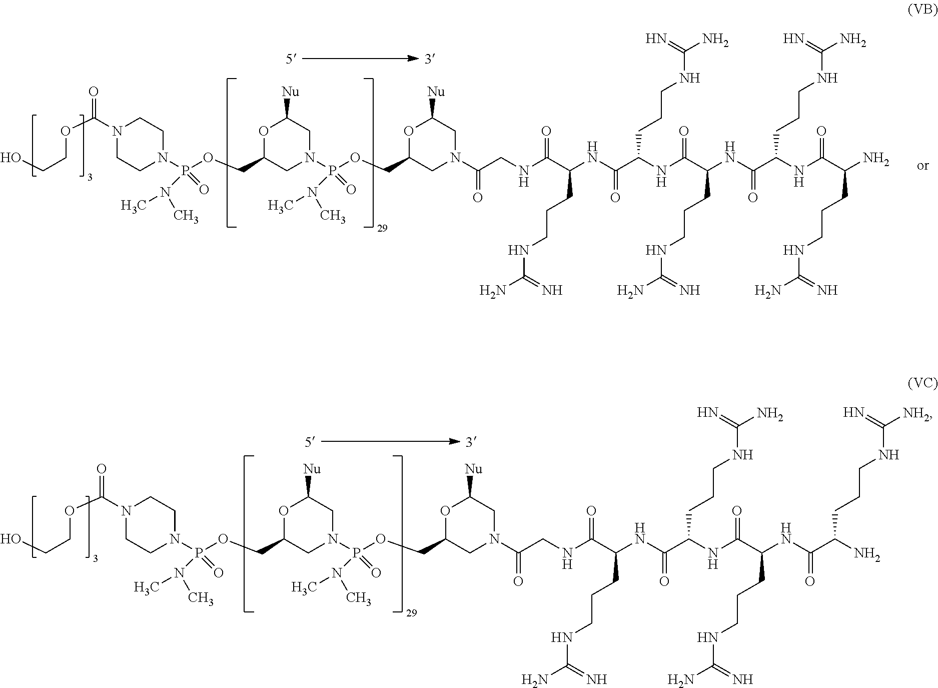

or a pharmaceutically acceptable salt thereof, such as a HCl salt, wherein each Nu is a nucleobase which taken together to form a targeting sequence that is complementary to an exon 51 annealing site in the dystrophin pre-mRNA designated as H51A(+66+95).

In some embodiments, including, for example, some embodiments of Formula (VA), an antisense oligomer conjugate of the disclosure is according to Formula (VD) or Formula (VE):

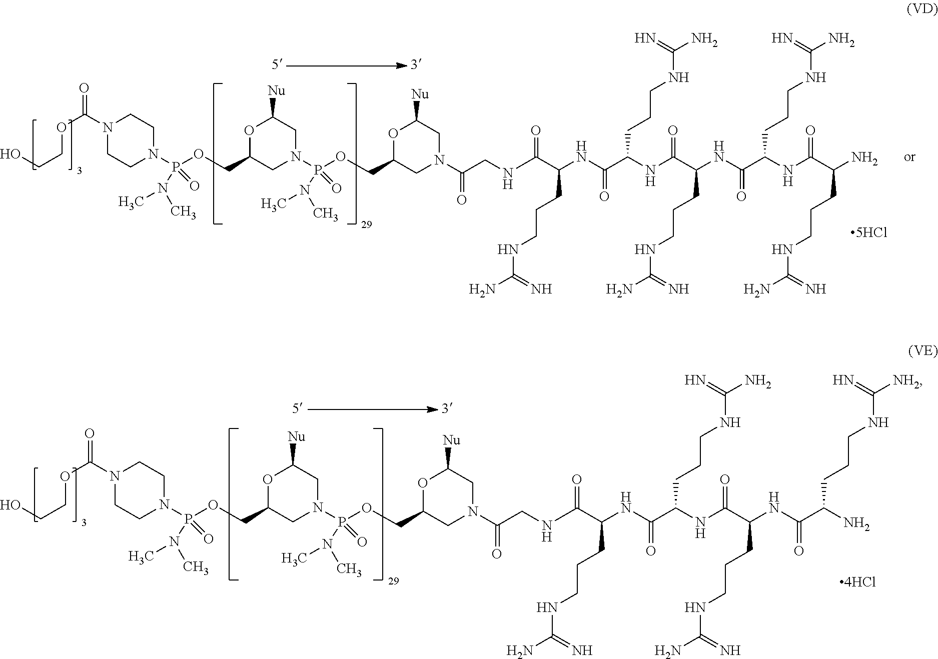

wherein each Nu is a nucleobase which taken together to form a targeting sequence that is complementary to an exon 51 annealing site in the dystrophin pre-mRNA designated as H51A(+66+95).

In some embodiments, each Nu in any of Formulae (VB), (VC), (VD), and (VE) is independently selected from cytosine (C), guanine (G), thymine (T), adenine (A), 5-methylcytosine (5mC), uracil (U), and hypoxanthine (I).

In various embodiments, each Nu from 1 to 30 and 5′ to 3′ is:

| Position | |
|---|---|
| No. 5′ | |
| to 3′ | Nu |
| 1 | C |
| 2 | X |
| 3 | C |
| 4 | C |
| 5 | A |
| 6 | A |
| 7 | C |
| 8 | A |
| 9 | X |
| 10 | C |
| 11 | A |
| 12 | A |
| 13 | G |
| 14 | G |
| 15 | A |
| 16 | A |
| 17 | G |
| 18 | A |
| 19 | X |
| 20 | G |
| 21 | G |
| 22 | C |
| 23 | A |
| 24 | X |
| 25 | X |
| 26 | X |
| 27 | C |
| 28 | X |
| 29 | A |
| 30 | G |

wherein A is

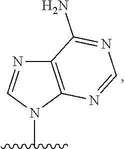 [SMILES: *n1cnc2c(N)ncnc21]

### C is

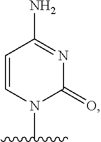 [SMILES: *n1ccc(N)nc1=O]

### G is

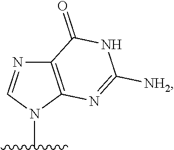 [SMILES: *n1cnc2c(=O)[nH]c(N)nc21]

### and X is

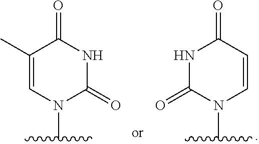

In certain embodiments, each X is

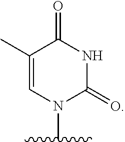 [SMILES: *n1cc(C)c(=O)[nH]c1=O]

In some embodiments including, for example, embodiments of antisense oligomer conjugates of Formula (V), Formula (VA), Formula (VB), Formula (VC), Formula (VD), and Formula (VE), the targeting sequence is 5′-CTCCAACATCAAGGAAGATGGCATTTCTAG-3′ (SEQ ID NO: 1) wherein each thymine (T) is optionally uracil (U). In various embodiments including, for example, embodiments of antisense oligomer conjugates of Formula (V), Formula (VA), Formula (VB), Formula (VC), Formula (VD), and Formula (VE), the targeting sequence is 5′-CTCCAACATCAAGGAAGATGGCATTTCTAG-3′ (SEQ ID NO: 1).

In some embodiments, including, for example, embodiments of antisense oligomer conjugates of Formula (I) or Formula (II), an antisense oligomer conjugate of the disclosure is according to Formula (V):

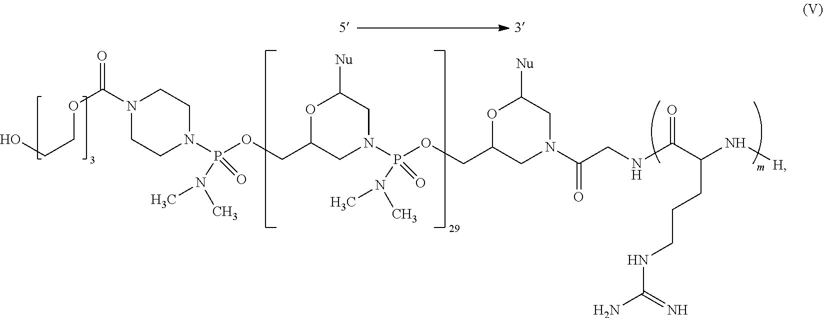

or a pharmaceutically acceptable salt thereof, wherein m is 0, 1, 2, 3, 4, or 5, and
each Nu from 1 to 30 and 5′ to 3′ is:

| Position | |
|---|---|
| No. 5′ | |
| to 3′ | Nu |
| 1 | C |
| 2 | T |
| 3 | C |
| 4 | C |
| 5 | A |
| 6 | A |
| 7 | C |
| 8 | A |
| 9 | T |
| 10 | C |
| 11 | A |
| 12 | A |
| 13 | G |
| 14 | G |
| 15 | A |
| 16 | A |
| 17 | G |
| 18 | A |
| 19 | T |
| 20 | G |
| 21 | G |
| 22 | C |
| 23 | A |
| 24 | T |
| 25 | T |
| 26 | T |
| 27 | C |
| 28 | T |
| 29 | A |
| 30 | G |

wherein A is

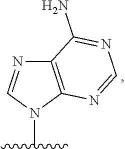 [SMILES: *n1cnc2c(N)ncnc21]

### C is

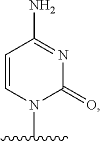 [SMILES: *n1ccc(N)nc1=O]

### G is

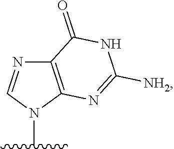 [SMILES: *n1cnc2c(=O)[nH]c(N)nc21]

### and T is

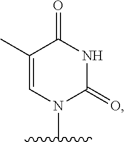 [SMILES: *n1cc(C)c(=O)[nH]c1=O]

represented by Formula (VI):

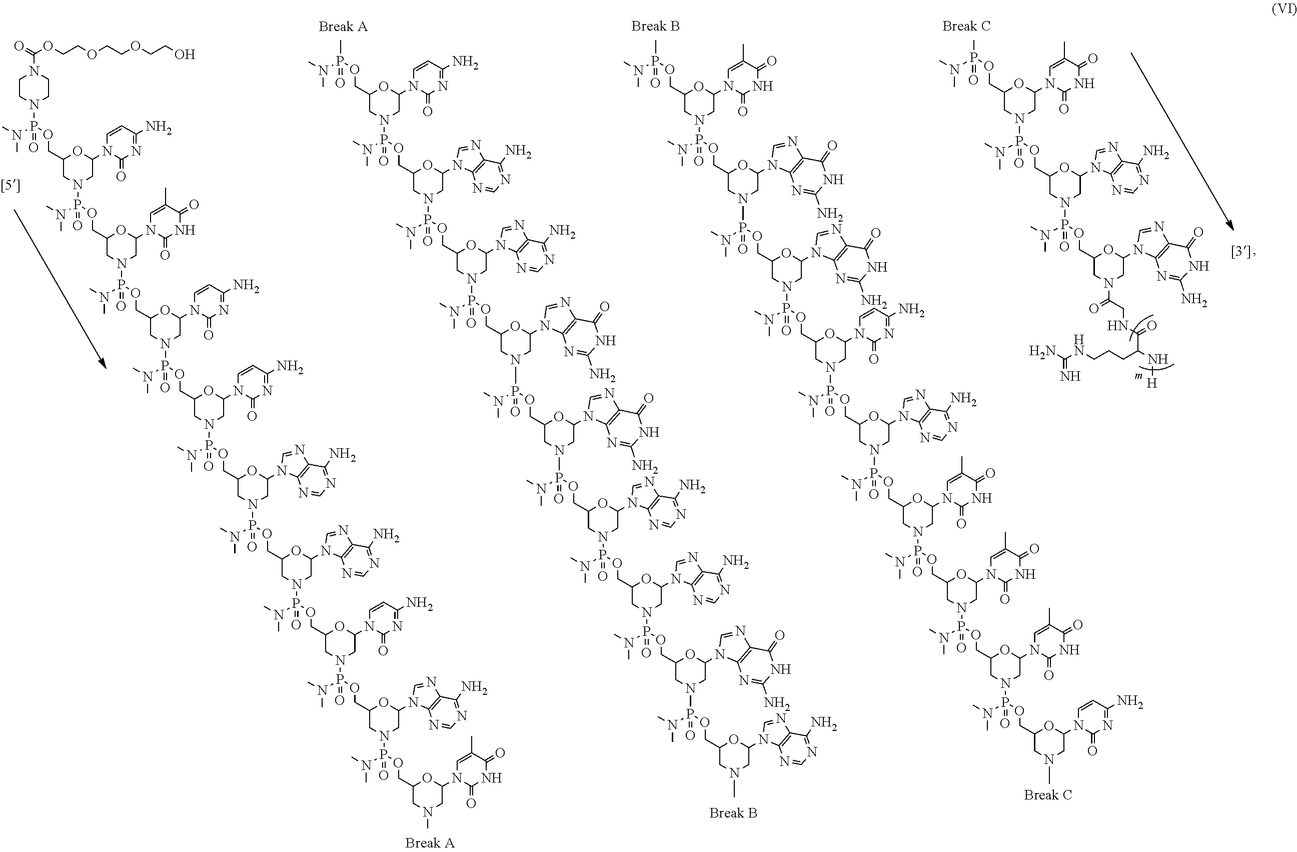

or a pharmaceutically acceptable salt thereof, wherein m is 0, 1, 2, 3, 4, or 5.

In some embodiments, an antisense oligomer conjugate of Formula (VI) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (VI), an antisense oligomer conjugate of the disclosure is according to Formula (VIA) or Formula (VIB):

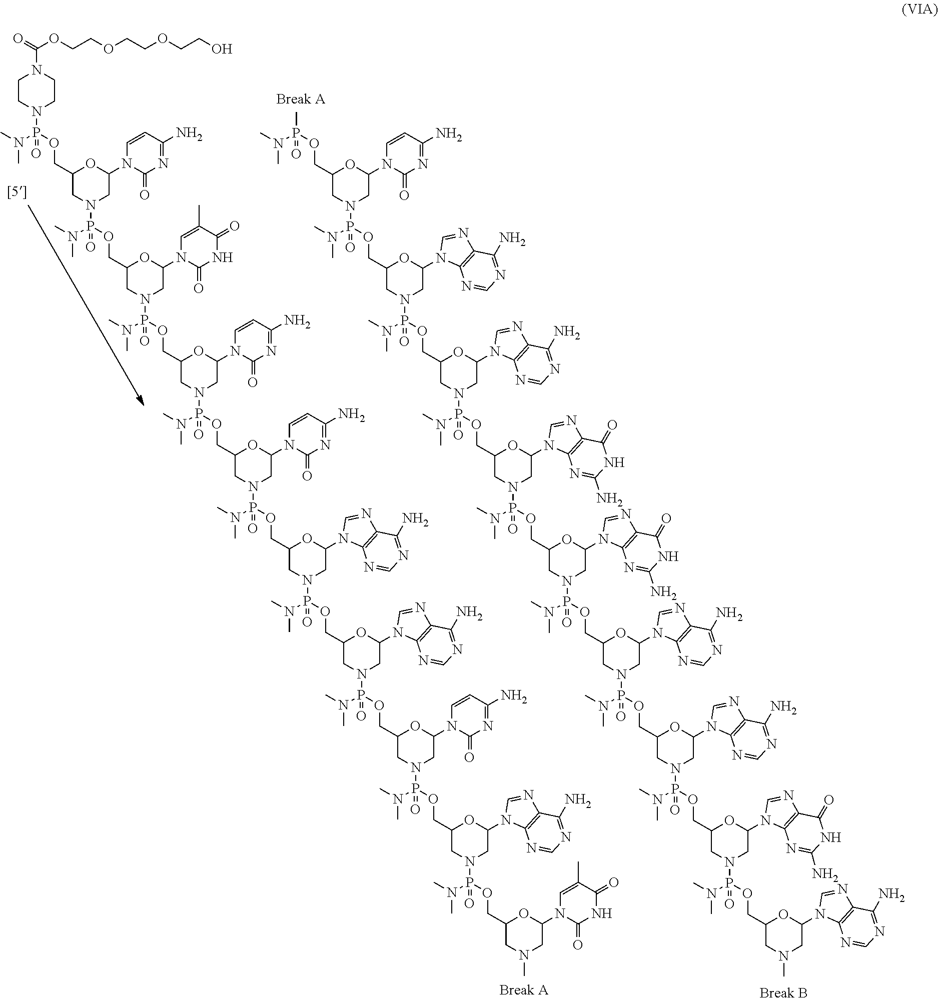

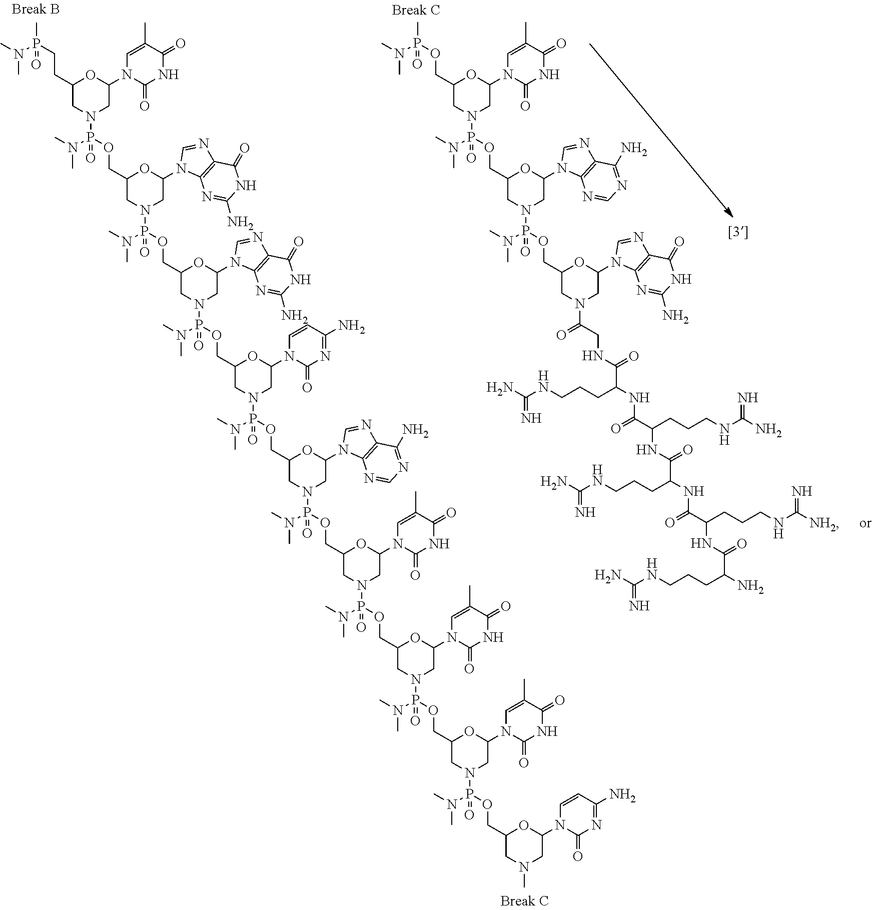

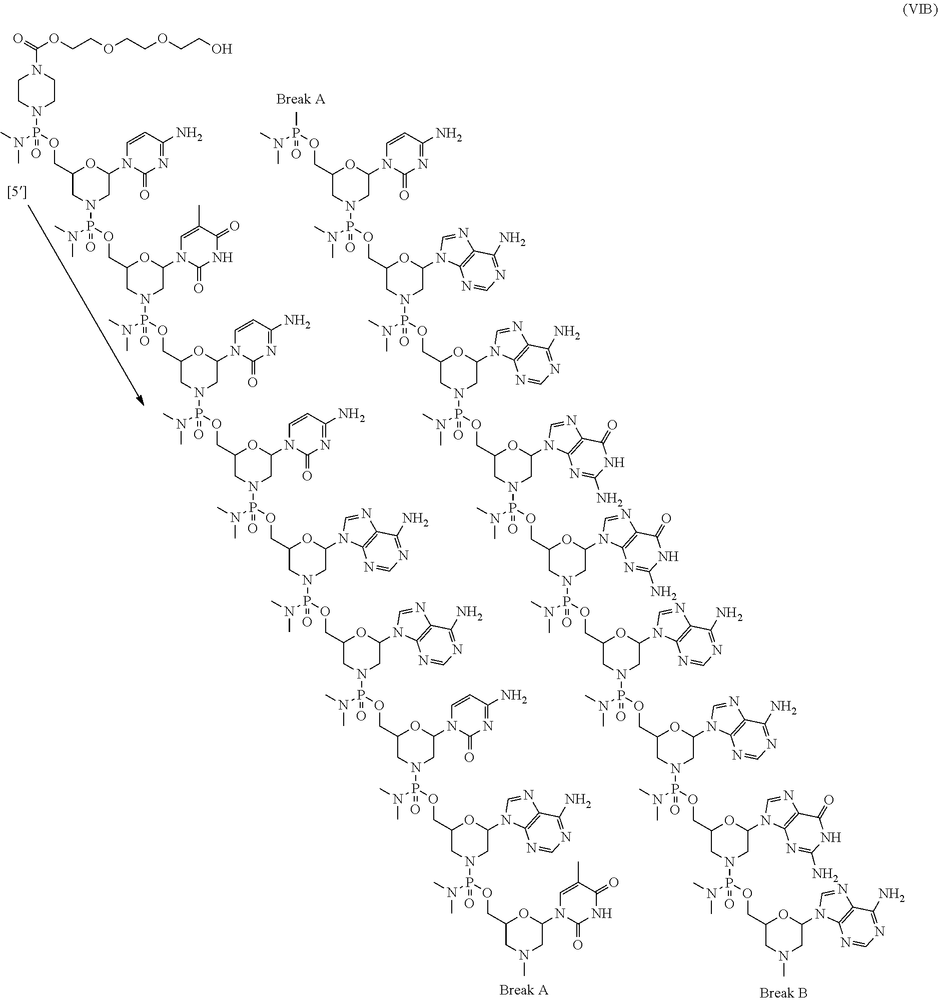

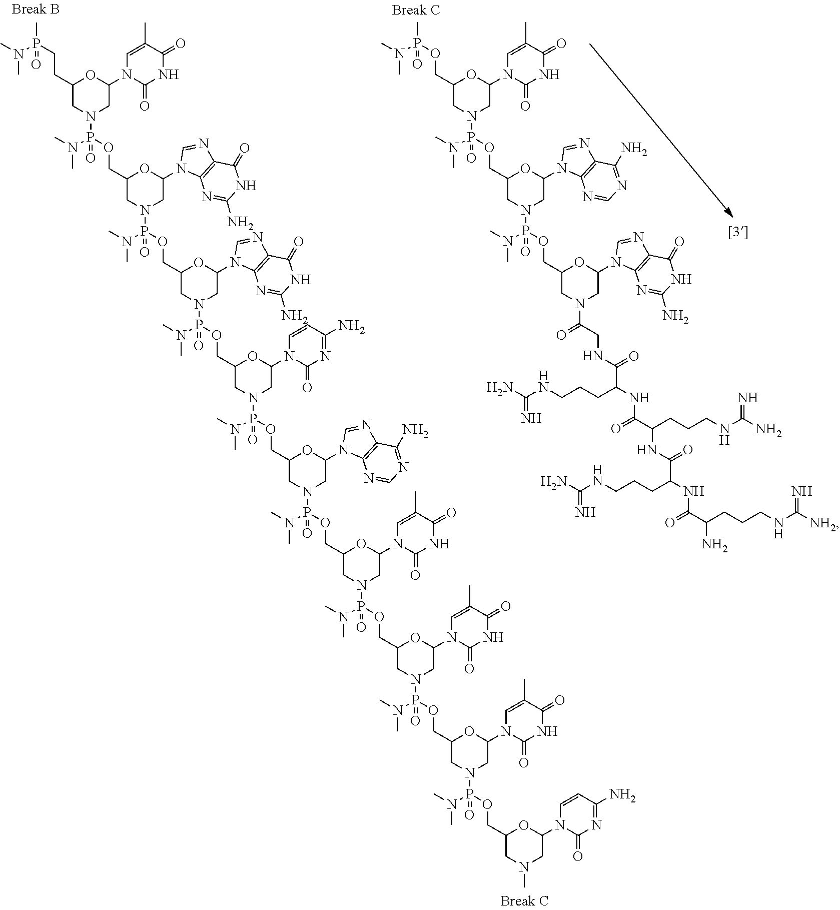

or a pharmaceutically acceptable salt thereof, such as a HCl salt.

In some embodiments of the disclosure, including some embodiments of antisense oligomer conjugates of Formula (I) and Formula (II), the antisense oligomer conjugate is according to Formula (VA):

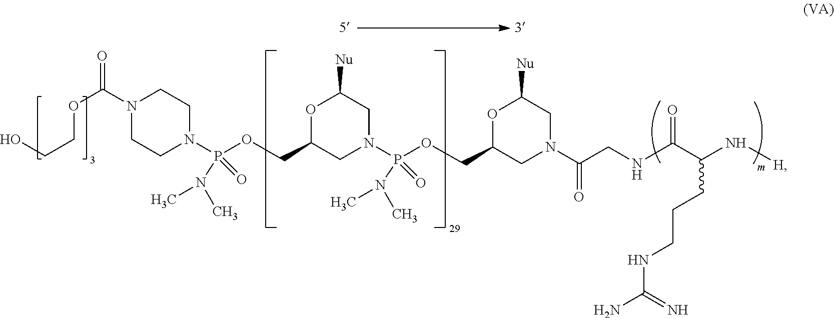

or a pharmaceutically acceptable salt thereof, wherein m is 0, 1, 2, 3, 4, or 5, and
each Nu from 1 to 30 and 5′ to 3′ is:

| Position | |
|---|---|
| No. 5′ | |
| to 3′ | Nu |
| 1 | C |
| 2 | T |
| 3 | C |
| 4 | C |
| 5 | A |
| 6 | A |
| 7 | C |
| 8 | A |
| 9 | T |
| 10 | C |
| 11 | A |
| 12 | A |
| 13 | G |
| 14 | G |
| 15 | A |
| 16 | A |
| 17 | G |
| 18 | A |
| 19 | T |
| 20 | G |
| 21 | G |
| 22 | C |
| 23 | A |
| 24 | T |
| 25 | T |
| 26 | T |
| 27 | C |
| 28 | T |
| 29 | A |
| 30 | G |

wherein A is

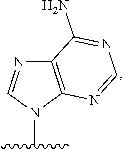 [SMILES: *n1cnc2c(N)ncnc21]

### C is

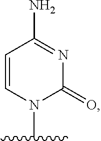 [SMILES: *n1ccc(N)nc1=O]

### G is

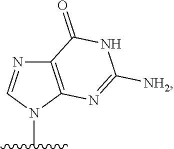 [SMILES: *n1cnc2c(=O)[nH]c(N)nc21]

### and T is

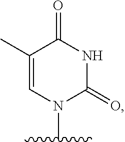 [SMILES: *n1cc(C)c(=O)[nH]c1=O]

represented by Formula (VIC):

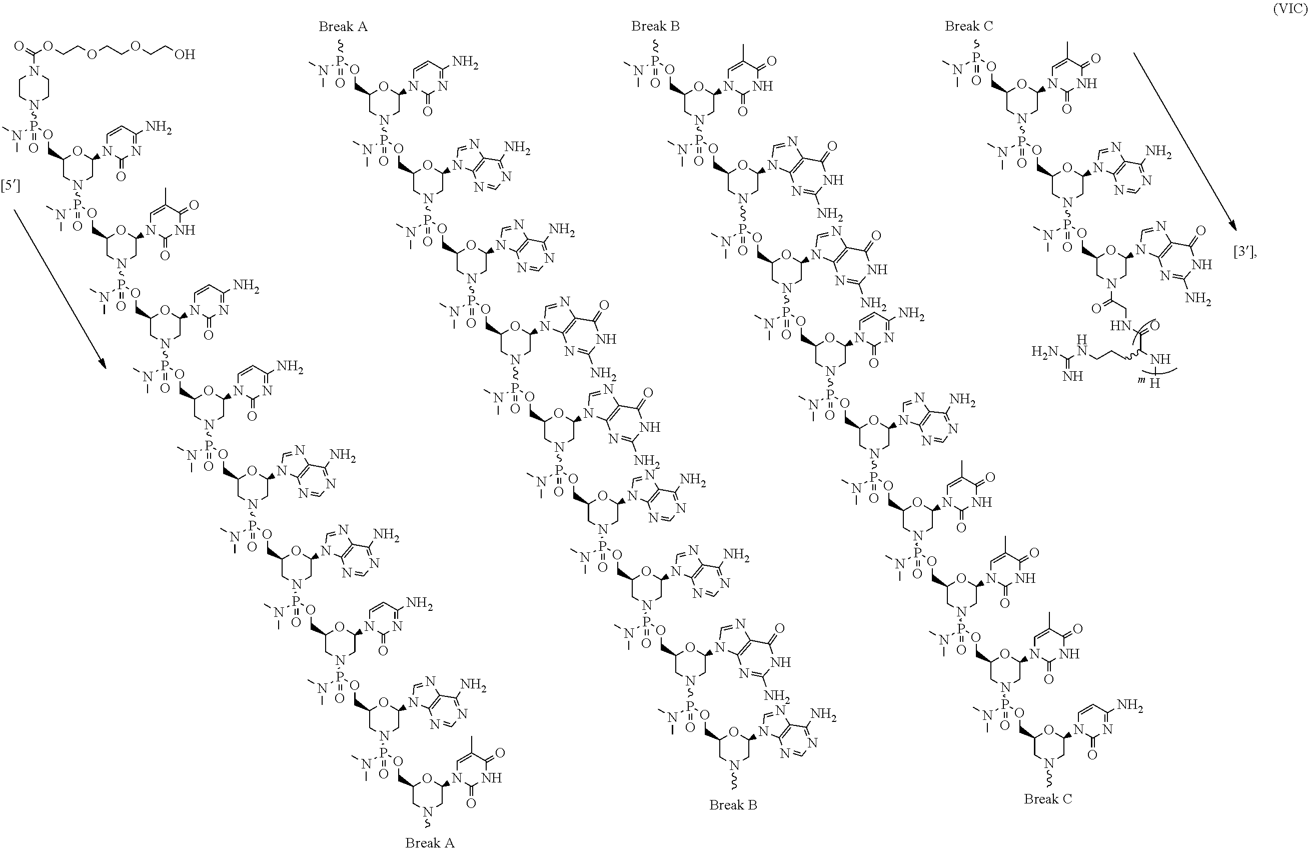

or a pharmaceutically acceptable salt thereof, wherein m is 0, 1, 2, 3, 4, or 5.

In some embodiments, an antisense oligomer conjugate of Formula (VIC) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (VIC), an antisense oligomer conjugate of the disclosure is according to Formula (VID) or Formula (VIE):

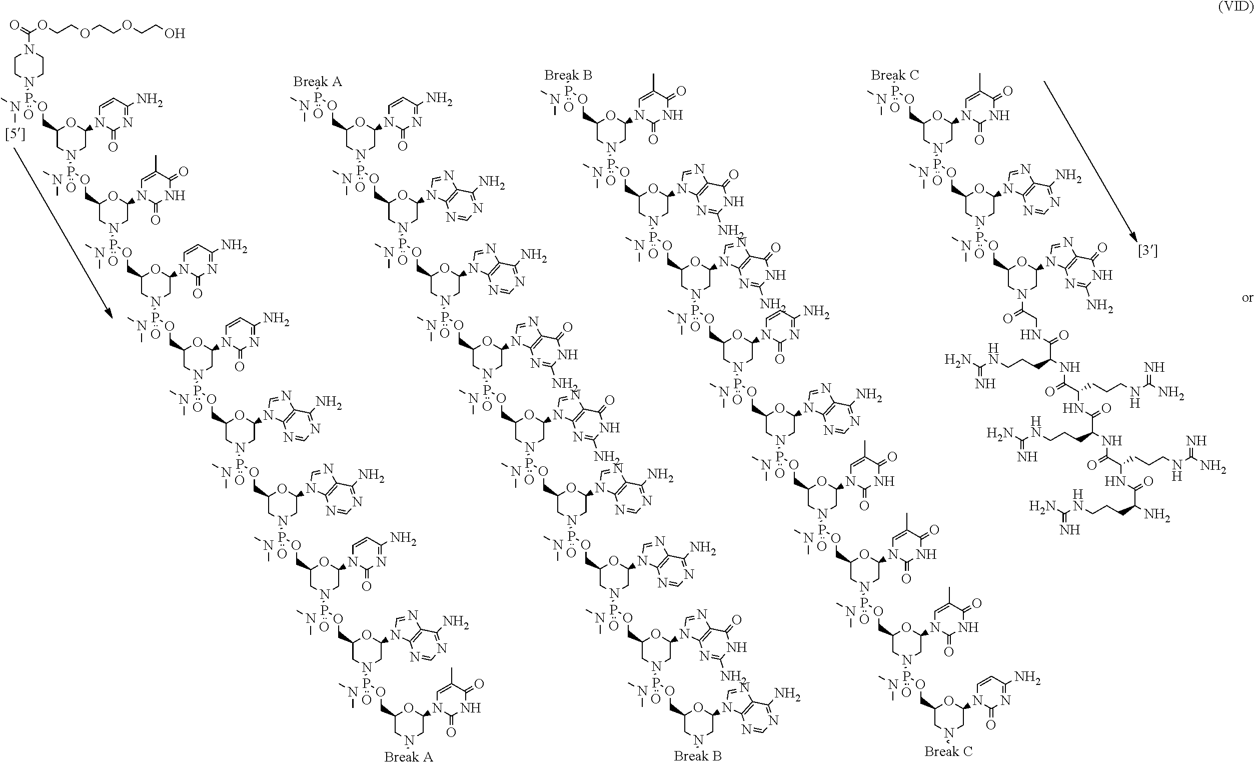

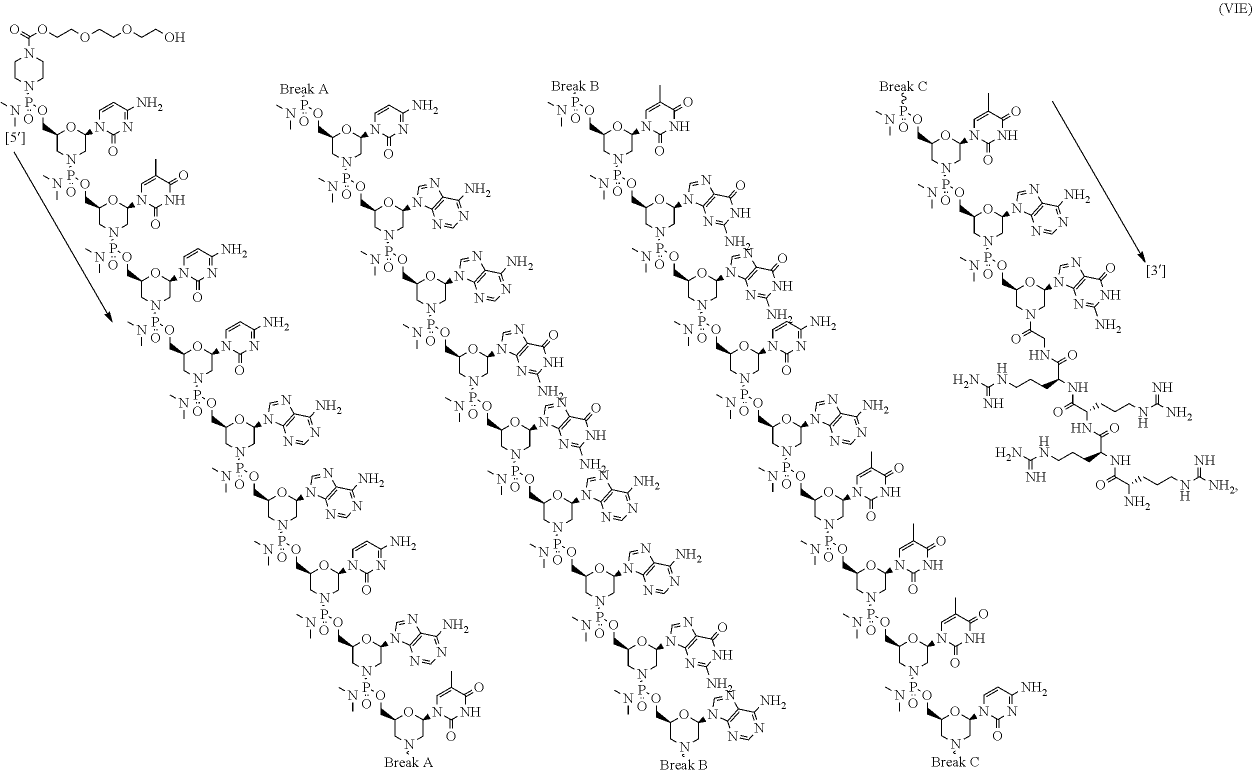

or a pharmaceutically acceptable salt thereof, such as a HCl salt.

In some embodiments, including, for example, embodiments of antisense oligomer conjugates of Formula (VIC), an antisense oligomer conjugate of the disclosure is according to Formula (VIF) or Formula (VIG):

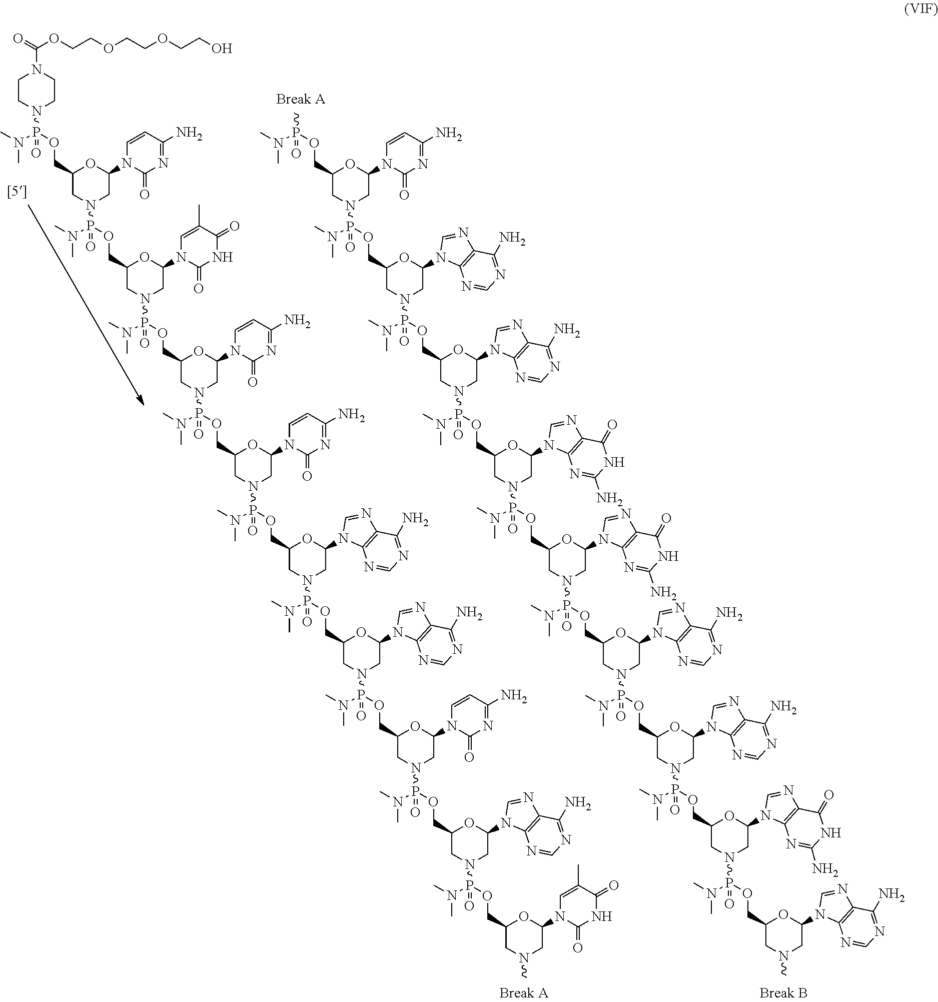

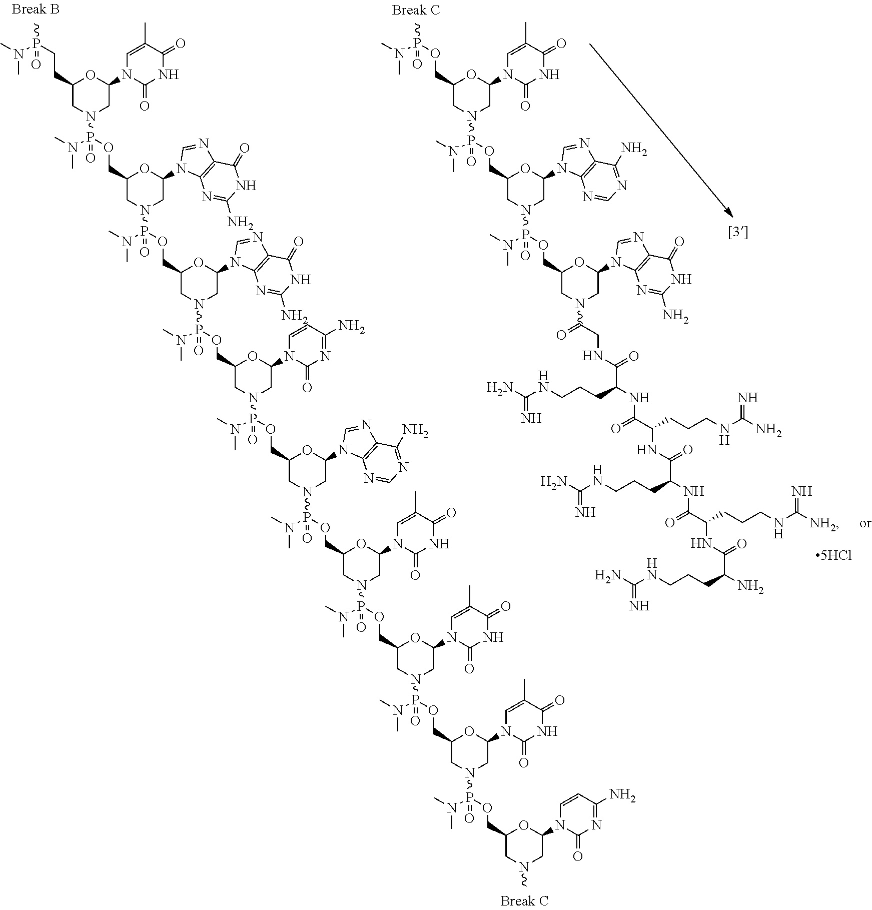

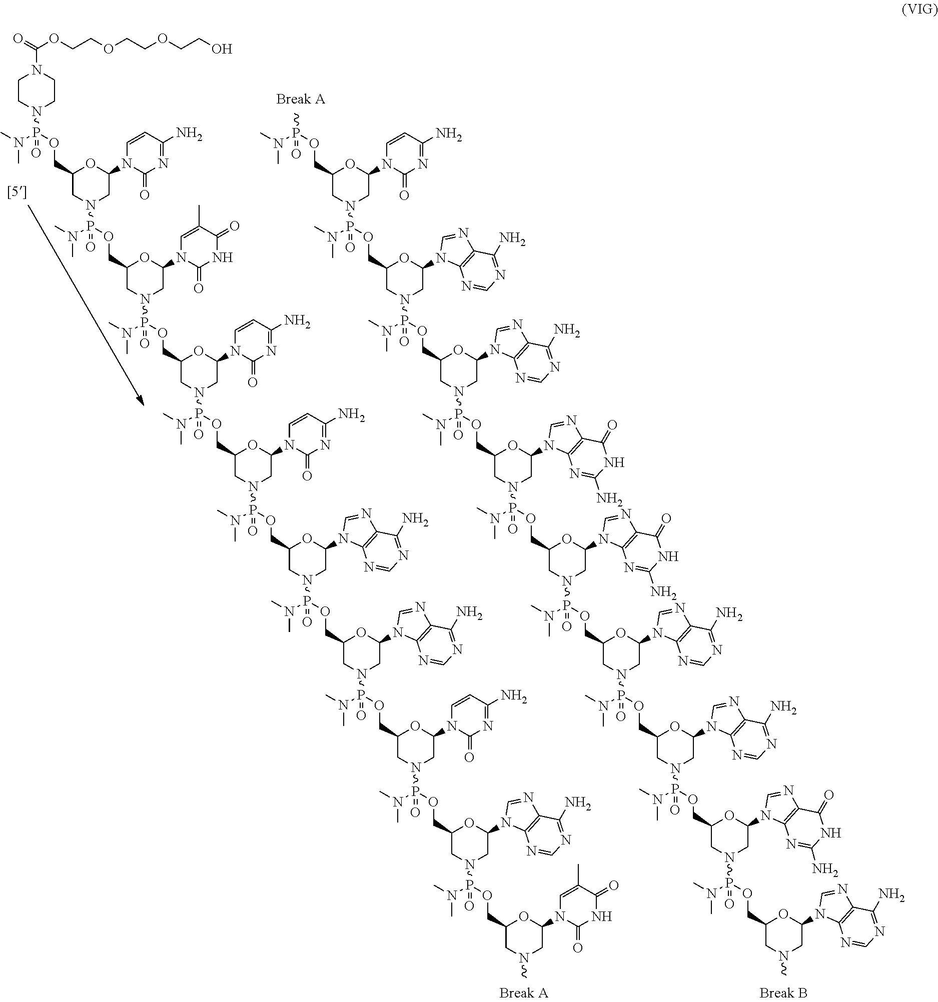

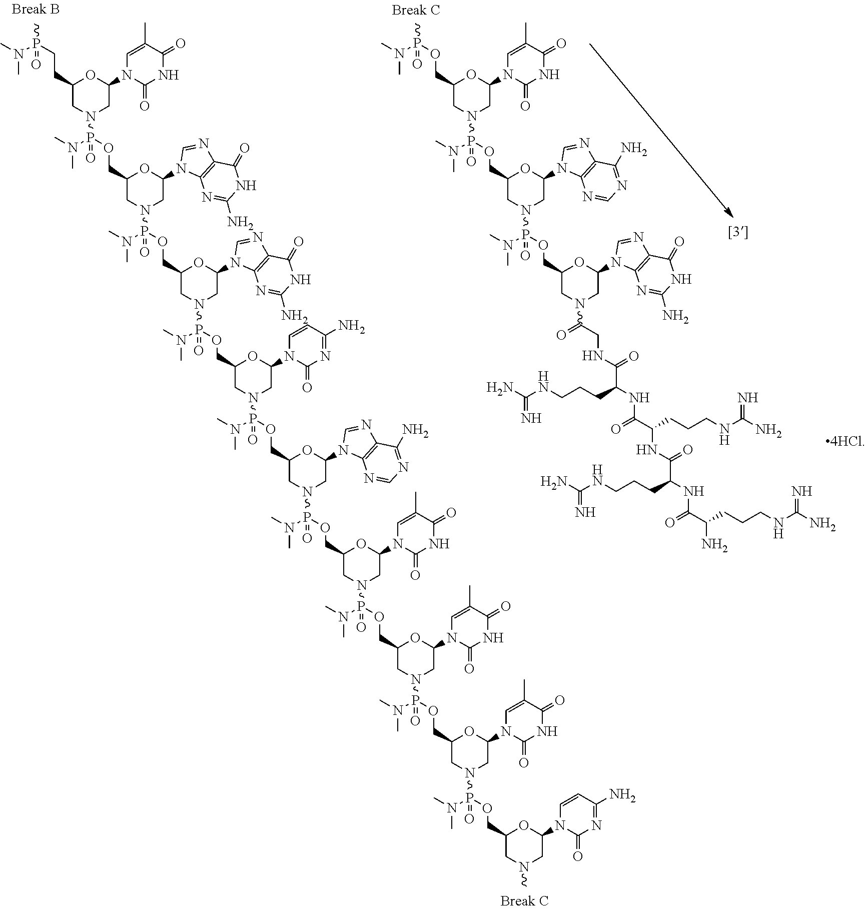

In some embodiments, including, for example, some embodiments of Formula (I) and Formula (III), an antisense oligomer conjugate of the disclosure is according to Formula (VII):

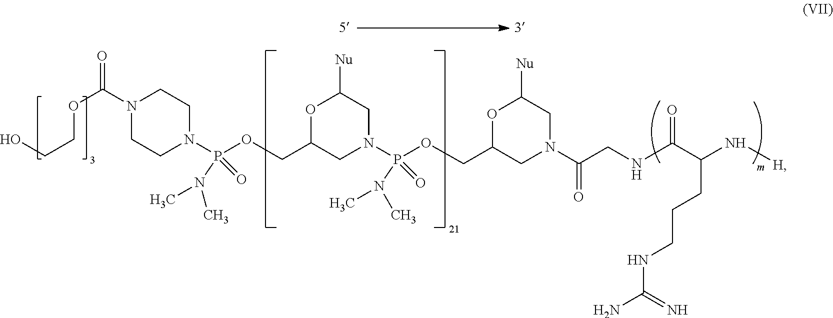

or a pharmaceutically acceptable salt thereof, wherein:
each Nu is a nucleobase which taken together form a targeting sequence that is complementary to an exon 45 annealing site in the dystrophin pre-mRNA designated as H45A(−03+19), and in is 0, 1, 2, 3, 4, or 5.

In some embodiments, including, for example, some embodiments of Formula (VII), an antisense oligomer conjugate of the disclosure is according to Formula (VIIA):

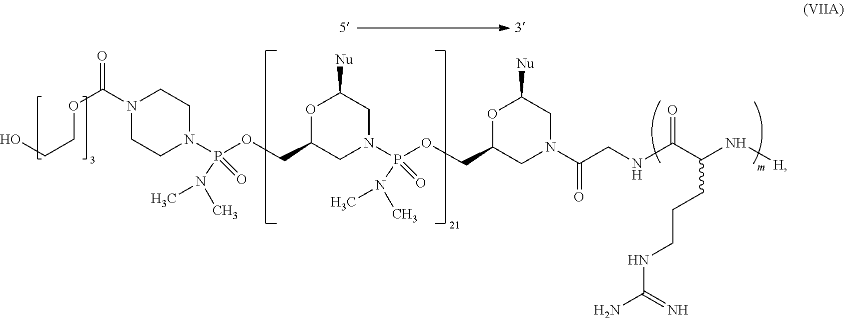

or a pharmaceutically acceptable salt thereof, wherein:
each Nu is a nucleobase which taken together form a targeting sequence that is complementary to an exon 45 annealing site in the dystrophin pre-mRNA designated as H45A(−03+19), and m is 0, 1, 2, 3, 4, or 5.

In some embodiments, each Nu in Formula (VII) or Formula (VIIA) is independently selected from cytosine (C), guanine (G), thymine (T), adenine (A), 5-methylcytosine (5mC), uracil (U), and hypoxanthine (I).

In various embodiments, each Nu from 1 to 22 and 5′ to 3′ is:

| Position | |
|---|---|
| No. 5′ | |
| to 3′ | Nu |
| 1 | C |
| 2 | A |
| 3 | A |
| 4 | X |
| 5 | G |
| 6 | C |
| 7 | C |
| 8 | A |
| 9 | X |
| 10 | C |
| 11 | C |
| 12 | X |
| 13 | G |
| 14 | G |
| 15 | A |
| 16 | G |
| 17 | X |
| 18 | X |
| 19 | C |
| 20 | C |
| 21 | X |
| 22 | G |

wherein A is

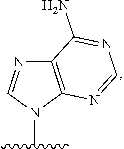 [SMILES: *n1cnc2c(N)ncnc21]

### C is

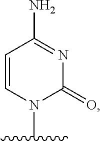 [SMILES: *n1ccc(N)nc1=O]

### G is

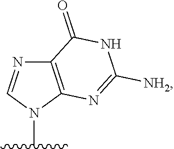 [SMILES: *n1cnc2c(=O)[nH]c(N)nc21]

### and X is

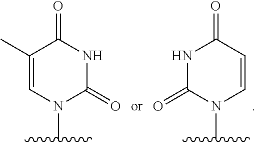

In certain embodiments, each X is independently

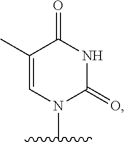 [SMILES: *n1cc(C)c(=O)[nH]c1=O]

In some embodiments, an antisense oligomer conjugate of Formula (VII) or Formula (VIIA) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, some embodiments of Formula (VIIA), an antisense oligomer conjugate of the disclosure is according to Formula (VIIB) or Formula (VIIC):

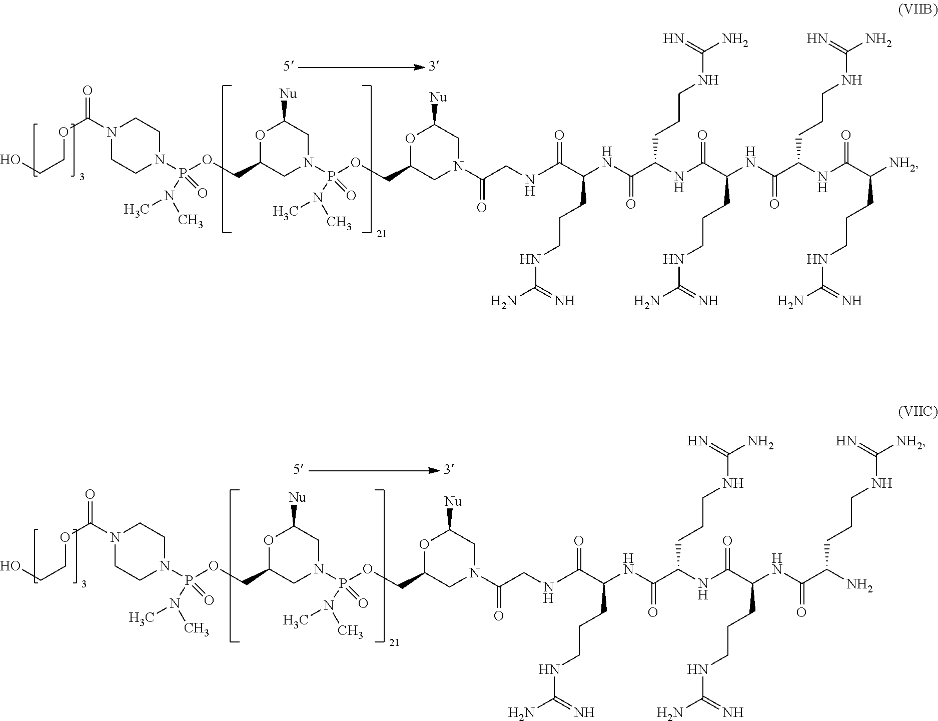

or a pharmaceutically acceptable salt thereof, such as a HCl-salt, wherein each Nu is a nucleobase which taken together to form a targeting sequence that is complementary to an exon 45 annealing site in the dystrophin pre-mRNA designated as H45A(−03+19).

In some embodiments, each Nu in Formulae (VIIB) or Formula (VIIC) is independently selected from cytosine (C), guanine (G), thymine (T), adenine (A), 5-methylcytosine (5mC), uracil (U), and hypoxanthine (I).

In various embodiments, each Nu from 1 to 22 and 5′ to 3′ is:

| Position | |
|---|---|
| No. 5′ | |
| to 3′ | Nu |
| 1 | C |
| 2 | A |
| 3 | A |
| 4 | X |
| 5 | G |
| 6 | C |
| 7 | C |
| 8 | A |
| 9 | X |
| 10 | C |
| 11 | C |
| 12 | X |
| 13 | G |
| 14 | G |
| 15 | A |
| 16 | G |
| 17 | X |
| 18 | X |
| 19 | C |
| 20 | C |
| 21 | X |
| 22 | G |

wherein A is

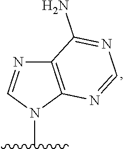 [SMILES: *n1cnc2c(N)ncnc21]

### C is

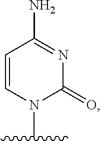 [SMILES: *n1ccc(N)nc1=O]

### G is

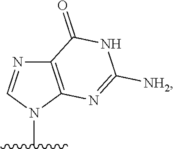 [SMILES: *n1cnc2c(=O)[nH]c(N)nc21]

### and X is

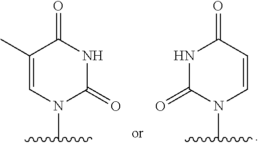

In certain embodiments, each X is

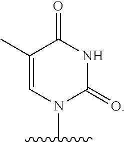 [SMILES: *n1cc(C)c(=O)[nH]c1=O]

In some embodiments including, for example, embodiments of antisense oligomer conjugates of Formula (VII), Formula (VIIA), Formula (VIIB), and Formula (VIIC), the targeting sequence is 5′-CAATGCCATCCTGGAGTTCCTG-3′ (SEQ ID NO: 2) wherein each thymine (T) is optionally uracil (U). In various embodiments including, for example, embodiments of antisense oligomer conjugates of Formula (VII), Formula (VIIA), Formula (VIIB), and Formula (VIIC), the targeting sequence is 5′-CAATGCCATCCTGGAGTTCCTG-3′ (SEQ ID NO: 2).

In some embodiments, including, for example, embodiments of antisense oligomer conjugates of Formula (I) or Formula (III), an antisense oligomer conjugate of the disclosure is according to Formula (VII):

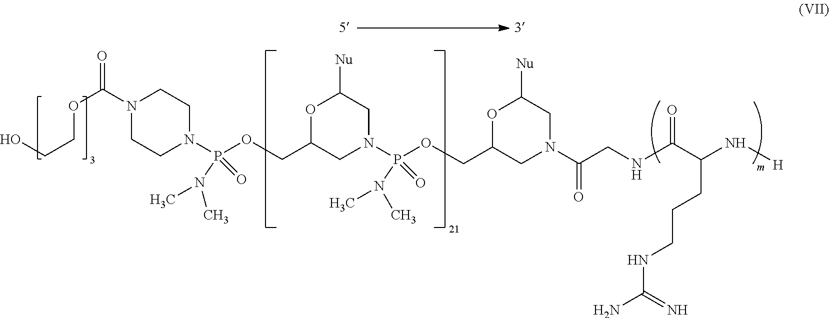

or a pharmaceutically acceptable salt thereof, wherein m is 0, 1, 2, 3, 4, or 5, and
each Nu from 1 to 22 and 5′ to 3′ is:

| Position | |
|---|---|
| No. 5′ | |
| to 3′ | Nu |
| 1 | C |
| 2 | A |
| 3 | A |
| 4 | T |
| 5 | G |
| 6 | C |
| 7 | C |
| 8 | A |
| 9 | T |
| 10 | C |
| 11 | C |
| 12 | T |
| 13 | G |
| 14 | G |
| 15 | A |
| 16 | G |
| 17 | T |
| 18 | T |
| 19 | C |
| 20 | C |
| 21 | T |
| 22 | G |

wherein A is

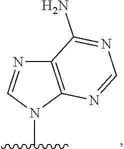 [SMILES: *n1cnc2c(N)ncnc21]

### C is

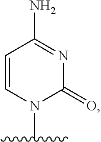 [SMILES: *n1ccc(N)nc1=O]

### G is

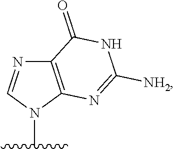 [SMILES: *n1cnc2c(=O)[nH]c(N)nc21]

### and T is

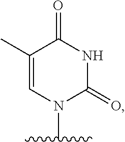 [SMILES: *n1cc(C)c(=O)[nH]c1=O]

represented by Formula (VIII):

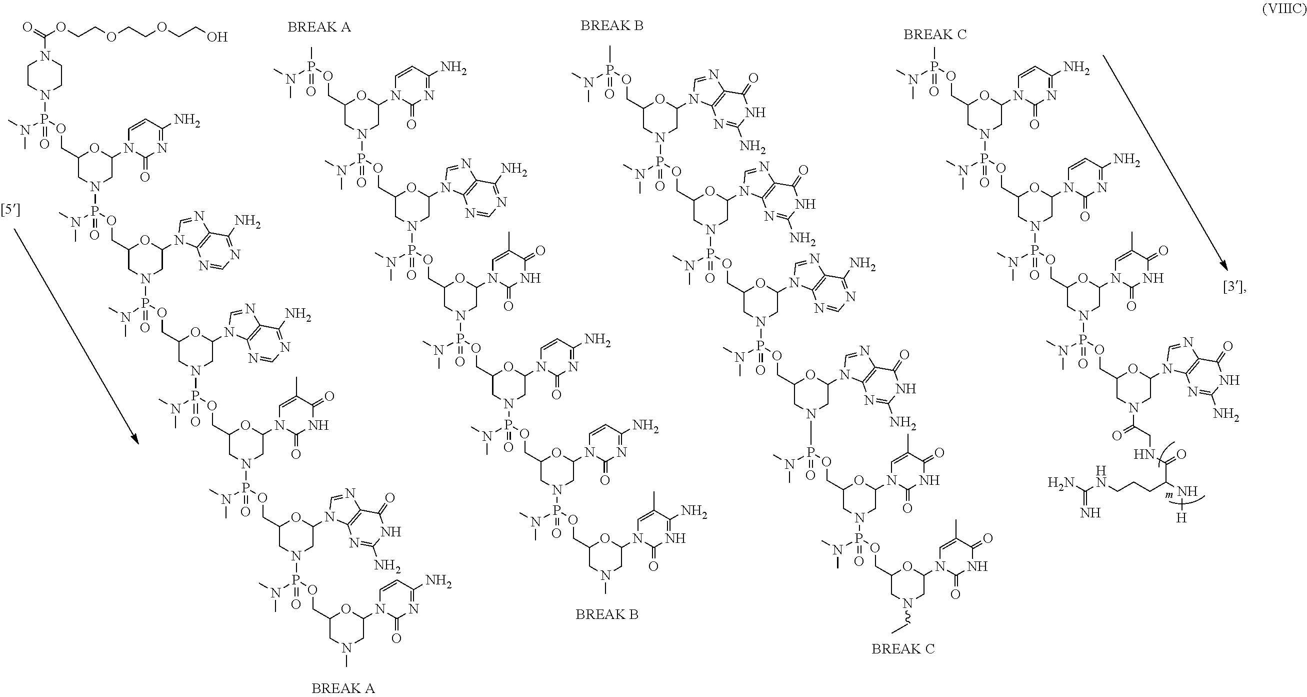

or a pharmaceutically acceptable salt thereof, wherein m is 0, 1, 2, 3, 4, or 5.

In some embodiments, an antisense oligomer conjugate of Formula (VIII) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (VIII), an antisense oligomer conjugate of the disclosure is according to Formula (VIIIA) or Formula (VIIIB):

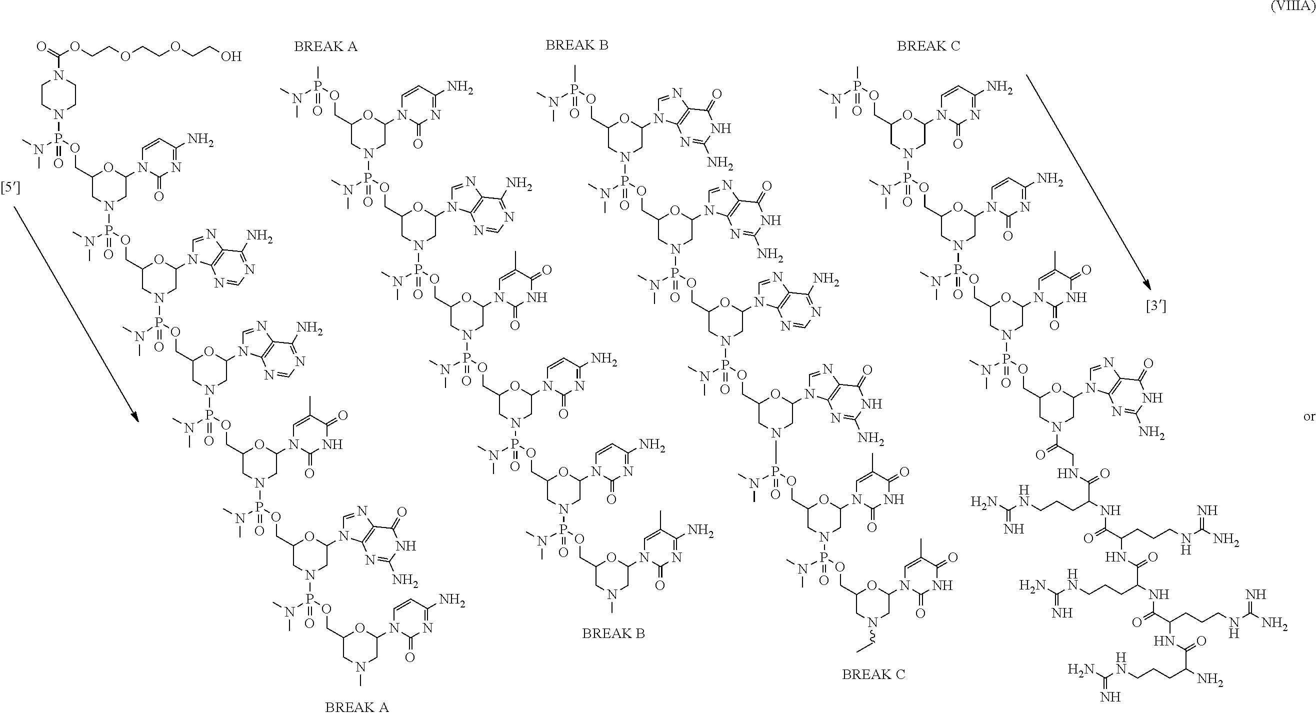

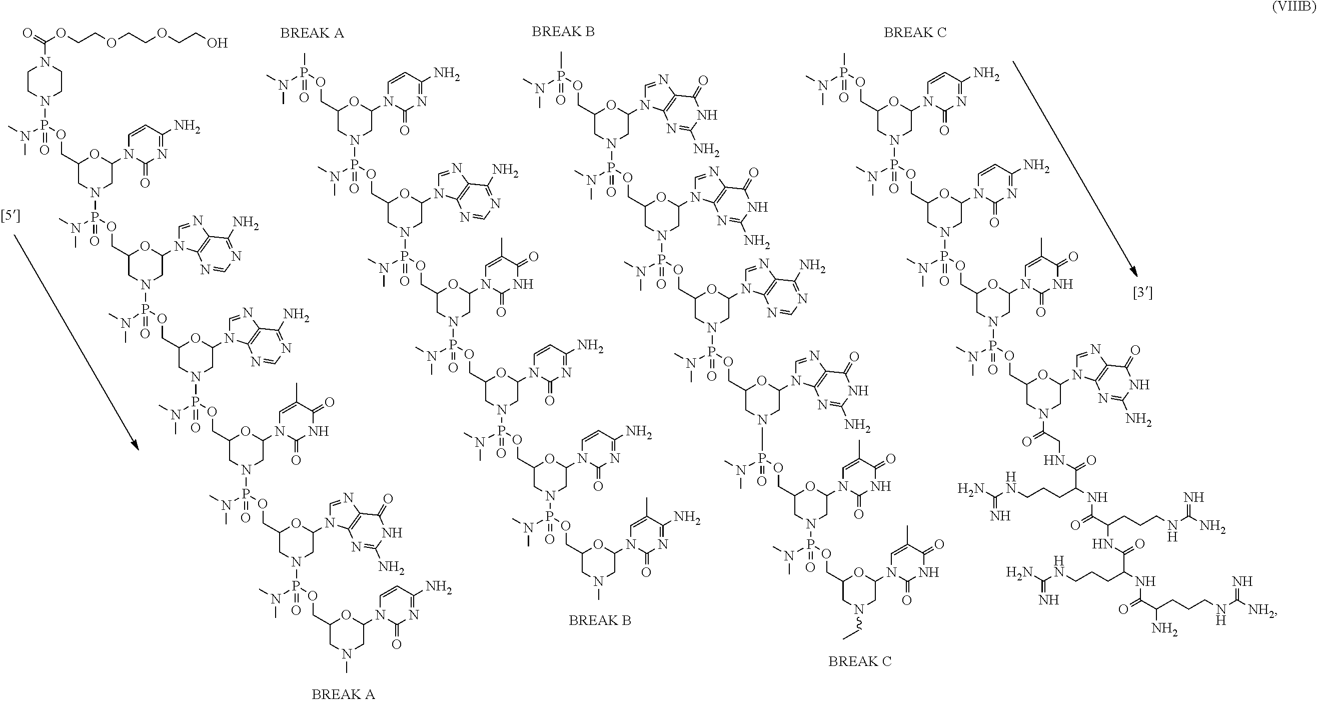

or a pharmaceutically acceptable salt thereof, such as a HCl salt.

In some embodiments of the disclosure, including some embodiments of antisense oligomer conjugates of Formula (I) and Formula (III), the antisense oligomer conjugate is according to Formula (VIIA):

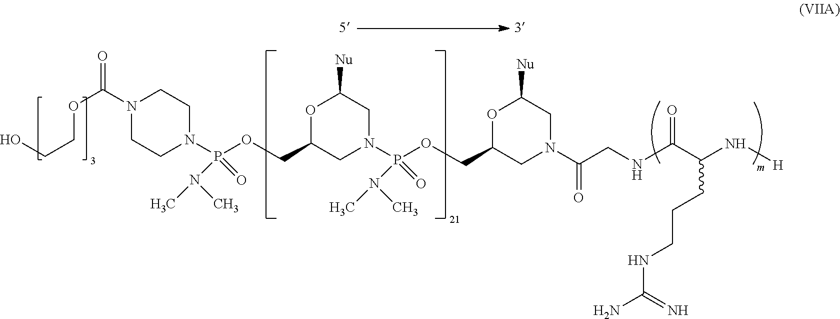

or a pharmaceutically acceptable salt thereof, wherein m is 0, 1, 2, 3, 4, or 5, and
each Nu from 1 to 22 and 5′ to 3′ is:

| Position | |
|---|---|
| No. 5′ | |
| to 3′ | Nu |
| 1 | C |
| 2 | A |
| 3 | A |
| 4 | T |
| 5 | G |
| 6 | C |
| 7 | C |
| 8 | A |
| 9 | T |
| 10 | C |
| 11 | C |
| 12 | T |
| 13 | G |
| 14 | G |
| 15 | A |
| 16 | G |
| 17 | T |
| 18 | T |
| 19 | C |
| 20 | C |
| 21 | T |
| 22 | G |

wherein A is

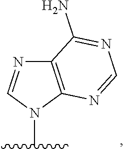 [SMILES: *n1cnc2c(N)ncnc21]

### C is

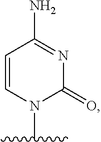 [SMILES: *n1ccc(N)nc1=O]

### G is

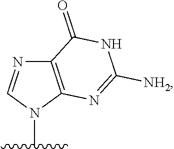 [SMILES: *n1cnc2c(=O)[nH]c(N)nc21]

### and T is

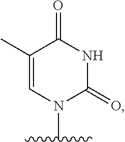 [SMILES: *n1cc(C)c(=O)[nH]c1=O]

represented by Formula (VIIIC):

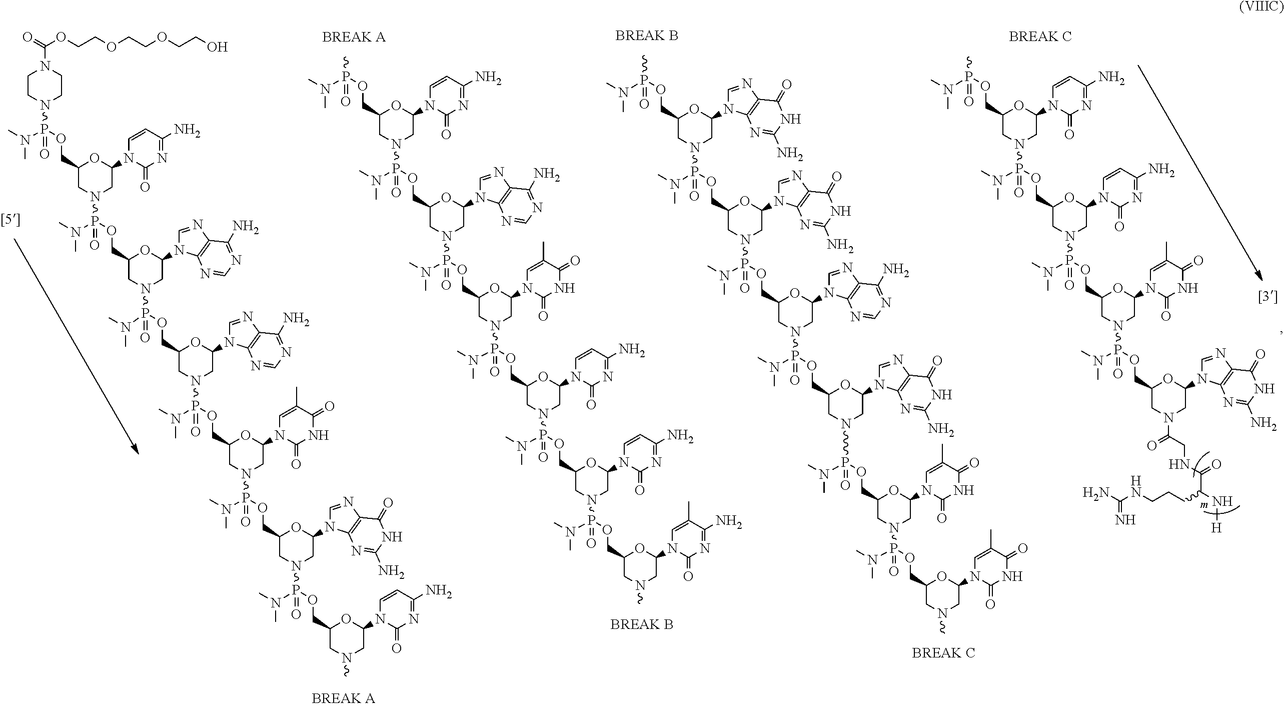

or a pharmaceutically acceptable salt thereof, wherein m is 0, 1, 2, 3, 4, or 5.

In some embodiments, an antisense oligomer conjugate of Formula (VIIIC) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (VIIIC), an antisense oligomer conjugate of the disclosure is according to Formula (VIIID) or Formula (VIIIE):

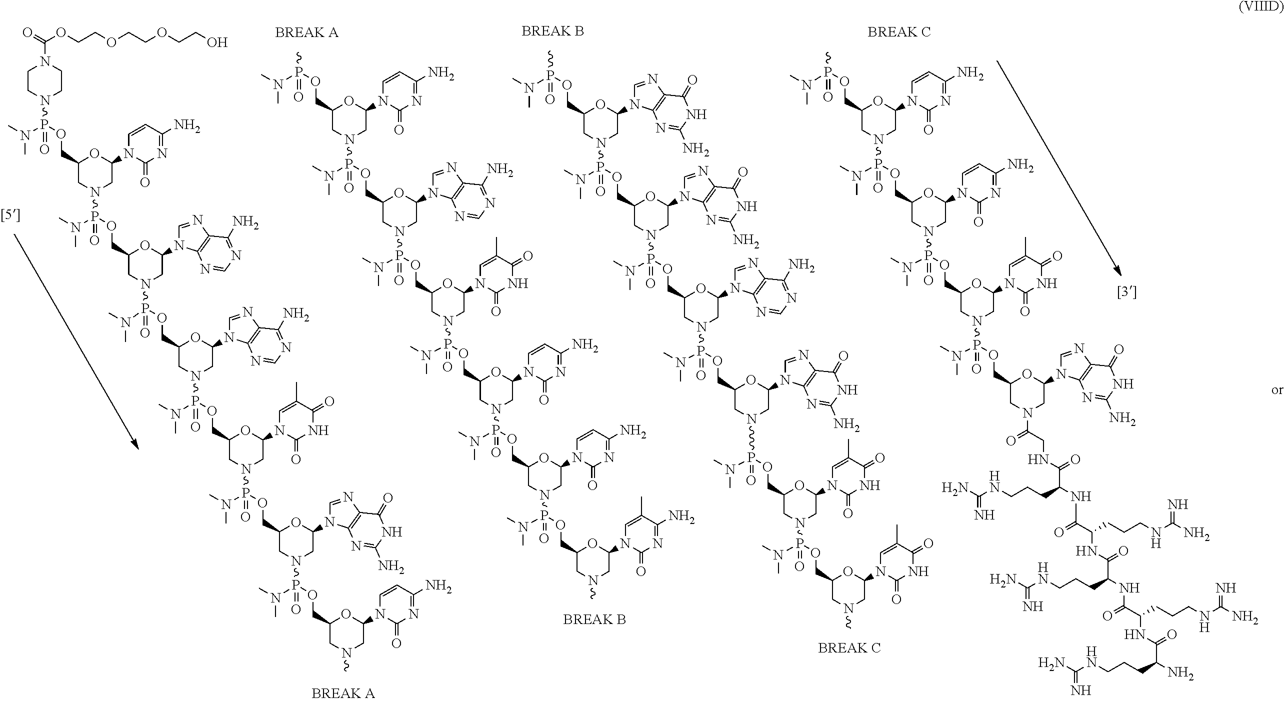

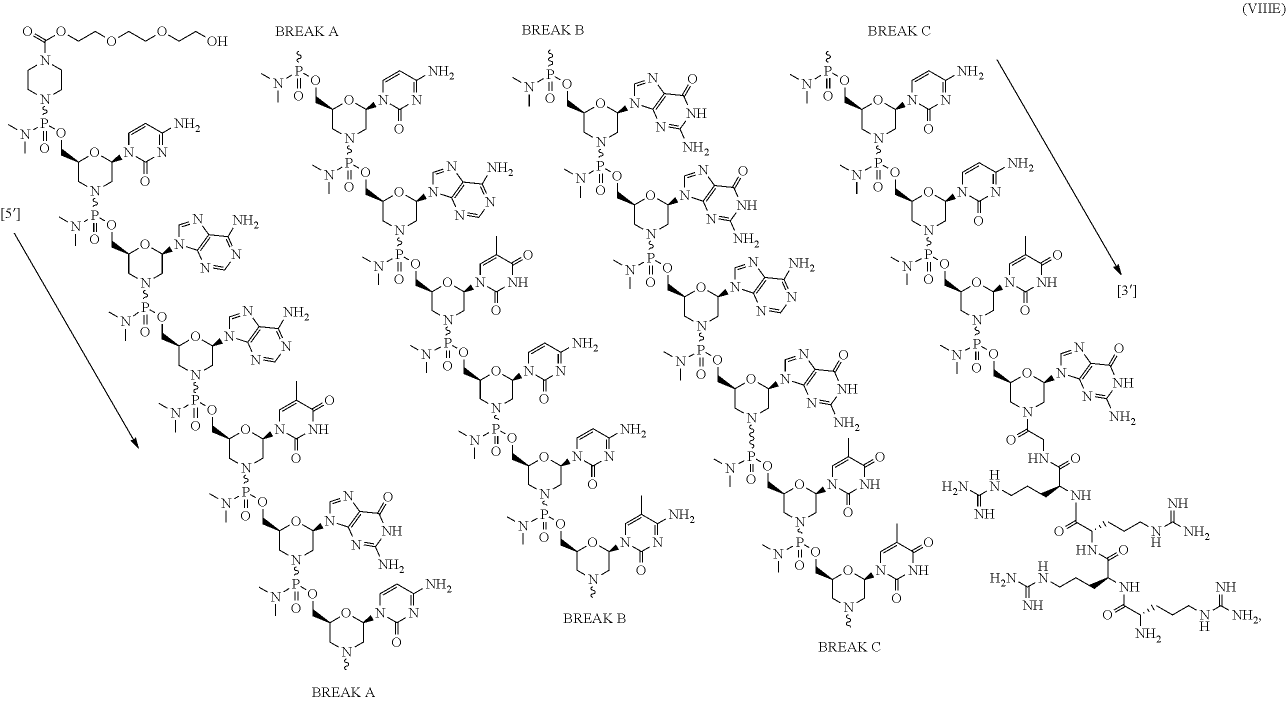

or a pharmaceutically acceptable salt, such as a HCl salt thereof.

In some embodiments, including, for example, embodiments of antisense oligomer conjugates of Formula (VIIIC), an antisense oligomer conjugate of the disclosure is according to Formula (VIIIF) or Formula (VIIIG):

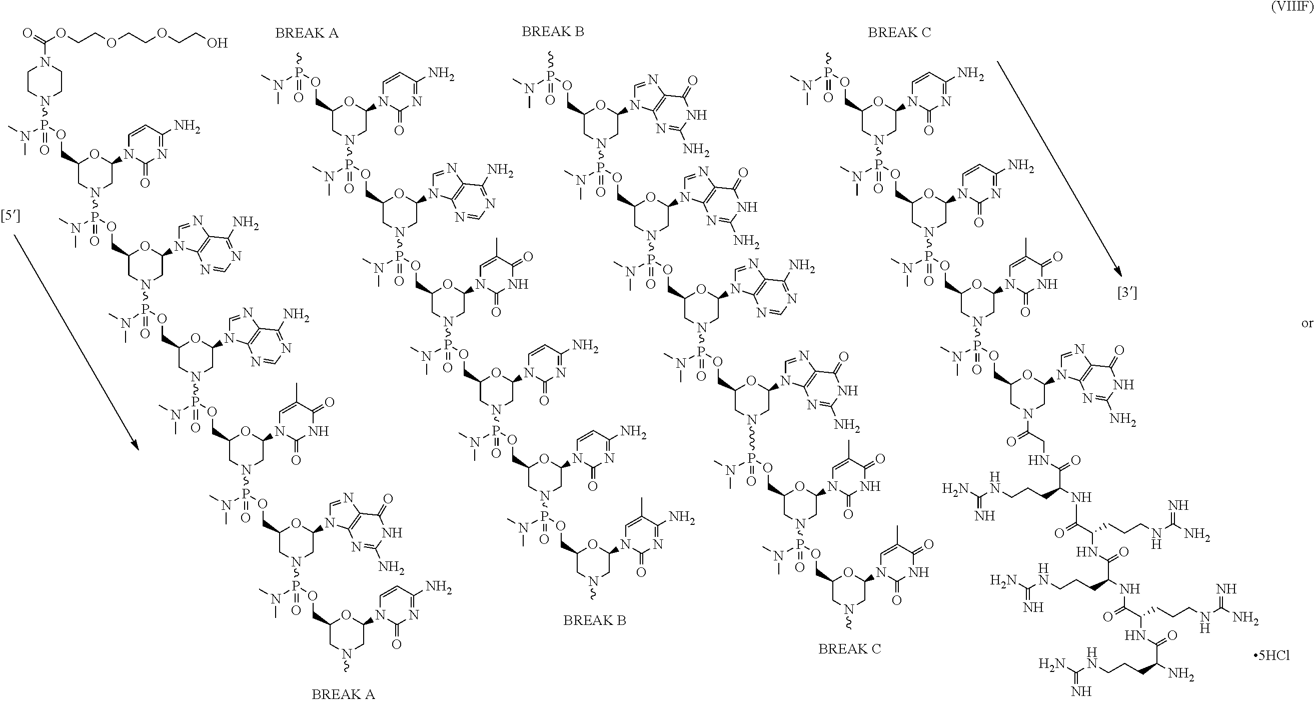

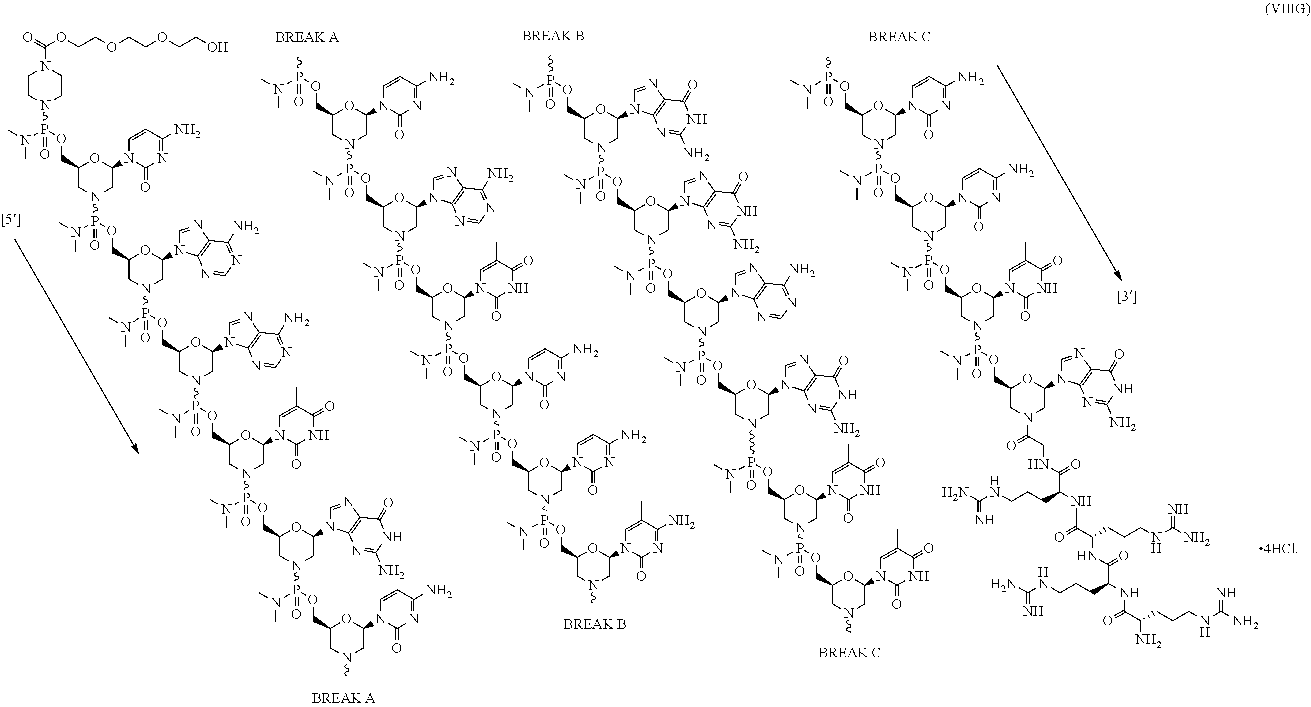

In some embodiments, including, for example, some embodiments of Formula (I) or Formula (IV), an antisense oligomer conjugate of the disclosure is according to Formula (IX):

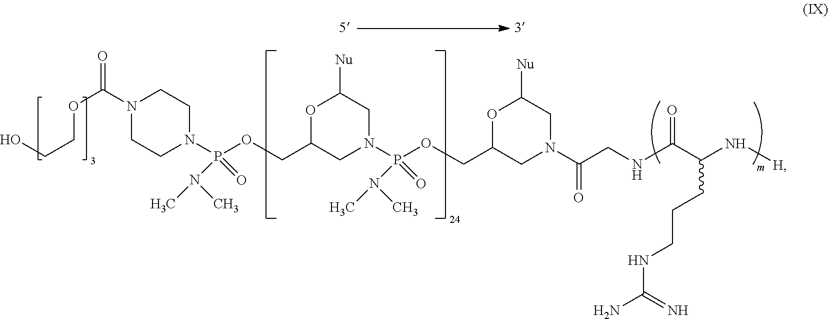

or a pharmaceutically acceptable salt thereof, wherein:
each Nu is a nucleobase which taken together form a targeting sequence that is complementary to an exon 53 annealing site in the dystrophin pre-mRNA designated as H53A(+36+60); and
m is 0, 1, 2, 3, 4, or 5.

In some embodiments, including, for example, some embodiments of Formula (IX), an antisense oligomer conjugate of the disclosure is according to Formula (IXA):

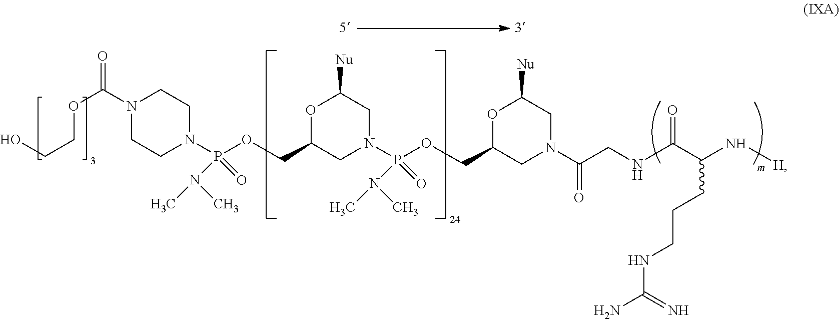

or a pharmaceutically acceptable salt thereof, wherein:
each Nu is a nucleobase which taken together form a targeting sequence that is complementary to an exon 53 annealing site in the dystrophin pre-mRNA designated as H53A(+36+60); and
m is 0, 1, 2, 3, 4, or 5.

In some embodiments, each Nu in Formula (IX) or Formula (IXA) is independently selected from cytosine (C), guanine (G), thymine (T), adenine (A), 5-methylcytosine (5mC), uracil (U), and hypoxanthine (I).

In various embodiments, each Nu from 1 to 25 and 5′ to 3′ is:

| Position | |
|---|---|
| No. 5′ | |
| to 3′ | Nu |
| 1 | G |
| 2 | X |
| 3 | X |
| 4 | G |
| 5 | C |
| 6 | C |
| 7 | X |
| 8 | C |
| 9 | C |
| 10 | G |
| 11 | G |
| 12 | X |
| 13 | X |
| 14 | C |
| 15 | X |
| 16 | G |
| 17 | A |
| 18 | A |
| 19 | G |
| 20 | G |
| 21 | X |
| 22 | G |
| 23 | X |
| 24 | X |
| 25 | C |

wherein A is

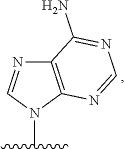 [SMILES: *n1cnc2c(N)ncnc21]

### C is

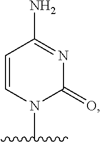 [SMILES: *n1ccc(N)nc1=O]

### G is

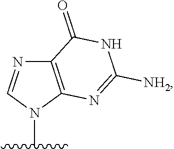 [SMILES: *n1cnc2c(=O)[nH]c(N)nc21]

### and X is

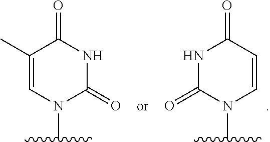

In certain embodiments, each X is independently

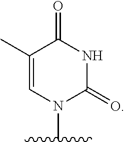 [SMILES: *n1cc(C)c(=O)[nH]c1=O]

In some embodiments, an antisense oligomer conjugate of Formula (IX) or Formula (IXA) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, some embodiments of Formula (IXA), an antisense oligomer conjugate of the disclosure is according to Formula (IXB) or Formula (IXC):

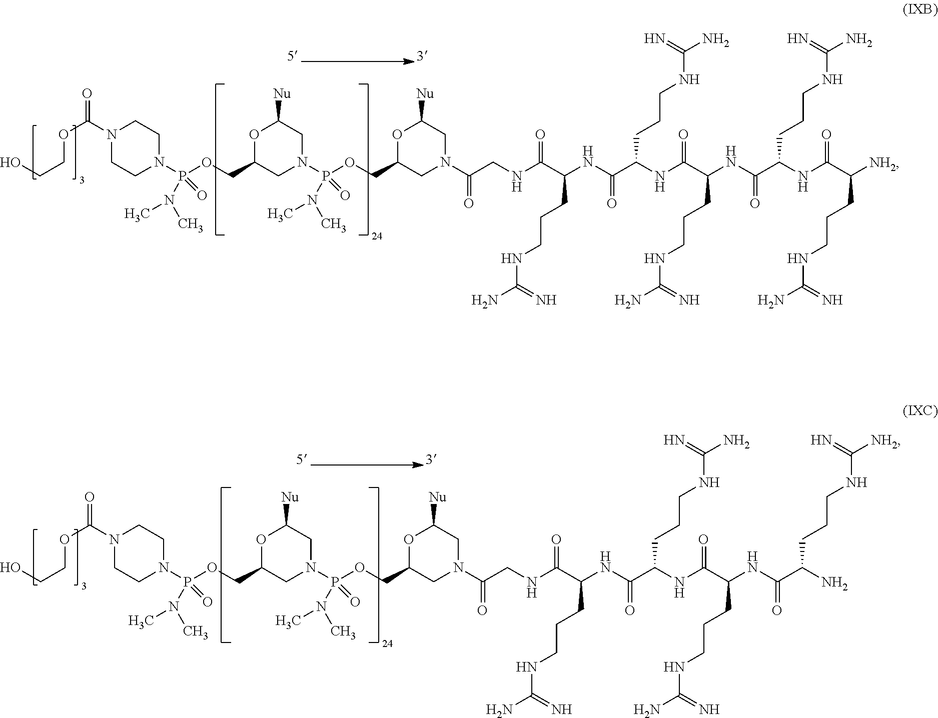

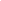

or a pharmaceutically acceptable salt thereof, such as a HCl-salt, wherein each Nu is a nucleobase which taken together form a targeting sequence that is complementary to an exon 53 annealing site in the dystrophin pre-mRNA designated as H53A(+36+60).

In some embodiments, each Nu in Formula (IXB) or (IXC) is independently selected from cytosine (C), guanine (G), thymine (T), adenine (A), 5-methylcytosine (5mC), uracil (U), and hypoxanthine (I).

In various embodiments, each Nu from 1 to 25 and 5′ to 3′ is:

| Position | |
|---|---|
| No. 5′ | |
| to 3′ | Nu |
| 1 | G |
| 2 | X |
| 3 | X |
| 4 | G |
| 5 | C |
| 6 | C |
| 7 | X |
| 8 | C |
| 9 | C |
| 10 | G |
| 11 | G |
| 12 | X |
| 13 | X |
| 14 | C |
| 15 | X |
| 16 | G |
| 17 | A |
| 18 | A |
| 19 | G |
| 20 | G |
| 21 | X |
| 22 | G |
| 23 | X |
| 24 | X |
| 25 | C |

wherein A is

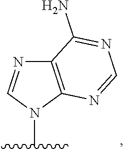 [SMILES: *n1cnc2c(N)ncnc21]

### C is

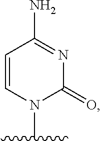 [SMILES: *n1ccc(N)nc1=O]

### G is

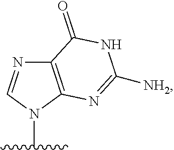 [SMILES: *n1cnc2c(=O)[nH]c(N)nc21]

### and X is

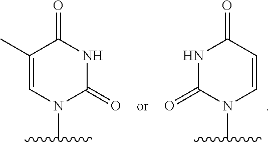

In certain embodiments, each X is

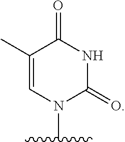 [SMILES: *n1cc(C)c(=O)[nH]c1=O]

In some embodiments including, for example, embodiments of antisense oligomer conjugates of Formula (IX), Formula (IXA), Formula (IXB), and Formula (IXC), the targeting sequence is 5′-GTTGCCTCCGGTTCTGAAGGTGTTC-3′ (SEQ ID NO: 3) wherein each thymine (T) is optionally uracil (U). In various embodiments including, for example, embodiments of antisense oligomer conjugates of Formula (IX), Formula (IXA), Formula (IXB), and Formula (IXC), the targeting sequence is 5′-GTTGCCTCCGGTTCTGAAGGTGTTC-3′ (SEQ ID NO: 3).

In some embodiments, including, for example, embodiments of antisense oligomer conjugates of Formula (I) or Formula (IV), an antisense oligomer conjugate of the disclosure is according to Formula (IX):

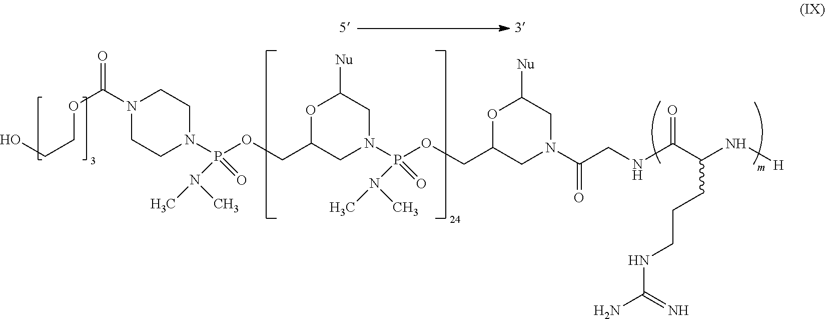

or a pharmaceutically acceptable salt thereof, wherein m is 0, 1, 2, 3, 4, or 5, and
each Nu from 1 to 25 and 5′ to 3′ is:

| Position | |
|---|---|
| No. 5′ | |
| to 3′ | Nu |
| 1 | G |
| 2 | T |
| 3 | T |
| 4 | G |
| 5 | C |
| 6 | C |
| 7 | T |
| 8 | C |
| 9 | C |
| 10 | G |
| 11 | G |
| 12 | T |
| 13 | T |
| 14 | C |
| 15 | T |
| 16 | G |
| 17 | A |
| 18 | A |
| 19 | G |
| 20 | G |
| 21 | T |
| 22 | G |
| 23 | T |
| 24 | T |
| 25 | C |

wherein A is

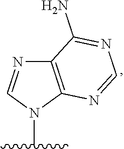 [SMILES: *n1cnc2c(N)ncnc21]

### C is

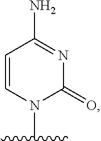 [SMILES: *n1ccc(N)nc1=O]

### G is

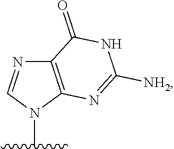 [SMILES: *n1cnc2c(=O)[nH]c(N)nc21]

### and T is

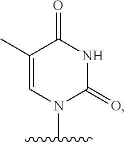 [SMILES: *n1cc(C)c(=O)[nH]c1=O]

represented by Formula (X):

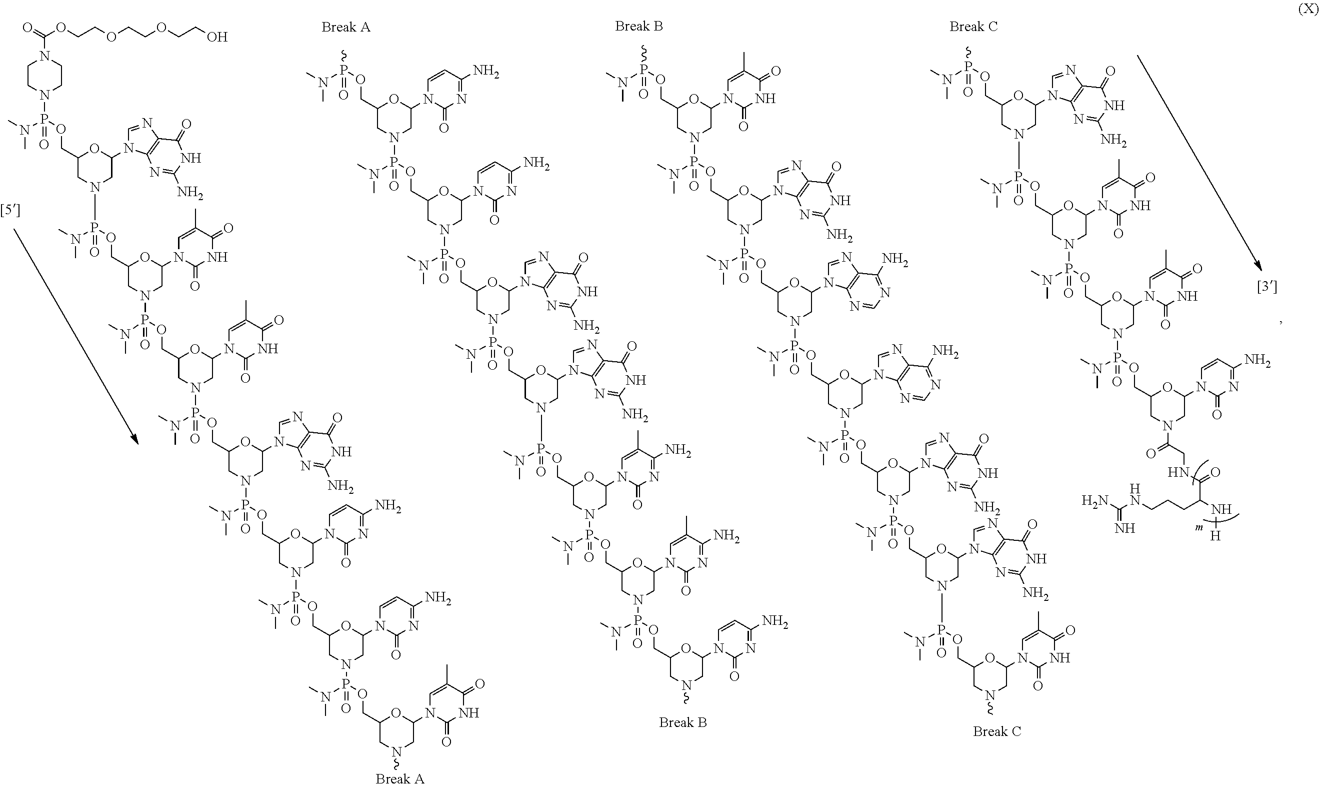

or a pharmaceutically acceptable salt thereof, wherein m is 0, 1, 2, 3, 4, or 5.

In some embodiments, an antisense oligomer conjugate of Formula (X) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (X), an antisense oligomer conjugate of the disclosure is according to Formula (XA) or Formula (XIB):

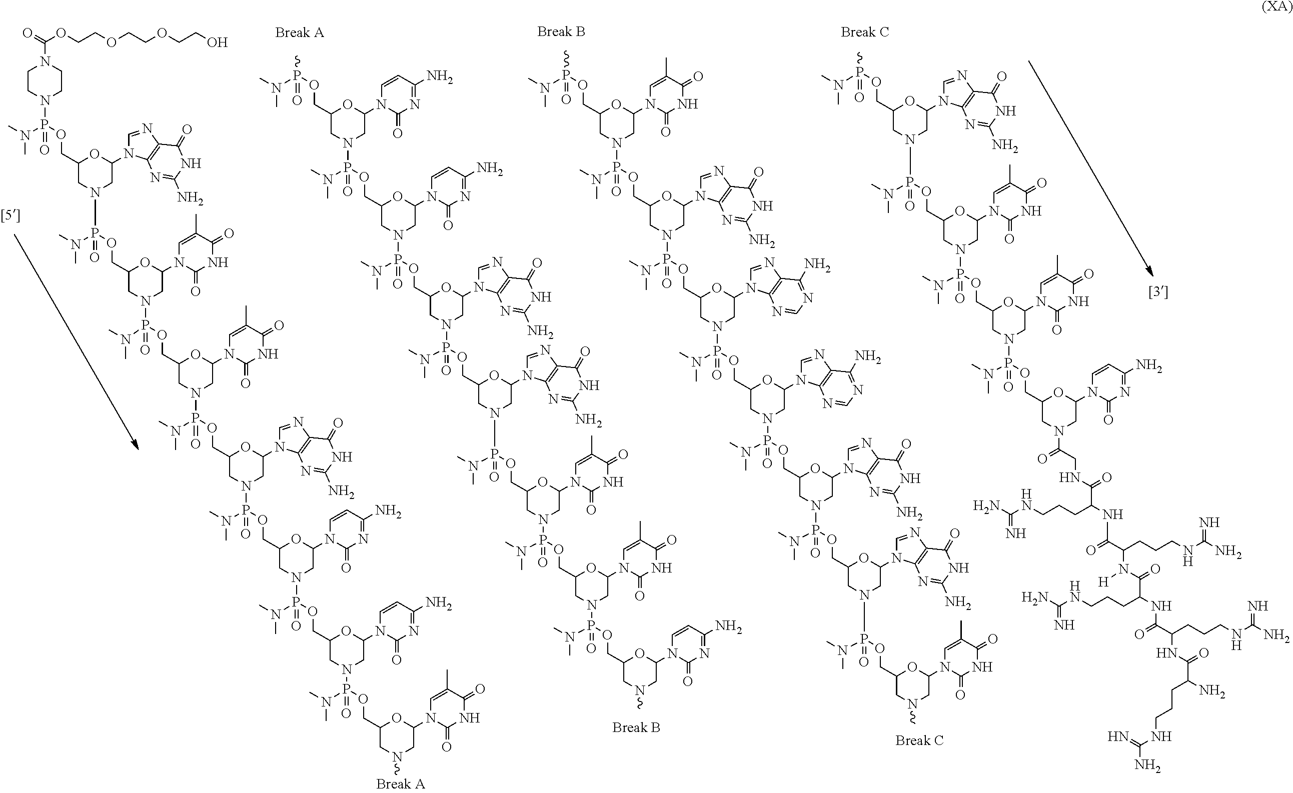

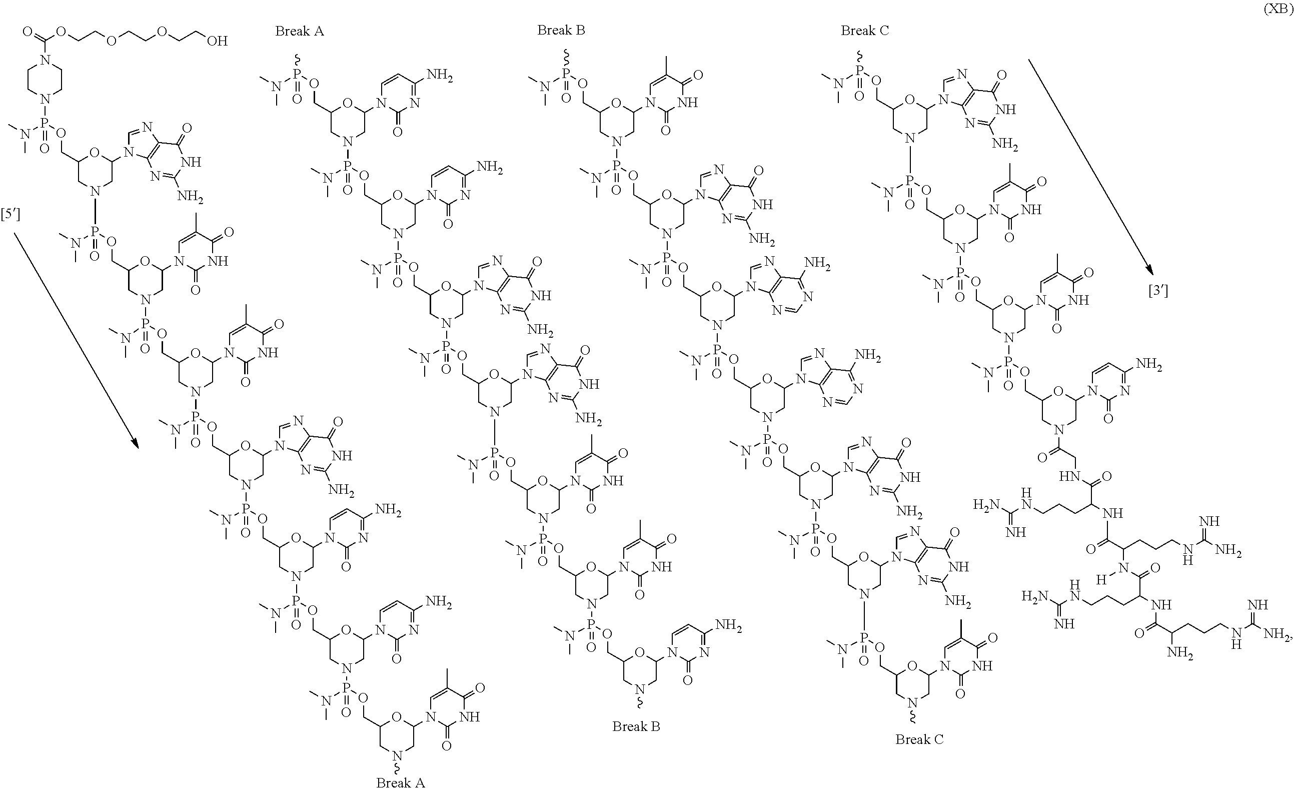

or a pharmaceutically acceptable salt thereof, such as a HCl-salt.

In some embodiments of the disclosure, including some embodiments of antisense oligomer conjugates of Formula (I) and Formula (IV), the antisense oligomer conjugate is according to Formula (IXA):

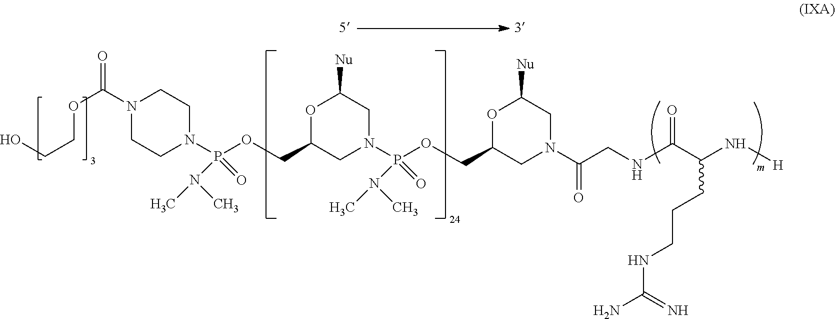

or a pharmaceutically acceptable salt thereof, wherein m is 0, 1, 2, 3, 4, or 5, and
each Nu from 1 to 25 and 5′ to 3′ is:

| Position | |
|---|---|
| No. 5′ | |
| to 3′ | Nu |
| 1 | G |
| 2 | T |
| 3 | T |
| 4 | G |
| 5 | C |
| 6 | C |
| 7 | T |
| 8 | C |
| 9 | C |
| 10 | G |
| 11 | G |
| 12 | T |
| 13 | T |
| 14 | C |
| 15 | T |
| 16 | G |
| 17 | A |
| 18 | A |
| 19 | G |
| 20 | G |
| 21 | T |
| 22 | G |
| 23 | T |
| 24 | T |
| 25 | C |

wherein A is

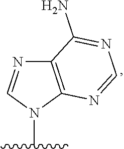 [SMILES: *n1cnc2c(N)ncnc21]

### C is

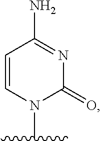 [SMILES: *n1ccc(N)nc1=O]

### G is

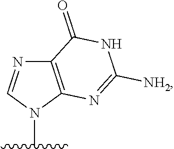 [SMILES: *n1cnc2c(=O)[nH]c(N)nc21]

### and T is

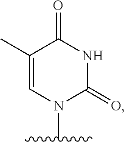 [SMILES: *n1cc(C)c(=O)[nH]c1=O]

represented by Formula (XC):

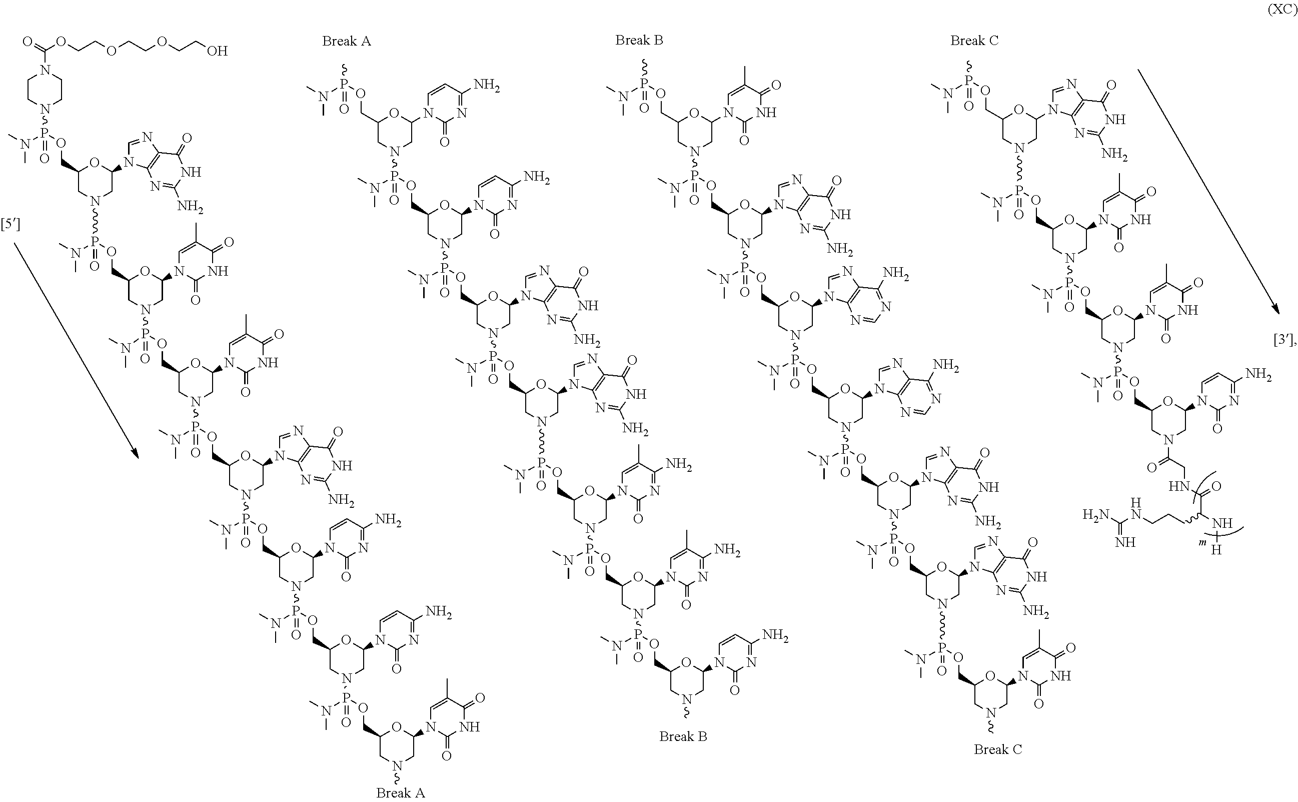

or a pharmaceutically acceptable salt thereof, wherein m is 0, 1, 2, 3, 4, or 5.

In some embodiments, an antisense oligomer conjugate of Formula (XC) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In some embodiments, including, for example, embodiments of antisense oligomer conjugates of Formula (XC), an antisense oligomer conjugate of the disclosure is according to Formula (XD) or Formula (XE):

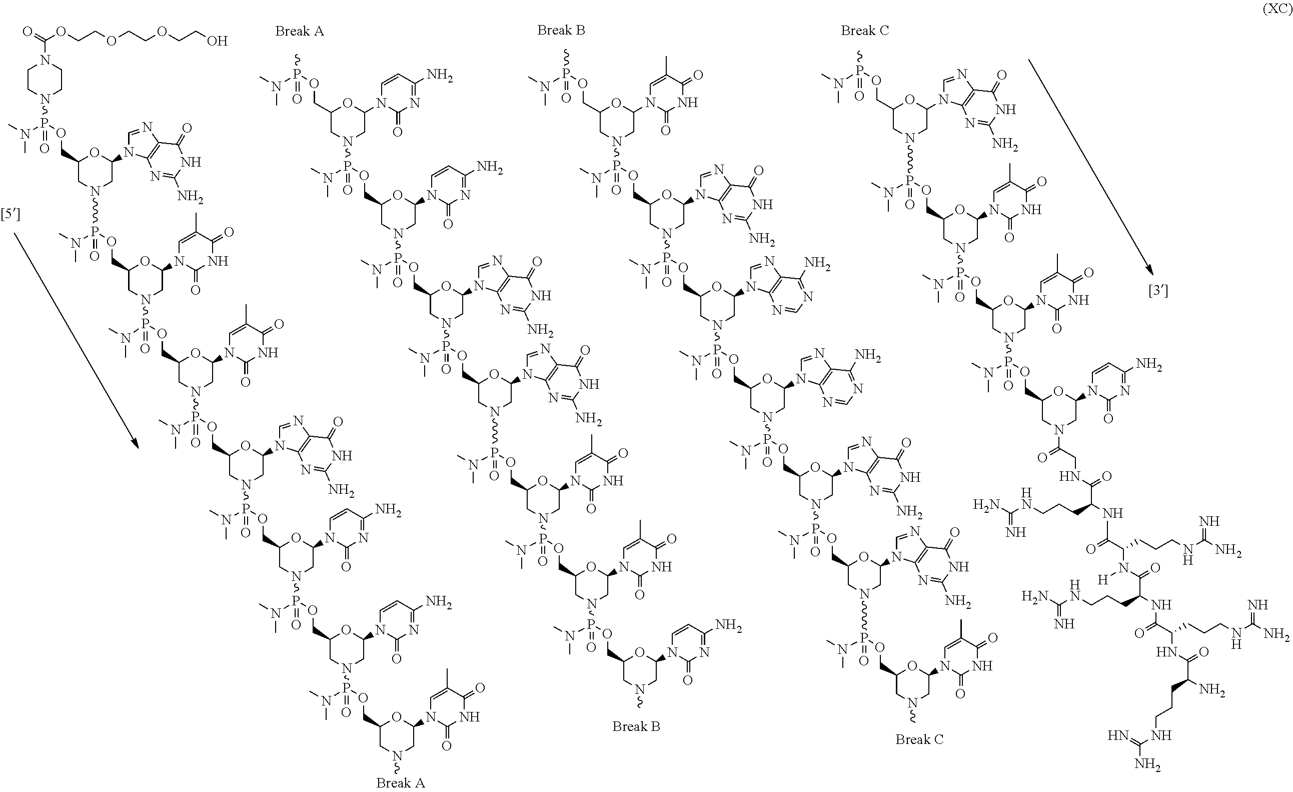

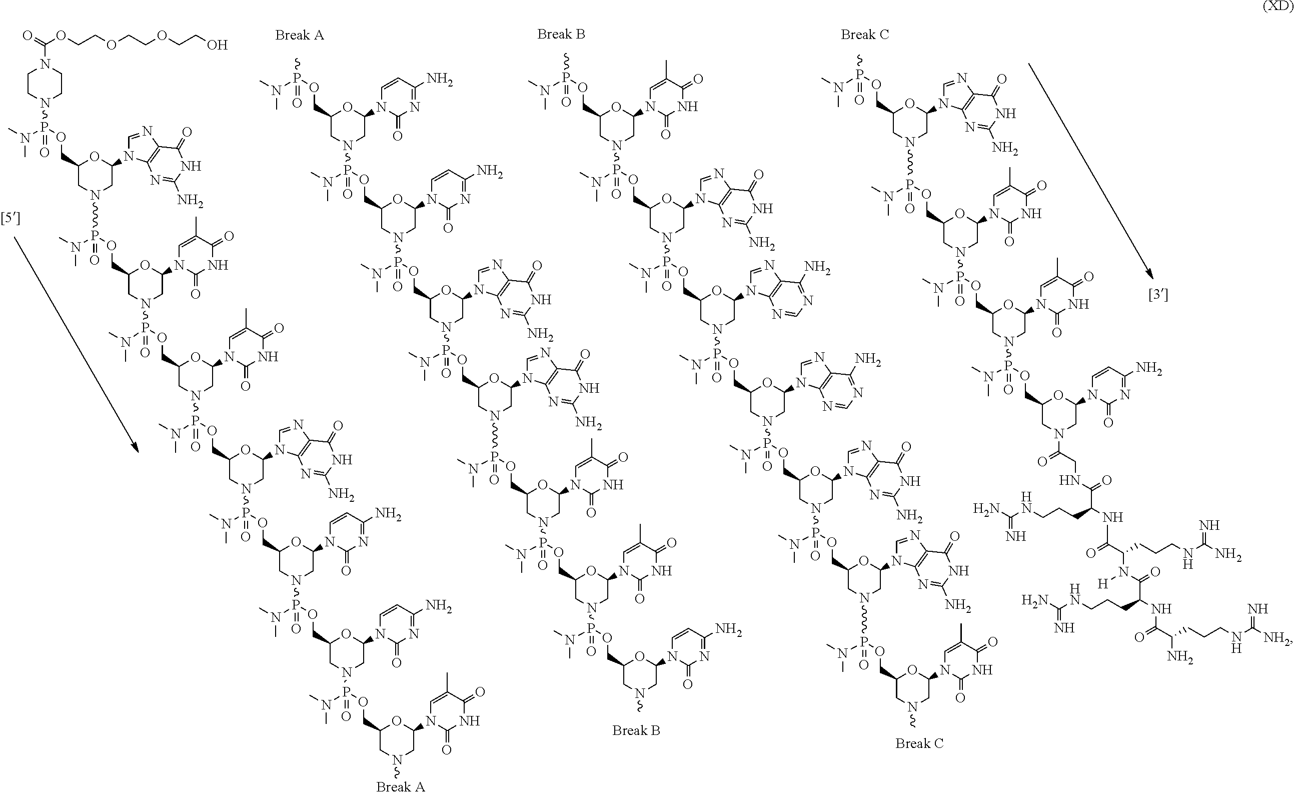

or a pharmaceutically acceptable salt thereof, such as a HCl-salt.

In some embodiments, including, for example, embodiments of antisense oligomer conjugates of Formula (XC), an antisense oligomer conjugate of the disclosure is according to Formula (XF) or Formula (XG):

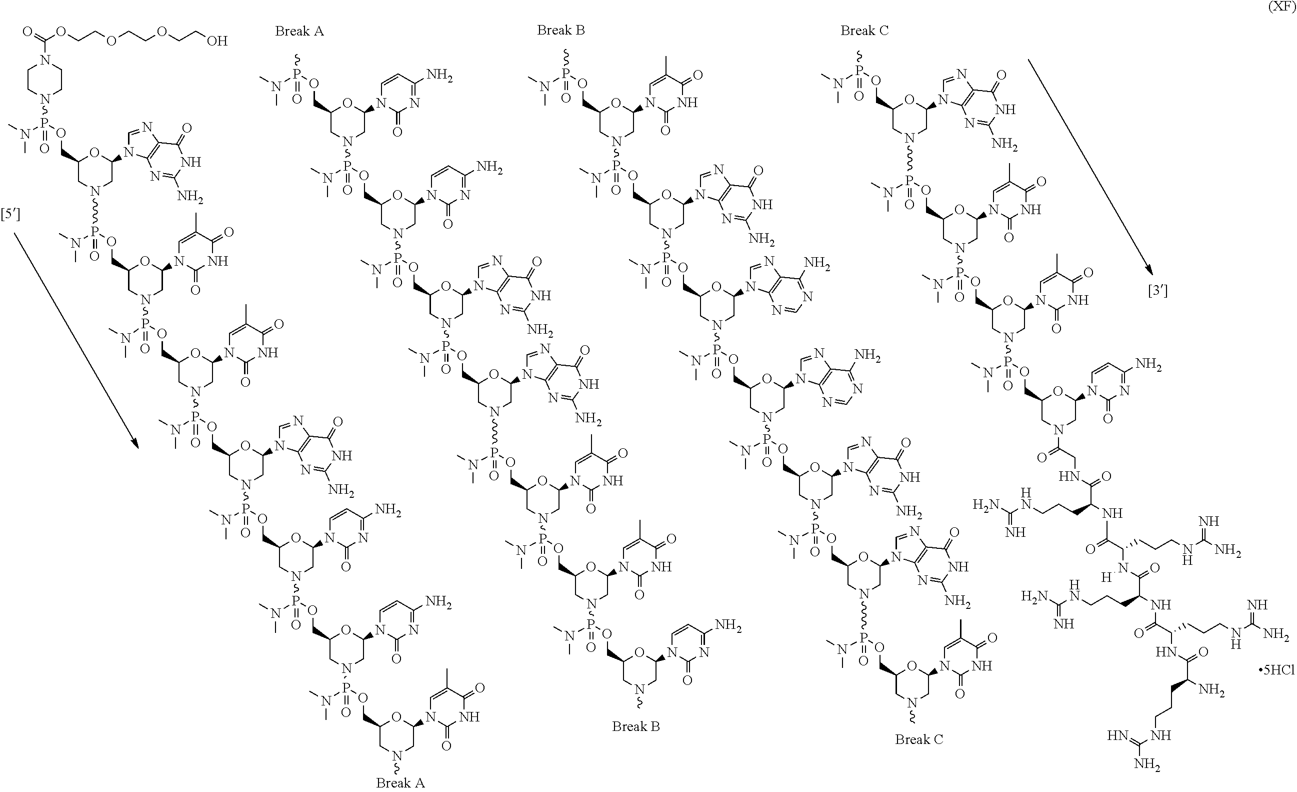

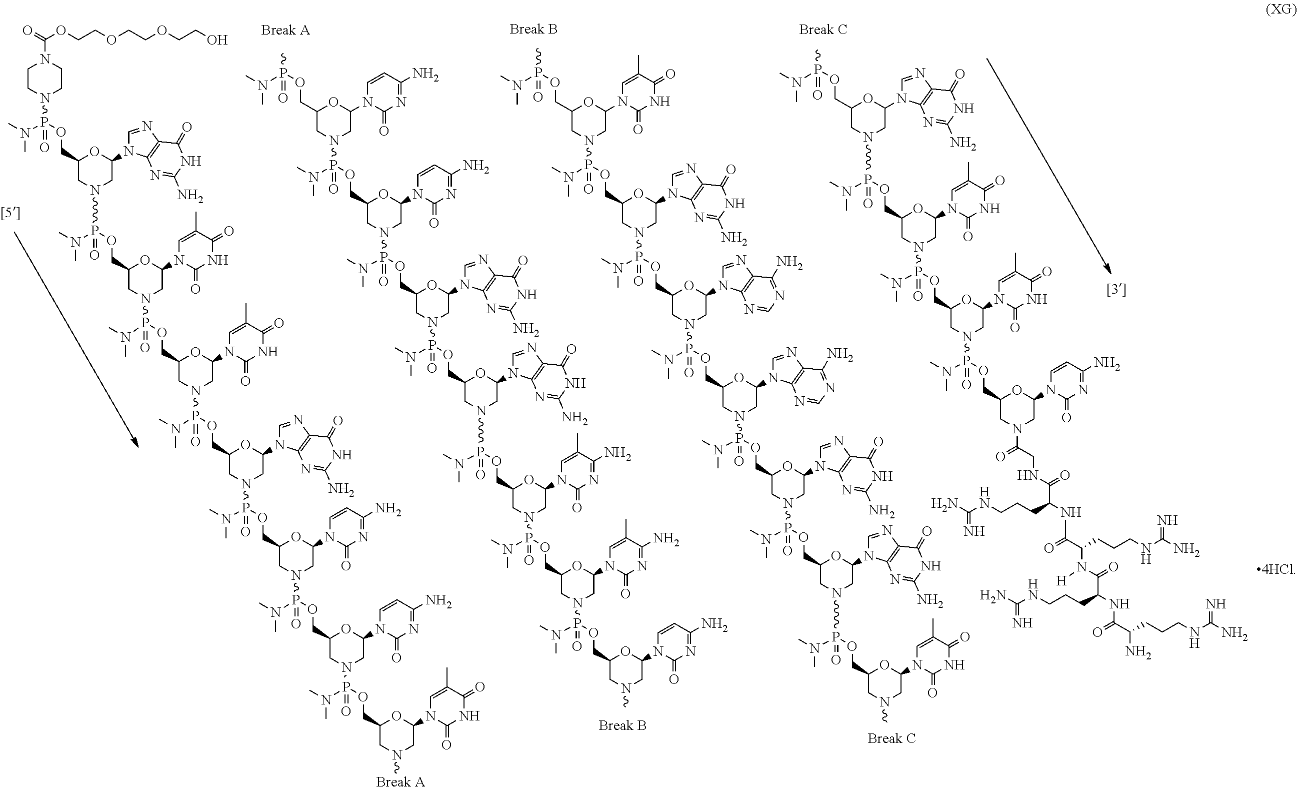

In one aspect, the disclosure provides an antisense oligomer conjugate, or a pharmaceutically acceptable salt thereof, capable of binding a selected target to induce exon skipping in the human dystrophin gene, wherein the antisense oligomer conjugate, or a pharmaceutically acceptable salt thereof, comprises a sequence of bases that is complementary to an exon 51 target region of the dystrophin pre-mRNA designated as an annealing site; wherein the base sequence and annealing site are selected from one of the following:

| Annealing Site | Base Sequence [5′ to 3′] |
|---|---|
| H51D(+16-07) | CTC ATA CCT TCT GCT TGA TGA TC |
| SEQ ID NO: 4 | |
| H50D(+103+127) | GGG ATC CAG TAT ACT TAC AGG CTC C |
| SEQ ID NO: 5 | |
| H51A(+61+82) | GAA GAT GGC ATT TCT AGT TTG G |
| SEQ ID NO: 6 | |
| H51A(+61+83) | GGA AGA TGG CAT TTC TAG TTT GG |
| SEQ ID NO: 7 | |
| H51A(+61+89) | CAT CAA GGA AGA TGG CAT TTC TAG TTT GG |
| SEQ ID NO: 8 | |
| H51A(+66+89) | CAT CAA GGA AGA TGG CAT TTC TAG |
| SEQ ID NO: 9 | |
| H51A(+66+93) | CCA ACA TCA AGG AAG ATG GCA TTT CTA G |
| SEQ ID NO: 10 | |
| H51A(+69+92) | CAA CAT CAA GGA AGA TGG CAT TTC |
| SEQ ID NO: 11 | |
| H51A(+69+96) | CCT CCA ACA TCA AGG AAG ATG GCA TTT C |
| SEQ ID NO: 12 | |
| H51A(+74+96) | CCT CCA ACA TCA AGG AAG ATG GC |
| SEQ ID NO: 13 | |
| H51A(+74+99) | GTA CCT CCA ACA TCA AGG AAG ATG GC |
| SEQ ID NO: 14 | |
| H51A(+74+100) | GGT ACC TCC AAC ATC AAG GAA GAT GGC |
| SEQ ID NO: 15 | |
| H51A(+74+102) | CAG GTA CCT CCA ACA TCA AGG AAG ATG GC |
| SEQ ID NO: 16 | |
| H51A(+74+103) | GCA GGT ACC TCC AAC ATC AAG GAA GAT GGC |
| SEQ ID NO: 17 | |
| H51A(+75+96) | CCT CCA ACA TCA AGG AAG ATG G |
| SEQ ID NO: 18 | |
| H51A(+75+99) | GTA CCT CCA ACA TCA AGG AAG ATG G |
| SEQ ID NO: 19 | |
| H51A(+76+99) | GTA CCT CCA ACA TCA AGG AAG ATG |
| SEQ ID NO: 20 | |
| H51A(+76+105) | GAG CAG GTA CCT CCA ACA TCA AGG AAG ATG |
| SEQ ID NO: 21 | |
| H51A(+80+103) | GCA GGT ACC TCC AAC ATC AAG GAA G |
| SEQ ID NO: 22 | |
| H51A(+80+105) | GAG CAG GTA CCT CCA ACA TCA AGG AAG |
| SEQ ID NO: 23 | |
| H51A(+80+107) | CAG AGC AGG TAC CTC CAA CAT CAA GGA AG |
| SEQ ID NO: 24 | |
| H51A(+80+108) | CCA GAG CAG GTA CCT CCA ACA TCA AGG AAG |
| SEQ ID NO: 25 | |
| H51A(+83+103) | GCA GGT ACC TCC AAC ATC AAG G |
| SEQ ID NO: 26 | |
| H51A(+83+105) | GAG CAG GTA CCT CCA ACA TCA AGG |
| SEQ ID NO: 27 | |
| H51A(+83+107) | CAG AGC AGG TAC CTC CAA CAT CAA GG |
| SEQ ID NO: 28 | |
| H51A(+83+109) | GCC AGA GCA GGT ACC TCC AAC ATC AAG G |
| SEQ ID NO: 29 | |
| H51A(+84+107) | CAG AGC AGG TAC CTC CAA CAT CAA G |
| SEQ ID NO: 30 | |
| H51A(+84+111) | CTG CCA GAG CAG GTA CCT CCA ACA TCA AG |
| SEQ ID NO: 31 | |
| H51A(+84+105) | GAG CAG GTA CCT CCA ACA TCA AG |
| SEQ ID NO: 32 | |
| H51A(+87+109) | GCC AGA GCA GGT ACC TCC AAC ATC |
| SEQ ID NO: 33 | |
| H51A(+93+116) | GAA ATC TGC CAG AGC AGG TAC CTC |
| SEQ ID NO: 34 | |
| H51A(+75+100) | GGT ACC TCC AAC ATC AAG GAA GAT GG |
| SEQ ID NO: 35 | |
| H51A(+74+101) | AGG TAC CTC CAA CAT CAA GGA AGA TGG C |
| SEQ ID NO: 36 | |
| H51A(+74+98) | TAC CTC CAA CAT CAA GGA AGA TGG C |
| SEQ ID NO: 37 | |
| H51A(+74+97) | ACC TCC AAC ATC AAG GAA GAT GGC |
| SEQ ID NO: 38 | |
| H51A(+74+94) | TCC AAC ATC AAG GAA GAT GGC |
| SEQ ID NO: 39 | |
| H51A(+74+93) | CCA ACA TCA AGG AAG ATG GC |
| SEQ ID NO: 40 | |
| H51A(+74+92) | CAA CAT CAA GGA AGA TGG C |
| SEQ ID NO: 41 | |
| H51A(+69+99) | GTA CCT CCA ACA TCA AGG AAG ATG GCA TTT C |
| SEQ ID NO: 42 | |
| H51A(+70+99) | GTA CCT CCA ACA TCA AGG AAG ATG GCA TTT |
| SEQ ID NO: 43 | |
| H51A(+71+99) | GTA CCT CCA ACA TCA AGG AAG ATG GCA TT |
| SEQ ID NO: 44 | |
| H51A(+72+99) | GTA CCT CCA ACA TCA AGG AAG ATG GCA T |
| SEQ ID NO: 45 | |
| H51A(+73+99) | GTA CCT CCA ACA TCA AGG AAG ATG GCA |
| SEQ ID NO: 46 | |
| H51A(+77+99) | GTA CCT CCA ACA TCA AGG AAG AT |
| SEQ ID NO: 47 | |
| H51A(+78+99) | GTA CCT CCA ACA TCA AGG AAG A |
| SEQ ID NO: 48 | |
| H51A(+79+99) | GTA CCT CCA ACA TCA AGG AAG |
| SEQ ID NO: 49 | |
| H51.SA.(−60−36) | GAA GAA AAA GAA AAA TTA GAA ACA C |
| SEQ ID NO: 50 | |
| H51.SA.(−50−26) | AAG GAA AAA AGA AGA AAA AGA AAA A |
| SEQ ID NO: 51 | |
| H51.SA.(−45−21) | GCA AAA AGG AAA AAA GAA GAA AAA G |
| SEQ ID NO: 52 | |
| H51.SA.(−40−16) | TTT TTG CAA AAA GGA AAA AAG AAG A |
| SEQ ID NO: 53 | |
| H51.SA.(−35−11) | TTG GGT TTT TGC AAA AAG GAA AAA A |
| SEQ ID NO: 54 | |
| H51.SA.(−30−6) | ATA TTT TGG GTT TTT GCA AAA AGG A |
| SEQ ID NO: 55 | |
| H51.SA.(−25−1) | CTA AAA TAT TTT GGG TTT TTG CAA A |
| SEQ ID NO: 56 | |
| H51.SA.(−20+5) | AGG AGC TAA AAT ATT TTG GGT TTT T |
| SEQ ID NO: 57 | |
| H51.SA.(−15+10) | TGA GTA GGA GCT AAA ATA TTT TGG G |
| SEQ ID NO: 58 | |
| H51.SA.(−10+15) | CAG TCT GAG TAG GAG CTA AAA TATT |
| SEQ ID NO: 59 | |
| H51.SA.(−5+20) | AGT AAC AGT CTG AGT AGG AGC TAA A |
| SEQ ID NO: 60 | |
| H51.SA.(−1+24) | CCA GAG TAA CAG TCT GAG TAG GAG C |
| SEQ ID NO: 61 | |
| H51.SA.(−65−41) | AAA AGA AAA ATT AGA AAC ACA AGC T |
| SEQ ID NO: 62 | |
| H51.SA.(−70−46) | AAA AAT TAG AAA CAC AAG CTA AAG A |
| SEQ ID NO: 63 | |
| H51.SA.(−75−51) | TTA GAA ACA CAA GCT AAA GAG CCA A |
| SEQ ID NO: 64 | |
| H51.SA.(−80−56) | AAC ACA AGC TAA AGA GCC AAT TTC A |
| SEQ ID NO: 65 | |
| H51.SA.(−85−61) | AAG CTA AAG AGC CAA TTT CAA TAA C |
| SEQ ID NO: 66 | |
| H51.SA.(−90−66) | AAA GAG CCA ATT TCA ATA ACA ATA A |
| SEQ ID NO: 67 | |
| H51.SA.(−95−71) | GCC AAT TTC AAT AAC AAT AAG TCA A |
| SEQ ID NO: 68 | |
| H51.SA.(−100−76) | TTT CAA TAA CAA TAA GTC AAA TTT A |
| SEQ ID NO: 69 | |
| H51A(+1+30) | GTG TCA CCA GAG TAA CAG TCT GAG TAG GAG |
| SEQ ID NO: 70 | |
| H51A(+10+39) | CCA CAG GTT GTG TCA CCA GAG TAA CAG TCT |
| SEQ ID NO: 71 | |
| H51A(+6+35) | AG GTT GTG TCA CCA GA GTA ACA GTC TGA GT |
| SEQ ID NO: 72 | |
| H51A(+49+78) | ATG GCA TTT CTA GTT TGG AGA TGG CAG TTT |
| SEQ ID NO: 73 | |
| H51A(+1+25) | ACC AGA GTA ACA GTC TGA GTA GGA G |
| SEQ ID NO: 74 | |
| H51A(+4+28) | GTC ACC AGA GTA ACA GTC TGA GTA G |
| SEQ ID NO: 75 | |
| H51A(+16+40) | ACC ACA GGT TGT GTC ACC AGA GTA A |
| SEQ ID NO: 76 | |
| H51A(+21+45) | TAG TAA CCA CAG GTT GTG TCA CCA G |
| SEQ ID NO: 77 | |
| H51A(+26+50) | TTC CTT AGT AAC CAC AGG TTG TGT C |
| SEQ ID NO: 78 | |
| H51A(+31+55) | GCA GTT TCC TTA GTA ACC ACA GGT T |
| SEQ ID NO: 79 | |
| H51A(+36+60) | AGA TGG CAG TTT CCT TAG TAA CCA C |
| SEQ ID NO: 80 | |
| H51A(+41+65) | TTT GGA GAT GGC AGT TTC CTT AGT A |
| SEQ ID NO: 81 | |
| H51A(+86+110) | TGC CAG AGC AGG TAC CTC CAA CAT C |
| SEQ ID NO: 82 | |
| H51A(+91+115) | AAA TCT GCC AGA GCA GGT ACC TCC A |
| SEQ ID NO: 83 | |
| H51A(+96+120) | GGT TGA AAT CTG CCA GAG CAG GTA C |
| SEQ ID NO: 84 | |
| H51A(+101+125) | AGC CCG GTT GAA ATC TGC CAG AGC A |
| SEQ ID NO: 85 | |
| H51A(+106+130) | GTC CAA GCC CGG TTG AAA TCT GCC A |
| SEQ ID NO: 86 | |
| H51A(+111+135) | GTT CTG TCC AAG CCC GGT TGA AAT C |
| SEQ ID NO: 87 | |
| H51A(+116+140) | GGT AAG TTC TGT CCA AGC CCG GTT G |
| SEQ ID NO: 88 | |
| H51A(+121+145) | CAG TCG GTA AGT TCT GTC CAA GCC C |
| SEQ ID NO: 89 | |
| H51A(+126+150) | AAA GCC AGT CGG TAA GTT CTG TCC A |
| SEQ ID NO: 90 | |
| H51A(+131+155) | CAG AGA AAG CCA GTC GGT AAG TTC T |
| SEQ ID NO: 91 | |
| H51A(+136+160) | TCA AGC AGA GAA AGC CAG TCG GTA A |
| SEQ ID NO: 92 | |
| H51A(+141+165) | CTT GAT CAA GCA GAG AAA GCC AGT C |
| SEQ ID NO: 93 | |
| H51A(+146+170) | TAT AAC TTG ATC AAG CAG AGA AAG C |
| SEQ ID NO: 94 | |
| H51A(+151+175) | GAT TTT ATA ACT TGA TCA AGC AGA G |
| SEQ ID NO: 95 | |
| H51A(+156+180) | TCT GTG ATT TTA TAA CTT GAT CAA G |
| SEQ ID NO: 96 | |
| H51A(+161+185) | CAC CCT CTG TGA TTT TAT AAC TTG A |
| SEQ ID NO: 97 | |
| H51A(+166+190) | ACC ATC ACC CTC TGT GAT TTT ATA A |
| SEQ ID NO: 98 | |
| H51A(+171+195) | CAC CCA CCA TCA CCC TCT GTG ATT T |
| SEQ ID NO: 99 | |
| H51A(+176+200) | AAG GTC ACC CAC CAT CAC CCT CTG T |
| SEQ ID NO: 100 | |
| H51A(+181+205) | TCC TCA AGG TCA CCC ACC ATC ACC C |
| SEQ ID NO: 101 | |
| H51A(+186+210) | TGA TAT CCT CAA GGT CAC CCA CCA T |
| SEQ ID NO: 102 | |
| H51A(+191+215) | CTC GTT GAT ATC CTC AAG GTC ACC C |
| SEQ ID NO: 103 | |
| H51A(+196+220) | ATC ATC TCG TTG ATA TCC TCA AGG T |
| SEQ ID NO: 104 | |
| H51A(+201+225) | TGA TGA TCA TCT CGT TGA TAT CCT C |
| SEQ ID NO: 105 | |
| H51A(+206+230) | CTG CTT GAT GAT CAT CTC GTT GAT A |
| SEQ ID NO: 106 | |
| H51D(+211−02) | ACC TTC TGC TTG ATG ATC ATC TCG T |
| SEQ ID NO: 107 | |
| H51D(+214−05) | CAT ACC TTC TGC TTG ATG ATC ATC T |
| SEQ ID NO: 108 | |
| H51D(+217−08) | TCT CAT ACC TTC TGC TTG ATG ATC A |
| SEQ ID NO: 109 | |
| H51D(+220−11) | TTT TCT CAT ACC TTC TGC TTG ATG A |
| SEQ ID NO: 110 | |
| H51D(+223−14) | ATT TTT TCT CAT ACC TTC TGC TTG A |
| SEQ ID NO: 111 | |
| H51D(+226−17) | ATC ATT TTT TCT CAT ACC TTC TGC T |
| SEQ ID NO: 112 | |
| H51D(+229−20) | TTT ATC ATT TTT TCT CAT ACC TTC T |
| SEQ ID NO: 113 | |
| H51D(+232−23) | ACT TTT ATC ATT TTT TCT CAT ACC T |
| SEQ ID NO: 114 | |
| H51D(−02−26) | CCA ACT TTT ATC ATT TTT TCT CAT A |
| SEQ ID NO: 115 | |
| H51A(+1+30) | GTG TCA CCA GAG TAA CAG TCT GAG TAG GAG |
| SEQ ID NO: 116 | |

wherein A is

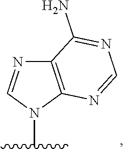 [SMILES: *n1cnc2c(N)ncnc21]

### C is

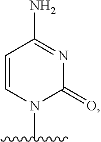 [SMILES: *n1ccc(N)nc1=O]

### G is

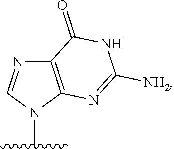 [SMILES: *n1cnc2c(=O)[nH]c(N)nc21]

### and T is

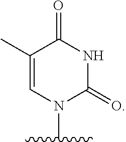 [SMILES: *n1cc(C)c(=O)[nH]c1=O]

In one aspect, the base sequence and annealing site are selected from one of the following:

| Annealing | |
|---|---|
| Site | Base Sequence [5′ to 3′] |
| H51A(+66+95) | CTC CAA CAT CAA GGA AGA TGG CAT TTC TAG |
| SEQ ID NO: 117 | |
| H51A(+74+97) | ACC TCC AAC ATC AAG GAA GAT GGC |
| SEQ ID NO: 118 | |
| H51A(+70+99) | GTA CCT CCA ACA TCA AGG AAG ATG GCA TTT |
| SEQ ID NO: 119 | |
| H51A(+72+99) | GTA CCT CCA ACA TCA AGG AAG ATG GCA T |
| SEQ ID NO: 120 | |
| H51A(+68+87) | TCA AGG AAG ATG GCA TTT CT |
| SEQ ID NO: 121 | |
| H51A(+68+87) | UCA AGG AmAGm AmUGm GmCA UUU CU |
| SEQ ID NO: 122 | |
| H53A(+36+60) | GTT GCC TCC GGT TCT GAA GGT GTT C |
| SEQ ID NO: 123 | |
| H53A(+36+60) | GTT G5mC5mC T5mC5mC GGT T5mC T GAA GGT GTT 5mC |
| SEQ ID NO: 124 | |
| H53A(+36+56) | CCT CCG GTT CTG AAG GTG TTC |
| SEQ ID NO: 125 | |
| H53A(+23+47) | CTG AAG GTG TTC TTG TAC TTC ATC C |
| SEQ ID NO: 126 | |
| H53A(+32+56) | CCT CCG GTT CTG AAG GTG TTC TTG T |
| SEQ ID NO: 127 | |
| H53A(+33+60) | GTT GCC TCC GGT TCT GAA GGT GTT CTT G |
| SEQ ID NO: 128 | |
| H53A(+30+59) | TTG CCT CCG GTT CTG AAG GTG TTC TTG TAC |
| SEQ ID NO: 129 | |
| H53A(+39+62) | CTG TTG CCT CCG GTT CTG AAG GTG |
| SEQ ID NO: 130 | |
| H53A(+36+69) | CAT TCA ACT GTT GCC TCC GGT TCT GAA GGT G |
| SEQ ID NO: 131 | |
| H53A(+45+62) | CTG TTG CCT CCG GTT CTG |
| SEQ ID NO: 132 | |
| H45A(−03+19) | CAA TGC CAT CCT GGA GTT CCT G |
| SEQ ID NO: 133 | |
| H45A(−09+25) | GCT GCC CAA TGC CAT CCT GGA GTT CCT GTA AGA T |
| SEQ ID NO: 134 | |
| H45A(−03+25) | GCT GCC CAA TGC CAT CCT GGA GTT CCT G |
| SEQ ID NO: 135 | |
| H45A(−06+25) | GCT GCC CAA TGC CAT CCT GGA GTT CCT GTA A |
| SEQ ID NO: 136 | |
| H45A(−12+19) | CAA TGC CAT CCT GGA GTT CCT GTA AGA TAC C |
| SEQ ID NO: 137 | |
| H45A(−09+19) | CAA TGC CAT CCT GGA GTT CCT GTA AGA T |
| SEQ ID NO: 138 | |
| H45A(−12+16) | TGC CAT CCT GGA GTT CCT GTA AGA TAC C |
| SEQ ID NO: 139 | |
| H45A(−14+25) | GCT GCC CAA TGC CAT CCT GGA GTT CCT GTA AGA TAC CAA |
| SEQ ID NO: 140 | |
| H45A(−08+19) | CAA TGC CAT CCT GGA GTT CCT GTA AGA |
| SEQ ID NO: 141 | |
| HM45A(−07+25) | GCT GCC CAA TGC CAT CCT GGA GTT CCT GTA AG |
| SEQ ID NO: 142 | |
| H45A(−12+22) | GCC CAA TGC CAT CCT GGA GTT CCT GTA AGA TAC C |
| SEQ ID NO: 143 | |
| H45A(−09+22) | GCC CAA TGC CAT CCT GGA GTT CCT GTA AGA T |
| SEQ ID NO: 144 | |
| H45A(−09+30) | TTG CCG CTG CCC AAT GCC ATC CTG GAG TTC CTG TAA GAT |
| SEQ ID NO: 145 | |
| H45A(−06+22) | GCC CAA TGC CAT CCT GGA GTT CCT GTA A |
| SEQ ID NO: 146 | |
| H45A(−06+28) | GCC GCT GCC CAA TGC CAT CCT GGA GTT CCT GTA A |
| SEQ ID NO: 147 | |
| H45A(−03+22) | GCC CAA TGC CAT CCT GGA GTT CCT G |
| SEQ ID NO: 148 | |
| H45A(−03+28) | GCC GCT GCC CAA TGC CAT CCT GGA GTT CCT G |
| SEQ ID NO: 149 | |
| H45A(+9+26) | m5CGm5C TGC m5Cm5CA ATG m5Cm5CA Um5Cm5C |
| SEQ ID NO: 150 | |
| H44A(−10+15) | GAT CTG TCA AAT CGC CTG CAG GTA A |
| SEQ ID NO: 151 | |
| H44A(−07+15) | GAT CTG TCA AAT CGC CTG CAG G |
| SEQ ID NO: 152 | |
| H44M(−07+17) | CAG ATC TGT CAA ATC GCC TGC AGG |
| SEQ ID NO: 153 | |
| H44A(−08+15) | GAT CTG TCA AAT CGC CTG CAG GT |
| SEQ ID NO: 154 | |
| H44A(−06+15) | GAT CTG TCA AAT CGC CTG CAG |
| SEQ ID NO: 155 | |
| H44A(−08+17) | CAG ATC TGT CAA ATC GCC TGC AGG T |
| SEQ ID NO: 156 | |
| H44A(−06+17) | CAG ATC TGT CAA ATC GCC TGC AG |
| SEQ ID NO: 157 | |
| H50D(+04−18) | GGG ATC CAG TAT ACT TAC AGG C |
| SEQ ID NO: 158 | |
| H50D(+07−18) | GGG ATC CAG TAT ACT TAC AGG CTC C |
| SEQ ID NO: 159 | |
| H50D(+07−16) | GAT CCA GTA TAC TTA CAG GCT CC |
| SEQ ID NO: 160 | |
| H50D(+07−17) | GGA TCC AGT ATA CTT ACA GGC TCC |
| SEQ ID NO: 161 | |
| H50A(−19+07) | ACT TCC TCT TTA ACA GAA AAG CAT AC |
| SEQ ID NO: 162 | |
| H50D(+07−15) | ATC CAG TAT ACT TAC AGG CTC C |
| SEQ ID NO: 163 | |
| H50A(−02+23) | GAG CTC AGA TCT TCT AAC TTC CTC T |
| SEQ ID NO: 164 | |
| H50D(+06−18) | GGG ATC CAG TAT ACT TAC AGG CTC |
| SEQ ID NO: 165 | |
| H50D(+07−20) | ATG GGA TCC AGT ATA CTT ACA GGC TCC |
| SEQ ID NO: 166 | |
| H52A(−01+24) | CTG TTC CAA ATC CTG CAT TGT TGC C |
| SEQ ID NO: 167 | |

wherein A is

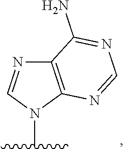 [SMILES: *n1cnc2c(N)ncnc21]

### C is

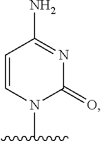 [SMILES: *n1ccc(N)nc1=O]

### G is

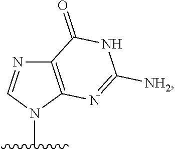 [SMILES: *n1cnc2c(=O)[nH]c(N)nc21]

### T is

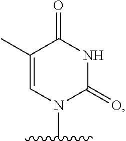 [SMILES: *n1cc(C)c(=O)[nH]c1=O]

### U is

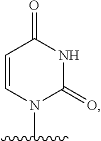 [SMILES: *n1ccc(=O)[nH]c1=O]

Gm is methylated guanine, Am is methylated adenine, and m5C is

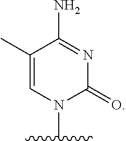 [SMILES: *n1cc(C)c(N)nc1=O]

In another aspect, the disclosure provides antisense oligomer conjugates of Formula (XI):

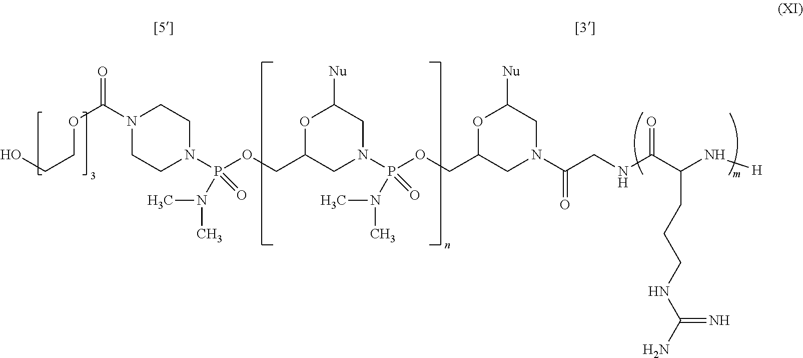

or a pharmaceutically acceptable salt thereof, where m is 0, 1, 2, 3, 4, or 5 and each Nu from 1 to (n+1) and 5′ to 3′ corresponds to the nucleobases in the following sequences:

| Annealing Site | Sequence [5′ to 3′] |
|---|---|
| H51A(+61+90) | ACA TCA AGG AAG ATG GCA TTT CTA GTT TGG |
| SEQ ID NO: 168 | |
| H51D(+16−07) | CTC ATA CCT TCT GCT TGA TGA TC |
| SEQ ID NO: 169 | |
| H50D(+103+127) | GGG ATC CAG TAT ACT TAC AGG CTC C |
| SEQ ID NO: 170 | |
| H51A(+81+105) | GAG CAG GTA CCT CCA ACA TCA AGG AA |
| SEQ ID NO: 171 | |
| H51A(+71+100) | GGT ACC TCC AAC ATC AAG GAA GAT GGC ATT |
| SEQ ID NO: 172 | |
| H51A(+48+73) | ATT TCT AGT TTG GAG ATG GCA GTT TC |
| SEQ ID NO: 173 | |
| H51A(+59+84) | GGA AGA TGG CAT TTC TAG TTT GGA G |
| SEQ ID NO: 174 | |
| H51A(+64+88) | CAT CAA GGA AGA TGG CAT TTC TAG TT |
| SEQ ID NO: 175 | |
| H51A(+89+113) | ATC TGC CAG AGC AGG TAC CTC CAA C |
| SEQ ID NO: 176 | |
| H51A(+49+68) | TAG TTT GGA GAT GGC AGT TT |
| SEQ ID NO: 177 | |
| H51A(+64+83) | GGA AGA TGG CAT TTC TAG TT |
| SEQ ID NO: 178 | |
| H51A(+80+98) | TAC CTC CAA CAT CAA GGA AG |
| SEQ ID NO: 179 | |
| H51A(+94+113) | ATC TGC CAG AGC AGG TAC CT |
| SEQ ID NO: 180 | |
| H51A(+109+128) | CCA AGC CCG GTT GAA ATC TG |
| SEQ ID NO: 181 | |
| H51A(+61+82) | GAA GAT GGC ATT TCT AGT TTG G |
| SEQ ID NO: 182 | |
| H51A(+61+83) | GGA AGA TGG CAT TTC TAG TTT GG |
| SEQ ID NO: 183 | |
| H51A(+61+89) | CAT CAA GGA AGA TGG CAT TTC TAG TTT GG |
| SEQ ID NO: 184 | |
| H51A(+66+89) | CAT CAA GGA AGA TGG CAT TTC TAG |
| SEQ ID NO: 185 | |
| H51A(+66+93) | CCA ACA TCA AGG AAG ATG GCA TTT CTA G |
| SEQ ID NO: 186 | |
| H51A(+69+92) | CAA CAT CAA GGA AGA TGG CAT TTC |
| SEQ ID NO: 187 | |
| H51A(+69+96) | CCT CCA ACA TCA AGG AAG ATG GCA TTT C |
| SEQ ID NO: 188 | |
| H51A(+74+96) | CCT CCA ACA TCA AGG AAG ATG GC |
| SEQ ID NO: 189 | |
| H51A(+74+99) | GTA CCT CCA ACA TCA AGG AAG ATG GC |
| SEQ ID NO: 190 | |
| H51A(+74+100) | GGT ACC TCC AAC ATC AAG GAA GAT GGC |
| SEQ ID NO: 191 | |
| H51A(+74+102) | CAG GTA CCT CCA ACA TCA AGG AAG ATG GC |
| SEQ ID NO: 192 | |
| H51A(+74+103) | GCA GGT ACC TCC AAC ATC AAG GAA GAT GGC |
| SEQ ID NO: 193 | |
| H51A(+75+96) | CCT CCA ACA TCA AGG AAG ATG G |
| SEQ ID NO: 194 | |
| H51A(+75+99) | GTA CCT CCA ACA TCA AGG AAG ATG G |
| SEQ ID NO: 195 | |
| H51A(+76+99) | GTA CCT CCA ACA TCA AGG AAG ATG |
| SEQ ID NO: 196 | |
| H51A(+76+105) | GAG CAG GTA CCT CCA ACA TCA AGG AAG ATG |
| SEQ ID NO: 197 | |
| H51A(+80+103) | GCA GGT ACC TCC AAC ATC AAG GAA G |
| SEQ ID NO: 198 | |
| H51A(+80+105) | GAG CAG GTA CCT CCA ACA TCA AGG AAG |
| SEQ ID NO: 199 | |
| H51A(+80+107) | CAG AGC AGG TAC CTC CAA CAT CAA GGA AG |
| SEQ ID NO: 200 | |
| H51A(+80+108) | CCA GAG CAG GTA CCT CCA ACA TCA AGG AAG |
| SEQ ID NO: 201 | |
| H51A(+83+103) | GCA GGT ACC TCC AAC ATC AAG G |
| SEQ ID NO: 202 | |
| H51A(+83+105) | GAG CAG GTA CCT CCA ACA TCA AGG |
| SEQ ID NO: 203 | |
| H51A(+83+107) | CAG AGC AGG TAC CTC CAA CAT CAA GG |
| SEQ ID NO: 204 | |
| H51A(+83+109) | GCC AGA GCA GGT ACC TCC AAC ATC AAG G |
| SEQ ID NO: 205 | |
| H51A(+84+107) | CAG AGC AGG TAC CTC CAA CAT CAA G |
| SEQ ID NO: 206 | |
| H51A(+84+111) | CTG CCA GAG CAG GTA CCT CCA ACA TCA AG |
| SEQ ID NO: 207 | |
| H51A(+84+105) | GAG CAG GTA CCT CCA ACA TCA AG |
| SEQ ID NO: 208 | |
| H51A(+87+109) | GCC AGA GCA GGT ACC TCC AAC ATC |
| SEQ ID NO: 209 | |
| H51A(+93+116) | GAA ATC TGC CAG AGC AGG TAC CTC |
| SEQ ID NO: 210 | |
| H51A(+75+100) | GGT ACC TCC AAC ATC AAG GAA GAT GG |
| SEQ ID NO: 211 | |
| H51A(+74+101) | AGG TAC CTC CAA CAT CAA GGA AGA TGG C |
| SEQ ID NO: 212 | |
| H51A(+74+98) | TAC CTC CAA CAT CAA GGA AGA TGG C |
| SEQ ID NO: 213 | |
| H51A(+74+97) | ACC TCC AAC ATC AAG GAA GAT GGC |
| SEQ ID NO: 214 | |
| H51A(+74+94) | TCC AAC ATC AAG GAA GAT GGC |
| SEQ ID NO: 215 | |
| H51A(+74+93) | CCA ACA TCA AGG AAG ATG GC |
| SEQ ID NO: 216 | |
| H51A(+74+92) | CAA CAT CAA GGA AGA TGG C |
| SEQ ID NO: 217 | |
| H51A(+69+99) | GTA CCT CCA ACA TCA AGG AAG ATG GCA TTT C |
| SEQ ID NO: 218 | |
| H51A(+70+99) | GTA CCT CCA ACA TCA AGG AAG ATG GCA TTT |
| SEQ ID NO: 219 | |
| H51A(+71+99) | GTA CCT CCA ACA TCA AGG AAG ATG GCA TT |
| SEQ ID NO: 220 | |
| H51A(+72+99) | GTA CCT CCA ACA TCA AGG AAG ATG GCA T |
| SEQ ID NO: 221 | |
| H51A(+73+99) | GTA CCT CCA ACA TCA AGG AAG ATG GCA |
| SEQ ID NO: 222 | |
| H51A(+77+99) | GTA CCT CCA ACA TCA AGG AAG AT |
| SEQ ID NO: 223 | |
| H51A(+78+99) | GTA CCT CCA ACA TCA AGG AAG A |
| SEQ ID NO: 224 | |
| H51A(+79+99) | GTA CCT CCA ACA TCA AGG AAG |
| SEQ ID NO: 225 | |
| H51.SA.(−60−36) | GAA GAA AAA GAA AAA TTA GAA ACA C |
| SEQ ID NO: 226 | |
| H51.SA.(−50−26) | AAG GAA AAA AGA AGA AAA AGA AAA A |
| SEQ ID NO: 227 | |
| H51.SA.(−45−21) | GCA AAA AGG AAA AAA GAA GAA AAA G |
| SEQ ID NO: 228 | |
| H51.SA.(−40−16) | TTT TTG CAA AAA GGA AAA AAG AAG A |
| SEQ ID NO: 229 | |
| H51.SA.(−35−11) | TTG GGT TTT TGC AAA AAG GAA AAA A |
| SEQ ID NO: 230 | |
| H51.SA.(−30−6) | ATA TTT TGG GTT TTT GCA AAA AGG A |
| SEQ ID NO: 231 | |
| H51.SA.(−25−1) | CTA AAA TAT TTT GGG TTT TTG CAA A |
| SEQ ID NO: 232 | |
| H51.SA.(−20+5) | AGG AGC TAA AAT ATT TTG GGT TTT T |
| SEQ ID NO: 233 | |
| H51.SA.(−15+10) | TGA GTA GGA GCT AAA ATA TTT TGG G |
| SEQ ID NO: 234 | |
| H51.SA.(−10+15) | CAG TCT GAG TAG GAG CTA AAA TATT |
| SEQ ID NO: 235 | |
| H51.SA.(−5+20) | AGT AAC AGT CTG AGT AGG AGC TAA A |
| SEQ ID NO: 236 | |
| H51.SA.(−1+24) | CCA GAG TAA CAG TCT GAG TAG GAG C |
| SEQ ID NO: 237 | |
| H51.SA.(−65−41) | AAA AGA AAA ATT AGA AAC ACA AGC T |
| SEQ ID NO: 238 | |
| H51.SA.(−70−46) | AAA AAT TAG AAA CAC AAG CTA AAG A |
| SEQ ID NO: 239 | |
| H51.SA.(−75−51) | TTA GAA ACA CAA GCT AAA GAG CCA A |
| SEQ ID NO: 240 | |
| H51.SA.(−80−56) | AAC ACA AGC TAA AGA GCC AAT TTC A |
| SEQ ID NO: 241 | |
| H51.SA.(−85−61) | AAG CTA AAG AGC CAA TTT CAA TAA C |
| SEQ ID NO: 242 | |
| H51.SA.(−90−66) | AAA GAG CCA ATT TCA ATA ACA ATA A |
| SEQ ID NO: 243 | |
| H51.SA.(−95−71) | GCC AAT TTC AAT AAC AAT AAG TCA A |
| SEQ ID NO: 244 | |
| H51.SA.(−100−76) | TTT CAA TAA CAA TAA GTC AAA TTT A |
| SEQ ID NO: 245 | |
| H51A(+1+30) | GTG TCA CCA GAG TAA CAG TCT GAG TAG GAG |
| SEQ ID NO: 246 | |
| H51A(+10+39) | CCA CAG GTT GTG TCA CCA GAG TAA CAG TCT |
| SEQ ID NO: 247 | |
| H51A(+6+35) | AGG TTG TGT CAC CAG AGT AAC AGT CTG AGT |
| SEQ ID NO: 248 | |
| H51A(+49+78) | ATG GCA TTT CTA GTT TGG AGA TGG CAG TTT |
| SEQ ID NO: 249 | |
| H51A(+1+25) | ACC AGA GTA ACA GTC TGA GTA GGA G |
| SEQ ID NO: 250 | |
| H51A(+4+28) | GTC ACC AGA GTA ACA GTC TGA GTA G |
| SEQ ID NO: 251 | |
| H51A(+16+40) | ACC ACA GGT TGT GTC ACC AGA GTA A |
| SEQ ID NO: 252 | |
| H51A(+21+45) | TAG TAA CCA CAG GTT GTG TCA CCA G |
| SEQ ID NO: 253 | |
| H51A(+26+50) | TTC CTT AGT AAC CAC AGG TTG TGT C |
| SEQ ID NO: 254 | |
| H51A(+31+55) | GCA GTT TCC TTA GTA ACC ACA GGT T |
| SEQ ID NO: 255 | |
| H51A(+36+60) | AGA TGG CAG TTT CCT TAG TAA CCA C |
| SEQ ID NO: 256 | |
| H51A(+41+65) | TTT GGA GAT GGC AGT TTC CTT AGT A |
| SEQ ID NO: 257 | |
| H51A(+86+110) | TGC CAG AGC AGG TAC CTC CAA CAT C |
| SEQ ID NO: 258 | |
| H51A(+91+115) | AAA TCT GCC AGA GCA GGT ACC TCC A |
| SEQ ID NO: 259 | |
| H51A(+96+120) | GGT TGA AAT CTG CCA GAG CAG GTA C |
| SEQ ID NO: 260 | |
| H51A(+101+125) | AGC CCG GTT GAA ATC TGC CAG AGC A |
| SEQ ID NO: 261 | |
| H51A(+106+130) | GTC CAA GCC CGG TTG AAA TCT GCC A |
| SEQ ID NO: 262 | |
| H51A(+111+135) | GTT CTG TCC AAG CCC GGT TGA AAT C |
| SEQ ID NO: 263 | |
| H51A(+116+140) | GGT AAG TTC TGT CCA AGC CCG GTT G |
| SEQ ID NO: 264 | |
| H51A(+121+145) | CAG TCG GTA AGT TCT GTC CAA GCC C |
| SEQ ID NO: 265 | |
| H51A(+126+150) | AAA GCC AGT CGG TAA GTT CTG TCC A |
| SEQ ID NO: 266 | |
| H51A(+131+155) | CAG AGA AAG CCA GTC GGTA AGT TCT |
| SEQ ID NO: 267 | |
| H51A(+136+160) | TCA AGC AGA GAA AGC CAG TCG GTA A |
| SEQ ID NO: 268 | |
| H51A(+141+165) | CTT GAT CAA GCA GAG AAA GCC AGT C |
| SEQ ID NO: 269 | |
| H51A(+146+170) | TAT AAC TTGA TCA AGC AGA GAA AGC |
| SEQ ID NO: 270 | |
| H51A(+151+175) | GAT TTT ATA ACT TGA TCA AGC AGA G |
| SEQ ID NO: 271 | |
| H51A(+156+180) | TCT GTG ATT TTA TAA CTT GAT CAA G |
| SEQ ID NO: 272 | |
| H51A(+161+185) | CAC CCT CTG TGA TTT TAT AAC TTG A |
| SEQ ID NO: 273 | |
| H51A(+166+190) | ACC ATC ACC CTC TGT GAT TTT ATA A |
| SEQ ID NO: 274 | |
| H51A(+171+195) | CAC CCA CCA TCA CCC TCT GTG ATT T |
| SEQ ID NO: 275 | |
| H51A(+176+200) | AAG GTC ACC CAC CAT CAC CCT CTG T |
| SEQ ID NO: 276 | |
| H51A(+181+205) | TCC TCA AGG TCA CCC ACC ATC ACC C |
| SEQ ID NO: 277 | |
| H51A(+186+210) | TGA TAT CCT CAA GGT CAC CCA CCA T |
| SEQ ID NO: 278 | |
| H51A(+191+215) | CTC GTT GAT ATC CTC AAG GTC ACC C |
| SEQ ID NO: 279 | |
| H51A(+196+220) | ATC ATC TCG TTG ATA TCC TCA AGG T |
| SEQ ID NO: 280 | |
| H51A(+201+225) | TGA TGA TCA TCT CGT TGA TAT CCT C |
| SEQ ID NO: 281 | |
| H51A(+206+230) | CTG CTT GAT GAT CAT CTC GTT GAT A |
| SEQ ID NO: 282 | |
| H51D(+211−02) | ACC TTC TGC TTG ATG ATC ATC TCG T |
| SEQ ID NO: 283 | |
| H51D(+214−05) | CAT ACC TTC TGC TTG ATG ATC ATC T |
| SEQ ID NO: 284 | |
| H51D(+217−08) | TCT CAT ACC TTC TGC TTG ATG ATC A |
| SEQ ID NO: 285 | |
| H51D(+220−11) | TTT TCT CAT ACC TTC TGC TTG ATG A |
| SEQ ID NO: 286 | |
| H51D(+223−14) | ATT TTT TCT CAT ACC TTC TGC TTG A |
| SEQ ID NO: 287 | |
| H51D(+226−17) | ATC ATT TTT TCT CAT ACC TTC TGC T |
| SEQ ID NO: 288 | |
| H51D(+229−20) | TTT ATC ATT TTT TCT CAT ACC TTC T |
| SEQ ID NO: 289 | |
| H51D(+232−23) | ACT TTT ATC ATT TTT TCT CAT ACC T |
| SEQ ID NO: 290 | |
| H51D(−02−26) | CCA ACT TTT ATC ATT TTT TCT CAT A |
| SEQ ID NO: 291 | |
| H51A(+1+30) | GTG TCA CCA GAG TAA CAG TCT GAG TAG GAG |
| SEQ ID NO: 292 | |

wherein A is

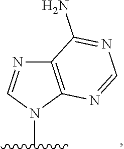 [SMILES: *n1cnc2c(N)ncnc21]

### C is

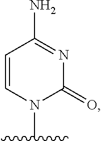 [SMILES: *n1ccc(N)nc1=O]

### G is

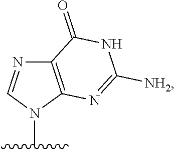 [SMILES: *n1cnc2c(=O)[nH]c(N)nc21]

### and T is

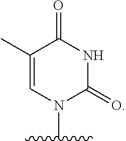 [SMILES: *n1cc(C)c(=O)[nH]c1=O]

In one aspect, the base sequence and annealing site are selected from one of the following:

| Annealing Site | Base Sequence [5′ to 3′] |
|---|---|
| H51A(+66+95) | CTC CAA CAT CAA GGA AGA TGG CAT TTC TAG |
| SEQ ID NO: 293 | |
| H51A(+74+97) | ACC TCC AAC ATC AAG GAA GAT GGC |
| SEQ ID NO: 294 | |
| H51A(+70+99) | GTA CCT CCA ACA TCA AGG AAG ATG GCA TTT |
| SEQ ID NO: 295 | |
| H51A(+72+99) | GTA CCT CCA ACA TCA AGG AAG ATG GCA T |
| SEQ ID NO: 296 | |
| H51A(+68+87) | TCA AGG AAG ATG GCA TTT CT |
| SEQ ID NO: 297 | |
| H51A(+68+87) | UCA AGG AmAGm AmUGm GmCA UUU CU |
| SEQ ID NO: 298 | |
| H53A(+36+60) | GTT GCC TCC GGT TCT GAA GGT GTT C |
| SEQ ID NO: 299 | |
| H53A(+36+60) | GTT G5mC5mC T5mC5mC GGT T5mC T GAA GGT GTT 5mC |
| SEQ ID NO: 300 | |
| H53A(+36+56) | CCT CCG GTT CTG AAG GTG TTC |
| SEQ ID NO: 301 | |
| H53A(+23+47) | CTG AAG GTG TTC TTG TAC TTC ATC C |
| SEQ ID NO: 302 | |
| H53A(+32+56) | CCT CCG GTT CTG AAG GTG TTC TTG T |
| SEQ ID NO: 303 | |
| H53A(+33+60) | GTT GCC TCC GGT TCT GAA GGT GTT CTT G |
| SEQ ID NO: 304 | |
| H53A(+30+59) | TTG CCT CCG GTT CTG AAG GTG TTC TTG TAC |
| SEQ ID NO: 305 | |
| H53A(+39+62) | CTG TTG CCT CCG GTT CTG AAG GTG |
| SEQ ID NO: 306 | |
| H53A(+36+69) | CAT TCA ACT GTT GCC TCC GGT TCT GAA GGT G |
| SEQ ID NO: 307 | |
| H53A(+45+62) | CTG TTG CCT CCG GTT CTG |
| SEQ ID NO: 308 | |
| H45A(−03+19) | CAA TGC CAT CCT GGA GTT CCT G |
| SEQ ID NO: 309 | |
| H45A(−09+25) | GCT GCC CAA TGC CAT CCT GGA GTT CCT GTA AGA T |
| SEQ ID NO: 310 | |
| H45A(−03+25) | GCT GCC CAA TGC CAT CCT GGA GTT CCT G |
| SEQ ID NO: 311 | |
| H45A(−06+25) | GCT GCC CAA TGC CAT CCT GGA GTT CCT GTA A |
| SEQ ID NO: 312 | |
| H45A(−12+19) | CAA TGC CAT CCT GGA GTT CCT GTA AGA TAC C |
| SEQ ID NO: 313 | |
| H45A(−09+19) | CAA TGC CAT CCT GGA GTT CCT GTA AGA T |
| SEQ ID NO: 314 | |
| H45A(−12+16) | TGC CAT CCT GGA GTT CCT GTA AGA TAC C |
| SEQ ID NO: 315 | |
| H45A(−14+25) | GCT GCC CAA TGC CAT CCT GGA GTT CCT GTA AGA TAC CAA |
| SEQ ID NO: 316 | |
| H45A(−08+19) | CAA TGC CAT CCT GGA GTT CCT GTA AGA |
| SEQ ID NO: 317 | |
| HM45A(−07+25) | GCT GCC CAA TGC CAT CCT GGA GTT CCT GTA AG |
| SEQ ID NO: 318 | |
| H45A(−12+22) | GCC CAA TGC CAT CCT GGA GTT CCT GTA AGA TAC C |
| SEQ ID NO: 319 | |
| H45A(−09+22) | GCC CAA TGC CAT CCT GGA GTT CCT GTA AGA T |
| SEQ ID NO: 320 | |
| H45A(−09+30) | TTG CCG CTG CCC AAT GCC ATC CTG GAG TTC CTG TAA GAT |
| SEQ ID NO: 321 | |
| H45A(−06+22) | GCC CAA TGC CAT CCT GGA GTT CCT GTA A |
| SEQ ID NO: 322 | |
| H45A(−06+28) | GCC GCT GCC CAA TGC CAT CCT GGA GTT CCT GTA A |
| SEQ ID NO: 323 | |
| H45A(−03+22) | GCC CAA TGC CAT CCT GGA GTT CCT G |
| SEQ ID NO: 324 | |
| H45A(−03+28) | GCC GCT GCC CAA TGC CAT CCT GGA GTT CCT G |
| SEQ ID NO: 325 | |
| H45A(+9+26) | m5C-G-m5C-T-G-C-m5C-m5C-A-A-T-G-m5C-m5C-A-U-m5C-m5C |
| SEQ ID NO: 326 | |
| H44A(−10+15) | GAT CTG TCA AAT CGC CTG CAG GTA A |
| SEQ ID NO: 327 | |
| H44A(−07+15) | GAT CTG TCA AAT CGC CTG CAG G |
| SEQ ID NO: 328 | |
| H44M(−07+17) | CAG ATC TGT CAA ATC GCC TGC AGG |
| SEQ ID NO: 329 | |
| H44A(−08+15) | GAT CTG TCA AAT CGC CTG CAG GT |
| SEQ ID NO: 330 | |
| H44A(−06+15) | GAT CTG TCA AAT CGC CTG CAG |
| SEQ ID NO: 331 | |
| H44A(−08+17) | CAG ATC TGT CAA ATC GCC TGC AGG T |
| SEQ ID NO: 332 | |
| H44A(−06+17) | CAG ATC TGT CAA ATC GCC TGC AG |
| SEQ ID NO: 333 | |
| H50D(+04−18) | GGG ATC CAG TAT ACT TAC AGG C |
| SEQ ID NO: 334 | |
| H50D(+07−18) | GGG ATC CAG TAT ACT TAC AGG CTC C |
| SEQ ID NO: 335 | |
| H50D(+07−16) | GAT CCA GTA TAC TTA CAG GCT CC |
| SEQ ID NO: 336 | |
| H50D(+07−17) | GGA TCC AGT ATA CTT ACA GGC TCC |
| SEQ ID NO: 337 | |
| H50A(−19+07) | ACT TCC TCT TTA ACA GAA AAG CAT AC |
| SEQ ID NO: 338 | |
| H50D(+07−15) | ATC CAG TAT ACT TAC AGG CTC C |
| SEQ ID NO: 339 | |
| H50A(−02+23) | GAG CTC AGA TCT TCT AAC TTC CTC T |
| SEQ ID NO: 340 | |
| H50D(+06−18) | GGG ATC CAG TAT ACT TAC AGG CTC |
| SEQ ID NO: 341 | |
| H50D(+07−20) | ATG GGA TCC AGT ATA CTT ACA GGC TCC |
| SEQ ID NO: 342 | |
| H52A(−01+24) | CTG TTC CAA ATC CTG CAT TGT TGC C |
| SEQ ID NO: 343 | |

wherein A is

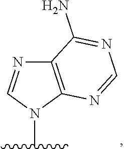 [SMILES: *n1cnc2c(N)ncnc21]

### C is

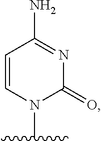 [SMILES: *n1ccc(N)nc1=O]

### G is

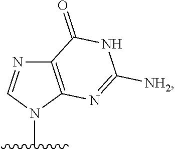 [SMILES: *n1cnc2c(=O)[nH]c(N)nc21]

### T is

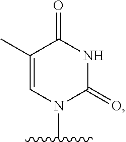 [SMILES: *n1cc(C)c(=O)[nH]c1=O]

### U is

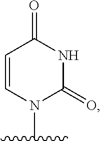 [SMILES: *n1ccc(=O)[nH]c1=O]

Gm is methylated guanine, Am is methylated adenine, and m5C is

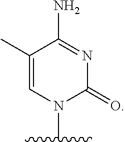 [SMILES: *n1cc(C)c(N)nc1=O]

In some embodiments, an antisense oligomer conjugate of Formula (XI) is an HCl (hydrochloric acid) salt thereof. In certain embodiments, m is 5 and the HCl salt is a ·5HCl salt. In certain embodiments, m is 4 and the HCl salt is a ·4HCl salt. In certain embodiments, m is 3 and the HCl salt is a ·3HCl salt. In certain embodiments, m is 2 and the HCl salt is a ·2HCl salt. In certain embodiments, m is 1 and the HCl salt is a ·HCl salt.

In another aspect, the disclosure provides antisense oligomers of Formula (XIA) or Formula (XIB):

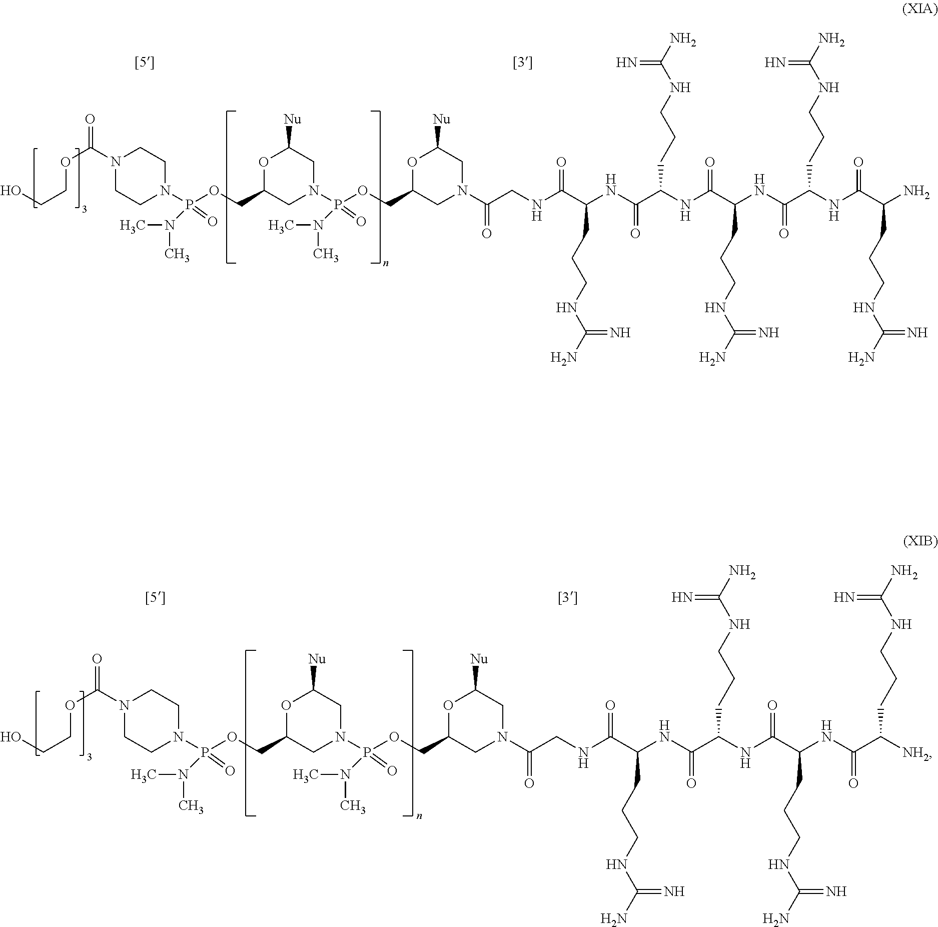

or a pharmaceutically acceptable salt thereof, such as a HCl-salt, where each Nu from 1 to (n+1) and 5′ to 3′ corresponds to the nucleobases in the following sequences:

| Annealing Site | Sequence [5′ to 3′] |
|---|---|
| H51A(+61+90) SEQ ID NO: 344 | ACA TCA AGG AAG ATG GCA TTT CTA GTT TGG |
| H51D(+16−07) SEQ ID NO: 345 | CTC ATA CCT TCT GCT TGA TGA TC |
| H50D(+103+127) SEQ ID NO: 346 | GGG ATC CAG TAT ACT TAC AGG CTC C |
| H51A(+81+105) SEQ ID NO: 347 | GAG CAG GTA CCT CCA ACA TCA AGG AA |
| H51A(+71+100) SEQ ID NO: 348 | GGT ACC TCC AAC ATC AAG GAA GAT GGC ATT |
| H51A(+48+73) SEQ ID NO: 349 | ATT TCT AGT TTG GAG ATG GCA GTT TC |
| H51A(+59+84) SEQ ID NO: 350 | GGA AGA TGG CAT TTC TAG TTT GGA G |
| H51A(+64+88) SEQ ID NO: 351 | CAT CAA GGA AGA TGG CAT TTC TAG TT |
| H51A(+89+113) SEQ ID NO: 352 | ATC TGC CAG AGC AGG TAC CTC CAA C |
| H51A(+49+68) SEQ ID NO: 353 | TAG TTT GGA GAT GGC AGT TT |
| H51A(+64+83) SEQ ID NO: 354 | GGA AGA TGG CAT TTC TAG TT |
| H51A(+80+98) SEQ ID NO: 355 | TAC CTC CAA CAT CAA GGA AG |
| H51A(+94+113) SEQ ID NO: 356 | ATC TGC CAG AGC AGG TAC CT |
| H51A(+109+128) SEQ ID NO: 357 | CCA AGC CCG GTT GAA ATC TG |
| H51A(+61+82) SEQ ID NO: 358 | GAA GAT GGC ATT TCT AGT TTG G |
| H51A(+61+83) SEQ ID NO: 359 | GGA AGA TGG CAT TTC TAG TTT GG |
| H51A(+61+89) SEQ ID NO: 360 | CAT CAA GGA AGA TGG CAT TTC TAG TTT GG |
| H51A(+66+89) SEQ ID NO: 361 | CAT CAA GGA AGA TGG CAT TTC TAG |
| H51A(+66+93) SEQ ID NO: 362 | CCA ACA TCA AGG AAG ATG GCA TTT CTA G |
| H51A(+69+92) SEQ ID NO: 363 | CAA CAT CAA GGA AGA TGG CAT TTC |
| H51A(+69+96) SEQ ID NO: 364 | CCT CCA ACA TCA AGG AAG ATG GCA TTT C |
| H51A(+74+96) SEQ ID NO: 365 | CCT CCA ACA TCA AGG AAG ATG GC |
| H51A(+74+99) SEQ ID NO: 366 | GTA CCT CCA ACA TCA AGG AAG ATG GC |
| H51A(+74+100) SEQ ID NO: 367 | GGT ACC TCC AAC ATC AAG GAA GAT GGC |
| H51A(+74+102) SEQ ID NO: 368 | CAG GTA CCT CCA ACA TCA AGG AAG ATG GC |
| H51A(+74+103) SEQ ID NO: 369 | GCA GGT ACC TCC AAC ATC AAG GAA GAT GGC |
| H51A(+75+96) SEQ ID NO: 370 | CCT CCA ACA TCA AGG AAG ATG G |
| H51A(+75+99) SEQ ID NO: 371 | GTA CCT CCA ACA TCA AGG AAG ATG G |
| H51A(+76+99) SEQ ID NO: 372 | GTA CCT CCA ACA TCA AGG AAG ATG |
| H51A(+76+105) SEQ ID NO: 373 | GAG CAG GTA CCT CCA ACA TCA AGG AAG ATG |
| H51A(+80+103) SEQ ID NO: 374 | GCA GGT ACC TCC AAC ATC AAG GAA G |
| H51A(+80+105) SEQ ID NO: 375 | GAG CAG GTA CCT CCA ACA TCA AGG AAG |
| H51A(+80+107) SEQ ID NO: 376 | CAG AGC AGG TAC CTC CAA CAT CAA GGA AG |
| H51A(+80+108) SEQ ID NO: 377 | CCA GAG CAG GTA CCT CCA ACA TCA AGG AAG |
| H51A(+83+103) SEQ ID NO: 378 | GCA GGT ACC TCC AAC ATC AAGG |
| H51A(+83+105) SEQ ID NO: 379 | GAG CAG GTA CCT CCA ACA TCA AGG |
| H51A(+83+107) SEQ ID NO: 380 | CAG AGC AGG TAC CTC CAA CAT CAA GG |
| H51A(+83+109) SEQ ID NO: 381 | GCC AGA GCA GGT ACC TCC AAC ATC AAG G |
| H51A(+84+107) SEQ ID NO: 382 | CAG AGC AGG TAC CTC CAA CAT CAA G |
| H51A(+84+111) SEQ ID NO: 383 | CTG CCA GAG CAG GTA CCT CCA ACA TCA AG |
| H51A(+84+105) SEQ ID NO: 384 | GAG CAG GTA CCT CCA ACA TCA AG |
| H51A(+87+109) SEQ ID NO: 385 | GCC AGA GCA GGT ACC TCC AAC ATC |
| H51A(+93+116) SEQ ID NO: 386 | GAA ATC TGC CAG AGC AGG TAC CTC |
| H51A(+75+100) SEQ ID NO: 387 | GGT ACC TCC AAC ATC AAG GAA GAT GG |
| H51A(+74+101) SEQ ID NO: 388 | AGG TAC CTC CAA CAT CAA GGA AGA TGG C |
| H51A(+74+98) SEQ ID NO: 389 | TAC CTC CAA CAT CAA GGA AGA TGG C |
| H51A(+74+97) SEQ ID NO: 390 | ACC TCC AAC ATC AAG GAA GAT GGC |
| H51A(+74+94) SEQ ID NO: 391 | TCC AAC ATC AAG GAA GAT GGC |
| H51A(+74+93) SEQ ID NO: 392 | CCA ACA TCA AGG AAG ATG GC |
| H51A(+74+92) SEQ ID NO: 393 | CAA CAT CAA GGA AGA TGG C |
| H51A(+69+99) SEQ ID NO: 394 | GTA CCT CCA ACA TCA AGG AAG ATG GCA TTT C |
| H51A(+70+99) SEQ ID NO: 395 | GTA CCT CCA ACA TCA AGG AAG ATG GCA TTT |
| H51A(+71+99) SEQ ID NO: 396 | GTA CCT CCA ACA TCA AGG AAG ATG GCA TT |
| H51A(+72+99) SEQ ID NO: 397 | GTA CCT CCA ACA TCA AGG AAG ATG GCA T |
| H51A(+73+99) SEQ ID NO: 398 | GTA CCT CCA ACA TCA AGG AAG ATG GCA |
| H51A(+77+99) SEQ ID NO: 399 | GTA CCT CCA ACA TCA AGG AAG AT |
| H51A(+78+99) SEQ ID NO: 400 | GTA CCT CCA ACA TCA AGG AAG A |
| H51A(+79+99) SEQ ID NO: 401 | GTA CCT CCA ACA TCA AGG AAG |
| H51.SA.(−60−36) SEQ ID NO: 402 | GAA GAA AAA GAA AAA TTA GAA ACA C |
| H51.SA.(−50−26) SEQ ID NO: 403 | AAG GAA AAA AGA AGA AAA AGA AAA A |
| H51.SA.(−45−21) SEQ ID NO: 404 | GCA AAA AGG AAA AAA GAA GAA AAA G |
| H51.SA.(−40−16) SEQ ID NO: 405 | TTT TTG CAA AAA GGA AAA AAG AAG A |
| H51.SA.(−35−11) SEQ ID NO: 406 | TTG GGT TTT TGC AAA AAG GAA AAA A |
| H51.SA.(−30−6) SEQ ID NO: 407 | ATA TTT TGG GTT TTT GCA AAA AGG A |
| H51.SA.(−25−1) SEQ ID NO: 408 | CTA AAA TAT TTT GGG TTT TTG CAA A |
| H51.SA.(−20+5) SEQ ID NO: 409 | AGG AGC TAA AAT ATT TTG GGT TTT T |
| H51.SA.(−15+10) SEQ ID NO: 410 | TGA GTA GGA GCT AAA ATA TTT TGG G |
| H51.SA.(−10+15) SEQ ID NO: 411 | CAG TCT GAG TAG GAG CTA AAA TAT T |
| H51.SA.(−5+20) SEQ ID NO: 412 | AGT AAC AGT CTG AGT AGG AGC TAA A |
| H51.SA.(−1+24) SEQ ID NO: 413 | CCA GAG TAA CAG TCT GAG TAG GAG C |
| H51.SA.(−65−41) SEQ ID NO: 414 | AAA AGA AAA ATT AGA AAC ACA AGC T |
| H51.SA.(−70−46) SEQ ID NO: 415 | AAA AAT TAG AAA CAC AAG CTA AAG A |
| H51.SA.(−75−51) SEQ ID NO: 416 | TTA GAA ACA CAA GCT AAA GAG CCA A |
| H51.SA.(−80−56) SEQ ID NO: 417 | AAC ACA AGC TAA AGA GCC AAT TTC A |
| H51.SA.(−85−61) SEQ ID NO: 418 | AAG CTA AAG AGC CAA TTT CAA TAA C |
| H51.SA.(−90−66) SEQ ID NO: 419 | AAA GAG CCA ATT TCA ATA ACA ATA A |
| H51.SA.(−95−71) SEQ ID NO: 420 | GCC AAT TTC AAT AAC AAT AAG TCA A |
| H51.SA.(−100−76) SEQ ID NO: 421 | TTT CAA TAA CAA TAA GTC AAA TTT A |
| H51A(+1+30) SEQ ID NO: 422 | GTG TCA CCA GAG TAA CAG TCT GAG TAG GAG |
| H51A(+10+39) SEQ ID NO: 423 | CCA CAG GTT GTG TCA CCA GAG TAA CAG TCT |
| H51A(+6+35) SEQ ID NO: 424 | AGG TTG TGT CAC CAG AGT AAC AGT CTG AGT |
| H51A(+49+78) SEQ ID NO: 425 | ATG GCA TTT CTA GTT TGG AGA TGG CAG TTT |
| H51A(+1+25) SEQ ID NO: 426 | ACC AGA GTA ACA GTC TGA GTA GGA G |
| H51A(+4+28) SEQ ID NO: 427 | GTC ACC AGA GTA ACA GTC TGA GTA G |
| H51A(+16+40) SEQ ID NO: 428 | ACC ACA GGT TGT GTC ACC AGA GTA A |
| H51A(+21+45) SEQ ID NO: 429 | TAG TAA CCA CAG GTT GTG TCA CCA G |
| H51A(+26+50) SEQ ID NO: 430 | TTC CTT AGT AAC CAC AGG TTG TGT C |
| H51A(+31+55) SEQ ID NO: 431 | GCA GTT TCC TTA GTA ACC ACA GGT T |
| H51A(+36+60) SEQ ID NO: 432 | AGA TGG CAG TTT CCT TAG TAA CCA C |
| H51A(+41+65) SEQ ID NO: 433 | TTT GGA GAT GGC AGT TTC CTT AGT A |
| H51A(+86+110) SEQ ID NO: 434 | TGC CAG AGC AGG TAC CTC CAA CAT C |
| H51A(+91+115) SEQ ID NO: 435 | AAA TCT GCC AGA GCA GGT ACC TCC A |
| H51A(+96+120) SEQ ID NO: 436 | GGT TGA AAT CTG CCA GAG CAG GTA C |
| H51A(+101+125) SEQ ID NO: 437 | AGC CCG GTT GAA ATC TGC CAG AGC A |
| H51A(+106+130) SEQ ID NO: 438 | GTC CAA GCC CGG TTG AAA TCT GCC A |
| H51A(+111+135) SEQ ID NO: 439 | GTT CTG TCC AAG CCC GGT TGA AAT C |
| H51A(+116+140) SEQ ID NO: 440 | GGT AAG TTC TGT CCA AGC CCG GTT G |
| H51A(+121+145) SEQ ID NO: 441 | CAG TCG GTA AGT TCT GTC CAA GCC C |
| H51A(+126+150) SEQ ID NO: 442 | AAA GCC AGT CGG TAA GTT CTG TCC A |
| H51A(+131+155) SEQ ID NO: 443 | CAG AGA AAG CCA GTC GGTA AGT TCT |
| H51A(+136+160) SEQ ID NO: 444 | TCA AGC AGA GAA AGC CAG TCG GTA A |
| H51A(+141+165) SEQ ID NO: 445 | CTT GAT CAA GCA GAG AAA GCC AGT C |
| H51A(+146+170) SEQ ID NO: 446 | TAT AAC TTGA TCA AGC AGA GAA AGC |
| H51A(+151+175) SEQ ID NO: 447 | GAT TTT ATA ACT TGA TCA AGC AGA G |
| H51A(+156+180) SEQ ID NO: 448 | TCT GTG ATT TTA TAA CTT GAT CAA G |
| H51A(+161+185) SEQ ID NO: 449 | CAC CCT CTG TGA TTT TAT AAC TTG A |
| H51A(+166+190) SEQ ID NO: 450 | ACC ATC ACC CTC TGT GAT TTT ATA A |
| H51A(+171+195) SEQ ID NO: 451 | CAC CCA CCA TCA CCC TCT GTG ATT T |
| H51A(+176+200) SEQ ID NO: 452 | AAG GTC ACC CAC CAT CAC CCT CTG T |
| H51A(+181+205) SEQ ID NO: 453 | TCC TCA AGG TCA CCC ACC ATC ACC C |
| H51A(+186+210) SEQ ID NO: 454 | TGA TAT CCT CAA GGT CAC CCA CCA T |
| H51A(+191+215) SEQ ID NO: 455 | CTC GTT GAT ATC CTC AAG GTC ACC C |
| H51A(+196+220) SEQ ID NO: 456 | ATC ATC TCG TTG ATA TCC TCA AGG T |
| H51A(+201+225) SEQ ID NO: 457 | TGA TGA TCA TCT CGT TGA TAT CCT C |
| H51A(+206+230) SEQ ID NO: 458 | CTG CTT GAT GAT CAT CTC GTT GAT A |
| H51D(+211−02) SEQ ID NO: 459 | ACC TTC TGC TTG ATG ATC ATC TCG T |
| H51D(+214−05) SEQ ID NO: 460 | CAT ACC TTC TGC TTG ATG ATC ATC T |
| H51D(+217−08) SEQ ID NO: 461 | TCT CAT ACC TTC TGC TTG ATG ATC A |
| H51D(+220−11) SEQ ID NO: 462 | TTT TCT CAT ACC TTC TGC TTG ATG A |
| H51D(+223−14) SEQ ID NO: 463 | ATT TTT TCT CAT ACC TTC TGC TTG A |
| H51D(+226−17) SEQ ID NO: 464 | ATC ATT TTT TCT CAT ACC TTC TGC T |
| H51D(+229−20) SEQ ID NO: 465 | TTT ATC ATT TTT TCT CAT ACC TTC T |
| H51D(+232−23) SEQ ID NO: 466 | ACT TTT ATC ATT TTT TCT CAT ACC T |
| H51D(−02−26) SEQ ID NO: 467 | CCA ACT TTT ATC ATT TTT TCT CAT A |
| H51A(+1+30) SEQ ID NO: 468 | GTG TCA CCA GAG TAA CAG TCT GAG TAG GAG |

wherein A is

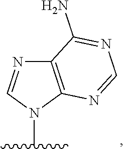 [SMILES: *n1cnc2c(N)ncnc21]

### C is

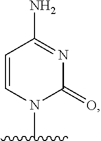 [SMILES: *n1ccc(N)nc1=O]

### G is

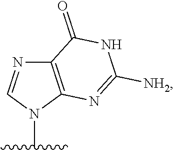 [SMILES: *n1cnc2c(=O)[nH]c(N)nc21]

### and T is

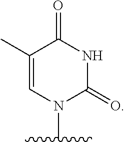 [SMILES: *n1cc(C)c(=O)[nH]c1=O]

In one aspect, the base sequence and annealing site are selected from one of the following:

| Annealing Site | Base Sequence [5′ to 3′] |
|---|---|
| H51A(+66+95) | CTC CAA CAT CAA GGA AGA TGG CAT TTC TAG |
| SEQ ID NO: 469 | |
| H51A(+74+97) | ACC TCC AAC ATC AAG GAA GAT GGC |
| SEQ ID NO: 470 | |
| H51A(+70+99) | GTA CCT CCA ACA TCA AGG AAG ATG GCA TTT |
| SEQ ID NO: 471 | |
| H51A(+72+99) | GTA CCT CCA ACA TCA AGG AAG ATG GCA T |
| SEQ ID NO: 472 | |
| H51A(+68+87) | TCA AGG AAG ATG GCA TTT CT |
| SEQ ID NO: 473 | |
| H51A(+68+87) | UCA AGG AmAGm AmUGm GmCA UUU CU |
| SEQ ID NO: 474 | |
| H53A(+36+60) | GTT GCC TCC GGT TCT GAA GGT GTT C |
| SEQ ID NO: 475 | |
| H53A(+36+60) | GTT G5mC5mC T5mC5mC GGT T5mC T GAA GGT GTT 5mC |
| SEQ ID NO: 476 | |
| H53A(+36+56) | CCT CCG GTT CTG AAG GTG TTC |
| SEQ ID NO: 477 | |
| H53A(+23+47) | CTG AAG GTG TTC TTG TAC TTC ATC C |
| SEQ ID NO: 478 | |
| H53A(+32+56) | CCT CCG GTT CTG AAG GTG TTC TTG T |
| SEQ ID NO: 479 | |
| H53A(+33+60) | GTT GCC TCC GGT TCT GAA GGT GTT CTT G |
| SEQ ID NO: 480 | |
| H53A(+30+59) | TTG CCT CCG GTT CTG AAG GTG TTC TTG TAC |
| SEQ ID NO: 481 | |
| H53A(+39+62) | CTG TTG CCT CCG GTT CTG AAG GTG |
| SEQ ID NO: 482 | |
| H53A(+36+69) | CAT TCA ACT GTT GCC TCC GGT TCT GAA GGT G |
| SEQ ID NO: 483 | |
| H53A(+45+62) | CTG TTG CCT CCG GTT CTG |
| SEQ ID NO: 484 | |
| H45A(−03+19) | CAA TGC CAT CCT GGA GTT CCT G |
| SEQ ID NO: 485 | |
| H45A(−09+25) | GCT GCC CAA TGC CAT CCT GGA GTT CCT GTA AGA T |
| SEQ ID NO: 486 | |
| H45A(−03+25) | GCT GCC CAA TGC CAT CCT GGA GTT CCT G |
| SEQ ID NO: 487 | |
| H45A(−12+19) | CAA TGC CAT CCT GGA GTT CCT GTA AGA TAC C |
| SEQ ID NO: 488 | |
| H45A(−06+25) | GCT GCC CAA TGC CAT CCT GGA GTT CCT GTA A |
| SEQ ID NO: 489 | |
| H45A(−09+19) | CAA TGC CAT CCT GGA GTT CCT GTA AGA T |
| SEQ ID NO: 490 | |
| H45A(−12+16) | TGC CAT CCT GGA GTT CCT GTA AGA TAC C |
| SEQ ID NO: 491 | |
| H45A(−14+25) | GCT GCC CAA TGC CAT CCT GGA GTT CCT GTA AGA TAC CAA |
| SEQ ID NO: 492 | |
| H45A(−08+19) | CAA TGC CAT CCT GGA GTT CCT GTA AGA |
| SEQ ID NO: 493 | |
| HM45A(−07+25) | GCT GCC CAA TGC CAT CCT GGA GTT CCT GTA AG |
| SEQ ID NO: 494 | |
| H45A(−12+22) | GCC CAA TGC CAT CCT GGA GTT CCT GTA AGA TAC C |
| SEQ ID NO: 495 | |
| H45A(−09+22) | GCC CAA TGC CAT CCT GGA GTT CCT GTA AGA T |
| SEQ ID NO: 496 | |
| H45A(−09+30) | TTG CCG CTG CCC AAT GCC ATC CTG GAG TTC CTG TAA GAT |
| SEQ ID NO: 497 | |
| H45A(−06+22) | GCC CAA TGC CAT CCT GGA GTT CCT GTA A |
| SEQ ID NO: 498 | |
| H45A(−06+28) | GCC GCT GCC CAA TGC CAT CCT GGA GTT CCT GTA A |
| SEQ ID NO: 499 | |
| H45A(−03+22) | GCC CAA TGC CAT CCT GGA GTT CCT G |
| SEQ ID NO: 500 | |
| H45A(−03+28) | GCC GCT GCC CAA TGC CAT CCT GGA GTT CCT G |
| SEQ ID NO: 501 | |
| H45A(+9+26) | m5CGm5C TGC m5Cm5CA ATG m5Cm5CA Um5Cm5C |
| SEQ ID NO: 502 | |
| H44A(−10+15) | GAT CTG TCA AAT CGC CTG CAG GTA A |
| SEQ ID NO: 503 | |
| H44A(−07+15) | GAT CTG TCA AAT CGC CTG CAG G |
| SEQ ID NO: 504 | |
| H44M(−07+17) | CAG ATC TGT CAA ATC GCC TGC AGG |
| SEQ ID NO: 505 | |
| H44A(−08+15) | GAT CTG TCA AAT CGC CTG CAG GT |
| SEQ ID NO: 506 | |
| H44A(−06+15) | GAT CTG TCA AAT CGC CTG CAG |
| SEQ ID NO: 507 | |
| H44A(−08+17) | CAG ATC TGT CAA ATC GCC TGC AGG T |
| SEQ ID NO: 508 | |
| H44A(−06+17) | CAG ATC TGT CAA ATC GCC TGC AG |
| SEQ ID NO: 509 | |
| H50D(+04−18) | GGG ATC CAG TAT ACT TAC AGG C |
| SEQ ID NO: 510 | |
| H50D(+07−18) | GGG ATC CAG TAT ACT TAC AGG CTC C |
| SEQ ID NO: 511 | |
| H50D(+07−16) | GAT CCA GTA TAC TTA CAG GCT CC |
| SEQ ID NO: 512 | |
| H50D(+07−17) | GGA TCC AGT ATA CTT ACA GGC TCC |
| SEQ ID NO: 513 | |
| H50A(−19+07) | ACT TCC TCT TTA ACA GAA AAG CAT AC |
| SEQ ID NO: 514 | |
| H50D(+07−15) | ATC CAG TAT ACT TAC AGG CTC C |
| SEQ ID NO: 515 | |
| H50A(−02+23) | GAG CTC AGA TCT TCT AAC TTC CTC T |
| SEQ ID NO: 516 | |
| H50D(+06−18) | GGG ATC CAG TAT ACT TAC AGG CTC |
| SEQ ID NO: 517 | |
| H50D(+07−20) | ATG GGA TCC AGT ATA CTT ACA GGC TCC |
| SEQ ID NO: 518 | |
| H52A(−01+24) | CTG TTC CAA ATC CTG CAT TGT TGC C |
| SEQ ID NO: 519 | |

wherein A is

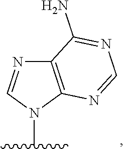 [SMILES: *n1cnc2c(N)ncnc21]

### C is

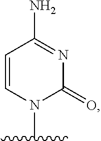 [SMILES: *n1ccc(N)nc1=O]

### G is

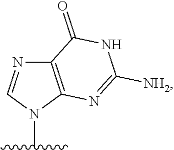 [SMILES: *n1cnc2c(=O)[nH]c(N)nc21]

### T is

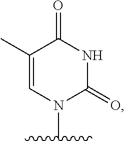 [SMILES: *n1cc(C)c(=O)[nH]c1=O]

### U is

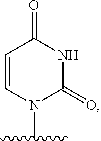 [SMILES: *n1ccc(=O)[nH]c1=O]

Gm is methylated guanine, Am is methylated adenine, and m5C is

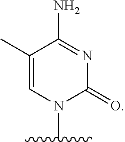 [SMILES: *n1cc(C)c(N)nc1=O]

### Nucleobase Modifications and Substitutions

In certain embodiments, antisense oligomer conjugates of the disclosure are composed of RNA nucleobases and DNA nucleobases (often referred to in the art simply as “base”). RNA bases are commonly known as adenine (A), uracil (U), cytosine (C) and guanine (G). DNA bases are commonly known as adenine (A), thymine (T), cytosine (C) and guanine (G). In various embodiments, antisense oligomer conjugates of the disclosure are composed of cytosine (C), guanine (G), thymine (T), adenine (A), 5-methylcytosine (5mC), uracil (U), and hypoxanthine (I).

In certain embodiments, one or more RNA bases or DNA bases in an oligomer may be modified or substituted with a base other than a RNA base or DNA base. Oligomers containing a modified or substituted base include oligomers in which one or more purine or pyrimidine bases most commonly found in nucleic acids are replaced with less common or non-natural bases.

Purine bases comprise a pyrimidine ring fused to an imidazole ring, as described by the following general formula.

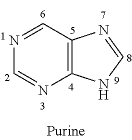

Adenine and guanine are the two purine nucleobases most commonly found in nucleic acids. Other naturally-occurring purines include, but not limited to, N⁶-methyladenine, N²-methylguanine, hypoxanthine, and 7-methylguanine.

Pyrimidine bases comprise a six-membered pyrimidine ring as described by the following general formula.

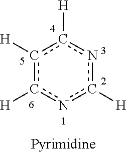

Cytosine, uracil, and thymine are the pyrimidine bases most commonly found in nucleic acids. Other naturally-occurring pyrimidines include, but not limited to, 5-methylcytosine, 5-hydroxymethylcytosine, pseudouracil, and 4-thiouracil. In one embodiment, the oligomers described herein contain thymine bases in place of uracil.

Other suitable bases include, but are not limited to: 2,6-diaminopurine, orotic acid, agmatidine, lysidine, 2-thiopyrimidines (e.g. 2-thiouracil, 2-thiothymine), G-clamp and its derivatives, 5-substituted pyrimidines (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof, N²-cyclopentylguanine (cPent-G), N²-cyclopentyl-2-aminopurine (cPent-AP), and N²-propyl-2-aminopurine (Pr-AP), pseudouracil, or derivatives thereof, and degenerate or universal bases, like 2,6-difluorotoluene or absent bases like a basic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G, and Super T can be found in U.S. Pat. No. 6,683,173 (Epoch Biosciences), which is incorporated here entirely by reference. cPent-G, cPent-AP, and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (WO 2009127230, incorporated here in its entirety by reference).

Certain nucleobases are particularly useful for increasing the binding affinity of the antisense oligomer conjugates of the disclosure. These include 5-substituted pyrimidines, 6-azapyrimidines, and N-2, N-6, and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications. Additional exemplary modified nucleobases include those wherein at least one hydrogen atom of the nucleobase is replaced with fluorine.

### Pharmaceutically Acceptable Salts of Antisense Oligomer Conjugates of the Disclosure

Certain embodiments of antisense oligomer conjugates described herein may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of antisense oligomer conjugates of the present disclosure. These salts can be prepared in situ in the administration vehicle or the dosage form manufacturing process, or by separately reacting a purified antisense oligomer conjugate of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject antisense oligomer conjugates include the conventional nontoxic salts or quaternary ammonium salts of the antisense oligomer conjugates, e.g., from non-toxic organic or inorganic acids. For example, such conventional nontoxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the antisense oligomer conjugates of the present disclosure may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of antisense oligomer conjugates of the present disclosure. These salts can likewise be prepared in situ in the administration vehicle or the dosage form manufacturing process, or by separately reacting the purified antisense oligomer conjugate in its free acid form with a suitable base, such as the hydroxide, carbonate, or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

### Formulations and Modes of Administration

In certain embodiments, the present disclosure provides formulations or pharmaceutical compositions suitable for the therapeutic delivery of antisense oligomer conjugates, as described herein. Pharmaceutical formulations comprising antisense oligomers conjugated to cell-penetrating peptides (e.g., PPMOs) for DMD have been described in, e.g., U.S. Pat. No. 10,888,578, the disclosure of which is incorporated by reference herein. In certain embodiments, the present disclosure provides pharmaceutically acceptable compositions that comprise a therapeutically-effective amount of one or more of the antisense oligomer conjugates described herein, formulated together with one or more pharmaceutically acceptable carriers (additives) and/or diluents. While it is possible for an antisense oligomer conjugate of the present disclosure to be administered alone, it is preferable to administer the antisense oligomer conjugate as a pharmaceutical formulation (composition). In an embodiment, the antisense oligomer conjugate of the formulation is according to Formula (I).

In another aspect, the disclosure provides pharmaceutical compositions that include the antisense oligomers, or a pharmaceutically acceptable salt thereof, of the disclosure, and a pharmaceutically acceptable carrier. In some embodiments, the pharmaceutically acceptable carrier is a saline solution that includes a phosphate buffer.

The phrase “pharmaceutically acceptable” means the substance or composition must be compatible, chemically and/or toxicologically, with the other ingredients comprising a formulation, and/or the subject being treated therewith.

The phrase “pharmaceutically-acceptable carrier” as used herein means a non-toxic, inert solid, semi-solid or liquid filler, diluent, encapsulating material, or formulation auxiliary of any type. Some examples of materials which can serve as pharmaceutically acceptable carriers are: sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening agents; flavoring agents; perfuming agents; preservatives; and antioxidants; according to the judgment of the formulator.

Methods for the delivery of nucleic acid molecules, which can be applicable to the antisense oligomer conjugates of the present disclosure, are described, for example, in: Akhtar et al., 1992, Trends Cell Bio., 2:139; Delivery Strategies for Antisense Oligonucleotide Therapeutics, ed. Akhtar, 1995, CRC Press; and Sullivan et al., PCT WO 94/02595. These and other protocols can be utilized for the delivery of virtually any nucleic acid molecule, including the antisense oligomer conjugates of the present disclosure.

The pharmaceutical compositions of the present disclosure may be specially formulated for administration in solid or liquid form, including those adapted for the following: (1) oral administration, for example, drenches (aqueous or non-aqueous solutions or suspensions), tablets (targeted for buccal, sublingual, or systemic absorption), boluses, powders, granules, pastes for application to the tongue; (2) parenteral administration, for example, by subcutaneous, intramuscular, intravenous, or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream, or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; and (22) other non-toxic compatible substances employed in pharmaceutical formulations.

Additional non-limiting examples of agents suitable for formulation with the antisense oligomer conjugates of the instant disclosure include: PEG conjugated nucleic acids; phospholipid conjugated nucleic acids; nucleic acids containing lipophilic moieties; phosphorothioates; P-glycoprotein inhibitors (such as Pluronic P85) which can enhance entry of drugs into various tissues; biodegradable polymers, such as poly (D,L-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The disclosure also features the use of the composition comprising surface-modified liposomes containing poly(ethylene glycol) (“PEG”) lipids (PEG-modified, branched and unbranched or combinations thereof, or long-circulating liposomes or stealth liposomes). Oligomer conjugates of the disclosure can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the present disclosure includes antisense oligomer conjugate pharmaceutical compositions prepared for delivery as described in U.S. Pat. Nos. 6,692,911; 7,163,695; and 7,070,807. In this regard, in one embodiment, the present disclosure provides an antisense oligomer conjugate of the present disclosure in a composition comprising copolymers of lysine and histidine (HK) (as described in U.S. Pat. Nos. 7,163,695; 7,070,807; and 6,692,911) either alone or in combination with PEG (e.g., branched or unbranched PEG or a mixture of both), in combination with PEG and a targeting moiety, or any of the foregoing in combination with a crosslinking agent. In certain embodiments, the present disclosure provides antisense oligomer conjugates in pharmaceutical compositions comprising gluconic-acid-modified polyhistidine or gluconylated-polyhistidine/transferrin-polylysine. One skilled in the art will also recognize that amino acids with properties similar to His and Lys may be substituted within the composition.

Wetting agents, emulsifiers and lubricants (such as sodium lauryl sulfate and magnesium stearate), coloring agents, release agents, coating agents, sweetening agents, flavoring agents, perfuming agents, preservatives, and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these formulations or pharmaceutical compositions include the step of bringing into association an antisense oligomer conjugate of the present disclosure with the carrier and, optionally, one or more accessory ingredients. In general, the formulations are prepared by uniformly and intimately bringing into association an antisense oligomer conjugate of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an antisense oligomer conjugate of the present disclosure as an active ingredient. An antisense oligomer conjugate of the present disclosure may also be administered as a bolus, electuary, or paste.

Pharmaceutical compositions suitable for parenteral administration may comprise one or more oligomer conjugates of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In an embodiment, the antisense oligomer conjugate of the pharmaceutical composition is according to Formula (I).

These pharmaceutical compositions may also contain adjuvants such as preservatives, wetting agents, emulsifying agents, and dispersing agents. Prevention of the action of microorganisms upon the subject oligomer conjugates may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject oligomer conjugates in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also prepared by entrapping the drug in liposomes or microemulsions that are compatible with body tissues.

When the antisense oligomer conjugates of the present disclosure are administered as pharmaceuticals, to humans and animals, they can be given per se or as a pharmaceutical composition containing, for example, 0.1 to 99% (more preferably, 10 to 30%) of the antisense oligomer conjugate in combination with a pharmaceutically acceptable carrier.

The formulations or preparations of the present disclosure may be given orally, parenterally, topically, or rectally. They are typically given in forms suitable for each administration route. For example, they are administered in tablets or capsule form, by injection, inhalation, eye lotion, ointment, suppository, or infusion; topically by lotion or ointment; or rectally by suppositories.

Regardless of the route of administration selected, the antisense oligomer conjugates of the present disclosure, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular antisense oligomer conjugate of the present disclosure employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the antisense oligomer conjugates of the disclosure employed in the pharmaceutical composition at levels lower than that required in order to achieve the desired therapeutic effect and gradually increase the dosage until the desired effect is achieved. In general, a suitable daily dose of an antisense oligomer conjugate of the disclosure will be that amount of the antisense oligomer conjugate which is the lowest dose effective to produce a therapeutic effect. Such an effective dose will generally depend upon the factors described herein. Generally, oral, intravenous, intracerebroventricular and subcutaneous doses of the antisense oligomer conjugates of this disclosure for a patient, when used for the indicated effects, will range from about 0.0001 to about 100 mg per kilogram of body weight per day.

In some embodiments, the antisense oligomer conjugates of the present disclosure are administered in doses generally from about 10-160 mg/kg or 20-160 mg/kg. In some cases, doses of greater than 160 mg/kg may be necessary. In some embodiments, doses for i.v. administration are from about 0.5 mg to 160 mg/kg. In some embodiments, the antisense oligomer conjugates are administered at doses of about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg. In some embodiments, the antisense oligomer conjugates are administered at doses of about 10 mg/kg, 11 mg/kg, 12 mg/kg, 15 mg/kg, 18 mg/kg, 20 mg/kg, 21 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, including all integers in between. In some embodiments, the oligomer is administered at 10 mg/kg. In some embodiments, the oligomer is administered at 20 mg/kg. In some embodiments, the oligomer is administered at 30 mg/kg. In some embodiments, the oligomer is administered at 40 mg/kg. In some embodiments, the oligomer is administered at 60 mg/kg. In some embodiments, the oligomer is administered at 80 mg/kg. In some embodiments, the oligomer is administered at 160 mg/kg. In some embodiments, the oligomer is administered at 50 mg/kg.

In some embodiments, the antisense oligomer conjugate of Formula (VI), Formula (VIII), or Formula (X), or a pharmaceutically acceptable salt thereof, is administered in doses generally from about 10-160 mg/kg or 20-160 mg/kg. In some embodiments, doses of the antisense oligomer conjugate of Formula (VI), Formula (VIII), or Formula (X), or a pharmaceutically acceptable salt thereof, for i.v. administration are from about 0.5 mg to 160 mg/kg. In some embodiments, the antisense oligomer conjugate of Formula (VI), Formula (VIII), or Formula (X), or a pharmaceutically acceptable salt thereof, is administered at doses of about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg. In some embodiments, the antisense oligomer conjugate of Formula (VI), Formula (VIII), or Formula (X), or a pharmaceutically acceptable salt thereof, is administered at doses of about 10 mg/kg, 11 mg/kg, 12 mg/kg, 15 mg/kg, 18 mg/kg, 20 mg/kg, 21 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, including all integers in between. In some embodiments, the antisense oligomer conjugate of Formula (VI), Formula (VIII), or Formula (X), or a pharmaceutically acceptable salt thereof, is administered at 10 mg/kg. In some embodiments, the antisense oligomer conjugate of Formula (VI), Formula (VIII), or Formula (X), or a pharmaceutically acceptable salt thereof, is administered at 20 mg/kg. In some embodiments, the antisense oligomer conjugate of Formula (VI), Formula (VIII), or Formula (X), or a pharmaceutically acceptable salt thereof, is administered at 30 mg/kg. In some embodiments, the antisense oligomer conjugate of Formula (VI), Formula (VIII), or Formula (X), or a pharmaceutically acceptable salt thereof, is administered at 40 mg/kg. In some embodiments, the antisense oligomer conjugate of Formula (VI), Formula (VIII), or Formula (X), or a pharmaceutically acceptable salt thereof, is administered at 60 mg/kg. In some embodiments, the antisense oligomer conjugate of Formula (VI), Formula (VIII), or Formula (X), or a pharmaceutically acceptable salt thereof, is administered at 80 mg/kg. In some embodiments, the antisense oligomer conjugate of Formula (VI), Formula (VIII), or Formula (X), or a pharmaceutically acceptable salt thereof, is administered at 160 mg/kg. In some embodiments, the antisense oligomer conjugate of Formula (VI), Formula (VIII), or Formula (X), or a pharmaceutically acceptable salt thereof, is administered at 50 mg/kg.

If desired, the effective daily dose of the active compound may be administered as two, three, four, five, six or more sub-doses administered separately at appropriate intervals throughout the day, optionally, in unit dosage forms. In certain situations, dosing is one administration per day. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression of a functional dystrophin protein. In certain embodiments, dosing is one or more administrations once every two weeks. In some embodiments, dosing is one administration once every two weeks. In various embodiments, dosing is one or more administrations every month. In certain embodiments, dosing is one administration every month.

In various embodiments, the antisense oligomer conjugates are administered weekly at 10 mg/kg. In various embodiments, the antisense oligomer conjugates are administered weekly at 20 mg/kg. In various embodiments, the antisense oligomer conjugates are administered weekly at 30 mg/kg. In various embodiments, the antisense oligomer conjugates are administered weekly at 40 mg/kg. In some embodiments, the antisense oligomer conjugates are administered weekly at 60 mg/kg. In some embodiments, the antisense oligomer conjugates are administered weekly at 80 mg/kg. In some embodiments, the antisense oligomer conjugates are administered weekly at 100 mg/kg. In some embodiments, the antisense oligomer conjugates are administered weekly at 160 mg/kg. As used herein, weekly is understood to have the art-accepted meaning of every week.

In various embodiments, the antisense oligomer conjugates are administered biweekly at 10 mg/kg. In various embodiments, the antisense oligomer conjugates are administered biweekly at 20 mg/kg. In various embodiments, the antisense oligomer conjugates are administered biweekly at 30 mg/kg. In various embodiments, the antisense oligomer conjugates are administered biweekly at 40 mg/kg. In some embodiments, the antisense oligomer conjugates are administered biweekly at 60 mg/kg. In some embodiments, the antisense oligomer conjugates are administered biweekly at 80 mg/kg. In some embodiments, the antisense oligomer conjugates are administered biweekly at 100 mg/kg. In some embodiments, the antisense oligomer conjugates are administered biweekly at 160 mg/kg. As used herein, biweekly is understood to have the art-accepted meaning of every two weeks.

In various embodiments, the antisense oligomer conjugates are administered every third week at 10 mg/kg. In various embodiments, the antisense oligomer conjugates are administered every third week at 20 mg/kg. In various embodiments, the antisense oligomer conjugates are administered every third week at 30 mg/kg. In various embodiments, the antisense oligomer conjugates are administered every third week at 40 mg/kg. In some embodiments, the antisense oligomer conjugates are administered every third week at 60 mg/kg. In some embodiments, the antisense oligomer conjugates are administered every third week at 80 mg/kg. In some embodiments, the antisense oligomer conjugates are administered every third week at 100 mg/kg. In some embodiments, the antisense oligomer conjugates are administered every third week at 160 mg/kg. As used herein, every third week is understood to have the art-accepted meaning of once every three weeks.

In various embodiments, the antisense oligomer conjugates are administered monthly at 10 mg/kg. In various embodiments, the antisense oligomer conjugates are administered monthly at 20 mg/kg. In various embodiments, the antisense oligomer conjugates are administered monthly at 30 mg/kg. In various embodiments, the antisense oligomer conjugates are administered monthly at 40 mg/kg. In some embodiments, the antisense oligomer conjugates are administered monthly at 60 mg/kg. In some embodiments, the antisense oligomer conjugates are administered monthly at 80 mg/kg. In some embodiments, the antisense oligomer conjugates are administered monthly at 100 mg/kg. In some embodiments, the antisense oligomer conjugates are administered monthly at 160 mg/kg. As used herein, monthly is understood to have the art-accepted meaning of every month.

As would be understood in the art, weekly, biweekly, every third week, or monthly administrations may be in one or more administrations or sub-doses as discussed herein.

Nucleic acid molecules and antisense oligomer conjugates described herein can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991) and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In one aspect of disclosure, the formulations contain micelles formed from an oligomer as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize an antisense oligomer conjugate of the present disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-poly(ethylene glycol) esters of the corresponding fatty acids, with a particularly preferred fatty acid composition including capric acid 4-10%, capric acid 3-9%, lauric acid 40-50%, myristic acid 14-24%, palmitic acid 4-14%, and stearic acid 5-15%. Another useful class of amphiphilic carriers includes partially esterified sorbitan and/or sorbitol, with saturated or monounsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

In certain embodiments, the delivery may occur by use of liposomes, nanocapsules, microparticles, microspheres, lipid particles, vesicles, and the like, for the introduction of the pharmaceutical compositions of the present disclosure into suitable host cells. In particular, the pharmaceutical compositions of the present disclosure may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present disclosure are those which are readily water-soluble, can be covalently attached to a vesicle-forming lipid, and which are tolerated in vivo without toxic effects (i.e., are biocompatible). Suitable polymers include poly(ethylene glycol) (PEG), polylactic (also termed polylactide), polyglycolic acid (also termed polyglycolide), a polylactic-polyglycolic acid copolymer, and polyvinyl alcohol. In certain embodiments, polymers have a weight average molecular weight of from about 100 or 120 daltons up to about 5,000 or 10,000 daltons, or from about 300 daltons to about 5,000 daltons. In other embodiments, the polymer is poly(ethylene glycol) having a weight average molecular weight of from about 100 to about 5,000 daltons, or having a weight average molecular weight of from about 300 to about 5,000 daltons. In certain embodiments, the polymer is a poly(ethylene glycol) having a weight average molecular weight of about 750 daltons, for example PEG(750). Polymers may also be defined by the number of monomers therein; a preferred embodiment of the present disclosure utilizes polymers of at least about three monomers, such PEG polymers consisting of three monomers have a molecular weight of approximately 132 daltons.

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7, or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present disclosure relates to formulations comprising liposomes containing an antisense oligomer conjugate of the present disclosure, where the liposome membrane is formulated to provide a liposome with increased carrying capacity. Alternatively or in addition, the antisense oligomer conjugate of the present disclosure may be contained within, or adsorbed onto, the liposome bilayer of the liposome. An antisense oligomer conjugate of the present disclosure may be aggregated with a lipid surfactant and carried within the liposome's internal space; in these cases, the liposome membrane is formulated to resist the disruptive effects of the active agent-surfactant aggregate.

According to one embodiment of the present disclosure, the lipid bilayer of a liposome contains lipids derivatized with poly(ethylene glycol) (PEG), such that the PEG chains extend from the inner surface of the lipid bilayer into the interior space encapsulated by the liposome, and extend from the exterior of the lipid bilayer into the surrounding environment.

Active agents contained within liposomes of the present disclosure are in solubilized form. Aggregates of surfactant and active agent (such as emulsions or micelles containing the active agent of interest) may be entrapped within the interior space of liposomes according to the present disclosure. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present disclosure.

Liposomes according to the present disclosure may be prepared by any of a variety of techniques that are known in the art. See, e.g., U.S. Pat. No. 4,235,871; Published PCT application WO 96/14057; New RRC, Liposomes: A practical approach, TRL Press, Oxford (1990), pages 33-104; and Lasic D D, Liposomes from physics to applications, Elsevier Science Publishers BV, Amsterdam, 1993. For example, liposomes of the present disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present disclosure, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the present disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and 30 percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An antisense oligomer conjugate may be formulated to be contained within, or, adapted to release by a surgical or medical device or implant. In certain aspects, an implant may be coated or otherwise treated with an antisense oligomer conjugate. For example, hydrogels, or other polymers, such as biocompatible and/or biodegradable polymers, may be used to coat an implant with the pharmaceutical compositions of the present disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the antisense oligomer conjugates for use according to the disclosure may be formulated for administration in any convenient way for use in human or veterinary medicine, by analogy with other pharmaceuticals. The antisense oligomer conjugates and their corresponding formulations may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

In some embodiments, the additional therapeutic may be administered prior, concurrently, or subsequently to the administration of the antisense oligomer conjugate of the present disclosure. For example, the antisense oligomer conjugates may be administered in combination with a steroid and/or antibiotic. In certain embodiments, the antisense oligomer conjugates are administered to a patient that is on background steroid theory (e.g., intermittent or chronic/continuous background steroid therapy). For example, in some embodiments the patient has been treated with a corticosteroid prior to administration of an antisense oligomer and continues to receive the steroid therapy. In some embodiments, the steroid is glucocorticoid or prednisone.

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra; Wolff et al. (1990) Science, 247:1465-1468). Direct injection of transgenes into tissue produces only localized expression (Rosenfeld (1992) supra; Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

In a further embodiment, pharmaceutical compositions of the disclosure may additionally comprise a carbohydrate as provided in Han et al., Nat. Comms. 7, 10981 (2016) the entirety of which is incorporated herein by reference. In some embodiments, pharmaceutical compositions of the disclosure may comprise 5% of a hexose carbohydrate. For example, pharmaceutical composition of the disclosure may comprise 5% glucose, 5% fructose, or 5% mannose. In certain embodiments, pharmaceutical compositions of the disclosure may comprise 2.5% glucose and 2.5% fructose. In some embodiments, pharmaceutical compositions of the disclosure may comprises a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

In certain aspects, an antisense oligomer conjugate described herein is administered in a liquid pharmaceutical formulation, wherein the concentration of the conjugate is about 50 mg/ml.

Regardless of the route of administration selected, the antisense oligomer conjugates of the present disclosure, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration, without being unacceptably toxic to the patient.

### Methods of Use

Dosage regimens described in the present disclosure can be used to treat a patient with an antisense oligomer conjugate described herein in need of such treatment.

In one aspect, the disclosure provides a method for treating DMD in a subject in need thereof wherein the subject has a mutation of the dystrophin gene that is amenable to exon skipping, the method comprising administering to the subject an antisense oligomer conjugate described herein. In some aspects, the exon is exon 44, exon 45, exon 50, exon 51, exon 52, or exon 53 of the human dystrophin gene.

In another aspect, the disclosure provides a method of restoring an mRNA reading frame to induce dystrophin production in a subject having a mutation of the dystrophin gene that is amenable to exon skipping (for example, exon 44, exon 45, exon 50, exon 51, exon 52, exon 53 skipping), the method comprising administering to the subject an antisense oligomer conjugate described herein.

In another aspect, the disclosure provides a method of excluding an exon (for example, exon 44, exon 45, exon 50, exon 51, exon 52, exon 53) from dystrophin pre-mRNA during mRNA processing in a subject having a mutation of the dystrophin gene that is amenable to exon skipping, the method comprising administering to the subject an antisense oligomer conjugate described herein. In another aspect, the disclosure provides a method of binding exon (for example, exon 44, exon 45, exon 50, exon 51, exon 52, exon 53) of dystrophin pre-mRNA in a subject having a mutation of the dystrophin gene that is amenable to exon skipping (for example, exon 44, exon 45, exon 50, exon 51, exon 52, exon 53 skipping), the method comprising administering to the subject an antisense oligomer conjugate described herein.

The term “restoration” with respect to dystrophin synthesis or production refers generally to the production of a dystrophin protein including truncated forms of dystrophin in a patient with muscular dystrophy following treatment with an antisense oligomer conjugate described herein. In some embodiments, treatment results in an increase in novel dystrophin production in a patient by 1%, 5%, 10%, 20%, 30%, 40%, 50%, 60%, 70%, 80%, 90%, or 100% (including all integers in between). In some embodiments, treatment increases the number of dystrophin-positive fibers to at least about 20%, about 30%, about 40%, about 50%, about 60%, about 70%, about 80%, about 90%, or about 95% to 100% of normal in the subject. In other embodiments, treatment increases the number of dystrophin-positive fibers to about 20% to about 60%, or about 30% to about 50%, of normal in the subject. The percent of dystrophin-positive fibers in a patient following treatment can be determined by a muscle biopsy using known techniques. For example, a muscle biopsy may be taken from a suitable muscle, such as the biceps brachii muscle in a patient.

Analysis of the percentage of positive dystrophin fibers may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression analysis may be performed using any suitable assay for dystrophin. In some embodiments, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle, as well as revertant fibers, a baseline can be set using sections of pre-treatment muscles from a patient when counting dystrophin-positive fibers in post-treatment muscles. This may be used as a threshold for counting dystrophin-positive fibers in sections of post-treatment muscle in that patient. In other embodiments, antibody-stained tissue sections can also be used for dystrophin quantification using Bioquant image analysis software (Bioquant Image Analysis Corporation, Nashville, TN). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Leica Biosystems may be used. The percentage of dystrophin-positive fibers can also be analyzed by determining the expression of the components of the sarcoglycan complex (β,γ) and/or neuronal NOS.

In some embodiments, treatment with an antisense oligomer conjugate of the disclosure slows or reduces the progressive respiratory muscle dysfunction and/or failure in patients with DMD that would be expected without treatment. In some embodiments, treatment with an antisense oligomer conjugate of the disclosure may reduce or eliminate the need for ventilation assistance that would be expected without treatment. In some embodiments, measurements of respiratory function for tracking the course of the disease, as well as the evaluation of potential therapeutic interventions include maximum inspiratory pressure (MIP), maximum expiratory pressure (MEP), and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some embodiments, MEP may decline before changes in other pulmonary function tests, including MIP and FVC. In certain embodiments, MEP may be an early indicator of respiratory dysfunction. In certain embodiments, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain embodiments, MIP percent predicted (MIP adjusted for weight), MEP percent predicted (MEP adjusted for age), and FVC percent predicted (FVC adjusted for age and height) are supportive analyses.

The terms “subject” and “patient” as used herein include any animal that exhibits a symptom, or is at risk for exhibiting a symptom, which can be treated with an antisense oligomer conjugate of the disclosure, such as a subject (or patient) that has or is at risk for having DMD or BMD, or any of the symptoms associated with these conditions (e.g., muscle fiber loss). Suitable subjects (or patients) include laboratory animals (such as mouse, rat, rabbit, or guinea pig), farm animals, and domestic animals or pets (such as a cat or dog). Non-human primates and, preferably, human patients (or subjects), are included. Also included are methods of producing dystrophin in a subject (or patient) having a mutation of the dystrophin gene that is amenable to exon skipping (for example, exon 44, exon 45, exon 50, exon 51, exon 52, exon 53 skipping).

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phase “targeting sequence” refers to a sequence of nucleobases of an oligomer that is complementary to a sequence of nucleotides in a target pre-mRNA. In some aspects of the disclosure, the sequence of nucleotides in the target pre-mRNA is an exon 51 annealing site in the dystrophin pre-mRNA designated as H51A(+66+95). In some aspect of the disclosure, the sequence of nucleotides in the target pre-mRNA is an exon 45 annealing site in the dystrophin pre-mRNA designated as H45A(−03+19). In some aspect, the sequence of nucleotides in the target pre-mRNA is an exon 53 annealing site in the dystrophin pre-mRNA designated as H53A(+36+60).

“Treatment” of a subject (e.g. a mammal, such as a human) is any type of intervention used in an attempt to alter the natural course of the subject. Treatment includes, but is not limited to, administration of an antisense oligomer conjugate or a pharmaceutical composition thereof, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some embodiments, treatment with an antisense oligomer of the disclosure increases novel dystrophin production, delays disease progression, slows or reduces the loss of ambulation, reduces muscle inflammation, reduces muscle damage, improves muscle function, reduces loss of pulmonary function, and/or enhances muscle regeneration that would be expected without treatment. In some embodiments, treatment maintains, delays, or slows disease progression. In some embodiments, treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains or reduces the time to walk/run 10 meters (i.e., the 10 meter walk/run test). In some embodiments, treatment maintains or reduces the time to stand from supine (i.e, time to stand test). In some embodiments, treatment maintains or reduces the time to climb four standard stairs (i.e., the four-stair climb test). In some embodiments, treatment maintains or reduces muscle inflammation in the patient, as measured by, for example, MRI (e.g., MRI of the leg muscles). In some embodiments, MRI measures T2 and/or fat fraction to identify muscle degeneration. MRI can identify changes in muscle structure and composition caused by inflammation, edema, muscle damage, and fat infiltration.

In some embodiments, treatment with an antisense oligomer conjugate of the disclosure increases novel dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value. In some embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. PLoS Curr., 2012, version 2, herein incorporated by reference). In some embodiments, treatment with an antisense oligomer increases the stable walking distance in the patient from baseline to greater than 3, 5, 6, 7, 8, 9, 10, 15, 20, 25, 30, or 50 meters (including all integers in between).

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLoS Curr., 2012, version 2, herein incorporated by reference).

An antisense molecule nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:
H #A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

### Restoration of the Dystrophin Reading Frame Using Exon Skipping

A potential therapeutic approach to the treatment of DMID caused by out-of-frame mutations in the dystrophin gene is suggested by the milder form of dystrophinopathy known as BMD, which is caused by in-frame mutations. The ability to convert an out-of-frame mutation to an in-frame mutation would hypothetically preserve the mRNA reading frame and produce an internally shortened yet functional dystrophin protein. Antisense oligomer conjugates of the disclosure were designed to accomplish this.

Clinical outcomes for analyzing the effect of an antisense oligomer conjugate that is complementary to a target region of the human dystrophin pre-mRNA and induces exon skipping include percent dystrophin positive fibers (PDPF), six-minute walk test (6MWT), loss of ambulation (LOA), North Star Ambulatory Assessment (NSAA), pulmonary function tests (PFT), ability to rise (from a supine position) without external support, de novo dystrophin production, and other functional measures.

In some embodiments, the present disclosure provides methods for producing dystrophin in a subject having a mutation of the dystrophin gene that is amenable to exon skipping (e.g., exon 44, 45, 50, 51, 52, 53), the method comprising administering to the subject an antisense oligomer conjugate, or pharmaceutically acceptable salt thereof, as described herein. In certain embodiments, the present disclosure provides methods for restoring an mRNA reading frame to induce dystrophin protein production in a subject with Duchenne muscular dystrophy (DMD) who has a mutation of the dystrophin gene that is amenable to exon skipping (e.g., exon 44, 45, 50, 51, 52, 53). Protein production can be measured by reverse-transcription polymerase chain reaction (RT-PCR), western blot analysis, or immunohistochemistry (IHC).

In some embodiments, the present disclosure provides methods for treating DMD in a subject in need thereof, wherein the subject has a mutation of the dystrophin gene that is amenable to exon skipping (e.g., exon 44, 45, 50, 51, 52, 53), the method comprising administering to the subject an antisense oligomer conjugate, or pharmaceutically acceptable salt thereof, as described herein. In various embodiments, treatment of the subject is measured by delay of disease progression. In some embodiments, treatment of the subject is measured by maintenance of ambulation in the subject or reduction of loss of ambulation in the subject. In some embodiments, ambulation is measured using the 6 Minute Walk Test (6MWT). In certain embodiments, ambulation is measured using the North Start Ambulatory Assessment (NSAA).

In various embodiments, the present disclosure provides methods for maintaining pulmonary function or reducing loss of pulmonary function in a subject with DMD, wherein the subject has a mutation of the DMD gene that is amenable to exon skipping (e.g., exon 44, 45, 50, 51, 52, 53), the method comprising administering to the subject an antisense oligomer conjugate, or pharmaceutically acceptable salt thereof, as described herein. In some embodiments, pulmonary function is measured as Maximum Expiratory Pressure (MEP). In certain embodiments, pulmonary function is measured as Maximum Inspiratory Pressure (MIP). In some embodiments, pulmonary function is measured as Forced Vital Capacity (FVC).

In certain aspects, the methods of the present disclosure include administering to a subject with DMD a pharmaceutical formulation comprising an antisense oligomer conjugate, or pharmaceutically acceptable salt thereof, as described herein, wherein the concentration of the conjugate in the formulation is about 50 mg/ml.

In certain embodiments, there is described an antisense oligomer conjugate as described herein for use in therapy. In certain embodiments, there is described an antisense oligomer conjugate as described herein for use in the treatment of Duchenne muscular dystrophy. In certain embodiments, there is described an antisense oligomer conjugate as described herein for use in the manufacture of a medicament for use in therapy. In certain embodiments, there is described an antisense oligomer conjugate as described herein for use in the manufacture of a medicament for the treatment of Duchenne muscular dystrophy.

### EXAMPLES

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1. In Vitro Evaluation of Exon 51 Skipping Activity by PPMOs of Formula (VIC)

The exon 51 skipping activity of all the six PPMOs within the following structure of Formula (VIC) was assessed:

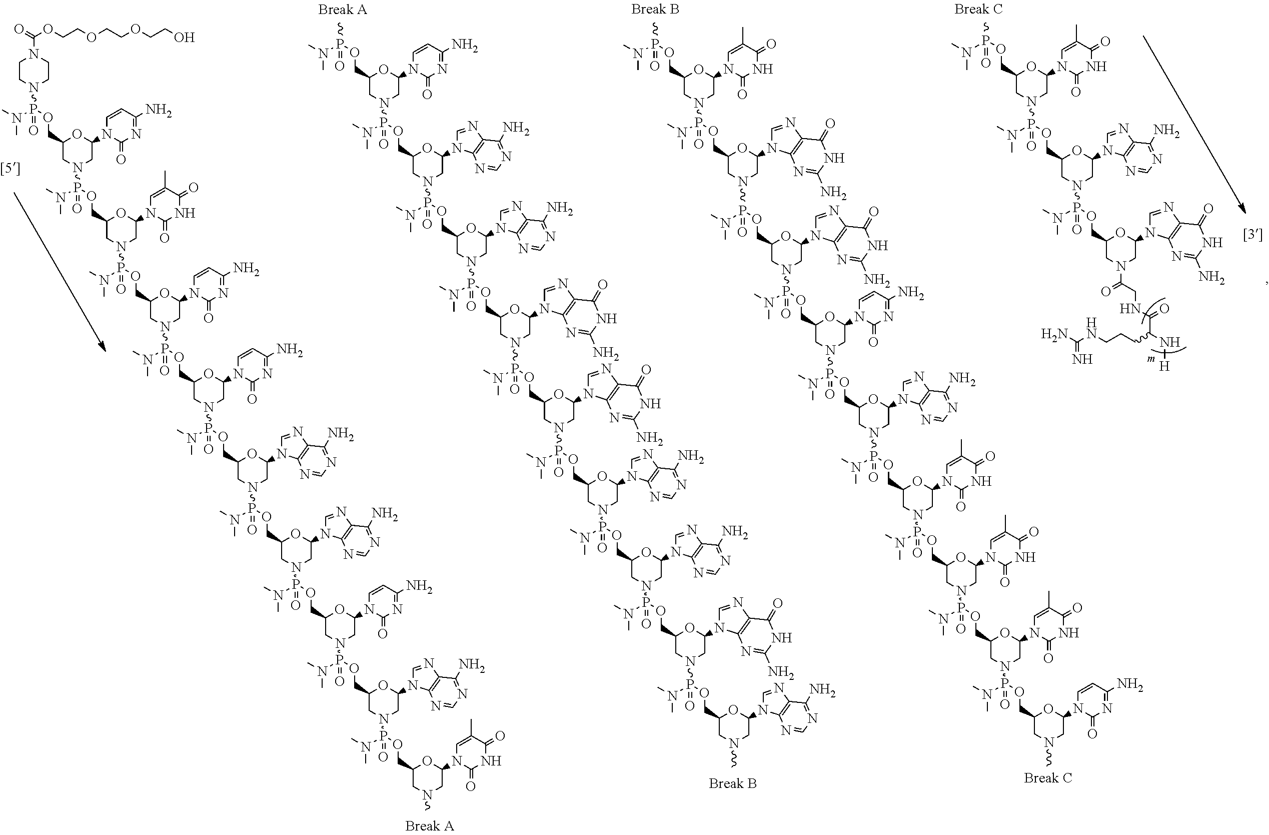

wherein M is 0, 1, 2, 3, 4, or 5 and the targeting sequence is 5′-CTCCAACATCAAGGAATGGCATTTCTAG-3′ (SEQ ID NO: 1).

The six PPMOs tested were synthesized and characterized in house following the protocol described, e.g., in U.S. Pat. No. 10,888,578, and are described in Table 1 (G is glycine and R is arginine).

TABLE 1

| PPMO | m | Conjugate attached to PMO | MW |
|---|---|---|---|
| PPMO-10 | 0 | G- | 10362.99 |
| PPMO-11 | 1 | RG- | 10555.65 |
| PPMO-12 | 2 | RRG- | 10748.3 |
| PPMO-13 | 3 | RRRG- | 10940.95 |
| PPMO-14 | 4 | RRRRG- | 11133.6 |
| PPMO-15 | 5 | RRRRRG- | 11326.25 |

### Exon Skipping Analysis

The in vitro exon skipping activity of the six PPMOs were measured in a cellular model of DMD using immortalized myoblasts derived from a donor with an exon 51-skip amenable deletion in exon 52 of the DMD gene. For the assay, myoblasts were differentiated into myotubes and exon skipping activity was measured by ddPCR after a 96 hr treatment with concentrations of the PPMOs ranging from 0.1-100 M. The potency and maximum exon skipping activity of the tested PPMOs, measured at 100 M, are shown in Table 2 below. The maximum exon skipping activity is the mean and standard deviation of the percentage of skipped copies of four technical replicates measured at the concentration that demonstrated the greatest activity,

TABLE 2

| PPMO | EC₅₀ (μM) | Maximum ± SD (%) |
|---|---|---|
| PPMO-15 | 11.0 | 92 ± 0.9 |
| PPMO-14 | 7.2 | 92 ± 1.3 |
| PPMO-13 | 20.5 | 82 ± 0.5 |
| PPMO-12 | 51.8 | 74 ± 2.2 |
| PPMO-11 | ND | 50 ± 0.6 |
| PPMO-10 | ND | 36 ± 2.7 |

EC₅₀ = half-maximal effective concentration
ND = not determined
SD = standard deviation

EC₅₀ values for PPMO-11 and PPMO-10 could not be accurately determined due to incomplete concentration response curves.

The results show that all the tested PPMOs are pharmacologically active exhibiting exon skipping activity. All the tested PPMOs had measurable, concentration-dependent exon skipping activity in the assay.

### Materials and Methods

### Cell Lines and Culturing Condition

Myoblasts isolated from the paravertebral muscles of a 16 year old healthy donor (Reference AB1190C16PV) and 16 year old DMD patient with a deletion in exon 52 (Reference KM1328DMD16PV) and immortalized by the Institute of Myology by ectopic expression of hTERT and CDK4 as previously described (Mamchaoui, K. et al., Skeletal Muscle 1(1):34, 2011) were used in this assay. Cells were maintained in proliferation medium containing 1 volume medium 199, 4 volumes Dulbecco's modified Eagle's medium (DMEM), 20% fetal bovine serum, 50 μg/ml gentamycin, 25 μg/ml fetuin, 0.5 μg/ml bFGF, 5 ng/ml EGF, 0.2 μg/ml dexamethasone, 5 μg/ml insulin on tissue culture plates coated with 1% collagen I and 0.5% MaxGel (Sigma-Aldrich E0282) at 50 μl/cm² for 3 hr at 37° C.

### Compound Testing

Immediately prior to testing, all compounds were dissolved in sterile water and the concentration was confirmed by spectrophotometry. Myoblasts were plated in proliferation medium at 6000 cells/well in a 96-well clear bottom imaging plate (Perkin Elmer #6055300) coated with 1% collagen I and 0.5% MaxGel (Sigma-Aldrich E0282) at 50 μl/well for 3 hr at 37° C. Twenty-four hours after plating cultures were switched to a differentiation medium containing DMEM, 2% heat inactivated FBS, 50 μg/ml gentamycin and 10 μg/ml insulin. 48 hours after the switch to differentiation medium, PPMOs were added and cultures were incubated an additional 4 days prior to analysis, for a total of approximately 96 hr of continuous compound exposure.

ddPCR Analysis for Human DMD Exon51 Skipping

RNA was isolated using RN easy Micro-column (QIAGEN cat #74004) with DNAase treatment per manufacturer's recommendation. 30 ng of isolated RNA was first denatured at 70° C. for 2 min and mixed with reagents from One-step RT-ddPCR Advanced Kit for Probes (BioRad cat #1864021) and PNP mix according to the Table 3 below. Droplets were generated from the prepared RNA sample mix using an automated droplet generator. After droplet generation plate was sealed and run on a C 1000 thermocycler (BioRad) following the thermocycler program in Table 4 below. Copy number of FAM and HEX positive droplet is determined by QX200 droplet reader. Percentage of exon skipping is determined as the copy number of FAM positive droplet (copy number of FAM positive droplet+ copy number of BEX positive droplet)*100. All data was analyzed using GraphPad Prism 8 and EC50s were determined based on a four-parameter logistic curve fit.

TABLE 3
A. Recipe to prepare RNA mix

| Reagent | 1 RXN (μl) | |
|---|---|---|
| Water | 9.1 | PnP mix are aloquoted as |
| Supermix | 5.5 | 20X in 200 μl, final conc. |
| Reverse Transcriptase | 2.2 | is 900 nM primer and |
| 300 nM DTT | 1.1 | 250 nM probes. |
| PnP | 1.1 | |
| 30 ng RNA/sample | 3 | |

B. Primer and probe sequence

| | Primer name | Sequence |
|---|---|---|
| Del52 | DYS-d52-F006 | AAAGCAGCCTGACCTAGCTCCTG |
| SKP51 | SEQ ID NO: | |
| | 520 | |
| | DYS-d52-F006 | TCAGCTTCTTCCTTAGCTTCCAGCC |
| | SEQ ID NO: | |
| | 521 | |
| | DYS-WT- | /5HEX/AC ACA ACC T/ZEN/G TGG TTA CTA AGG AAA CTG |
| | exon51-P001 | CCA TCT CC/3IABkFO/ |
| | SEQ ID NO: | |
| | 522 | |
| | DYS- | /56- |
| | d525KP51- | FAM/TGACCACTA/ZEN/TTGGAGCCTTTGAAAGAATTCAGAA |
| | P003 SEQ ID | TCAGT/3IABkFO/ |
| | NO: 523 | |

TABLE 4
Thermocycler program used

| Cycling Step | Temp. (° C.) | Time | | Cycles |
|---|---|---|---|---|
| RT | 43 | 60 | min | 1 |
| Enzyme activation | 95 | 10 | min | 1 |
| Denaturation | 95 | 30 | sec | 70 |
| Annealing/extension | 58 | 60 | sec | |
| (ramp 2 C/sec) | | | | |
| | 72 | 2 | min | |
| Enzyme deactivation | 98 | 10 | min | 1 |

| | | |
|---|---|---|
| Hold | 4 | ∞ |

### Example 2. In Vivo Study of Exposure of PPMOs of Formula (VIC) in Non-Human Primates (NHP) and mdx Mice after Administration of PPMO-1

1. NHP Study. Assessment of plasma exposure of PPMOs of Formula (VIC) after Intravenous (IV) Administration of PPMO-1 to Cynomolgus Monkeys.

Non-human primates (NHPs) received a 1-hour IV infusions of PPMO-1 at dose levels of 30, or 60 mg/kg once every 4 weeks on days 1, 29, 57 and 85. Blood samples were collected on day 1 at pre-dose, 1, 2, 4, 8, 12, 16 and 24 hours after the start of each infusion. Blood was processed to plasma for concentration analysis of PPMO-1 and its metabolites by Liquid Chromatography Mass Spectrometer (LC/MS/MS). PPMO-1 is an antisense oligomer conjugate with the following structure:

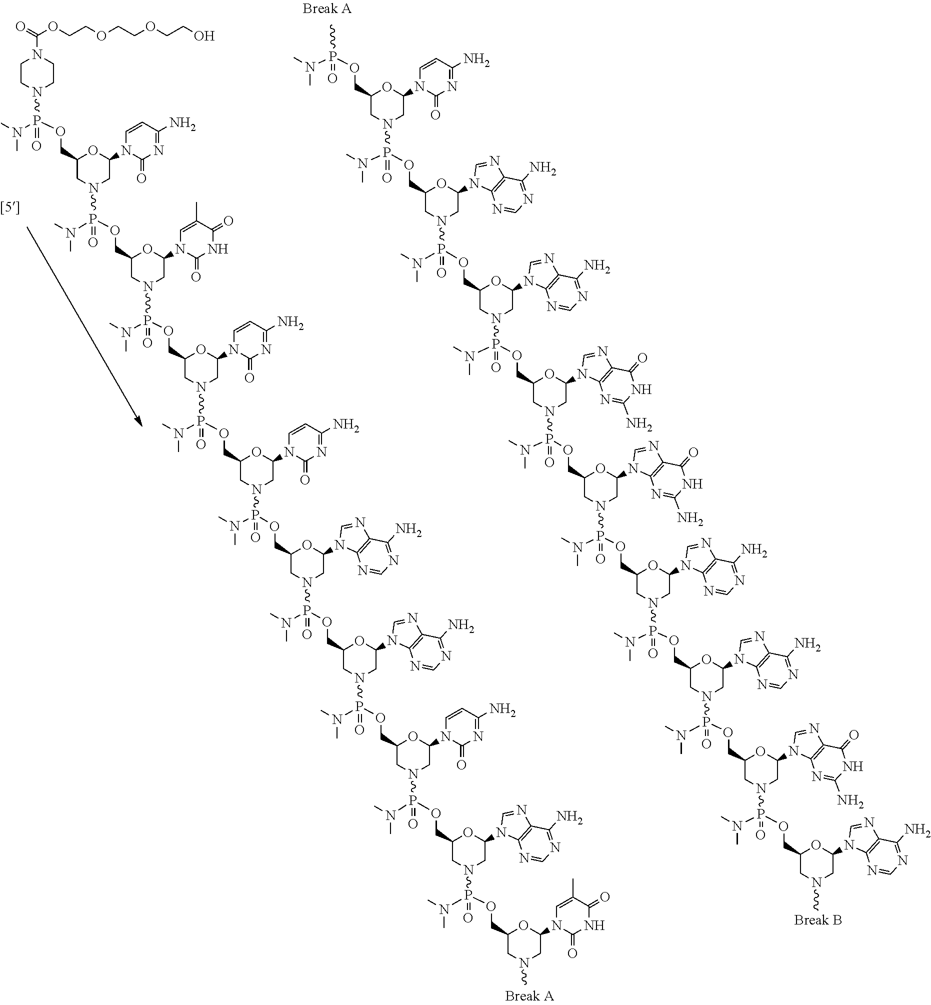

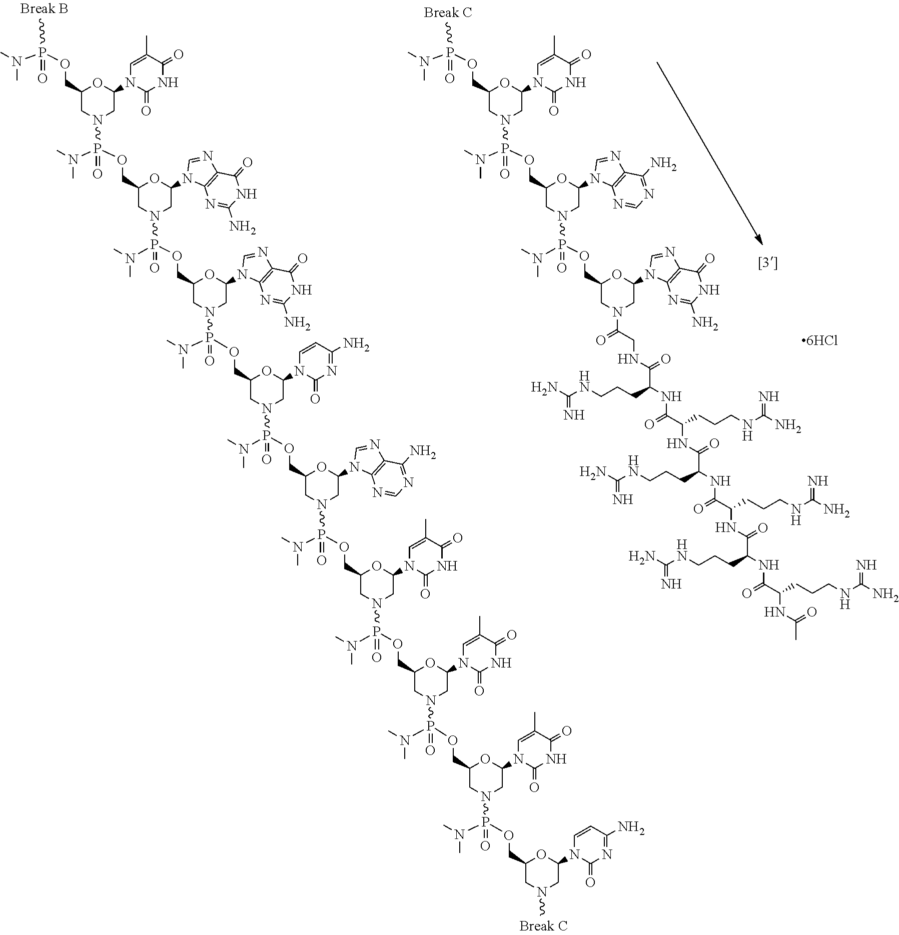

2. Study in mdx Mice. Assessment of Distribution of PPMOs of Formula (VIC) after Single Intravenous (IV) Administration of ¹⁴C-PPMO-1 to Male Dystrophic (mdx) Mice.

Male mdx mice received single intravenous injection of ¹⁴C-PPMO-1 at mean dose of 51.6 mg/kg. ¹⁴C-PPMO-1 was formulated as an aqueous solution in 0.9% (w/v) Sodium Chloride for injection at 10 mg/mL and was administered at radioactivity level of 220 μCi/kg of animal weight. Blood samples were collected from each mouse by heart puncture at approximately 0.083, 0.25, 0.5, 1, 2, 4, 6, 8, and 24 hours post-dose. Plasma samples obtained from male mice in Group 2 at 0.083, 0.25, 0.5, 1, 2, 4, 6, 8, and 24 hours postdose were pooled by time point to generate nine pooled samples, including 0.3 g of each sample. The nine pools were subsequently pooled to generate a single 0.083- to 24-hour AUC-representative pooled sample, including between 2.84 to 306 μL of each time point pool determined by using a time-weighted pooling method (Hop et al., 19981. Urine samples collected from male mice at 0-24 and 24-48 hours post-dose were pooled across all animals, including 0.3 g to 0.5 g of each sample. Samples were pooled using a constant percentage (10%) of sample weight.

Feces samples collected from male mice in at 0-24, 24-48, 48-72 hours postdose were pooled across all animals, as applicable, including 1.1 to 1.6 g of each sample. Samples were pooled using a constant percentage (5%) of sample weight.

The radioactivity of each pooled sample was determined by liquid scintillation counting (LSC) and LC/MS for PPMO-1 and its metabolites quantification analysis.

### Results

PPMO-1 was identified as the main analyte in NHP plasma and PPMO-10 and PPMO11 as the major metabolite at 10.5 and 6.7% and 3.7% and 3.1% of the AUCₗₐₛₜ of PPMO-1 dosed with PPMO-1 at 30 and 60 mg/kg, respectively. The total exposure (AUCₗₐₛₜ) was 257±138 h*ug/mL, 20±7 h*ug/mL and 8±4 h*ug/mL for PPMO-1, PPMO-10 and PPMO-11 respectively. Five other metabolites (PPMO-12, PPMO-13, PPMO-14, PPMO-15, and PMO) were detected and identified at lower levels than PPMO-10 and PPMO-11. It is of note that the observed PPMO-15 data are at least partially attributable to the spillover of LC-MS/MS signal, due to the similar retention time of PPMO-1; thus, the PPMO-15 level may be overestimated.

In mdx mice, PPMO-10 and PPMO-11 metabolites were identified in all matrices (plasma, urine and feces). Radiochemical and LC-MS analyses of plasma extracts identified SRP-5051 as the most abundant plasma component after an intravenous dose of ¹⁴C-PPMO-1 to male mice. Under the initial LC-MS conditions (Gradient 1), the concentration of ¹⁴C-PPMO-1 in AUC-pooled plasma was 1510 ng equivalents ¹⁴C-PPMO-1/g (ng eq./g) or 66.6% of sample radioactivity. The concentration of co-eluting PPMO-11 and PPMO-10 was 547 ng eq./g or 24.1% of the sample radioactivity. M3, which was not identified by LC-MS, had a plasma concentration of 69.7 ng eq./g or 3.1% of sample radioactivity. Under the revised LC-MS conditions (Gradient 2), concentrations of PPMO-1, PPMO-11 and PPMO-10 were 1370, 251, and 258 ng. eq/g, respectively, or 60.5%, 11.1%, and 11.4% of the sample radioactivity, respectively. Unidentified M4 had a plasma concentration of 138 ng. eq/g or 6.1% of plasma radioactivity.

In urine, radiochemical and LC-MS analyses identified PPMO-1, PPMO10 and PPMO-11. Two additional radiolabeled components (M4 and M5) were quantified by radiochemical analysis but could not be identified by LC-MS. Under initial LC-MS conditions (Gradient 1), PPMO-1 accounted for 24.3% of the radioactive dose, and co-eluting PPMO-11 and PPMO-10 accounted for 32.1% of dose. Under revised LC-MS conditions (Gradient 2), PPMO-1, PPMO11 and PPMO-10 were partially separated and accounted for 22.0%, 14.6%, and 17.3% of the dose, respectively. Unidentified peaks M4 and M5 were trace- to minor-level components that accounted for approximately 0.35% and 1.1% of dose, respectively. Radiochemical and LC-MS analyses of fecal extracts quantified and identified PPMO-11 and PPMO-10 as minor metabolites; PPMO-1 was not detected in feces. M5 was quantified by radiochemical analysis but was not identified by LC-MS. Co-eluting (Gradient 1) metabolites PPMO-11 and PPMO-10 accounted for 6.3% of the dose. Under revised LC-MS conditions (Gradient 2), PPMO-11 and PPMO-10 accounted for approximately 1.6% and 4.5% of the dose, respectively, while unidentified trace component M5 accounted for less than 0.5% of dose.

### Conclusions

PPMO-1 and its seven hydrolytic metabolites were detected and quantified using LC/MS method after IV infusion of PPMO-1 to NHPs at 30 and 60 mg/kg. PPMO-1 was identified as the main component with the highest levels observed at the end of infusion, then declined in bi-exponential fashion. The magnitude of all metabolites was much less than that of PPMO-1, especially during the first 8 hours postdose. PPMO-10 was found to be the most abundant metabolite, followed by PPMO-11 at 7-10% and 3-4% of PPMO-1 AUC respectively, while the remaining 5 metabolites were detected at much lower concentrations and were only occasionally quantifiable.

Following a single intravenous administration of ¹⁴C-PPMO-1 to male mdx mice, PPMO-1 was the most abundant component in plasma and urine, accounting for 60-67% of plasma samples radioactivity and 22% of dose in urine. PPMO-1 was not detected in feces. PPMO-11 and PPMO-10 were identified as the main metabolites in plasma, urine and feces. Three additional radiolabeled peaks were quantified by radioactiochemical detection but could not be identified by LC-MS.

### Example 3. In Vivo Study of Plasma and Tissue Distribution of PPMOs of Formula (VIIIC) in mdx Mice after Administration of ¹⁴C-PPMO-2

Study in mdx Mice. Assessment of Distribution of PPMOs of Formula (VIIIC) after Single Intravenous (IV) Administration of ¹⁴C-PPMO-2 to Male Dystrophic (mdx) Mice.

Mdx mice received a single IV bolus injection of ¹⁴C-PPMO-2 at a mean dose of 53.6 mg/kg. ¹⁴C-PPMO-2 was formulated in aqueous solution in 0.9 Sodium Chloride at 10 mg/mL and was administered at mean radioactive level of 228 μCi/kg of animal weight. Samples of whole blood and selected tissues were collected at approximately 0.083, 0.25, 0.5, 1, 2, 4, 8, 24, 48, 96, 144, 288, 360 and 432 hours post-dose. Plasma samples obtained from male mdx mice at 0.083, 0.25, 0.5, 1, 2 and 4-hours post-dose were pooled by time point to generate 0.083-, 0.25-, 0.5, 1-, 2-, and 4-hour pooled samples including 0.1 g of each sample. Urine samples collected at 0-24 and 24-72 hours post-dose were pooled to generate a 0- to 24- and 24- to 72-hour pooled samples, including 15% of each sample by weight. Feces samples collected at 0-24, 24-48, and 48-72 hours post-dose were pooled by collection interval to generate 0- to 24-hour and 24- to 72-pooled samples, including 6 to 10% (equivalent percent by interval) of each sample by weight.

Muscle and kidney samples collected from mice at 2, 24, 48, 96, 144, 288, 360 and 432 hours post-dose were pooled by collection interval to generate 2-, 24- to 96-, 144- to 288, and 360- to 432-hours pooled samples including the entire sample. The radioactivity of each pooled sample was determined by LSC. Pooled samples were analyzed by LC/MS to determine the concentration of PPMO-2 and its metabolites. PPMO-2 is an antisense oligomer conjugate with the following structure:

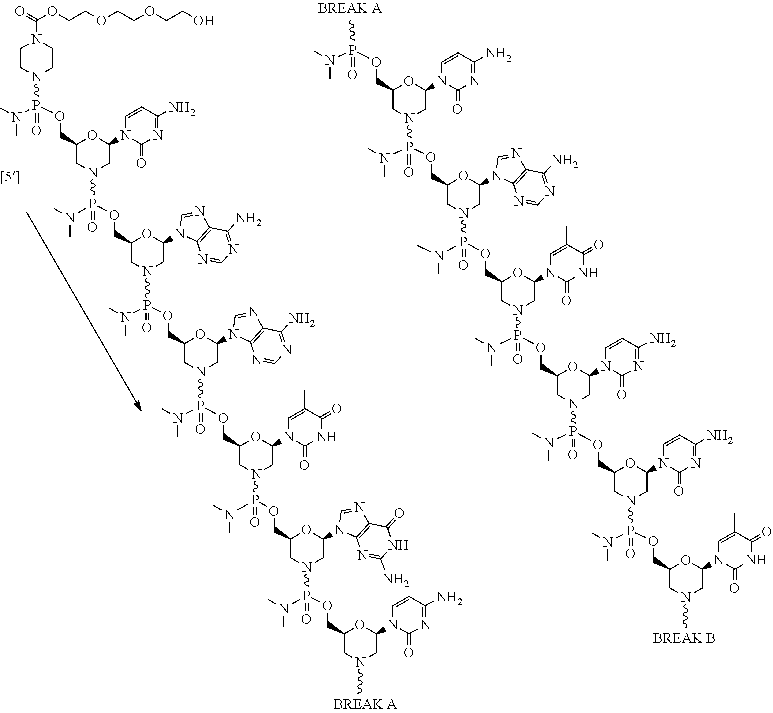

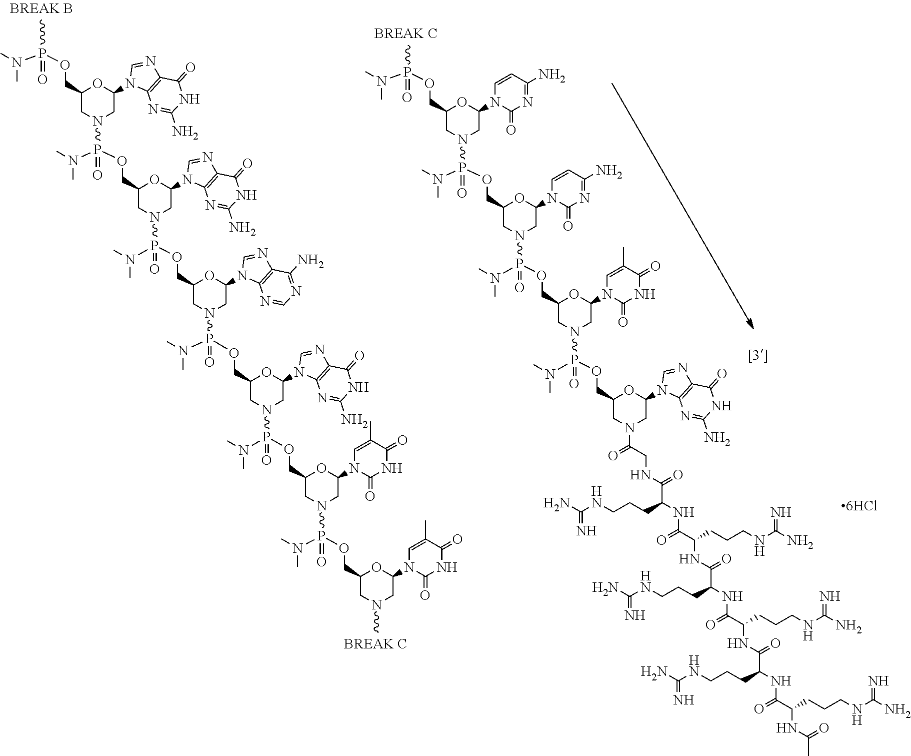

### Results

¹⁴C-PPMO-2 underwent metabolism in male mdx mice after a single intravenous dose of ¹⁴C-PPMO-2. Five metabolites were identified and characterized in plasma, urine, feces, muscle and kidneys by LC-MS. The identified compounds have the structure according to Formula (VIIIC):

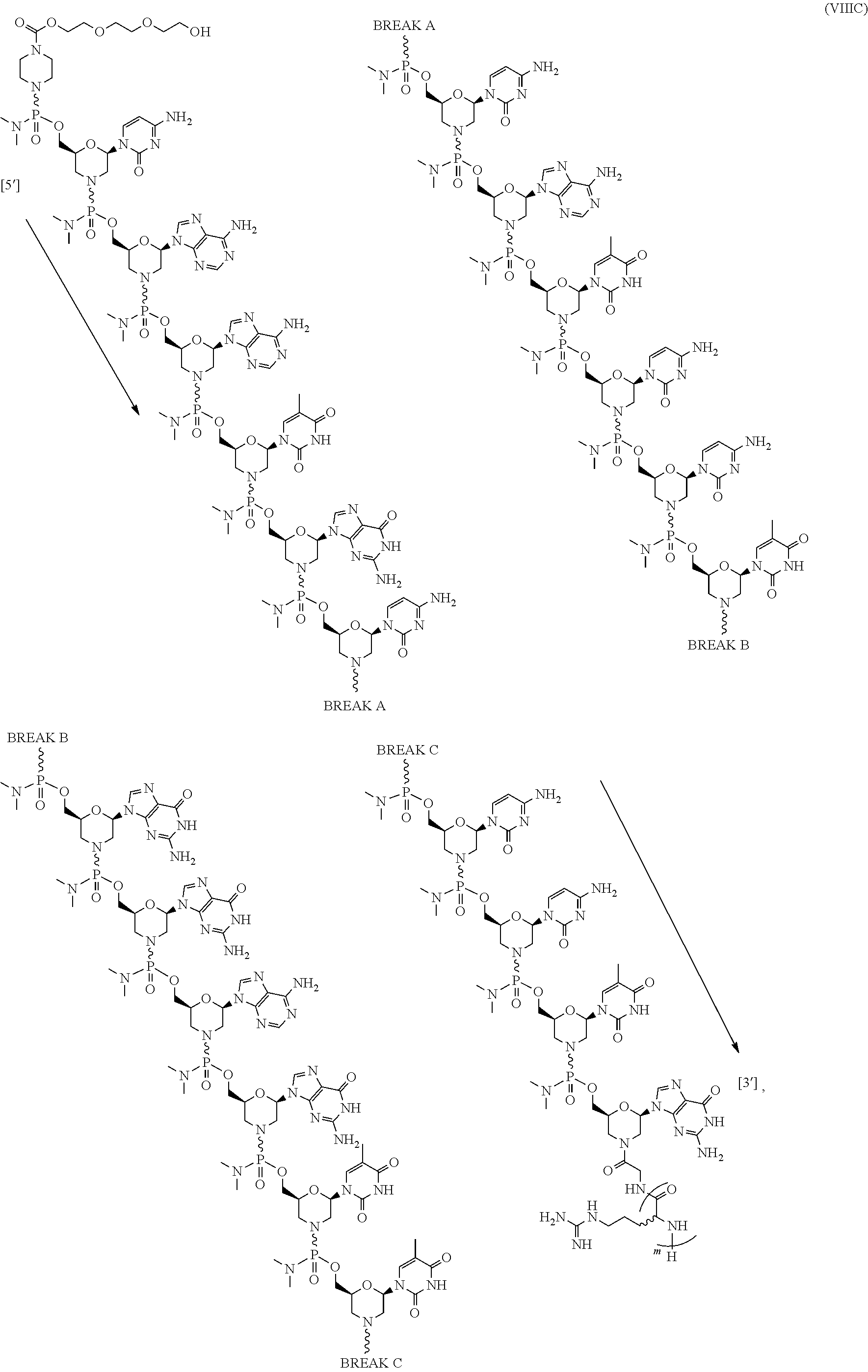

where the targeting sequence is 5′-CAATGCCATCCTGGAGTTCCTG-3′ (SEQ ID NO: 2) and m is as described below in Table 5:

TABLE 5

| PPMO | m | Conjugate attached to PMO |
|---|---|---|
| PPMO-20 | 0 | G- |
| PPMO-21 | 1 | RG- |
| PPMO-22 | 2 | RRG- |
| PPMO-23 | 3 | RRRG- |
| PPMO-24 | 4 | RRRRG- |

PPMO-24, PPMO-23, and PPMO-22 were present in all matrices with the exception of feces, and PPMO-21 and PPMO-20 were present in all matrices. PPMO-2 was identified in all matrices with the exception of feces.

The most abundant plasma component was PPMO-2; the peak concentration was 206 μg equivalents ¹⁴C-PPMO-2/g (representing 67.5% of the total AUC determined for PPMO-2 and identified metabolites and 33.48 to 91.52% of the total radioactivity injected on to the HPLC column). Peak concentrations for PPMO-24, PPMO-23, PPMO-22, PPMO-21, and PPMO-20 were 5.56, 4.86, 5.87, 10.8, and 9.40 g equivalents ¹⁴C-PPMO-2/g. Based on AUC₀₋ₜ all metabolites identified and quantifiable in plasma represented <10% of the total AUC for identified metabolites and were considered minor; the exception was PPMO-20 which represented 14.3% of the total. PPMO-20, accounting for 29.8% of the administered dose over 0 to 72 hours post-dose, was the most abundant component in urine. PPMO-2 accounted for 3.22% of the administered dose in urine over 0 to 24 hours post-dose, and was not detected in urine over 24 to 72 hours post-dose. PPMO-20 was also the most abundant component in feces accounting for 2.44% of the administered dose over 0 to 72 hours post-dose. PPMO-2 was not observed in feces.

For tissues (bicep muscle and kidneys), PPMO-20 was again the most abundant component and peak concentrations were 3.32 and 960 μg equivalents ¹⁴C-PPMO-2/g, respectively (representing between 12.03 and 15.28% of the total radioactivity injected on to the HPLC column for muscle and 83.73 and 92.32% for kidney). PPMO-2 was quantifiable at 2 hours post-dose in the bicep and was quantifiable in all sample pools through 432 hours post-dose for the kidneys; peak concentrations were 0.863 and 33.1 μg equivalents ¹⁴C-PPMO-2/g, respectively.

### Conclusion

Following a single IV administration of ¹⁴C-PPMO-2 to male mdx mice SRP-5045 underwent metabolism in male mice to produce up to 11 ¹⁴C-associated peaks, of which, five were identified by LC-MS. Hydrolysis of the terminal arginine (R) amino acids with loss of the N-acetyl group was the predominant biotransformation pathway for ¹⁴C-PPMO-2. PPMO-20 and PPMO-21 metabolites were identified in all matrices. Other metabolites were identified in all matrices with exception of feces. PPMO-2 was present in all matrices except for feces.

PPMO-2 was identified as the main component in plasma with peak concentration at 0.083 hours post-dose representing 91.52% of sample radioactivity. PPMO-20 was identified as the main metabolite accounting for 14.3% of total exposure (AUCₗₐₛₜ). All other metabolites exposures ranged between 2.98 and 7.52% of total AUC. PPMO-20 was identified as the main metabolite in urine, feces, biceps and kidney accounting for 29.8% (0-24 hours), 2.44% (0-24 hours) of sample radioactivity in urine and feces and peak concentrations at 37.5 and 87.67% of sample radioactivity in biceps and kidney. Other metabolites were identified as minor metabolites in all matrices.

### Example 4. In Vivo Study of Plasma and Tissue Distribution of PPMOs of Formula (XC) in mdx Mice after Administration of ¹⁴C-PPMO-3

Study in mdx Mice. Assessment of Distribution of PPMOs of Formula (XC) after Single Intravenous (IV) Administration of ¹⁴C-PPMO-3 to Male Dystrophic (mdx) Mice.

Mdx mice received a single IV bolus injection of ¹⁴C-PPMO-3 at a mean dose of 48.7 mg/kg. PPMO-3 was formulated as an aqueous solution in 0.9% (w/v) Sodium Chloride at 10 mg/mL and was administered at a mean radioactivity level of 215 μCi/kg of animal weight. Urine and feces were collected pre-dose (overnight) and at 24-hour intervals through 336 hours post-dose. Samples of whole blood and selected tissues were collected at approximately 0.083, 0.25, 0.5, 1, 2, 4, 6, 8, 24, 48, 72, 96, 120, and 144 hours post-dose.

Plasma samples obtained from male mice at 0.083, 0.25, 0.5, 1, and 2 hours post-dose were pooled by time point to generate 0.083-, 0.25-, 0.5-, 1-, and 2-hour pooled samples, including equal volumes of each sample. Urine samples collected from male mice at 0-24, 24-48, 48-72, 72-96, 96-120, 120-144, 144-168, 168-192, 192-216, 216-240, 288-312, and 312-336 hours post-dose were pooled to generate 0- to 24-, 24- to 48-, 48- to 72-, 72- to 144-, 144- to 240-, and 288- to 336-hour pooled samples, including 10 to 20% (equivalent percent by interval) of each sample by weight. Feces samples collected from male mice in at 0-24, 24-48, 48-72, 72-96, 96-120, 120-144, 288-312, and 312-336 hours post-dose were pooled to generate 0- to 72-, 72- to 144-, and 288- to 336-hour pooled samples, including 4 to 5% (equivalent percent by interval) of each sample by weight.

Bicep muscle samples obtained from male mice at 0.25, 1, 4, 8, 24, 48, 96, 120, and 144 hours post-dose were pooled to generate 0.25- to 4-, 8-, 24-, 48-, and 96- to 144-hour pooled samples, including 100% of each sample by weight.

Kidney samples obtained from male mice at 0.25, 1, 4, 8, 24, 48, 96, 120, and 144 hours post-dose were pooled by to generate 0.25- to 4-, 8-, 24-, 48-, and 96- to 144-hour pooled samples, including 100% of each sample by weight.

Samples from all matrices were analyzed by LSC to determine the radioactivity and by LC/MS to quantify PPMO-3 and its metabolites.

PPMO-3 is an antisense oligomer conjugate with the following structure:

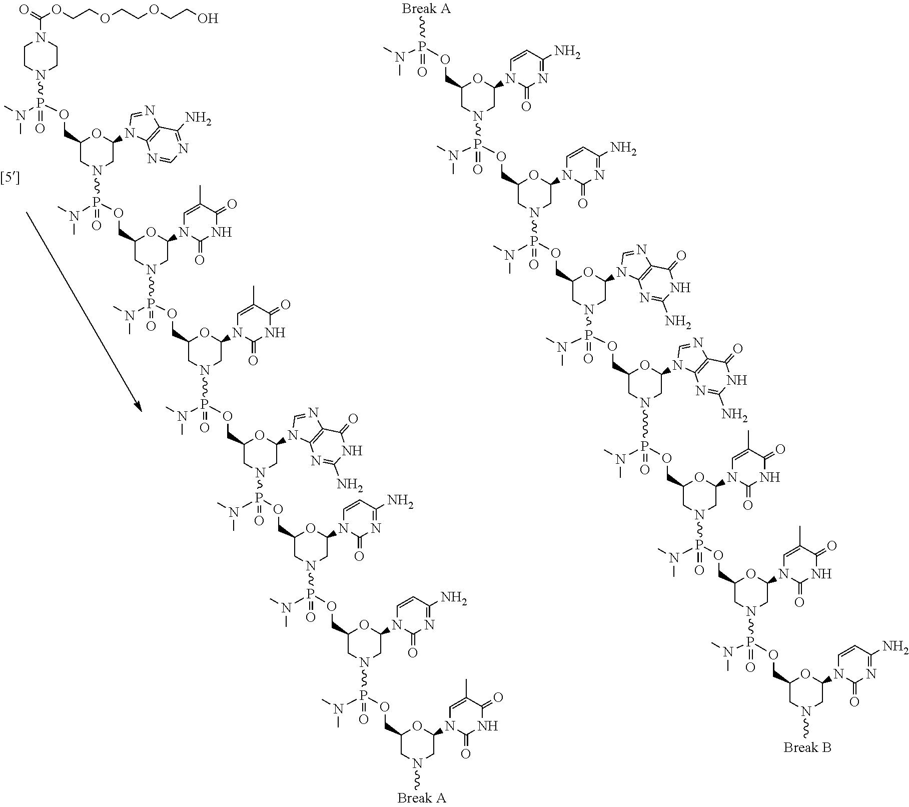

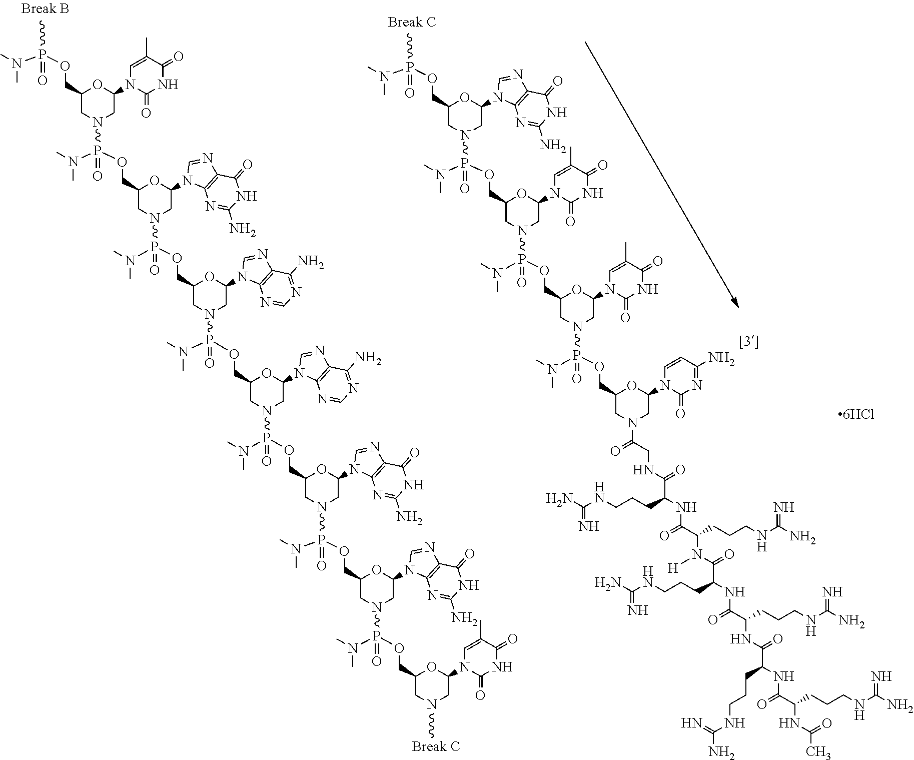

### Results

¹⁴C-PPMO-3 underwent metabolism in male mice after a single intravenous dose of ¹⁴C-PPMO-3, four metabolites were identified and characterized by LC-MS. The identified compounds have the structure according to Formula (XC):

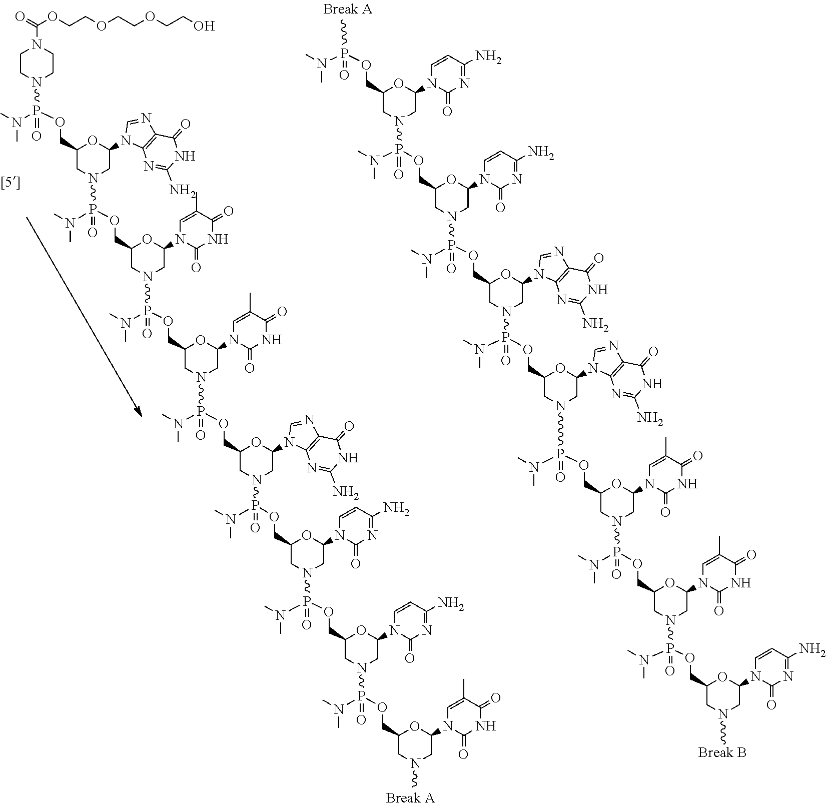

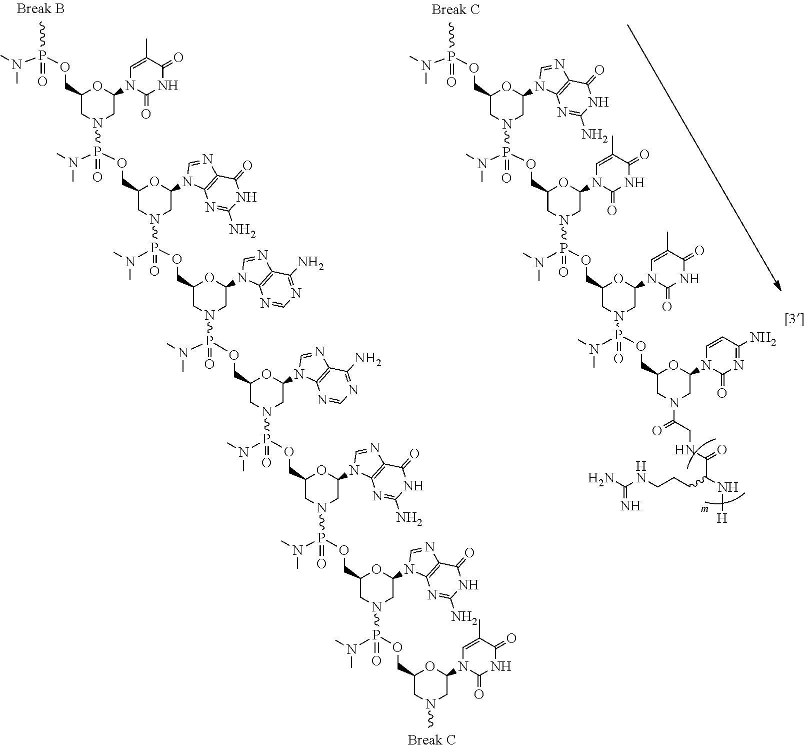

where the targeting sequence is 5′-GTTGCCTCCGGTTCTGAAGGTGTTC-3′ (SEQ ID NO: 3) and m is as described below in Table 6:

TABLE 6

| PPMO | m | Conjugate attached to PMO |
|---|---|---|
| PPMO-30 | 0 | G- |
| PPMO-31 | 1 | RG- |
| PPMO-33 | 3 | RRRG- |
| PPMO-34 | 4 | RRRRG- |

PPMO-30 and PPMO-31 were also synthetized in house. PPMO-33 and PPMO-34 were present in plasma and urine, PPMO-31 was present in plasma, urine, and feces, and PPMO-30 was present in plasma, urine, feces, and kidney. Metabolite PPMO-30 was the most abundant metabolite identified in urine and feces and PPMO-31 was also present at a notable level in urine. The most abundant plasma component was PPMO-3 and PPMO-33 which co-eluted for which the total peak concentration (Cmax) was 109000 ng-eq/g. Due to the broad chromatography peaks in plasma samples, PPMO-3 and PPMO-33 retention times are similar, therefore the PPMO-33 contribution may be overestimated and partially due to PPMO-3. PPMO-31 was the most abundant identified metabolite with a Cₘₐₓ of 13200 ng-eq/g. Another component of notable concentration was U5 (not identified) at a peak concentration of 33100 ng-ea/g. Mean Cmax of radioactivity in plasma for PPMO-3 and related compounds were observed at 0.083 hours post-dose. The highest C₀ value was observed for PPMO-33 and PPMO-3 (co-eluting peak) and was 209000 ng-eq/g with a plasma half-life of 0.502 hours and exposure (AUC₀₋ₜ) of 42000 ng-eq·h/g; representing 43.71% of total AUC₀₋ₜ (calculated based on the total AUC for identified metabolites and unidentified components for which pharmacokinetic parameters were calculable). PPMO-31 was of notable concentration and represented 11.34% of the total AUC₀₋ₜ, with a C0 of 14600 ng-eq/g and exposure of 10900 ng-eq·h/g.

The most abundant component in urine was PPMO-30 accounting for 27.3% of the administered dose over 0 to 336 hours post-dose. PPMO-31 was also notable, accounting for 15.3% of the administered dose over 0 to 336 hours post-dose. PPMO-33 accounted for 5.27% of the administered dose in urine over 0 to 336 hours post-dose and PPMO-3 accounted for 1.79%. In feces, the most abundant component was PPMO-30 accounting for 4.27% of the administered dose over 0 to 336 hours post-dose, PPMO-3 was not observed. The most abundant component in bicep muscle was U6 (unidentified) for which the peak concentration was 23100 ng equivalents ¹⁴C-PPMO-3/g; PPMO-3 was not observed in this tissue. PPMO-30 was the most abundant metabolite in the kidneys with a peak concentration of 419000 ng equivalents ¹⁴C-PPMO-3/g, representing 15.81% of the sample radioactivity. PPMO-3 was present in kidney at the first pooled time point analyzed (0.25 to 4 hours post-dose) at a concentration of 152000 ng equivalents ¹⁴C-PPMO-3/g and was also quantifiable at 48 hours and in the pooled sample of 96 to 144 hours post-dose.

### Conclusions

PPMO-3 underwent metabolism in male mdx mice after a single intravenous dose of ¹⁴C-PPMO-3, four metabolites were identified by LC-MS. Hydrolysis of the terminal arginine (R) amino acids was the predominant biotransformation pathway for ¹⁴C-PPMO-3.

For the four metabolites that were identified hydrolysis of the terminal arginine amino acids produced PPMO-33 and PPMO-34 present in plasma and urine, PPMO-31 present in plasma, urine, and feces, and PPMO-30 present in plasma, urine, feces, and kidney. PPMO-3 was identified in plasma, urine, and the kidney. Metabolite PPMO-30 was the most abundant metabolite identified in urine and feces and PPMO-31 in plasma.

What is claimed is:

1. An antisense oligomer conjugate of Formula (I): 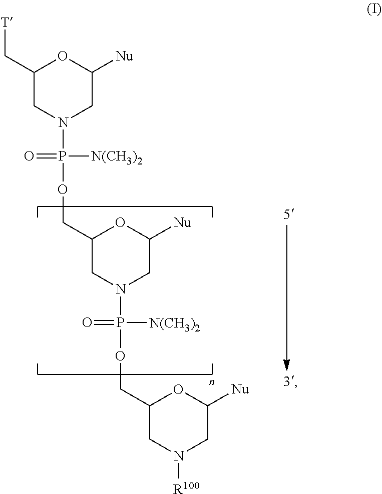
or a pharmaceutically acceptable salt thereof,
wherein:
n is 1-40;
each Nu is a nucleobase, which, taken together, form a targeting sequence complementary to an exon annealing site in the dystrophin pre-mRNA;
T′ is a moiety selected from: 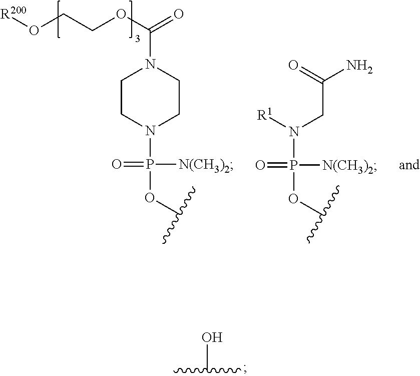
wherein
R¹⁰⁰ is selected from the group consisting of RRRRRG-, RRRRG-, RRRG-, RRG-, RG-, and G-, wherein R is arginine and G is glycine,
R²⁰⁰ is hydrogen, and
R¹ is C₁-C₆ alkyl.

2. The antisense oligomer conjugate of claim 1, or a pharmaceutically acceptable salt thereof, wherein the targeting sequence is complementary to an exon 51 annealing site in the dystrophin pre-mRNA designated as H51A(+66+95).

3. The antisense oligomer conjugate of claim 1, or a pharmaceutically acceptable salt thereof, wherein the targeting sequence is complementary to an exon 45 annealing site in the dystrophin pre-mRNA designated as H45A(−03+19).

4. The antisense oligomer conjugate of claim 1, or a pharmaceutically acceptable salt thereof, wherein the targeting sequence is complementary to an exon 53 annealing site in the dystrophin pre-mRNA designated as H53A(+36+60).

5. The antisense oligomer conjugate of any one of claims 1-4, or a pharmaceutically acceptable salt thereof, wherein each Nu is independently selected from cytosine (C), guanine (G), thymine (T), adenine (A), 5-methylcytosine (5mC), uracil (U), and hypoxanthine (I).

6. The antisense oligomer conjugate of any one of claims 1-5, or a pharmaceutically acceptable salt thereof, wherein T′ is a moiety: 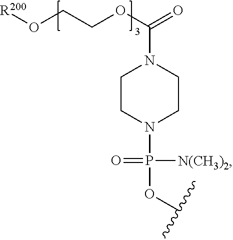
wherein R²⁰⁰ is hydrogen.

7. The antisense oligomer conjugate of any one of claims 1-6, or a pharmaceutically acceptable salt thereof, wherein R¹⁰⁰ is RRRRRG-.

8. The antisense oligomer conjugate of any one of claims 1-6, or a pharmaceutically acceptable salt thereof, wherein R¹⁰⁰ is RRRRG-.

9. The antisense oligomer conjugate of any one of claims 1-6, or a pharmaceutically acceptable salt thereof, wherein R¹⁰⁰ is RRRG-.

10. The antisense oligomer conjugate of any one of claims 1-6, or a pharmaceutically acceptable salt thereof, wherein R¹⁰⁰ is RRG-.

11. The antisense oligomer conjugate of any one of claims 1-6, or a pharmaceutically acceptable salt thereof, wherein R¹⁰⁰ is RG-.

12. The antisense oligomer conjugate of any one of claims 1-6, or a pharmaceutically acceptable salt thereof, wherein R¹⁰⁰ is G-.

13. The antisense oligomer conjugate of any one of claims 1-12, having the Formula (V): 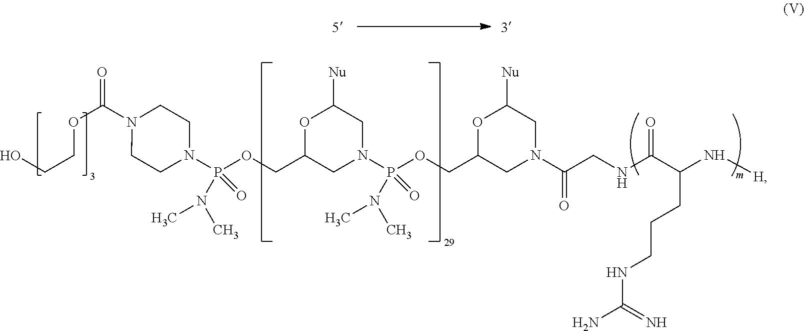
or a pharmaceutically acceptable salt thereof, wherein
each Nu is a nucleobase, which, taken together, form a targeting sequence that is complementary to an exon annealing site in the dystrophin pre-mRNA, and
m is 0, 1, 2, 3, 4, or 5.

14. The antisense oligomer conjugate of any one of claims 1-13, having the Formula (VA): 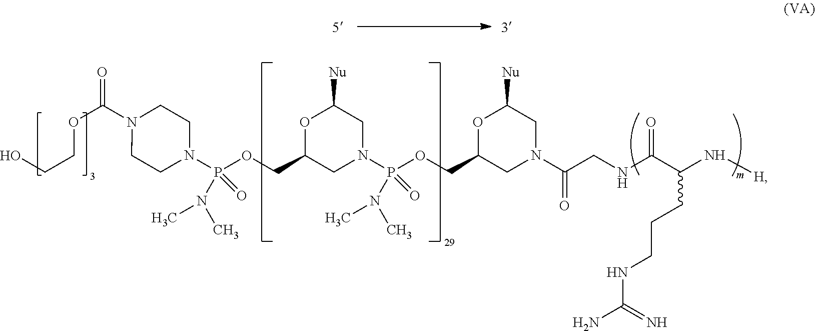
or a pharmaceutically acceptable salt thereof, wherein m is 0, 1, 2, 3, 4, or 5, and
each Nu from 1 to 30 and 5′ to 3′ is:

| Position | |
|---|---|
| No. 5′ | |
| to 3′ | Nu |
| 1 | C |
| 2 | T |
| 3 | C |
| 4 | C |
| 5 | A |
| 6 | A |
| 7 | C |
| 8 | A |
| 9 | T |
| 10 | C |
| 11 | A |
| 12 | A |
| 13 | G |
| 14 | G |
| 15 | A |
| 16 | A |
| 17 | G |
| 18 | A |
| 19 | T |
| 20 | G |
| 21 | G |
| 22 | C |
| 23 | A |
| 24 | T |
| 25 | T |
| 26 | T |
| 27 | C |
| 28 | T |
| 29 | A |
| 30 | G |

wherein A is 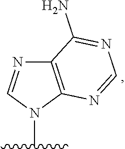 [SMILES: *n1cnc2c(N)ncnc21]
C is 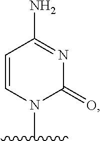 [SMILES: *n1ccc(N)nc1=O]
G is 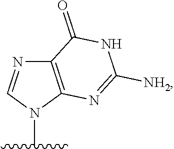 [SMILES: *n1cnc2c(=O)[nH]c(N)nc21]
and T is

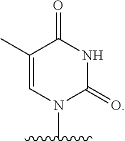 [SMILES: *n1cc(C)c(=O)[nH]c1=O]

15. The antisense oligomer conjugate of any one of claims 1-12, having the Formula (VII): 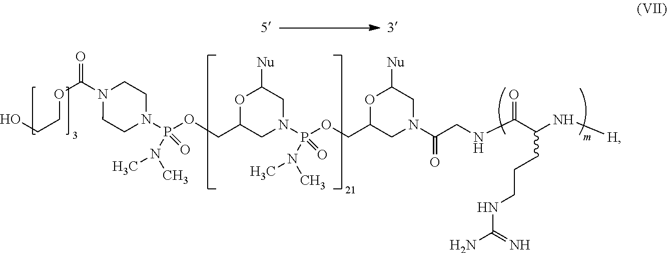
or a pharmaceutically acceptable salt thereof, wherein
each Nu is a nucleobase, which, taken together, form a targeting sequence that is complementary to an exon annealing site in the dystrophin pre-mRNA, and
m is 0, 1, 2, 3, 4, or 5.

16. The antisense oligomer conjugate of any one of claims 1-12 or 15, having the Formula (VIIA): 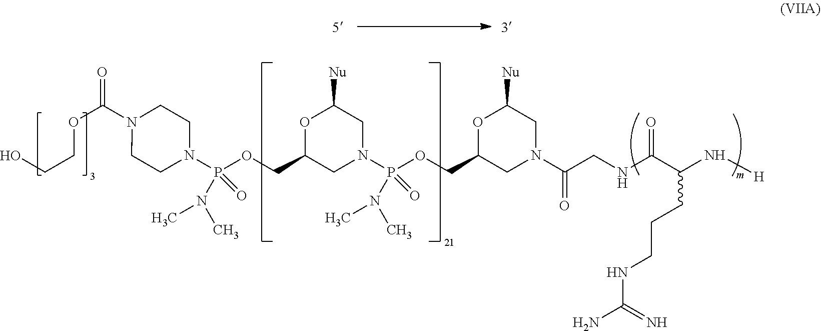
or a pharmaceutically acceptable salt thereof, wherein m is 0, 1, 2, 3, 4, or 5, and
each Nu from 1 to 22 and 5′ to 3′ is:

| Position | |
|---|---|
| No. 5′ | |
| to 3′ | Nu |
| 1 | C |
| 2 | A |
| 3 | A |
| 4 | T |
| 5 | G |
| 6 | C |
| 7 | C |
| 8 | A |
| 9 | T |
| 10 | C |
| 11 | C |
| 12 | T |
| 13 | G |
| 14 | G |
| 15 | A |
| 16 | G |
| 17 | T |
| 18 | T |
| 19 | C |
| 20 | C |
| 21 | T |
| 22 | G |

wherein A is 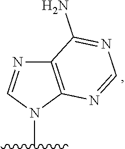 [SMILES: *n1cnc2c(N)ncnc21]
C is 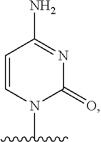 [SMILES: *n1ccc(N)nc1=O]
G is 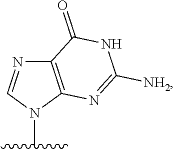 [SMILES: *n1cnc2c(=O)[nH]c(N)nc21]
and T is

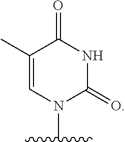 [SMILES: *n1cc(C)c(=O)[nH]c1=O]

17. The antisense oligomer conjugate of any one of claims 1-12, having the Formula (IX): 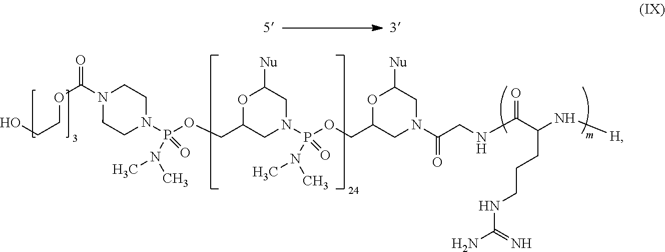
or a pharmaceutically acceptable salt thereof, wherein
each Nu is a nucleobase, which, taken together, form a targeting sequence that is complementary to an exon annealing site in the dystrophin pre-mRNA, and
m is 0, 1, 2, 3, 4, or 5.

18. The antisense oligomer conjugate of any one of claims 1-12 or 17, having the Formula (IXA): 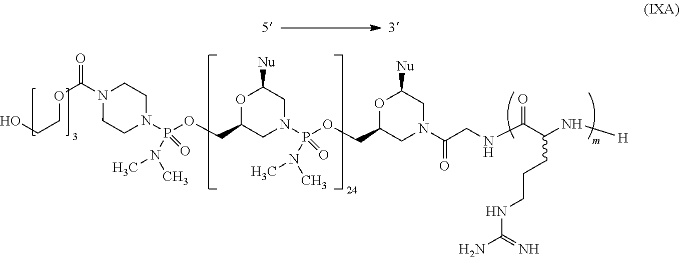
or a pharmaceutically acceptable salt thereof, wherein m is 0, 1, 2, 3, 4, or 5, and
each Nu from 1 to 25 and 5′ to 3′ is:

| Position | |
|---|---|
| No. 5′ | |
| to 3′ | Nu |
| 1 | G |
| 2 | T |
| 3 | T |
| 4 | G |
| 5 | C |
| 6 | C |
| 7 | T |
| 8 | C |
| 9 | C |
| 10 | G |
| 11 | G |
| 12 | T |
| 13 | T |
| 14 | C |
| 15 | T |
| 16 | G |
| 17 | A |
| 18 | A |
| 19 | G |
| 20 | G |
| 21 | T |
| 22 | G |
| 23 | T |
| 24 | T |
| 25 | C |

wherein A is 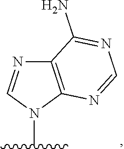 [SMILES: *n1cnc2c(N)ncnc21]
C is 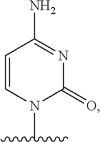 [SMILES: *n1ccc(N)nc1=O]
G is 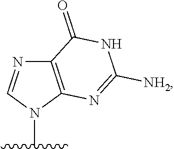 [SMILES: *n1cnc2c(=O)[nH]c(N)nc21]
and T is

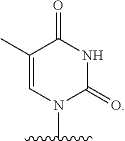 [SMILES: *n1cc(C)c(=O)[nH]c1=O]

19. The antisense oligomer conjugate of any one of claims 13-18, or a pharmaceutically acceptable salt thereof, wherein m is 0.

20. The antisense oligomer conjugate of any one of claims 13-18, or a pharmaceutically acceptable salt thereof, wherein m is 1.

21. The antisense oligomer conjugate of any one of claims 13-18, or a pharmaceutically acceptable salt thereof, wherein m is 2.

22. The antisense oligomer conjugate of any one of claims 13-18, or a pharmaceutically acceptable salt thereof, wherein m is 3.

23. The antisense oligomer conjugate of any one of claims 13-18, or a pharmaceutically acceptable salt thereof, wherein m is 4.

24. The antisense oligomer conjugate of any one of claims 13-18, or a pharmaceutically acceptable salt thereof, wherein m is 5.

25. The antisense oligomer conjugate of any one of claims 1-24, wherein the antisense oligomer conjugate is a free base.

26. The antisense oligomer conjugate of any one of claims 1-24, wherein the antisense oligomer conjugate is a pharmaceutically acceptable salt.

27. The antisense oligomer conjugate of any one of claims 1-24 or 26, wherein the antisense oligomer conjugate is a hydrochloride salt.

28. A pharmaceutical composition, comprising an antisense oligonucleotide conjugate of any one of claims 1-27, or a pharmaceutically acceptable salt thereof, and a pharmaceutically acceptable carrier.

29. The pharmaceutical composition of claim 28, wherein the pharmaceutical composition is formulated for parenteral use.

30. A method of treating a patient with Duchenne muscular dystrophy (DMD) in need thereof who has a mutation that is amenable to exon skipping, comprising administering to the patient an antisense oligomer conjugate of any one of claims 1-27, or a pharmaceutically acceptable salt thereof.

31. The method of claim 30, wherein the antisense oligomer conjugate causes skipping of an exon in the human dystrophin gene.

32. The method of claim 30 or 31, wherein the exon is chosen from exon 44, 45, 50, 51, 52, or 53.

33. The method of any one of claims 30-32, wherein the exon is chosen from exon 45, 51, or 53.

34. A method of treating a patient with Duchenne muscular dystrophy (DMD) with an antisense oligomer conjugate, comprising administering to the patient an antisense oligomer conjugate of any one of claims 1-27, or a pharmaceutically acceptable salt thereof.

35. A method of treating a patient with Duchenne muscular dystrophy (DMD) in need thereof who has a mutation that is amenable to exon skipping, comprising administering to the patient a composition of claim 28 or 29, and a pharmaceutically acceptable carrier.

36. The method of claim 35, wherein the antisense oligomer conjugate causes skipping of an exon in the human dystrophin gene.

37. The method of claim 35 or 36, wherein the exon is chosen from exon 44, 45, 50, 51, 52, or 53.

38. The method of any one of claims 35-37, wherein the exon is chosen from exon 45, 51, or 53.

39. A method of treating a patient with Duchenne muscular dystrophy (DMD) with an antisense oligomer conjugate, comprising administering to the patient a composition of claims 28 or 29, and a pharmaceutically acceptable carrier.

40. A method of treating a patient with DMD in need thereof who has a mutation that is amenable to exon 51 skipping, comprising administering to the patient an antisense oligomer conjugate having Formula (VI): 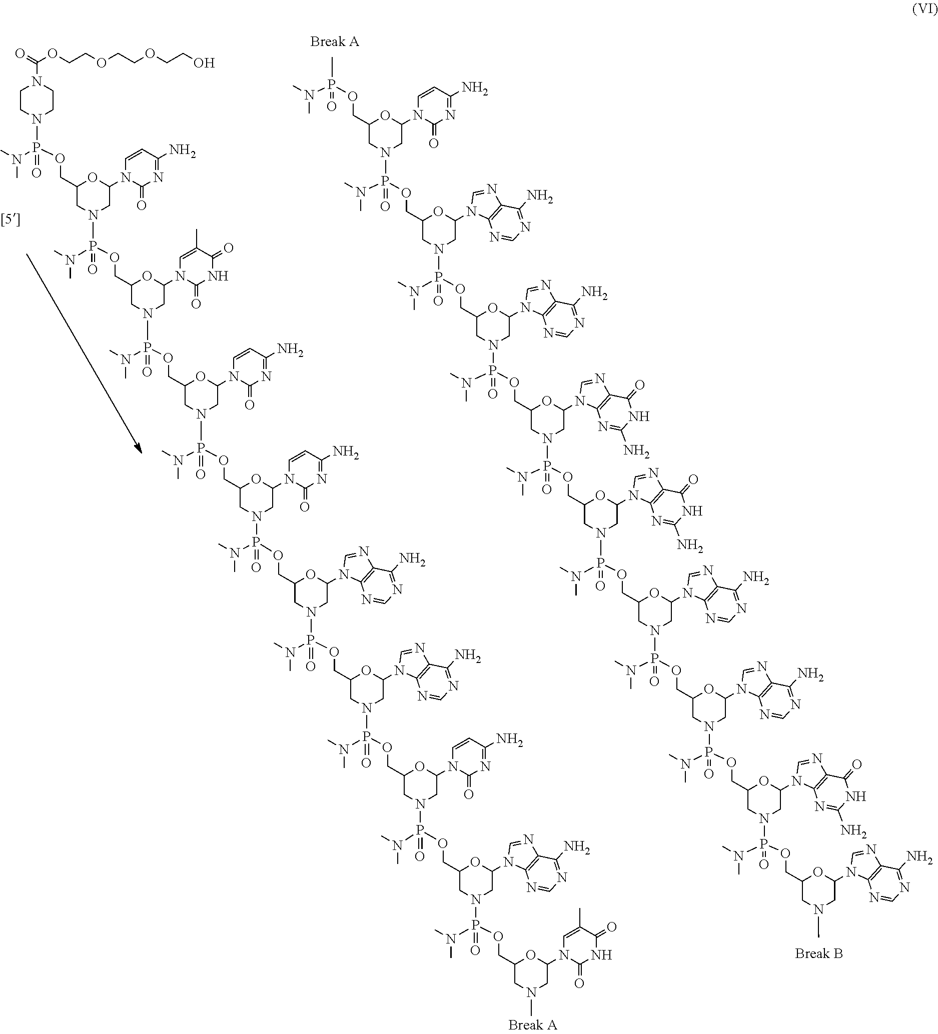 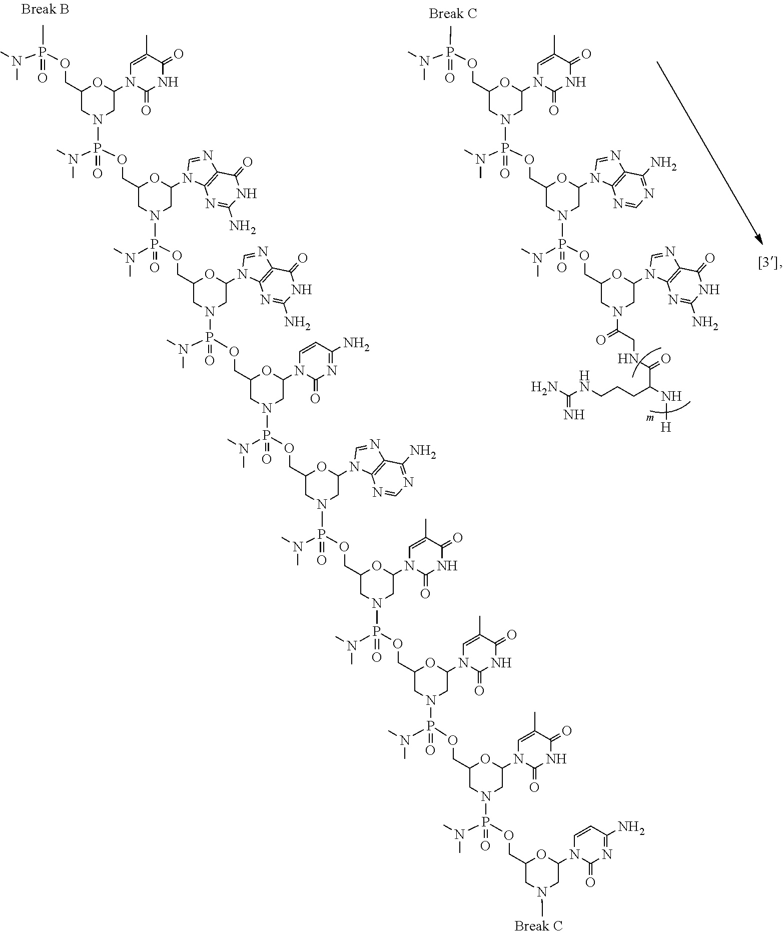
or a pharmaceutically acceptable salt thereof, wherein m is 0, 1, 2, 3, 4, or 5.

41. A method of treating a patient with DMD in need thereof who has a mutation that is amenable to exon 45 skipping, comprising administering to the patient an antisense oligomer conjugate having Formula (VIII): 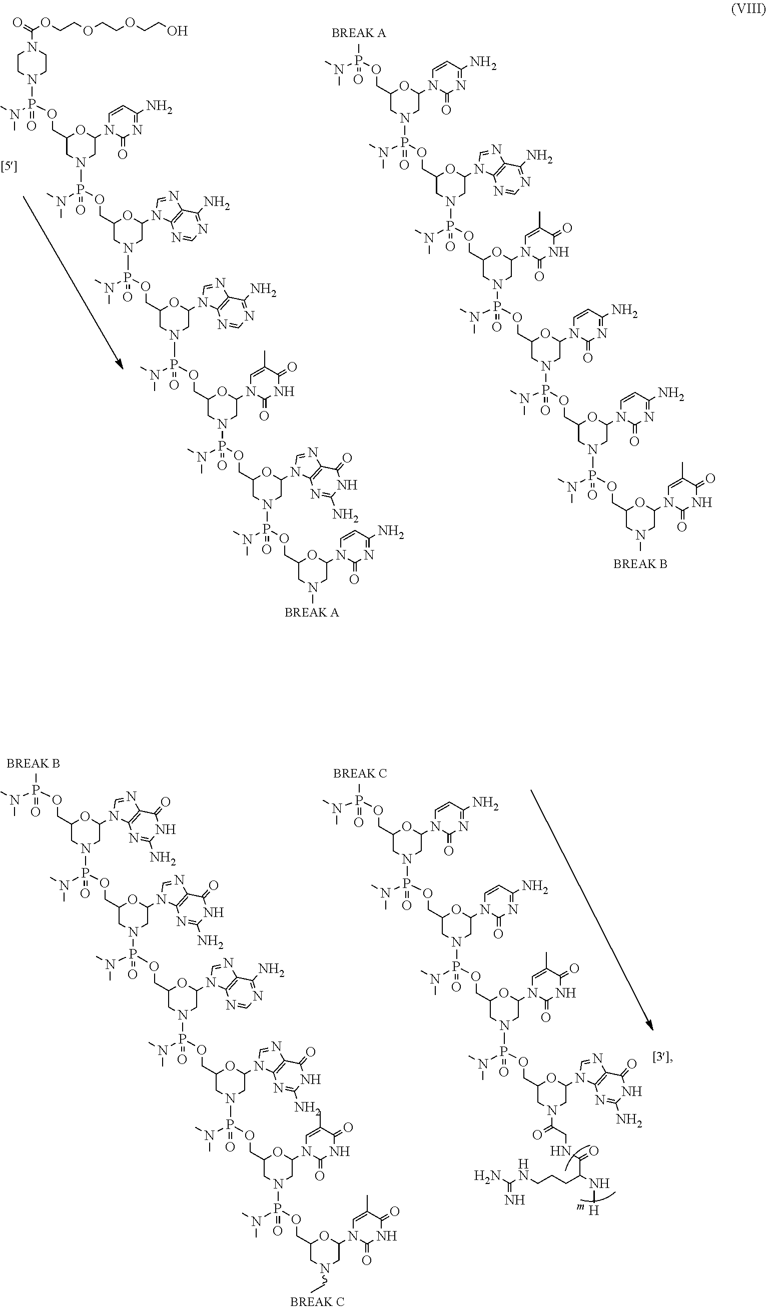
or a pharmaceutically acceptable salt thereof, wherein m is 0, 1, 2, 3, 4, or 5.

42. A method of treating a patient with DMID in need thereof who has a mutation that is amenable to exon 53 skipping, comprising administering to the patient an antisense oligomer conjugate having Formula (X): 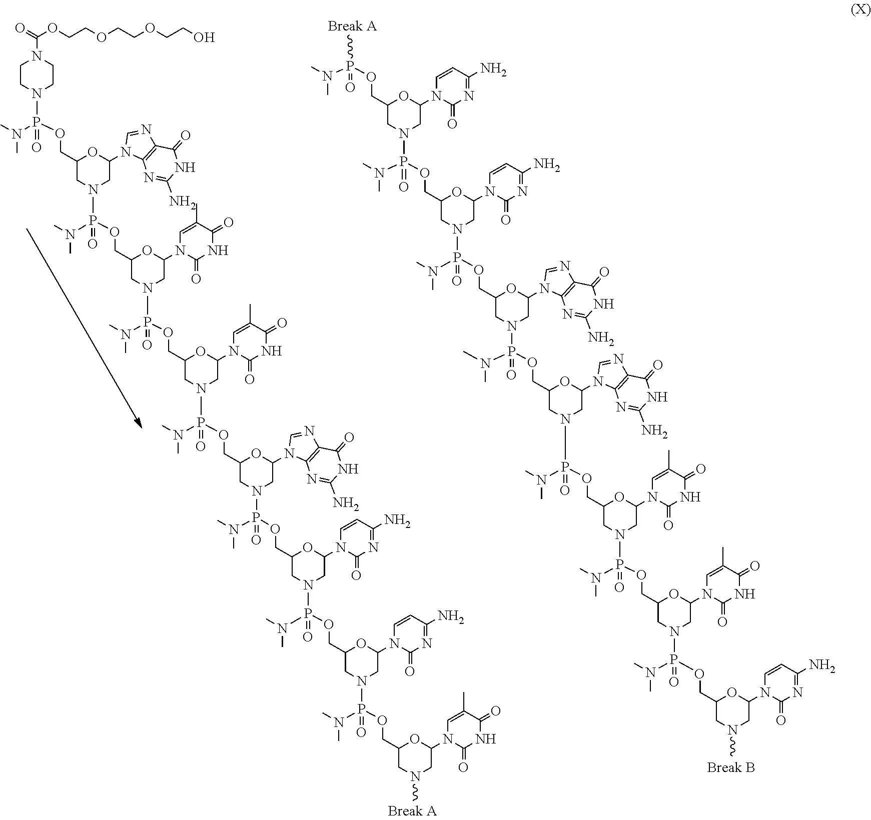 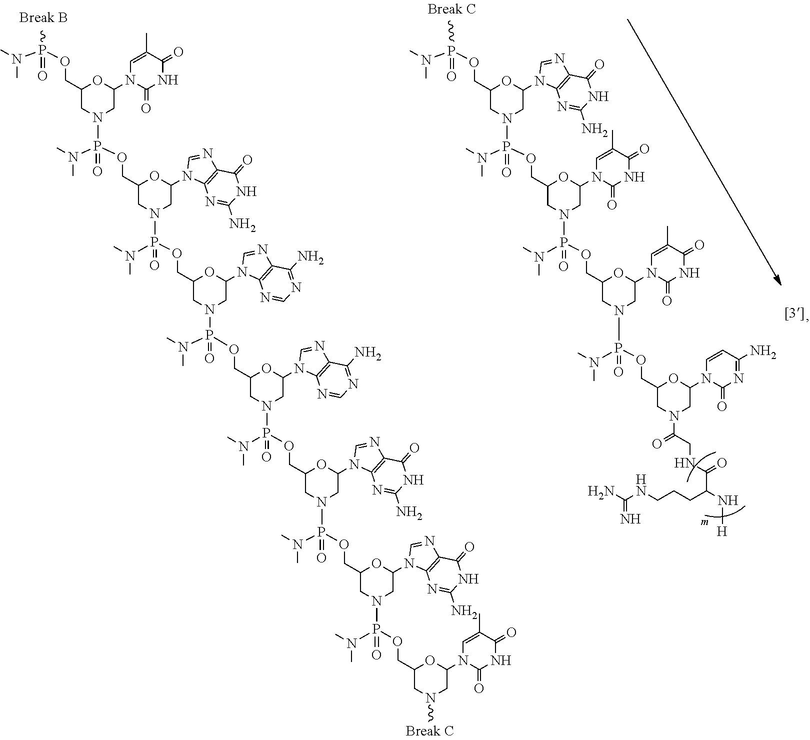
or a pharmaceutically acceptable salt thereof, wherein m is 0, 1, 2, 3, 4, or 5.

43. An antisense oligomer conjugate according to any one of claims 1-27, or a pharmaceutically acceptable salt thereof, for use in the treatment of Duchenne muscular dystrophy (DMD) in a patient who has a mutation that is amenable to exon skipping.

44. The antisense oligomer conjugate for use according to claim 43, wherein the antisense oligomer conjugate causes skipping of an exon in the human dystrophin gene.

45. The antisense oligomer conjugate for use according to claim 43 or 44, wherein the exon is chosen from exon 44, 45, 50, 51, 52, or 53.

46. The antisense oligomer conjugate for use according to any one of claims 43-45, wherein the exon is chosen from exon 45, 51, or 53.

47. A pharmaceutical composition comprising an antisense oligonucleotide conjugate according to any one of claims 1-27, or a pharmaceutically acceptable salt thereof, for use in the treatment of Duchenne muscular dystrophy (DMD) in a patient who has a mutation that is amenable to exon skipping.

48. The pharmaceutical composition for use according to claim 47, wherein the antisense oligomer conjugate causes skipping of an exon in the human dystrophin gene.

49. The pharmaceutical composition for use according to claim 47 or 48, wherein the exon is chosen from exon 44, 45, 50, 51, 52, or 53.

50. The pharmaceutical composition for use according to any one of claims 47-49, wherein the exon is chosen from exon 45, 51, or 53.

51. The pharmaceutical composition for use according to any one of claims 47-50, wherein the pharmaceutical composition is formulated for parenteral use.

52. An antisense oligomer conjugate according to any one of claims 1-27, or a pharmaceutically acceptable salt thereof, for use as a medicament.


SEQUENCE LISTING (523 sequences)

SEQ ID NO: 1 - DNA, 30 bp, synthetic construct
  source 1..30: mol_type = other DNA
ctccaacatcaaggaagatggcatttctag

SEQ ID NO: 2 - DNA, 22 bp, synthetic construct
  source 1..22: mol_type = other DNA
caatgccatcctggagttcctg

SEQ ID NO: 3 - DNA, 25 bp, synthetic construct
  source 1..25: mol_type = other DNA
gttgcctccggttctgaaggtgttc

SEQ ID NO: 4 - DNA, 23 bp, synthetic construct
  source 1..23: mol_type = other DNA; note = H51D(+16-07)
ctcataccttctgcttgatgatc

SEQ ID NO: 5 - DNA, 25 bp, synthetic construct
  source 1..25: mol_type = other DNA; note = H50D(+103+127)
gggatccagtatacttacaggctcc

SEQ ID NO: 6 - DNA, 22 bp, synthetic construct
  source 1..22: mol_type = other DNA; note = H51A(+61+82)
gaagatggcatttctagtttgg

SEQ ID NO: 7 - DNA, 23 bp, synthetic construct
  source 1..23: mol_type = other DNA; note = H51A(+61+83)
ggaagatggcatttctagtttgg

SEQ ID NO: 8 - DNA, 29 bp, synthetic construct
  source 1..29: mol_type = other DNA; note = H51A(+61+89)
catcaaggaagatggcatttctagtttgg

SEQ ID NO: 9 - DNA, 24 bp, synthetic construct
  source 1..24: mol_type = other DNA; note = H51A(+66+89)
catcaaggaagatggcatttctag

SEQ ID NO: 10 - DNA, 28 bp, synthetic construct
  source 1..28: mol_type = other DNA; note = H51A(+66+93)
ccaacatcaaggaagatggcatttctag

SEQ ID NO: 11 - DNA, 24 bp, synthetic construct
  source 1..24: mol_type = other DNA; note = H51A(+69+92)
caacatcaaggaagatggcatttc

SEQ ID NO: 12 - DNA, 28 bp, synthetic construct
  source 1..28: mol_type = other DNA; note = H51A(+69+96)
cctccaacatcaaggaagatggcatttc

SEQ ID NO: 13 - DNA, 23 bp, synthetic construct
  source 1..23: mol_type = other DNA; note = H51A(+74+96)
cctccaacatcaaggaagatggc

SEQ ID NO: 14 - DNA, 26 bp, synthetic construct
  source 1..26: mol_type = other DNA; note = H51A(+74+99)
gtacctccaacatcaaggaagatggc

SEQ ID NO: 15 - DNA, 27 bp, synthetic construct
  source 1..27: mol_type = other DNA; note = H51A(+74+100)
ggtacctccaacatcaaggaagatggc

SEQ ID NO: 16 - DNA, 29 bp, synthetic construct
  source 1..29: mol_type = other DNA; note = H51A(+74+102)
caggtacctccaacatcaaggaagatggc

SEQ ID NO: 17 - DNA, 30 bp, synthetic construct
  source 1..30: mol_type = other DNA; note = H51A(+74+103)
gcaggtacctccaacatcaaggaagatggc

SEQ ID NO: 18 - DNA, 22 bp, synthetic construct
  source 1..22: mol_type = other DNA; note = H51A(+75+96)
cctccaacatcaaggaagatgg

SEQ ID NO: 19 - DNA, 25 bp, synthetic construct
  source 1..25: mol_type = other DNA; note = H51A(+75+99)
gtacctccaacatcaaggaagatgg

SEQ ID NO: 20 - DNA, 24 bp, synthetic construct
  source 1..24: mol_type = other DNA; note = H51A(+76+99)
gtacctccaacatcaaggaagatg

SEQ ID NO: 21 - DNA, 30 bp, synthetic construct
  source 1..30: mol_type = other DNA; note = H51A(+76+105)
gagcaggtacctccaacatcaaggaagatg

SEQ ID NO: 22 - DNA, 25 bp, synthetic construct
  source 1..25: mol_type = other DNA; note = H51A(+80+103)
gcaggtacctccaacatcaaggaag

SEQ ID NO: 23 - DNA, 27 bp, synthetic construct
  source 1..27: mol_type = other DNA; note = H51A(+80+105)
gagcaggtacctccaacatcaaggaag

SEQ ID NO: 24 - DNA, 29 bp, synthetic construct
  source 1..29: mol_type = other DNA; note = H51A(+80+107)
cagagcaggtacctccaacatcaaggaag

SEQ ID NO: 25 - DNA, 30 bp, synthetic construct
  source 1..30: mol_type = other DNA; note = H51A(+80+108)
ccagagcaggtacctccaacatcaaggaag

SEQ ID NO: 26 - DNA, 22 bp, synthetic construct
  source 1..22: mol_type = other DNA; note = H51A(+83+103)
gcaggtacctccaacatcaagg

SEQ ID NO: 27 - DNA, 24 bp, synthetic construct
  source 1..24: mol_type = other DNA; note = H51A(+83+105)
gagcaggtacctccaacatcaagg

SEQ ID NO: 28 - DNA, 26 bp, synthetic construct
  source 1..26: mol_type = other DNA; note = H51A(+83+107)
cagagcaggtacctccaacatcaagg

SEQ ID NO: 29 - DNA, 28 bp, synthetic construct
  source 1..28: mol_type = other DNA; note = H51A(+83+109)
gccagagcaggtacctccaacatcaagg

SEQ ID NO: 30 - DNA, 25 bp, synthetic construct
  source 1..25: mol_type = other DNA; note = H51A(+84+107)
cagagcaggtacctccaacatcaag

SEQ ID NO: 31 - DNA, 29 bp, synthetic construct
  source 1..29: mol_type = other DNA; note = H51A(+84+111)
ctgccagagcaggtacctccaacatcaag

SEQ ID NO: 32 - DNA, 23 bp, synthetic construct
  source 1..23: mol_type = other DNA; note = H51A(+84+105)
gagcaggtacctccaacatcaag

SEQ ID NO: 33 - DNA, 24 bp, synthetic construct
  source 1..24: mol_type = other DNA; note = H51A(+87+109)
gccagagcaggtacctccaacatc

SEQ ID NO: 34 - DNA, 24 bp, synthetic construct
  source 1..24: mol_type = other DNA; note = H51A(+93+116)
gaaatctgccagagcaggtacctc

SEQ ID NO: 35 - DNA, 26 bp, synthetic construct
  source 1..26: mol_type = other DNA; note = H51A(+75+100)
ggtacctccaacatcaaggaagatgg

SEQ ID NO: 36 - DNA, 28 bp, synthetic construct
  source 1..28: mol_type = other DNA; note = H51A(+74+101)
aggtacctccaacatcaaggaagatggc

SEQ ID NO: 37 - DNA, 25 bp, synthetic construct
  source 1..25: mol_type = other DNA; note = H51A(+74+98)
tacctccaacatcaaggaagatggc

SEQ ID NO: 38 - DNA, 24 bp, synthetic construct
  source 1..24: mol_type = other DNA; note = H51A(+74+97)
acctccaacatcaaggaagatggc

SEQ ID NO: 39 - DNA, 21 bp, synthetic construct
  source 1..21: mol_type = other DNA; note = H51A(+74+94)
tccaacatcaaggaagatggc

SEQ ID NO: 40 - DNA, 20 bp, synthetic construct
  source 1..20: mol_type = other DNA; note = H51A(+74+93)
ccaacatcaaggaagatggc

SEQ ID NO: 41 - DNA, 19 bp, synthetic construct
  source 1..19: mol_type = other DNA; note = H51A(+74+92)
caacatcaaggaagatggc

SEQ ID NO: 42 - DNA, 31 bp, synthetic construct
  source 1..31: mol_type = other DNA; note = H51A(+69+99)
gtacctccaacatcaaggaagatggcatttc

SEQ ID NO: 43 - DNA, 30 bp, synthetic construct
  source 1..30: mol_type = other DNA; note = H51A(+70+99)
gtacctccaacatcaaggaagatggcattt

SEQ ID NO: 44 - DNA, 29 bp, synthetic construct
  source 1..29: mol_type = other DNA; note = H51A(+71+99)
gtacctccaacatcaaggaagatggcatt

SEQ ID NO: 45 - DNA, 28 bp, synthetic construct
  source 1..28: mol_type = other DNA; note = H51A(+72+99)
gtacctccaacatcaaggaagatggcat

SEQ ID NO: 46 - DNA, 27 bp, synthetic construct
  source 1..27: mol_type = other DNA; note = H51A(+73+99)
gtacctccaacatcaaggaagatggca

SEQ ID NO: 47 - DNA, 23 bp, synthetic construct
  source 1..23: mol_type = other DNA; note = H51A(+77+99)
gtacctccaacatcaaggaagat

SEQ ID NO: 48 - DNA, 22 bp, synthetic construct
  source 1..22: mol_type = other DNA; note = H51A(+78+99)
gtacctccaacatcaaggaaga

SEQ ID NO: 49 - DNA, 21 bp, synthetic construct
  source 1..21: mol_type = other DNA; note = H51A(+79+99)
gtacctccaacatcaaggaag

SEQ ID NO: 50 - DNA, 25 bp, synthetic construct
  source 1..25: mol_type = other DNA; note = H51.SA.(-60-36)
gaagaaaaagaaaaattagaaacac

SEQ ID NO: 51 - DNA, 25 bp, synthetic construct
  source 1..25: mol_type = other DNA; note = H51.SA.(-50-26)
aaggaaaaaagaagaaaaagaaaaa

SEQ ID NO: 52 - DNA, 25 bp, synthetic construct
  source 1..25: mol_type = other DNA; note = H51.SA.(-45-21)
gcaaaaaggaaaaaagaagaaaaag

SEQ ID NO: 53 - DNA, 25 bp, synthetic construct
  source 1..25: mol_type = other DNA; note = H51.SA.(-40-16)
tttttgcaaaaaggaaaaaagaaga

SEQ ID NO: 54 - DNA, 25 bp, synthetic construct
  source 1..25: mol_type = other DNA; note = H51.SA.(-35-11)
ttgggtttttgcaaaaaggaaaaaa

SEQ ID NO: 55 - DNA, 25 bp, synthetic construct
  source 1..25: mol_type = other DNA; note = H51.SA.(-30-6)
atattttgggtttttgcaaaaagga

SEQ ID NO: 56 - DNA, 25 bp, synthetic construct
  source 1..25: mol_type = other DNA; note = H51.SA.(-25-1)
ctaaaatattttgggtttttgcaaa

SEQ ID NO: 57 - DNA, 25 bp, synthetic construct
  source 1..25: mol_type = other DNA; note = H51.SA.(-20+5)
aggagctaaaatattttgggttttt

SEQ ID NO: 58 - DNA, 25 bp, synthetic construct
  source 1..25: mol_type = other DNA; note = H51.SA.(-15+10)
tgagtaggagctaaaatattttggg

SEQ ID NO: 59 - DNA, 25 bp, synthetic construct
  source 1..25: mol_type = other DNA; note = H51.SA.(-10+15)
cagtctgagtaggagctaaaatatt

SEQ ID NO: 60 - DNA, 25 bp, synthetic construct
  source 1..25: mol_type = other DNA; note = H51.SA.(-5+20)
agtaacagtctgagtaggagctaaa

SEQ ID NO: 61 - DNA, 25 bp, synthetic construct
  source 1..25: mol_type = other DNA; note = H51.SA.(-1+24)
ccagagtaacagtctgagtaggagc

SEQ ID NO: 62 - DNA, 25 bp, synthetic construct
  source 1..25: mol_type = other DNA; note = H51.SA.(-65-41)
aaaagaaaaattagaaacacaagct

SEQ ID NO: 63 - DNA, 25 bp, synthetic construct
  source 1..25: mol_type = other DNA; note = H51.SA.(-70-46)
aaaaattagaaacacaagctaaaga

SEQ ID NO: 64 - DNA, 25 bp, synthetic construct
  source 1..25: mol_type = other DNA; note = H51.SA.(-75-51)
ttagaaacacaagctaaagagccaa

SEQ ID NO: 65 - DNA, 25 bp, synthetic construct
  source 1..25: mol_type = other DNA; note = H51.SA.(-80-56)
aacacaagctaaagagccaatttca

SEQ ID NO: 66 - DNA, 25 bp, synthetic construct
  source 1..25: mol_type = other DNA; note = H51.SA.(-85-61)
aagctaaagagccaatttcaataac

SEQ ID NO: 67 - DNA, 25 bp, synthetic construct
  source 1..25: mol_type = other DNA; note = H51.SA.(-90-66)
aaagagccaatttcaataacaataa

SEQ ID NO: 68 - DNA, 25 bp, synthetic construct
  source 1..25: mol_type = other DNA; note = H51.SA.(-95-71)
gccaatttcaataacaataagtcaa

SEQ ID NO: 69 - DNA, 25 bp, synthetic construct
  source 1..25: mol_type = other DNA; note = H51.SA.(-100-76)
tttcaataacaataagtcaaattta

SEQ ID NO: 70 - DNA, 30 bp, synthetic construct
  source 1..30: mol_type = other DNA; note = H51A(+1+30)
gtgtcaccagagtaacagtctgagtaggag

SEQ ID NO: 71 - DNA, 30 bp, synthetic construct
  source 1..30: mol_type = other DNA; note = H51A(+10+39)
ccacaggttgtgtcaccagagtaacagtct

SEQ ID NO: 72 - DNA, 30 bp, synthetic construct
  source 1..30: mol_type = other DNA; note = H51A(+6+35)
aggttgtgtcaccagagtaacagtctgagt

SEQ ID NO: 73 - DNA, 30 bp, synthetic construct
  source 1..30: mol_type = other DNA; note = H51A(+49+78)
atggcatttctagtttggagatggcagttt

SEQ ID NO: 74 - DNA, 25 bp, synthetic construct
  source 1..25: mol_type = other DNA; note = H51A(+1+25)
accagagtaacagtctgagtaggag

SEQ ID NO: 75 - DNA, 25 bp, synthetic construct
  source 1..25: mol_type = other DNA; note = H51A(+4+28)
gtcaccagagtaacagtctgagtag

SEQ ID NO: 76 - DNA, 25 bp, synthetic construct
  source 1..25: mol_type = other DNA; note = H51A(+16+40)
accacaggttgtgtcaccagagtaa

SEQ ID NO: 77 - DNA, 25 bp, synthetic construct
  source 1..25: mol_type = other DNA; note = H51A(+21+45)
tagtaaccacaggttgtgtcaccag

SEQ ID NO: 78 - DNA, 25 bp, synthetic construct
  source 1..25: mol_type = other DNA; note = H51A(+26+50)
ttccttagtaaccacaggttgtgtc

SEQ ID NO: 79 - DNA, 25 bp, synthetic construct
  source 1..25: mol_type = other DNA; note = H51A(+31+55)
gcagtttccttagtaaccacaggtt

SEQ ID NO: 80 - DNA, 25 bp, synthetic construct
  source 1..25: mol_type = other DNA; note = H51A(+36+60)
agatggcagtttccttagtaaccac

SEQ ID NO: 81 - DNA, 25 bp, synthetic construct
  source 1..25: mol_type = other DNA; note = H51A(+41+65)
tttggagatggcagtttccttagta

SEQ ID NO: 82 - DNA, 25 bp, synthetic construct
  source 1..25: mol_type = other DNA; note = H51A(+86+110)
tgccagagcaggtacctccaacatc

SEQ ID NO: 83 - DNA, 25 bp, synthetic construct
  source 1..25: mol_type = other DNA; note = H51A(+91+115)
aaatctgccagagcaggtacctcca

SEQ ID NO: 84 - DNA, 25 bp, synthetic construct
  source 1..25: mol_type = other DNA; note = H51A(+96+120)
ggttgaaatctgccagagcaggtac

SEQ ID NO: 85 - DNA, 25 bp, synthetic construct
  source 1..25: mol_type = other DNA; note = H51A(+101+125)
agcccggttgaaatctgccagagca

SEQ ID NO: 86 - DNA, 25 bp, synthetic construct
  source 1..25: mol_type = other DNA; note = H51A(+106+130)
gtccaagcccggttgaaatctgcca

SEQ ID NO: 87 - DNA, 25 bp, synthetic construct
  source 1..25: mol_type = other DNA; note = H51A(+111+135)
gttctgtccaagcccggttgaaatc

SEQ ID NO: 88 - DNA, 25 bp, synthetic construct
  source 1..25: mol_type = other DNA; note = H51A(+116+140)
ggtaagttctgtccaagcccggttg

SEQ ID NO: 89 - DNA, 25 bp, synthetic construct
  source 1..25: mol_type = other DNA; note = H51A(+121+145)
cagtcggtaagttctgtccaagccc

SEQ ID NO: 90 - DNA, 25 bp, synthetic construct
  source 1..25: mol_type = other DNA; note = H51A(+126+150)
aaagccagtcggtaagttctgtcca

SEQ ID NO: 91 - DNA, 25 bp, synthetic construct
  source 1..25: mol_type = other DNA; note = H51A(+131+155)
cagagaaagccagtcggtaagttct

SEQ ID NO: 92 - DNA, 25 bp, synthetic construct
  source 1..25: mol_type = other DNA; note = H51A(+136+160)
tcaagcagagaaagccagtcggtaa

SEQ ID NO: 93 - DNA, 25 bp, synthetic construct
  source 1..25: mol_type = other DNA; note = H51A(+141+165)
cttgatcaagcagagaaagccagtc

SEQ ID NO: 94 - DNA, 25 bp, synthetic construct
  source 1..25: mol_type = other DNA; note = H51A(+146+170)
tataacttgatcaagcagagaaagc

SEQ ID NO: 95 - DNA, 25 bp, synthetic construct
  source 1..25: mol_type = other DNA; note = H51A(+151+175)
gattttataacttgatcaagcagag

SEQ ID NO: 96 - DNA, 25 bp, synthetic construct
  source 1..25: mol_type = other DNA; note = H51A(+156+180)
tctgtgattttataacttgatcaag

SEQ ID NO: 97 - DNA, 25 bp, synthetic construct
  source 1..25: mol_type = other DNA; note = H51A(+161+185)
caccctctgtgattttataacttga

SEQ ID NO: 98 - DNA, 25 bp, synthetic construct
  source 1..25: mol_type = other DNA; note = H51A(+166+190)
accatcaccctctgtgattttataa

SEQ ID NO: 99 - DNA, 25 bp, synthetic construct
  source 1..25: mol_type = other DNA; note = H51A(+171+195)
cacccaccatcaccctctgtgattt

SEQ ID NO: 100 - DNA, 25 bp, synthetic construct
  source 1..25: mol_type = other DNA; note = H51A(+176+200)
aaggtcacccaccatcaccctctgt

SEQ ID NO: 101 - DNA, 25 bp, synthetic construct
  source 1..25: mol_type = other DNA; note = H51A(+181+205)
tcctcaaggtcacccaccatcaccc

SEQ ID NO: 102 - DNA, 25 bp, synthetic construct
  source 1..25: mol_type = other DNA; note = H51A(+186+210)
tgatatcctcaaggtcacccaccat

SEQ ID NO: 103 - DNA, 25 bp, synthetic construct
  source 1..25: mol_type = other DNA; note = H51A(+191+215)
ctcgttgatatcctcaaggtcaccc

SEQ ID NO: 104 - DNA, 25 bp, synthetic construct
  source 1..25: mol_type = other DNA; note = H51A(+196+220)
atcatctcgttgatatcctcaaggt

SEQ ID NO: 105 - DNA, 25 bp, synthetic construct
  source 1..25: mol_type = other DNA; note = H51A(+201+225)
tgatgatcatctcgttgatatcctc

SEQ ID NO: 106 - DNA, 25 bp, synthetic construct
  source 1..25: mol_type = other DNA; note = H51A(+206+230)
ctgcttgatgatcatctcgttgata

SEQ ID NO: 107 - DNA, 25 bp, synthetic construct
  source 1..25: mol_type = other DNA; note = H51D(+211-02)
accttctgcttgatgatcatctcgt

SEQ ID NO: 108 - DNA, 25 bp, synthetic construct
  source 1..25: mol_type = other DNA; note = H51D(+214-05)
cataccttctgcttgatgatcatct

SEQ ID NO: 109 - DNA, 25 bp, synthetic construct
  source 1..25: mol_type = other DNA; note = H51D(+217-08)
tctcataccttctgcttgatgatca

SEQ ID NO: 110 - DNA, 25 bp, synthetic construct
  source 1..25: mol_type = other DNA; note = H51D(+220-11)
ttttctcataccttctgcttgatga

SEQ ID NO: 111 - DNA, 25 bp, synthetic construct
  source 1..25: mol_type = other DNA; note = H51D(+223-14)
attttttctcataccttctgcttga

SEQ ID NO: 112 - DNA, 25 bp, synthetic construct
  source 1..25: mol_type = other DNA; note = H51D(+226-17)
atcattttttctcataccttctgct

SEQ ID NO: 113 - DNA, 25 bp, synthetic construct
  source 1..25: mol_type = other DNA; note = H51D(+229-20)
tttatcattttttctcataccttct

SEQ ID NO: 114 - DNA, 25 bp, synthetic construct
  source 1..25: mol_type = other DNA; note = H51D(+232-23)
acttttatcattttttctcatacct

SEQ ID NO: 115 - DNA, 25 bp, synthetic construct
  source 1..25: mol_type = other DNA; note = H51D(-02-26)
ccaacttttatcattttttctcata

SEQ ID NO: 116 - DNA, 30 bp, synthetic construct; identical to SEQ ID NO: 70
  source 1..30: mol_type = other DNA; note = H51A(+1+30)

SEQ ID NO: 117 - DNA, 30 bp, synthetic construct; identical to SEQ ID NO: 1
  source 1..30: mol_type = other DNA; note = H51A(+66+95)

SEQ ID NO: 118 - DNA, 24 bp, synthetic construct
  source 1..24: mol_type = other DNA; note = H51A(+74+97)
acctccaacatcaaggaagatggc

SEQ ID NO: 119 - DNA, 30 bp, synthetic construct; identical to SEQ ID NO: 43
  source 1..30: mol_type = other DNA; note = H51A(+70+99)

SEQ ID NO: 120 - DNA, 28 bp, synthetic construct
  source 1..28: mol_type = other DNA; note = H51A(+72+99)
gtacctccaacatcaaggaagatggcat

SEQ ID NO: 121 - DNA, 20 bp, synthetic construct
  source 1..20: mol_type = other DNA; note = H51A(+68+87)
tcaaggaagatggcatttct

SEQ ID NO: 122 - RNA, 20 nt, synthetic construct
  source 1..20: mol_type = other RNA; note = H51A(+68+87)
  modified_base 7: mod_base = OTHER; note = methylated adenine
  modified_base 9: mod_base = OTHER; note = methylated guanine
  modified_base 10: mod_base = OTHER; note = methylated adenine
  modified_base 12..13: mod_base = OTHER; note = methylated guanine
tcaaggnanntnncatttct

SEQ ID NO: 123 - DNA, 25 bp, synthetic construct
  source 1..25: mol_type = other DNA; note = H53A(+36+60)
gttgcctccggttctgaaggtgttc

SEQ ID NO: 124 - DNA, 25 bp, synthetic construct
  source 1..25: mol_type = other DNA; note = H53A(+36+60)
  modified_base 5: mod_base = m5c
  modified_base 6: mod_base = m5c
  modified_base 8: mod_base = m5c
  modified_base 9: mod_base = m5c
  modified_base 14: mod_base = m5c
  modified_base 25: mod_base = m5c
gttgnntnnggttntgaaggtgttn

SEQ ID NO: 125 - DNA, 21 bp, synthetic construct
  source 1..21: mol_type = other DNA; note = H53A(+36+56)
cctccggttctgaaggtgttc

SEQ ID NO: 126 - DNA, 25 bp, synthetic construct
  source 1..25: mol_type = other DNA; note = H53A(+23+47)
ctgaaggtgttcttgtacttcatcc

SEQ ID NO: 127 - DNA, 25 bp, synthetic construct
  source 1..25: mol_type = other DNA; note = H53A(+32+56)
cctccggttctgaaggtgttcttgt

SEQ ID NO: 128 - DNA, 28 bp, synthetic construct
  source 1..28: mol_type = other DNA; note = H53A(+33+60)
gttgcctccggttctgaaggtgttcttg

SEQ ID NO: 129 - DNA, 30 bp, synthetic construct
  source 1..30: mol_type = other DNA; note = H53A(+30+59)
ttgcctccggttctgaaggtgttcttgtac

SEQ ID NO: 130 - DNA, 24 bp, synthetic construct
  source 1..24: mol_type = other DNA; note = H53A(+39+62)
ctgttgcctccggttctgaaggtg

SEQ ID NO: 131 - DNA, 31 bp, synthetic construct
  source 1..31: mol_type = other DNA; note = H53A(+36+69)
cattcaactgttgcctccggttctgaaggtg

SEQ ID NO: 132 - DNA, 18 bp, synthetic construct
  source 1..18: mol_type = other DNA; note = H53A(+45+62)
ctgttgcctccggttctg

SEQ ID NO: 133 - DNA, 22 bp, synthetic construct
  source 1..22: mol_type = other DNA; note = H45A(-03+19)
caatgccatcctggagttcctg

SEQ ID NO: 134 - DNA, 34 bp, synthetic construct
  source 1..34: mol_type = other DNA; note = H45A(-09+25)
gctgcccaatgccatcctggagttcctgtaagat

SEQ ID NO: 135 - DNA, 28 bp, synthetic construct
  source 1..28: mol_type = other DNA; note = H45A(-03+25)
gctgcccaatgccatcctggagttcctg

SEQ ID NO: 136 - DNA, 31 bp, synthetic construct
  source 1..31: mol_type = other DNA; note = H45A(-06+25)
gctgcccaatgccatcctggagttcctgtaa

SEQ ID NO: 137 - DNA, 31 bp, synthetic construct
  source 1..31: mol_type = other DNA; note = H45A(-12+19)
caatgccatcctggagttcctgtaagatacc

SEQ ID NO: 138 - DNA, 28 bp, synthetic construct
  source 1..28: mol_type = other DNA; note = H45A(-09+19)
caatgccatcctggagttcctgtaagat

SEQ ID NO: 139 - DNA, 28 bp, synthetic construct
  source 1..28: mol_type = other DNA; note = H45A(-12+16)
tgccatcctggagttcctgtaagatacc

SEQ ID NO: 140 - DNA, 39 bp, synthetic construct
  source 1..39: mol_type = other DNA; note = H45A(-14+25)
gctgcccaatgccatcctggagttcctgtaagataccaa

SEQ ID NO: 141 - DNA, 27 bp, synthetic construct
  source 1..27: mol_type = other DNA; note = H45A(-08+19)
caatgccatcctggagttcctgtaaga

SEQ ID NO: 142 - DNA, 32 bp, synthetic construct
  source 1..32: mol_type = other DNA; note = HM45A(-07+25)
gctgcccaatgccatcctggagttcctgtaag

SEQ ID NO: 143 - DNA, 34 bp, synthetic construct
  source 1..34: mol_type = other DNA; note = H45A(-12+22)
gcccaatgccatcctggagttcctgtaagatacc

SEQ ID NO: 144 - DNA, 31 bp, synthetic construct
  source 1..31: mol_type = other DNA; note = H45A(-09+22)
gcccaatgccatcctggagttcctgtaagat

SEQ ID NO: 145 - DNA, 39 bp, synthetic construct
  source 1..39: mol_type = other DNA; note = H45A(-09+30)
ttgccgctgcccaatgccatcctggagttcctgtaagat

SEQ ID NO: 146 - DNA, 28 bp, synthetic construct
  source 1..28: mol_type = other DNA; note = H45A(-06+22)
gcccaatgccatcctggagttcctgtaa

SEQ ID NO: 147 - DNA, 34 bp, synthetic construct
  source 1..34: mol_type = other DNA; note = H45A(-06+28)
gccgctgcccaatgccatcctggagttcctgtaa

SEQ ID NO: 148 - DNA, 25 bp, synthetic construct
  source 1..25: mol_type = other DNA; note = H45A(-03+22)
gcccaatgccatcctggagttcctg

SEQ ID NO: 149 - DNA, 31 bp, synthetic construct
  source 1..31: mol_type = other DNA; note = H45A(-03+28)
gccgctgcccaatgccatcctggagttcctg

SEQ ID NO: 150 - RNA, 18 nt, synthetic construct
  source 1..18: mol_type = other RNA
  modified_base 1: mod_base = m5c
  modified_base 3: mod_base = m5c
  modified_base 4: mod_base = m5u
  modified_base 7..8: mod_base = m5c
  modified_base 11: mod_base = m5u
  modified_base 13..14: mod_base = m5c
  modified_base 17..18: mod_base = m5c
ngntgcnnaatgnnatnn

SEQ ID NO: 151 - DNA, 25 bp, synthetic construct
  source 1..25: mol_type = other DNA; note = H44A(-10+15)
gatctgtcaaatcgcctgcaggtaa

SEQ ID NO: 152 - DNA, 22 bp, synthetic construct
  source 1..22: mol_type = other DNA; note = H44A(-07+15)
gatctgtcaaatcgcctgcagg

SEQ ID NO: 153 - DNA, 24 bp, synthetic construct
  source 1..24: mol_type = other DNA; note = H44M(-07+17)
cagatctgtcaaatcgcctgcagg

SEQ ID NO: 154 - DNA, 23 bp, synthetic construct
  source 1..23: mol_type = other DNA; note = H44A(-08+15)
gatctgtcaaatcgcctgcaggt

SEQ ID NO: 155 - DNA, 21 bp, synthetic construct
  source 1..21: mol_type = other DNA; note = H44A(-06+15)
gatctgtcaaatcgcctgcag

SEQ ID NO: 156 - DNA, 25 bp, synthetic construct
  source 1..25: mol_type = other DNA; note = H44A(-08+17)
cagatctgtcaaatcgcctgcaggt

SEQ ID NO: 157 - DNA, 23 bp, synthetic construct
  source 1..23: mol_type = other DNA; note = H44A(-06+17)
cagatctgtcaaatcgcctgcag

SEQ ID NO: 158 - DNA, 22 bp, synthetic construct
  source 1..22: mol_type = other DNA; note = H50D(+04-18)
gggatccagtatacttacaggc

SEQ ID NO: 159 - DNA, 25 bp, synthetic construct
  source 1..25: mol_type = other DNA; note = H50D(+07-18)
gggatccagtatacttacaggctcc

SEQ ID NO: 160 - DNA, 23 bp, synthetic construct
  source 1..23: mol_type = other DNA; note = H50D(+07-16)
gatccagtatacttacaggctcc

SEQ ID NO: 161 - DNA, 24 bp, synthetic construct
  source 1..24: mol_type = other DNA; note = H50D(+07-17)
ggatccagtatacttacaggctcc

SEQ ID NO: 162 - DNA, 26 bp, synthetic construct
  source 1..26: mol_type = other DNA; note = H50A(-19+07)
acttcctctttaacagaaaagcatac

SEQ ID NO: 163 - DNA, 22 bp, synthetic construct
  source 1..22: mol_type = other DNA; note = H50D(+07-15)
atccagtatacttacaggctcc

SEQ ID NO: 164 - DNA, 25 bp, synthetic construct
  source 1..25: mol_type = other DNA; note = H50A(-02+23)
gagctcagatcttctaacttcctct

SEQ ID NO: 165 - DNA, 24 bp, synthetic construct
  source 1..24: mol_type = other DNA; note = H50D(+06-18)
gggatccagtatacttacaggctc

SEQ ID NO: 166 - DNA, 27 bp, synthetic construct
  source 1..27: mol_type = other DNA; note = H50D(+07-20)
atgggatccagtatacttacaggctcc

SEQ ID NO: 167 - DNA, 25 bp, synthetic construct
  source 1..25: mol_type = other DNA; note = H52A(-01+24)
ctgttccaaatcctgcattgttgcc

SEQ ID NO: 168 - DNA, 30 bp, synthetic construct
  source 1..30: mol_type = other DNA; note = H51A(+61+90)
acatcaaggaagatggcatttctagtttgg

SEQ ID NO: 169 - DNA, 23 bp, synthetic construct
  source 1..23: mol_type = other DNA; note = H51D(+16-07)
ctcataccttctgcttgatgatc

SEQ ID NO: 170 - DNA, 25 bp, synthetic construct
  source 1..25: mol_type = other DNA; note = H50D(+103+127)
gggatccagtatacttacaggctcc

SEQ ID NO: 171 - DNA, 26 bp, synthetic construct
  source 1..26: mol_type = other DNA; note = H51A(+81+105)
gagcaggtacctccaacatcaaggaa

SEQ ID NO: 172 - DNA, 30 bp, synthetic construct
  source 1..30: mol_type = other DNA; note = H51A(+71+100)
ggtacctccaacatcaaggaagatggcatt

SEQ ID NO: 173 - DNA, 26 bp, synthetic construct
  source 1..26: mol_type = other DNA; note = H51A(+48+73)
atttctagtttggagatggcagtttc

SEQ ID NO: 174 - DNA, 25 bp, synthetic construct
  source 1..25: mol_type = other DNA; note = H51A(+59+84)
ggaagatggcatttctagtttggag

SEQ ID NO: 175 - DNA, 26 bp, synthetic construct
  source 1..26: mol_type = other DNA; note = H51A(+64+88)
catcaaggaagatggcatttctagtt

SEQ ID NO: 176 - DNA, 25 bp, synthetic construct
  source 1..25: mol_type = other DNA; note = H51A(+89+113)
atctgccagagcaggtacctccaac

SEQ ID NO: 177 - DNA, 20 bp, synthetic construct
  source 1..20: mol_type = other DNA; note = H51A(+49+68)
tagtttggagatggcagttt

SEQ ID NO: 178 - DNA, 20 bp, synthetic construct
  source 1..20: mol_type = other DNA; note = H51A(+64+83)
ggaagatggcatttctagtt

SEQ ID NO: 179 - DNA, 20 bp, synthetic construct
  source 1..20: mol_type = other DNA; note = H51A(+80+98)
tacctccaacatcaaggaag

SEQ ID NO: 180 - DNA, 20 bp, synthetic construct
  source 1..20: mol_type = other DNA; note = H51A(+94+113)
atctgccagagcaggtacct

SEQ ID NO: 181 - DNA, 20 bp, synthetic construct
  source 1..20: mol_type = other DNA; note = H51A(+109+128)
ccaagcccggttgaaatctg

SEQ ID NO: 182 - DNA, 22 bp, synthetic construct
  source 1..22: mol_type = other DNA; note = H51A(+61+82)
gaagatggcatttctagtttgg

SEQ ID NO: 183 - DNA, 23 bp, synthetic construct
  source 1..23: mol_type = other DNA; note = H51A(+61+83)
ggaagatggcatttctagtttgg

SEQ ID NO: 184 - DNA, 29 bp, synthetic construct
  source 1..29: mol_type = other DNA; note = H51A(+61+89)
catcaaggaagatggcatttctagtttgg

SEQ ID NO: 185 - DNA, 24 bp, synthetic construct
  source 1..24: mol_type = other DNA; note = H51A(+66+89)
catcaaggaagatggcatttctag

SEQ ID NO: 186 - DNA, 28 bp, synthetic construct
  source 1..28: mol_type = other DNA; note = H51A(+66+93)
ccaacatcaaggaagatggcatttctag

SEQ ID NO: 187 - DNA, 24 bp, synthetic construct
  source 1..24: mol_type = other DNA; note = H51A(+69+92)
caacatcaaggaagatggcatttc

SEQ ID NO: 188 - DNA, 28 bp, synthetic construct
  source 1..28: mol_type = other DNA; note = H51A(+69+96)
cctccaacatcaaggaagatggcatttc

SEQ ID NO: 189 - DNA, 23 bp, synthetic construct
  source 1..23: mol_type = other DNA; note = H51A(+74+96)
cctccaacatcaaggaagatggc

SEQ ID NO: 190 - DNA, 26 bp, synthetic construct
  source 1..26: mol_type = other DNA; note = H51A(+74+99)
gtacctccaacatcaaggaagatggc

SEQ ID NO: 191 - DNA, 27 bp, synthetic construct
  source 1..27: mol_type = other DNA; note = H51A(+74+100)
ggtacctccaacatcaaggaagatggc

SEQ ID NO: 192 - DNA, 29 bp, synthetic construct
  source 1..29: mol_type = other DNA; note = H51A(+74+102)
caggtacctccaacatcaaggaagatggc

SEQ ID NO: 193 - DNA, 30 bp, synthetic construct; identical to SEQ ID NO: 17
  source 1..30: mol_type = other DNA; note = H51A(+74+103)

SEQ ID NO: 194 - DNA, 22 bp, synthetic construct
  source 1..22: mol_type = other DNA; note = H51A(+75+96)
cctccaacatcaaggaagatgg

SEQ ID NO: 195 - DNA, 25 bp, synthetic construct
  source 1..25: mol_type = other DNA; note = H51A(+75+99)
gtacctccaacatcaaggaagatgg

SEQ ID NO: 196 - DNA, 24 bp, synthetic construct
  source 1..24: mol_type = other DNA; note = H51A(+76+99)
gtacctccaacatcaaggaagatg

SEQ ID NO: 197 - DNA, 30 bp, synthetic construct; identical to SEQ ID NO: 21
  source 1..30: mol_type = other DNA; note = H51A(+76+105)

SEQ ID NO: 198 - DNA, 25 bp, synthetic construct
  source 1..25: mol_type = other DNA; note = H51A(+80+103)
gcaggtacctccaacatcaaggaag

SEQ ID NO: 199 - DNA, 27 bp, synthetic construct
  source 1..27: mol_type = other DNA; note = H51A(+80+105)
gagcaggtacctccaacatcaaggaag

SEQ ID NO: 200 - DNA, 29 bp, synthetic construct
  source 1..29: mol_type = other DNA; note = H51A(+80+107)
cagagcaggtacctccaacatcaaggaag

SEQ ID NO: 201 - DNA, 30 bp, synthetic construct; identical to SEQ ID NO: 25
  source 1..30: mol_type = other DNA; note = H51A(+80+108)

SEQ ID NO: 202 - DNA, 22 bp, synthetic construct
  source 1..22: mol_type = other DNA; note = H51A(+83+103)
gcaggtacctccaacatcaagg

SEQ ID NO: 203 - DNA, 24 bp, synthetic construct
  source 1..24: mol_type = other DNA; note = H51A(+83+105)
gagcaggtacctccaacatcaagg

SEQ ID NO: 204 - DNA, 26 bp, synthetic construct
  source 1..26: mol_type = other DNA; note = H51A(+83+107)
cagagcaggtacctccaacatcaagg

SEQ ID NO: 205 - DNA, 28 bp, synthetic construct
  source 1..28: mol_type = other DNA; note = H51A(+83+109)
gccagagcaggtacctccaacatcaagg

SEQ ID NO: 206 - DNA, 25 bp, synthetic construct
  source 1..25: mol_type = other DNA; note = H51A(+84+107)
cagagcaggtacctccaacatcaag

SEQ ID NO: 207 - DNA, 29 bp, synthetic construct
  source 1..29: mol_type = other DNA; note = H51A(+84+111)
ctgccagagcaggtacctccaacatcaag

SEQ ID NO: 208 - DNA, 23 bp, synthetic construct
  source 1..23: mol_type = other DNA; note = H51A(+84+105)
gagcaggtacctccaacatcaag

SEQ ID NO: 209 - DNA, 24 bp, synthetic construct
  source 1..24: mol_type = other DNA; note = H51A(+87+109)
gccagagcaggtacctccaacatc

SEQ ID NO: 210 - DNA, 24 bp, synthetic construct
  source 1..24: mol_type = other DNA; note = H51A(+93+116)
gaaatctgccagagcaggtacctc

SEQ ID NO: 211 - DNA, 26 bp, synthetic construct
  source 1..26: mol_type = other DNA; note = H51A(+75+100)
ggtacctccaacatcaaggaagatgg

SEQ ID NO: 212 - DNA, 28 bp, synthetic construct
  source 1..28: mol_type = other DNA; note = H51A(+74+101)
aggtacctccaacatcaaggaagatggc

SEQ ID NO: 213 - DNA, 25 bp, synthetic construct
  source 1..25: mol_type = other DNA; note = H51A(+74+98)
tacctccaacatcaaggaagatggc

SEQ ID NO: 214 - DNA, 24 bp, synthetic construct
  source 1..24: mol_type = other DNA; note = H51A(+74+97)
acctccaacatcaaggaagatggc

SEQ ID NO: 215 - DNA, 21 bp, synthetic construct
  source 1..21: mol_type = other DNA; note = H51A(+74+94)
tccaacatcaaggaagatggc

SEQ ID NO: 216 - DNA, 20 bp, synthetic construct
  source 1..20: mol_type = other DNA; note = H51A(+74+93)
ccaacatcaaggaagatggc

SEQ ID NO: 217 - DNA, 19 bp, synthetic construct
  source 1..19: mol_type = other DNA; note = H51A(+74+92)
caacatcaaggaagatggc

SEQ ID NO: 218 - DNA, 31 bp, synthetic construct; identical to SEQ ID NO: 42
  source 1..31: mol_type = other DNA; note = H51A(+69+99)

SEQ ID NO: 219 - DNA, 30 bp, synthetic construct; identical to SEQ ID NO: 43
  source 1..30: mol_type = other DNA; note = H51A(+70+99)

SEQ ID NO: 220 - DNA, 29 bp, synthetic construct
  source 1..29: mol_type = other DNA; note = H51A(+71+99)
gtacctccaacatcaaggaagatggcatt

SEQ ID NO: 221 - DNA, 28 bp, synthetic construct
  source 1..28: mol_type = other DNA; note = H51A(+72+99)
gtacctccaacatcaaggaagatggcat

SEQ ID NO: 222 - DNA, 27 bp, synthetic construct
  source 1..27: mol_type = other DNA; note = H51A(+73+99)
gtacctccaacatcaaggaagatggca

SEQ ID NO: 223 - DNA, 23 bp, synthetic construct
  source 1..23: mol_type = other DNA; note = H51A(+77+99)
gtacctccaacatcaaggaagat

SEQ ID NO: 224 - DNA, 22 bp, synthetic construct
  source 1..22: mol_type = other DNA; note = H51A(+78+99)
gtacctccaacatcaaggaaga

SEQ ID NO: 225 - DNA, 21 bp, synthetic construct
  source 1..21: mol_type = other DNA; note = H51A(+79+99)
gtacctccaacatcaaggaag

SEQ ID NO: 226 - DNA, 25 bp, synthetic construct
  source 1..25: mol_type = other DNA; note = H51.SA.(-60-36)
gaagaaaaagaaaaattagaaacac

SEQ ID NO: 227 - DNA, 25 bp, synthetic construct
  source 1..25: mol_type = other DNA; note = H51.SA.(-50-26)
aaggaaaaaagaagaaaaagaaaaa

SEQ ID NO: 228 - DNA, 25 bp, synthetic construct
  source 1..25: mol_type = other DNA; note = H51.SA.(-45-21)
gcaaaaaggaaaaaagaagaaaaag

SEQ ID NO: 229 - DNA, 25 bp, synthetic construct
  source 1..25: mol_type = other DNA; note = H51.SA.(-40-16)
tttttgcaaaaaggaaaaaagaaga

SEQ ID NO: 230 - DNA, 25 bp, synthetic construct
  source 1..25: mol_type = other DNA; note = H51.SA.(-35-11)
ttgggtttttgcaaaaaggaaaaaa

SEQ ID NO: 231 - DNA, 25 bp, synthetic construct
  source 1..25: mol_type = other DNA; note = H51.SA.(-30-6)
atattttgggtttttgcaaaaagga

SEQ ID NO: 232 - DNA, 25 bp, synthetic construct
  source 1..25: mol_type = other DNA; note = H51.SA.(-25-1)
ctaaaatattttgggtttttgcaaa

SEQ ID NO: 233 - DNA, 25 bp, synthetic construct
  source 1..25: mol_type = other DNA; note = H51.SA.(-20+5)
aggagctaaaatattttgggttttt

SEQ ID NO: 234 - DNA, 25 bp, synthetic construct
  source 1..25: mol_type = other DNA; note = H51.SA.(-15+10)
tgagtaggagctaaaatattttggg

SEQ ID NO: 235 - DNA, 25 bp, synthetic construct
  source 1..25: mol_type = other DNA; note = H51.SA.(-10+15)
cagtctgagtaggagctaaaatatt

SEQ ID NO: 236 - DNA, 25 bp, synthetic construct
  source 1..25: mol_type = other DNA; note = H51.SA.(-5+20)
agtaacagtctgagtaggagctaaa

SEQ ID NO: 237 - DNA, 25 bp, synthetic construct
  source 1..25: mol_type = other DNA; note = H51.SA.(-1+24)
ccagagtaacagtctgagtaggagc

SEQ ID NO: 238 - DNA, 25 bp, synthetic construct
  source 1..25: mol_type = other DNA; note = H51.SA.(-65-41)
aaaagaaaaattagaaacacaagct

SEQ ID NO: 239 - DNA, 25 bp, synthetic construct
  source 1..25: mol_type = other DNA; note = H51.SA.(-70-46)
aaaaattagaaacacaagctaaaga

SEQ ID NO: 240 - DNA, 25 bp, synthetic construct
  source 1..25: mol_type = other DNA; note = H51.SA.(-75-51)
ttagaaacacaagctaaagagccaa

SEQ ID NO: 241 - DNA, 25 bp, synthetic construct
  source 1..25: mol_type = other DNA; note = H51.SA.(-80-56)
aacacaagctaaagagccaatttca

SEQ ID NO: 242 - DNA, 25 bp, synthetic construct
  source 1..25: mol_type = other DNA; note = H51.SA.(-85-61)
aagctaaagagccaatttcaataac

SEQ ID NO: 243 - DNA, 25 bp, synthetic construct
  source 1..25: mol_type = other DNA; note = H51.SA.(-90-66)
aaagagccaatttcaataacaataa

SEQ ID NO: 244 - DNA, 25 bp, synthetic construct
  source 1..25: mol_type = other DNA; note = H51.SA.(-95-71)
gccaatttcaataacaataagtcaa

SEQ ID NO: 245 - DNA, 25 bp, synthetic construct
  source 1..25: mol_type = other DNA; note = H51.SA.(-100-76)
tttcaataacaataagtcaaattta

SEQ ID NO: 246 - DNA, 30 bp, synthetic construct; identical to SEQ ID NO: 70
  source 1..30: mol_type = other DNA; note = H51A(+1+30)

SEQ ID NO: 247 - DNA, 30 bp, synthetic construct; identical to SEQ ID NO: 71
  source 1..30: mol_type = other DNA; note = H51A(+10+39)

SEQ ID NO: 248 - DNA, 30 bp, synthetic construct; identical to SEQ ID NO: 72
  source 1..30: mol_type = other DNA; note = H51A(+6+35)

SEQ ID NO: 249 - DNA, 30 bp, synthetic construct; identical to SEQ ID NO: 73
  source 1..30: mol_type = other DNA; note = H51A(+49+78)

SEQ ID NO: 250 - DNA, 25 bp, synthetic construct
  source 1..25: mol_type = other DNA; note = H51A(+1+25)
accagagtaacagtctgagtaggag

SEQ ID NO: 251 - DNA, 25 bp, synthetic construct
  source 1..25: mol_type = other DNA; note = H51A(+4+28)
gtcaccagagtaacagtctgagtag

SEQ ID NO: 252 - DNA, 25 bp, synthetic construct
  source 1..25: mol_type = other DNA; note = H51A(+16+40)
accacaggttgtgtcaccagagtaa

SEQ ID NO: 253 - DNA, 25 bp, synthetic construct
  source 1..25: mol_type = other DNA; note = H51A(+21+45)
tagtaaccacaggttgtgtcaccag

SEQ ID NO: 254 - DNA, 25 bp, synthetic construct
  source 1..25: mol_type = other DNA; note = H51A(+26+50)
ttccttagtaaccacaggttgtgtc

SEQ ID NO: 255 - DNA, 25 bp, synthetic construct
  source 1..25: mol_type = other DNA; note = H51A(+31+55)
gcagtttccttagtaaccacaggtt

SEQ ID NO: 256 - DNA, 25 bp, synthetic construct
  source 1..25: mol_type = other DNA; note = H51A(+36+60)
agatggcagtttccttagtaaccac

SEQ ID NO: 257 - DNA, 25 bp, synthetic construct
  source 1..25: mol_type = other DNA; note = H51A(+41+65)
tttggagatggcagtttccttagta

SEQ ID NO: 258 - DNA, 25 bp, synthetic construct
  source 1..25: mol_type = other DNA; note = H51A(+86+110)
tgccagagcaggtacctccaacatc

SEQ ID NO: 259 - DNA, 25 bp, synthetic construct
  source 1..25: mol_type = other DNA; note = H51A(+91+115)
aaatctgccagagcaggtacctcca

SEQ ID NO: 260 - DNA, 25 bp, synthetic construct
  source 1..25: mol_type = other DNA; note = H51A(+96+120)
ggttgaaatctgccagagcaggtac

SEQ ID NO: 261 - DNA, 25 bp, synthetic construct
  source 1..25: mol_type = other DNA; note = H51A(+101+125)
agcccggttgaaatctgccagagca

SEQ ID NO: 262 - DNA, 25 bp, synthetic construct
  source 1..25: mol_type = other DNA; note = H51A(+106+130)
gtccaagcccggttgaaatctgcca

SEQ ID NO: 263 - DNA, 25 bp, synthetic construct
  source 1..25: mol_type = other DNA; note = H51A(+111+135)
gttctgtccaagcccggttgaaatc

SEQ ID NO: 264 - DNA, 25 bp, synthetic construct
  source 1..25: mol_type = other DNA; note = H51A(+116+140)
ggtaagttctgtccaagcccggttg

SEQ ID NO: 265 - DNA, 25 bp, synthetic construct
  source 1..25: mol_type = other DNA; note = H51A(+121+145)
cagtcggtaagttctgtccaagccc

SEQ ID NO: 266 - DNA, 25 bp, synthetic construct
  source 1..25: mol_type = other DNA; note = H51A(+126+150)
aaagccagtcggtaagttctgtcca

SEQ ID NO: 267 - DNA, 25 bp, synthetic construct
  source 1..25: mol_type = other DNA; note = H51A(+131+155)
cagagaaagccagtcggtaagttct

SEQ ID NO: 268 - DNA, 25 bp, synthetic construct
  source 1..25: mol_type = other DNA; note = H51A(+136+160)
tcaagcagagaaagccagtcggtaa

SEQ ID NO: 269 - DNA, 25 bp, synthetic construct
  source 1..25: mol_type = other DNA; note = H51A(+141+165)
cttgatcaagcagagaaagccagtc

SEQ ID NO: 270 - DNA, 25 bp, synthetic construct
  source 1..25: mol_type = other DNA; note = H51A(+146+170)
tataacttgatcaagcagagaaagc

SEQ ID NO: 271 - DNA, 25 bp, synthetic construct
  source 1..25: mol_type = other DNA; note = H51A(+151+175)
gattttataacttgatcaagcagag

SEQ ID NO: 272 - DNA, 25 bp, synthetic construct
  source 1..25: mol_type = other DNA; note = H51A(+156+180)
tctgtgattttataacttgatcaag

SEQ ID NO: 273 - DNA, 25 bp, synthetic construct
  source 1..25: mol_type = other DNA; note = H51A(+161+185)
caccctctgtgattttataacttga

SEQ ID NO: 274 - DNA, 25 bp, synthetic construct
  source 1..25: mol_type = other DNA; note = H51A(+166+190)
accatcaccctctgtgattttataa

SEQ ID NO: 275 - DNA, 25 bp, synthetic construct
  source 1..25: mol_type = other DNA; note = H51A(+171+195)
cacccaccatcaccctctgtgattt

SEQ ID NO: 276 - DNA, 25 bp, synthetic construct
  source 1..25: mol_type = other DNA; note = H51A(+176+200)
aaggtcacccaccatcaccctctgt

SEQ ID NO: 277 - DNA, 25 bp, synthetic construct
  source 1..25: mol_type = other DNA; note = H51A(+181+205)
tcctcaaggtcacccaccatcaccc

SEQ ID NO: 278 - DNA, 25 bp, synthetic construct
  source 1..25: mol_type = other DNA; note = H51A(+186+210)
tgatatcctcaaggtcacccaccat

SEQ ID NO: 279 - DNA, 25 bp, synthetic construct
  source 1..25: mol_type = other DNA; note = H51A(+191+215)
ctcgttgatatcctcaaggtcaccc

SEQ ID NO: 280 - DNA, 25 bp, synthetic construct
  source 1..25: mol_type = other DNA; note = H51A(+196+220)
atcatctcgttgatatcctcaaggt

SEQ ID NO: 281 - DNA, 25 bp, synthetic construct
  source 1..25: mol_type = other DNA; note = H51A(+201+225)
tgatgatcatctcgttgatatcctc

SEQ ID NO: 282 - DNA, 25 bp, synthetic construct
  source 1..25: mol_type = other DNA; note = H51A(+206+230)
ctgcttgatgatcatctcgttgata

SEQ ID NO: 283 - DNA, 25 bp, synthetic construct
  source 1..25: mol_type = other DNA; note = H51D(+211-02)
accttctgcttgatgatcatctcgt

SEQ ID NO: 284 - DNA, 25 bp, synthetic construct
  source 1..25: mol_type = other DNA; note = H51D(+214-05)
cataccttctgcttgatgatcatct

SEQ ID NO: 285 - DNA, 25 bp, synthetic construct
  source 1..25: mol_type = other DNA; note = H51D(+217-08)
tctcataccttctgcttgatgatca

SEQ ID NO: 286 - DNA, 25 bp, synthetic construct
  source 1..25: mol_type = other DNA; note = H51D(+220-11)
ttttctcataccttctgcttgatga

SEQ ID NO: 287 - DNA, 25 bp, synthetic construct
  source 1..25: mol_type = other DNA; note = H51D(+223-14)
attttttctcataccttctgcttga

SEQ ID NO: 288 - DNA, 25 bp, synthetic construct
  source 1..25: mol_type = other DNA; note = H51D(+226-17)
atcattttttctcataccttctgct

SEQ ID NO: 289 - DNA, 25 bp, synthetic construct
  source 1..25: mol_type = other DNA; note = H51D(+229-20)
tttatcattttttctcataccttct

SEQ ID NO: 290 - DNA, 25 bp, synthetic construct
  source 1..25: mol_type = other DNA; note = H51D(+232-23)
acttttatcattttttctcatacct

SEQ ID NO: 291 - DNA, 25 bp, synthetic construct
  source 1..25: mol_type = other DNA; note = H51D(-02-26)
ccaacttttatcattttttctcata

SEQ ID NO: 292 - DNA, 30 bp, synthetic construct; identical to SEQ ID NO: 70
  source 1..30: mol_type = other DNA; note = H51A(+1+30)

SEQ ID NO: 293 - DNA, 30 bp, synthetic construct; identical to SEQ ID NO: 1
  source 1..30: mol_type = other DNA; note = H51A(+66+95)

SEQ ID NO: 294 - DNA, 24 bp, synthetic construct
  source 1..24: mol_type = other DNA; note = H51A(+74+97)
acctccaacatcaaggaagatggc

SEQ ID NO: 295 - DNA, 30 bp, synthetic construct; identical to SEQ ID NO: 43
  source 1..30: mol_type = other DNA; note = H51A(+70+99)

SEQ ID NO: 296 - DNA, 28 bp, synthetic construct
  source 1..28: mol_type = other DNA; note = H51A(+72+99)
gtacctccaacatcaaggaagatggcat

SEQ ID NO: 297 - DNA, 20 bp, synthetic construct
  source 1..20: mol_type = other DNA; note = H51A(+68+87)
tcaaggaagatggcatttct

SEQ ID NO: 298 - RNA, 20 nt, synthetic construct
  source 1..20: mol_type = other RNA; note = H51A(+68+87)
  modified_base 7: mod_base = OTHER; note = methylated adenine
  modified_base 9: mod_base = OTHER; note = methylated guanine
  modified_base 10: mod_base = OTHER; note = methylated adenine
  modified_base 12..13: mod_base = OTHER; note = methylated guanine
tcaaggnanntnncatttct

SEQ ID NO: 299 - DNA, 25 bp, synthetic construct
  source 1..25: mol_type = other DNA; note = H53A(+36+60)
gttgcctccggttctgaaggtgttc

SEQ ID NO: 300 - DNA, 25 bp, synthetic construct
  source 1..25: mol_type = other DNA; note = H53A(+36+60)
  modified_base 5: mod_base = m5c
  modified_base 6: mod_base = m5c
  modified_base 8: mod_base = m5c
  modified_base 9: mod_base = m5c
  modified_base 14: mod_base = m5c
  modified_base 25: mod_base = m5c
gttgnntnnggttntgaaggtgttn

SEQ ID NO: 301 - DNA, 21 bp, synthetic construct
  source 1..21: mol_type = other DNA; note = H53A(+36+56)
cctccggttctgaaggtgttc

SEQ ID NO: 302 - DNA, 25 bp, synthetic construct
  source 1..25: mol_type = other DNA; note = H53A(+23+47)
ctgaaggtgttcttgtacttcatcc

SEQ ID NO: 303 - DNA, 25 bp, synthetic construct
  source 1..25: mol_type = other DNA; note = H53A(+32+56)
cctccggttctgaaggtgttcttgt

SEQ ID NO: 304 - DNA, 28 bp, synthetic construct
  source 1..28: mol_type = other DNA; note = H53A(+33+60)
gttgcctccggttctgaaggtgttcttg

SEQ ID NO: 305 - DNA, 30 bp, synthetic construct; identical to SEQ ID NO: 129
  source 1..30: mol_type = other DNA; note = H53A(+30+59)

SEQ ID NO: 306 - DNA, 24 bp, synthetic construct
  source 1..24: mol_type = other DNA; note = H53A(+39+62)
ctgttgcctccggttctgaaggtg

SEQ ID NO: 307 - DNA, 31 bp, synthetic construct; identical to SEQ ID NO: 131
  source 1..31: mol_type = other DNA; note = H53A(+36+69)

SEQ ID NO: 308 - DNA, 18 bp, synthetic construct
  source 1..18: mol_type = other DNA; note = H53A(+45+62)
ctgttgcctccggttctg

SEQ ID NO: 309 - DNA, 22 bp, synthetic construct
  source 1..22: mol_type = other DNA; note = H45A(-03+19)
caatgccatcctggagttcctg

SEQ ID NO: 310 - DNA, 34 bp, synthetic construct; identical to SEQ ID NO: 134
  source 1..34: mol_type = other DNA; note = H45A(-09+25)

SEQ ID NO: 311 - DNA, 28 bp, synthetic construct
  source 1..28: mol_type = other DNA; note = H45A(-03+25)
gctgcccaatgccatcctggagttcctg

SEQ ID NO: 312 - DNA, 31 bp, synthetic construct; identical to SEQ ID NO: 136
  source 1..31: mol_type = other DNA; note = H45A(-06+25)

SEQ ID NO: 313 - DNA, 31 bp, synthetic construct; identical to SEQ ID NO: 137
  source 1..31: mol_type = other DNA; note = H45A(-12+19)

SEQ ID NO: 314 - DNA, 28 bp, synthetic construct
  source 1..28: mol_type = other DNA; note = H45A(-09+19)
caatgccatcctggagttcctgtaagat

SEQ ID NO: 315 - DNA, 28 bp, synthetic construct
  source 1..28: mol_type = other DNA; note = H45A(-12+16)
tgccatcctggagttcctgtaagatacc

SEQ ID NO: 316 - DNA, 39 bp, synthetic construct; identical to SEQ ID NO: 140
  source 1..39: mol_type = other DNA; note = H45A(-14+25)

SEQ ID NO: 317 - DNA, 27 bp, synthetic construct
  source 1..27: mol_type = other DNA; note = H45A(-08+19)
caatgccatcctggagttcctgtaaga

SEQ ID NO: 318 - DNA, 32 bp, synthetic construct; identical to SEQ ID NO: 142
  source 1..32: mol_type = other DNA; note = HM45A(-07+25)

SEQ ID NO: 319 - DNA, 34 bp, synthetic construct; identical to SEQ ID NO: 143
  source 1..34: mol_type = other DNA; note = H45A(-12+22)

SEQ ID NO: 320 - DNA, 31 bp, synthetic construct; identical to SEQ ID NO: 144
  source 1..31: mol_type = other DNA; note = H45A(-09+22)

SEQ ID NO: 321 - DNA, 39 bp, synthetic construct; identical to SEQ ID NO: 145
  source 1..39: mol_type = other DNA; note = H45A(-09+30)

SEQ ID NO: 322 - DNA, 28 bp, synthetic construct
  source 1..28: mol_type = other DNA; note = H45A(-06+22)
gcccaatgccatcctggagttcctgtaa

SEQ ID NO: 323 - DNA, 34 bp, synthetic construct; identical to SEQ ID NO: 147
  source 1..34: mol_type = other DNA; note = H45A(-06+28)

SEQ ID NO: 324 - DNA, 25 bp, synthetic construct
  source 1..25: mol_type = other DNA; note = H45A(-03+22)
gcccaatgccatcctggagttcctg

SEQ ID NO: 325 - DNA, 31 bp, synthetic construct; identical to SEQ ID NO: 149
  source 1..31: mol_type = other DNA; note = H45A(-03+28)

SEQ ID NO: 326 - skipped

SEQ ID NO: 327 - DNA, 25 bp, synthetic construct
  source 1..25: mol_type = other DNA; note = H44A(-10+15)
gatctgtcaaatcgcctgcaggtaa

SEQ ID NO: 328 - DNA, 22 bp, synthetic construct
  source 1..22: mol_type = other DNA; note = H44A(-07+15)
gatctgtcaaatcgcctgcagg

SEQ ID NO: 329 - DNA, 24 bp, synthetic construct
  source 1..24: mol_type = other DNA; note = H44M(-07+17)
cagatctgtcaaatcgcctgcagg

SEQ ID NO: 330 - DNA, 23 bp, synthetic construct
  source 1..23: mol_type = other DNA; note = H44A(-08+15)
gatctgtcaaatcgcctgcaggt

SEQ ID NO: 331 - DNA, 21 bp, synthetic construct
  source 1..21: mol_type = other DNA; note = H44A(-06+15)
gatctgtcaaatcgcctgcag

SEQ ID NO: 332 - DNA, 25 bp, synthetic construct
  source 1..25: mol_type = other DNA; note = H44A(-08+17)
cagatctgtcaaatcgcctgcaggt

SEQ ID NO: 333 - DNA, 23 bp, synthetic construct
  source 1..23: mol_type = other DNA; note = H44A(-06+17)
cagatctgtcaaatcgcctgcag

SEQ ID NO: 334 - DNA, 22 bp, synthetic construct
  source 1..22: mol_type = other DNA; note = H50D(+04-18)
gggatccagtatacttacaggc

SEQ ID NO: 335 - DNA, 25 bp, synthetic construct
  source 1..25: mol_type = other DNA; note = H50D(+07-18)
gggatccagtatacttacaggctcc

SEQ ID NO: 336 - DNA, 23 bp, synthetic construct
  source 1..23: mol_type = other DNA; note = H50D(+07-16)
gatccagtatacttacaggctcc

SEQ ID NO: 337 - DNA, 24 bp, synthetic construct
  source 1..24: mol_type = other DNA; note = H50D(+07-17)
ggatccagtatacttacaggctcc

SEQ ID NO: 338 - DNA, 26 bp, synthetic construct
  source 1..26: mol_type = other DNA; note = H50A(-19+07)
acttcctctttaacagaaaagcatac

SEQ ID NO: 339 - DNA, 22 bp, synthetic construct
  source 1..22: mol_type = other DNA; note = H50D(+07-15)
atccagtatacttacaggctcc

SEQ ID NO: 340 - DNA, 25 bp, synthetic construct
  source 1..25: mol_type = other DNA; note = H50A(-02+23)
gagctcagatcttctaacttcctct

SEQ ID NO: 341 - DNA, 24 bp, synthetic construct
  source 1..24: mol_type = other DNA; note = H50D(+06-18)
gggatccagtatacttacaggctc

SEQ ID NO: 342 - DNA, 27 bp, synthetic construct
  source 1..27: mol_type = other DNA; note = H50D(+07-20)
atgggatccagtatacttacaggctcc

SEQ ID NO: 343 - DNA, 25 bp, synthetic construct
  source 1..25: mol_type = other DNA; note = H52A(-01+24)
ctgttccaaatcctgcattgttgcc

SEQ ID NO: 344 - DNA, 30 bp, synthetic construct; identical to SEQ ID NO: 168
  source 1..30: mol_type = other DNA; note = H51A(+61+90)

SEQ ID NO: 345 - DNA, 23 bp, synthetic construct
  source 1..23: mol_type = other DNA; note = H51D(+16-07)
ctcataccttctgcttgatgatc

SEQ ID NO: 346 - DNA, 25 bp, synthetic construct
  source 1..25: mol_type = other DNA; note = H50D(+103+127)
gggatccagtatacttacaggctcc

SEQ ID NO: 347 - DNA, 26 bp, synthetic construct
  source 1..26: mol_type = other DNA; note = H51A(+81+105)
gagcaggtacctccaacatcaaggaa

SEQ ID NO: 348 - DNA, 30 bp, synthetic construct; identical to SEQ ID NO: 172
  source 1..30: mol_type = other DNA; note = H51A(+71+100)

SEQ ID NO: 349 - DNA, 26 bp, synthetic construct
  source 1..26: mol_type = other DNA; note = H51A(+48+73)
atttctagtttggagatggcagtttc

SEQ ID NO: 350 - DNA, 25 bp, synthetic construct
  source 1..25: mol_type = other DNA; note = H51A(+59+84)
ggaagatggcatttctagtttggag

SEQ ID NO: 351 - DNA, 26 bp, synthetic construct
  source 1..26: mol_type = other DNA; note = H51A(+64+88)
catcaaggaagatggcatttctagtt

SEQ ID NO: 352 - DNA, 25 bp, synthetic construct
  source 1..25: mol_type = other DNA; note = H51A(+89+113)
atctgccagagcaggtacctccaac

SEQ ID NO: 353 - DNA, 20 bp, synthetic construct
  source 1..20: mol_type = other DNA; note = H51A(+49+68)
tagtttggagatggcagttt

SEQ ID NO: 354 - DNA, 20 bp, synthetic construct
  source 1..20: mol_type = other DNA; note = H51A(+64+83)
ggaagatggcatttctagtt

SEQ ID NO: 355 - DNA, 20 bp, synthetic construct
  source 1..20: mol_type = other DNA; note = H51A(+80+98)
tacctccaacatcaaggaag

SEQ ID NO: 356 - DNA, 20 bp, synthetic construct
  source 1..20: mol_type = other DNA; note = H51A(+94+113)
atctgccagagcaggtacct

SEQ ID NO: 357 - DNA, 20 bp, synthetic construct
  source 1..20: mol_type = other DNA; note = H51A(+109+128)
ccaagcccggttgaaatctg

SEQ ID NO: 358 - DNA, 22 bp, synthetic construct
  source 1..22: mol_type = other DNA; note = H51A(+61+82)
gaagatggcatttctagtttgg

SEQ ID NO: 359 - DNA, 23 bp, synthetic construct
  source 1..23: mol_type = other DNA; note = H51A(+61+83)
ggaagatggcatttctagtttgg

SEQ ID NO: 360 - DNA, 29 bp, synthetic construct
  source 1..29: mol_type = other DNA; note = H51A(+61+89)
catcaaggaagatggcatttctagtttgg

SEQ ID NO: 361 - DNA, 24 bp, synthetic construct
  source 1..24: mol_type = other DNA; note = H51A(+66+89)
catcaaggaagatggcatttctag

SEQ ID NO: 362 - DNA, 28 bp, synthetic construct
  source 1..28: mol_type = other DNA; note = H51A(+66+93)
ccaacatcaaggaagatggcatttctag

SEQ ID NO: 363 - DNA, 24 bp, synthetic construct
  source 1..24: mol_type = other DNA; note = H51A(+69+92)
caacatcaaggaagatggcatttc

SEQ ID NO: 364 - DNA, 28 bp, synthetic construct
  source 1..28: mol_type = other DNA; note = H51A(+69+96)
cctccaacatcaaggaagatggcatttc

SEQ ID NO: 365 - DNA, 23 bp, synthetic construct
  source 1..23: mol_type = other DNA; note = H51A(+74+96)
cctccaacatcaaggaagatggc

SEQ ID NO: 366 - DNA, 26 bp, synthetic construct
  source 1..26: mol_type = other DNA; note = H51A(+74+99)
gtacctccaacatcaaggaagatggc

SEQ ID NO: 367 - DNA, 27 bp, synthetic construct
  source 1..27: mol_type = other DNA; note = H51A(+74+100)
ggtacctccaacatcaaggaagatggc

SEQ ID NO: 368 - DNA, 29 bp, synthetic construct
  source 1..29: mol_type = other DNA; note = H51A(+74+102)
caggtacctccaacatcaaggaagatggc

SEQ ID NO: 369 - DNA, 30 bp, synthetic construct; identical to SEQ ID NO: 17
  source 1..30: mol_type = other DNA; note = H51A(+74+103)

SEQ ID NO: 370 - DNA, 22 bp, synthetic construct
  source 1..22: mol_type = other DNA; note = H51A(+75+96)
cctccaacatcaaggaagatgg

SEQ ID NO: 371 - DNA, 25 bp, synthetic construct
  source 1..25: mol_type = other DNA; note = H51A(+75+99)
gtacctccaacatcaaggaagatgg

SEQ ID NO: 372 - DNA, 24 bp, synthetic construct
  source 1..24: mol_type = other DNA; note = H51A(+76+99)
gtacctccaacatcaaggaagatg

SEQ ID NO: 373 - DNA, 30 bp, synthetic construct; identical to SEQ ID NO: 21
  source 1..30: mol_type = other DNA; note = H51A(+76+105)

SEQ ID NO: 374 - DNA, 25 bp, synthetic construct
  source 1..25: mol_type = other DNA; note = H51A(+80+103)
gcaggtacctccaacatcaaggaag

SEQ ID NO: 375 - DNA, 27 bp, synthetic construct
  source 1..27: mol_type = other DNA; note = H51A(+80+105)
gagcaggtacctccaacatcaaggaag

SEQ ID NO: 376 - DNA, 29 bp, synthetic construct
  source 1..29: mol_type = other DNA; note = H51A(+80+107)
cagagcaggtacctccaacatcaaggaag

SEQ ID NO: 377 - DNA, 30 bp, synthetic construct; identical to SEQ ID NO: 25
  source 1..30: mol_type = other DNA; note = H51A(+80+108)

SEQ ID NO: 378 - DNA, 22 bp, synthetic construct
  source 1..22: mol_type = other DNA; note = H51A(+83+103)
gcaggtacctccaacatcaagg

SEQ ID NO: 379 - DNA, 24 bp, synthetic construct
  source 1..24: mol_type = other DNA; note = H51A(+83+105)
gagcaggtacctccaacatcaagg

SEQ ID NO: 380 - DNA, 26 bp, synthetic construct
  source 1..26: mol_type = other DNA; note = H51A(+83+107)
cagagcaggtacctccaacatcaagg

SEQ ID NO: 381 - DNA, 28 bp, synthetic construct
  source 1..28: mol_type = other DNA; note = H51A(+83+109)
gccagagcaggtacctccaacatcaagg

SEQ ID NO: 382 - DNA, 25 bp, synthetic construct
  source 1..25: mol_type = other DNA; note = H51A(+84+107)
cagagcaggtacctccaacatcaag

SEQ ID NO: 383 - DNA, 29 bp, synthetic construct
  source 1..29: mol_type = other DNA; note = H51A(+84+111)
ctgccagagcaggtacctccaacatcaag

SEQ ID NO: 384 - DNA, 23 bp, synthetic construct
  source 1..23: mol_type = other DNA; note = H51A(+84+105)
gagcaggtacctccaacatcaag

SEQ ID NO: 385 - DNA, 24 bp, synthetic construct
  source 1..24: mol_type = other DNA; note = H51A(+87+109)
gccagagcaggtacctccaacatc

SEQ ID NO: 386 - DNA, 24 bp, synthetic construct
  source 1..24: mol_type = other DNA; note = H51A(+93+116)
gaaatctgccagagcaggtacctc

SEQ ID NO: 387 - DNA, 26 bp, synthetic construct
  source 1..26: mol_type = other DNA; note = H51A(+75+100)
ggtacctccaacatcaaggaagatgg

SEQ ID NO: 388 - DNA, 28 bp, synthetic construct
  source 1..28: mol_type = other DNA; note = H51A(+74+101)
aggtacctccaacatcaaggaagatggc

SEQ ID NO: 389 - DNA, 25 bp, synthetic construct
  source 1..25: mol_type = other DNA; note = H51A(+74+98)
tacctccaacatcaaggaagatggc

SEQ ID NO: 390 - DNA, 24 bp, synthetic construct
  source 1..24: mol_type = other DNA; note = H51A(+74+97)
acctccaacatcaaggaagatggc

SEQ ID NO: 391 - DNA, 21 bp, synthetic construct
  source 1..21: mol_type = other DNA; note = H51A(+74+94)
tccaacatcaaggaagatggc

SEQ ID NO: 392 - DNA, 20 bp, synthetic construct
  source 1..20: mol_type = other DNA; note = H51A(+74+93)
ccaacatcaaggaagatggc

SEQ ID NO: 393 - DNA, 19 bp, synthetic construct
  source 1..19: mol_type = other DNA; note = H51A(+74+92)
caacatcaaggaagatggc

SEQ ID NO: 394 - DNA, 31 bp, synthetic construct; identical to SEQ ID NO: 42
  source 1..31: mol_type = other DNA; note = H51A(+69+99)

SEQ ID NO: 395 - DNA, 30 bp, synthetic construct; identical to SEQ ID NO: 43
  source 1..30: mol_type = other DNA; note = H51A(+70+99)

SEQ ID NO: 396 - DNA, 29 bp, synthetic construct
  source 1..29: mol_type = other DNA; note = H51A(+71+99)
gtacctccaacatcaaggaagatggcatt

SEQ ID NO: 397 - DNA, 28 bp, synthetic construct
  source 1..28: mol_type = other DNA; note = H51A(+72+99)
gtacctccaacatcaaggaagatggcat

SEQ ID NO: 398 - DNA, 27 bp, synthetic construct
  source 1..27: mol_type = other DNA; note = H51A(+73+99)
gtacctccaacatcaaggaagatggca

SEQ ID NO: 399 - DNA, 23 bp, synthetic construct
  source 1..23: mol_type = other DNA; note = H51A(+77+99)
gtacctccaacatcaaggaagat

SEQ ID NO: 400 - DNA, 22 bp, synthetic construct
  source 1..22: mol_type = other DNA; note = H51A(+78+99)
gtacctccaacatcaaggaaga

SEQ ID NO: 401 - DNA, 21 bp, synthetic construct
  source 1..21: mol_type = other DNA; note = H51A(+79+99)
gtacctccaacatcaaggaag

SEQ ID NO: 402 - DNA, 25 bp, synthetic construct
  source 1..25: mol_type = other DNA; note = H51.SA.(-60-36)
gaagaaaaagaaaaattagaaacac

SEQ ID NO: 403 - DNA, 25 bp, synthetic construct
  source 1..25: mol_type = other DNA; note = H51.SA.(-50-26)
aaggaaaaaagaagaaaaagaaaaa

SEQ ID NO: 404 - DNA, 25 bp, synthetic construct
  source 1..25: mol_type = other DNA; note = H51.SA.(-45-21)
gcaaaaaggaaaaaagaagaaaaag

SEQ ID NO: 405 - DNA, 25 bp, synthetic construct
  source 1..25: mol_type = other DNA; note = H51.SA.(-40-16)
tttttgcaaaaaggaaaaaagaaga

SEQ ID NO: 406 - DNA, 25 bp, synthetic construct
  source 1..25: mol_type = other DNA; note = H51.SA.(-35-11)
ttgggtttttgcaaaaaggaaaaaa

SEQ ID NO: 407 - DNA, 25 bp, synthetic construct
  source 1..25: mol_type = other DNA; note = H51.SA.(-30-6)
atattttgggtttttgcaaaaagga

SEQ ID NO: 408 - DNA, 25 bp, synthetic construct
  source 1..25: mol_type = other DNA; note = H51.SA.(-25-1)
ctaaaatattttgggtttttgcaaa

SEQ ID NO: 409 - DNA, 25 bp, synthetic construct
  source 1..25: mol_type = other DNA; note = H51.SA.(-20+5)
aggagctaaaatattttgggttttt

SEQ ID NO: 410 - DNA, 25 bp, synthetic construct
  source 1..25: mol_type = other DNA; note = H51.SA.(-15+10)
tgagtaggagctaaaatattttggg

SEQ ID NO: 411 - DNA, 25 bp, synthetic construct
  source 1..25: mol_type = other DNA; note = H51.SA.(-10+15)
cagtctgagtaggagctaaaatatt

SEQ ID NO: 412 - DNA, 25 bp, synthetic construct
  source 1..25: mol_type = other DNA; note = H51.SA.(-5+20)
agtaacagtctgagtaggagctaaa

SEQ ID NO: 413 - DNA, 25 bp, synthetic construct
  source 1..25: mol_type = other DNA; note = H51.SA.(-1+24)
ccagagtaacagtctgagtaggagc

SEQ ID NO: 414 - DNA, 25 bp, synthetic construct
  source 1..25: mol_type = other DNA; note = H51.SA.(-65-41)
aaaagaaaaattagaaacacaagct

SEQ ID NO: 415 - DNA, 25 bp, synthetic construct
  source 1..25: mol_type = other DNA; note = H51.SA.(-70-46)
aaaaattagaaacacaagctaaaga

SEQ ID NO: 416 - DNA, 25 bp, synthetic construct
  source 1..25: mol_type = other DNA; note = H51.SA.(-75-51)
ttagaaacacaagctaaagagccaa

SEQ ID NO: 417 - DNA, 25 bp, synthetic construct
  source 1..25: mol_type = other DNA; note = H51.SA.(-80-56)
aacacaagctaaagagccaatttca

SEQ ID NO: 418 - DNA, 25 bp, synthetic construct
  source 1..25: mol_type = other DNA; note = H51.SA.(-85-61)
aagctaaagagccaatttcaataac

SEQ ID NO: 419 - DNA, 25 bp, synthetic construct
  source 1..25: mol_type = other DNA; note = H51.SA.(-90-66)
aaagagccaatttcaataacaataa

SEQ ID NO: 420 - DNA, 25 bp, synthetic construct
  source 1..25: mol_type = other DNA; note = H51.SA.(-95-71)
gccaatttcaataacaataagtcaa

SEQ ID NO: 421 - DNA, 25 bp, synthetic construct
  source 1..25: mol_type = other DNA; note = H51.SA.(-100-76)
tttcaataacaataagtcaaattta

SEQ ID NO: 422 - DNA, 30 bp, synthetic construct; identical to SEQ ID NO: 70
  source 1..30: mol_type = other DNA; note = H51A(+1+30)

SEQ ID NO: 423 - DNA, 30 bp, synthetic construct; identical to SEQ ID NO: 71
  source 1..30: mol_type = other DNA; note = H51A(+10+39)

SEQ ID NO: 424 - DNA, 30 bp, synthetic construct; identical to SEQ ID NO: 72
  source 1..30: mol_type = other DNA; note = H51A(+6+35)

SEQ ID NO: 425 - DNA, 30 bp, synthetic construct; identical to SEQ ID NO: 73
  source 1..30: mol_type = other DNA; note = H51A(+49+78)

SEQ ID NO: 426 - DNA, 25 bp, synthetic construct
  source 1..25: mol_type = other DNA; note = H51A(+1+25)
accagagtaacagtctgagtaggag

SEQ ID NO: 427 - DNA, 25 bp, synthetic construct
  source 1..25: mol_type = other DNA; note = H51A(+4+28)
gtcaccagagtaacagtctgagtag

SEQ ID NO: 428 - DNA, 25 bp, synthetic construct
  source 1..25: mol_type = other DNA; note = H51A(+16+40)
accacaggttgtgtcaccagagtaa

SEQ ID NO: 429 - DNA, 25 bp, synthetic construct
  source 1..25: mol_type = other DNA; note = H51A(+21+45)
tagtaaccacaggttgtgtcaccag

SEQ ID NO: 430 - DNA, 25 bp, synthetic construct
  source 1..25: mol_type = other DNA; note = H51A(+26+50)
ttccttagtaaccacaggttgtgtc

SEQ ID NO: 431 - DNA, 25 bp, synthetic construct
  source 1..25: mol_type = other DNA; note = H51A(+31+55)
gcagtttccttagtaaccacaggtt

SEQ ID NO: 432 - DNA, 25 bp, synthetic construct
  source 1..25: mol_type = other DNA; note = H51A(+36+60)
agatggcagtttccttagtaaccac

SEQ ID NO: 433 - DNA, 25 bp, synthetic construct
  source 1..25: mol_type = other DNA; note = H51A(+41+65)
tttggagatggcagtttccttagta

SEQ ID NO: 434 - DNA, 25 bp, synthetic construct
  source 1..25: mol_type = other DNA; note = H51A(+86+110)
tgccagagcaggtacctccaacatc

SEQ ID NO: 435 - DNA, 25 bp, synthetic construct
  source 1..25: mol_type = other DNA; note = H51A(+91+115)
aaatctgccagagcaggtacctcca

SEQ ID NO: 436 - DNA, 25 bp, synthetic construct
  source 1..25: mol_type = other DNA; note = H51A(+96+120)
ggttgaaatctgccagagcaggtac

SEQ ID NO: 437 - DNA, 25 bp, synthetic construct
  source 1..25: mol_type = other DNA; note = H51A(+101+125)
agcccggttgaaatctgccagagca

SEQ ID NO: 438 - DNA, 25 bp, synthetic construct
  source 1..25: mol_type = other DNA; note = H51A(+106+130)
gtccaagcccggttgaaatctgcca

SEQ ID NO: 439 - DNA, 25 bp, synthetic construct
  source 1..25: mol_type = other DNA; note = H51A(+111+135)
gttctgtccaagcccggttgaaatc

SEQ ID NO: 440 - DNA, 25 bp, synthetic construct
  source 1..25: mol_type = other DNA; note = H51A(+116+140)
ggtaagttctgtccaagcccggttg

SEQ ID NO: 441 - DNA, 25 bp, synthetic construct
  source 1..25: mol_type = other DNA; note = H51A(+121+145)
cagtcggtaagttctgtccaagccc

SEQ ID NO: 442 - DNA, 25 bp, synthetic construct
  source 1..25: mol_type = other DNA; note = H51A(+126+150)
aaagccagtcggtaagttctgtcca

SEQ ID NO: 443 - DNA, 25 bp, synthetic construct
  source 1..25: mol_type = other DNA; note = H51A(+131+155)
cagagaaagccagtcggtaagttct

SEQ ID NO: 444 - DNA, 25 bp, synthetic construct
  source 1..25: mol_type = other DNA; note = H51A(+136+160)
tcaagcagagaaagccagtcggtaa

SEQ ID NO: 445 - DNA, 25 bp, synthetic construct
  source 1..25: mol_type = other DNA; note = H51A(+141+165)
cttgatcaagcagagaaagccagtc

SEQ ID NO: 446 - DNA, 25 bp, synthetic construct
  source 1..25: mol_type = other DNA; note = H51A(+146+170)
tataacttgatcaagcagagaaagc

SEQ ID NO: 447 - DNA, 25 bp, synthetic construct
  source 1..25: mol_type = other DNA; note = H51A(+151+175)
gattttataacttgatcaagcagag

SEQ ID NO: 448 - DNA, 25 bp, synthetic construct
  source 1..25: mol_type = other DNA; note = H51A(+156+180)
tctgtgattttataacttgatcaag

SEQ ID NO: 449 - DNA, 25 bp, synthetic construct
  source 1..25: mol_type = other DNA; note = H51A(+161+185)
caccctctgtgattttataacttga

SEQ ID NO: 450 - DNA, 25 bp, synthetic construct
  source 1..25: mol_type = other DNA; note = H51A(+166+190)
accatcaccctctgtgattttataa

SEQ ID NO: 451 - DNA, 25 bp, synthetic construct
  source 1..25: mol_type = other DNA; note = H51A(+171+195)
cacccaccatcaccctctgtgattt

SEQ ID NO: 452 - DNA, 25 bp, synthetic construct
  source 1..25: mol_type = other DNA; note = H51A(+176+200)
aaggtcacccaccatcaccctctgt

SEQ ID NO: 453 - DNA, 25 bp, synthetic construct
  source 1..25: mol_type = other DNA; note = H51A(+181+205)
tcctcaaggtcacccaccatcaccc

SEQ ID NO: 454 - DNA, 25 bp, synthetic construct
  source 1..25: mol_type = other DNA; note = H51A(+186+210)
tgatatcctcaaggtcacccaccat

SEQ ID NO: 455 - DNA, 25 bp, synthetic construct
  source 1..25: mol_type = other DNA; note = H51A(+191+215)
ctcgttgatatcctcaaggtcaccc

SEQ ID NO: 456 - DNA, 25 bp, synthetic construct
  source 1..25: mol_type = other DNA; note = H51A(+196+220)
atcatctcgttgatatcctcaaggt

SEQ ID NO: 457 - DNA, 25 bp, synthetic construct
  source 1..25: mol_type = other DNA; note = H51A(+201+225)
tgatgatcatctcgttgatatcctc

SEQ ID NO: 458 - DNA, 25 bp, synthetic construct
  source 1..25: mol_type = other DNA; note = H51A(+206+230)
ctgcttgatgatcatctcgttgata

SEQ ID NO: 459 - DNA, 25 bp, synthetic construct
  source 1..25: mol_type = other DNA; note = H51D(+211-02)
accttctgcttgatgatcatctcgt

SEQ ID NO: 460 - DNA, 25 bp, synthetic construct
  source 1..25: mol_type = other DNA; note = H51D(+214-05)
cataccttctgcttgatgatcatct

SEQ ID NO: 461 - DNA, 25 bp, synthetic construct
  source 1..25: mol_type = other DNA; note = H51D(+217-08)
tctcataccttctgcttgatgatca

SEQ ID NO: 462 - DNA, 25 bp, synthetic construct
  source 1..25: mol_type = other DNA; note = H51D(+220-11)
ttttctcataccttctgcttgatga

SEQ ID NO: 463 - DNA, 25 bp, synthetic construct
  source 1..25: mol_type = other DNA; note = H51D(+223-14)
attttttctcataccttctgcttga

SEQ ID NO: 464 - DNA, 25 bp, synthetic construct
  source 1..25: mol_type = other DNA; note = H51D(+226-17)
atcattttttctcataccttctgct

SEQ ID NO: 465 - DNA, 25 bp, synthetic construct
  source 1..25: mol_type = other DNA; note = H51D(+229-20)
tttatcattttttctcataccttct

SEQ ID NO: 466 - DNA, 25 bp, synthetic construct
  source 1..25: mol_type = other DNA; note = H51D(+232-23)
acttttatcattttttctcatacct

SEQ ID NO: 467 - DNA, 25 bp, synthetic construct
  source 1..25: mol_type = other DNA; note = H51D(-02-26)
ccaacttttatcattttttctcata

SEQ ID NO: 468 - DNA, 30 bp, synthetic construct; identical to SEQ ID NO: 70
  source 1..30: mol_type = other DNA; note = H51A(+1+30)

SEQ ID NO: 469 - DNA, 30 bp, synthetic construct; identical to SEQ ID NO: 1
  source 1..30: mol_type = other DNA; note = H51A(+66+95)

SEQ ID NO: 470 - DNA, 24 bp, synthetic construct
  source 1..24: mol_type = other DNA; note = H51A(+74+97)
acctccaacatcaaggaagatggc

SEQ ID NO: 471 - DNA, 30 bp, synthetic construct; identical to SEQ ID NO: 43
  source 1..30: mol_type = other DNA; note = H51A(+70+99)

SEQ ID NO: 472 - DNA, 28 bp, synthetic construct
  source 1..28: mol_type = other DNA; note = H51A(+72+99)
gtacctccaacatcaaggaagatggcat

SEQ ID NO: 473 - DNA, 20 bp, synthetic construct
  source 1..20: mol_type = other DNA; note = H51A(+68+87)
tcaaggaagatggcatttct

SEQ ID NO: 474 - RNA, 20 nt, synthetic construct
  source 1..20: mol_type = other RNA
  modified_base 7: mod_base = OTHER; note = methylated adenine
  modified_base 9: mod_base = OTHER; note = modified guanine
  modified_base 10: mod_base = OTHER; note = methylated adenine
  modified_base 12..13: mod_base = OTHER; note = modified guanine
tcaaggnanntnncatttct

SEQ ID NO: 475 - DNA, 25 bp, synthetic construct
  source 1..25: mol_type = other DNA; note = H53A(+36+60)
gttgcctccggttctgaaggtgttc

SEQ ID NO: 476 - DNA, 25 bp, synthetic construct
  source 1..25: mol_type = other DNA; note = H53A(+36+60); note = 5..6
  modified_base 5..6: mod_base = m5c
  modified_base 8..9: mod_base = m5c
  modified_base 14: mod_base = m5c
  modified_base 25: mod_base = m5c
gttgnntnnggttntgaaggtgttn

SEQ ID NO: 477 - DNA, 21 bp, synthetic construct
  source 1..21: mol_type = other DNA; note = H53A(+36+56)
cctccggttctgaaggtgttc

SEQ ID NO: 478 - DNA, 25 bp, synthetic construct
  source 1..25: mol_type = other DNA; note = H53A(+23+47)
ctgaaggtgttcttgtacttcatcc

SEQ ID NO: 479 - DNA, 25 bp, synthetic construct
  source 1..25: mol_type = other DNA; note = H53A(+32+56)
cctccggttctgaaggtgttcttgt

SEQ ID NO: 480 - DNA, 28 bp, synthetic construct
  source 1..28: mol_type = other DNA; note = H53A(+33+60)
gttgcctccggttctgaaggtgttcttg

SEQ ID NO: 481 - DNA, 30 bp, synthetic construct; identical to SEQ ID NO: 129
  source 1..30: mol_type = other DNA; note = H53A(+30+59)

SEQ ID NO: 482 - DNA, 24 bp, synthetic construct
  source 1..24: mol_type = other DNA; note = H53A(+39+62)
ctgttgcctccggttctgaaggtg

SEQ ID NO: 483 - DNA, 31 bp, synthetic construct; identical to SEQ ID NO: 131
  source 1..31: mol_type = other DNA; note = H53A(+36+69)

SEQ ID NO: 484 - DNA, 18 bp, synthetic construct
  source 1..18: mol_type = other DNA; note = H53A(+45+62)
ctgttgcctccggttctg

SEQ ID NO: 485 - DNA, 22 bp, synthetic construct
  source 1..22: mol_type = other DNA; note = H45A(-03+19)
caatgccatcctggagttcctg

SEQ ID NO: 486 - DNA, 34 bp, synthetic construct; identical to SEQ ID NO: 134
  source 1..34: mol_type = other DNA; note = H45A(-09+25)

SEQ ID NO: 487 - DNA, 28 bp, synthetic construct
  source 1..28: mol_type = other DNA; note = H45A(-03+25)
gctgcccaatgccatcctggagttcctg

SEQ ID NO: 488 - DNA, 31 bp, synthetic construct; identical to SEQ ID NO: 137
  source 1..31: mol_type = other DNA; note = H45A(-12+19)

SEQ ID NO: 489 - DNA, 31 bp, synthetic construct; identical to SEQ ID NO: 136
  source 1..31: mol_type = other DNA; note = H45A(-06+25)

SEQ ID NO: 490 - DNA, 28 bp, synthetic construct
  source 1..28: mol_type = other DNA; note = H45A(-09+19)
caatgccatcctggagttcctgtaagat

SEQ ID NO: 491 - DNA, 28 bp, synthetic construct
  source 1..28: mol_type = other DNA; note = H45A(-12+16)
tgccatcctggagttcctgtaagatacc

SEQ ID NO: 492 - DNA, 39 bp, synthetic construct; identical to SEQ ID NO: 140
  source 1..39: mol_type = other DNA; note = H45A(-14+25)

SEQ ID NO: 493 - DNA, 27 bp, synthetic construct
  source 1..27: mol_type = other DNA; note = H45A(-08+19)
caatgccatcctggagttcctgtaaga

SEQ ID NO: 494 - DNA, 32 bp, synthetic construct; identical to SEQ ID NO: 142
  source 1..32: mol_type = other DNA; note = HM45A(-07+25)

SEQ ID NO: 495 - DNA, 34 bp, synthetic construct; identical to SEQ ID NO: 143
  source 1..34: mol_type = other DNA; note = H45A(-12+22)

SEQ ID NO: 496 - DNA, 31 bp, synthetic construct; identical to SEQ ID NO: 144
  source 1..31: mol_type = other DNA; note = H45A(-09+22)

SEQ ID NO: 497 - DNA, 39 bp, synthetic construct; identical to SEQ ID NO: 145
  source 1..39: mol_type = other DNA; note = H45A(-09+30)

SEQ ID NO: 498 - DNA, 28 bp, synthetic construct
  source 1..28: mol_type = other DNA; note = H45A(-06+22)
gcccaatgccatcctggagttcctgtaa

SEQ ID NO: 499 - DNA, 34 bp, synthetic construct; identical to SEQ ID NO: 147
  source 1..34: mol_type = other DNA; note = H45A(-06+28)

SEQ ID NO: 500 - DNA, 25 bp, synthetic construct
  source 1..25: mol_type = other DNA; note = H45A(-03+22)
gcccaatgccatcctggagttcctg

SEQ ID NO: 501 - DNA, 31 bp, synthetic construct; identical to SEQ ID NO: 149
  source 1..31: mol_type = other DNA; note = H45A(-03+28)

SEQ ID NO: 502 - RNA, 18 nt, synthetic construct
  source 1..18: mol_type = other RNA
  modified_base 1: mod_base = m5c
  modified_base 3: mod_base = m5c
  modified_base 7..8: mod_base = m5c
  modified_base 11: mod_base = m5u
  modified_base 13..14: mod_base = m5c
  modified_base 4: mod_base = m5u
  modified_base 17..18: mod_base = m5c
ngntgcnnaatgnnatnn

SEQ ID NO: 503 - DNA, 25 bp, synthetic construct
  source 1..25: mol_type = other DNA; note = H44A(-10+15)
gatctgtcaaatcgcctgcaggtaa

SEQ ID NO: 504 - DNA, 22 bp, synthetic construct
  source 1..22: mol_type = other DNA; note = H44A(-07+15)
gatctgtcaaatcgcctgcagg

SEQ ID NO: 505 - DNA, 24 bp, synthetic construct
  source 1..24: mol_type = other DNA; note = H44M(-07+17)
cagatctgtcaaatcgcctgcagg

SEQ ID NO: 506 - DNA, 23 bp, synthetic construct
  source 1..23: mol_type = other DNA; note = H44A(-08+15)
gatctgtcaaatcgcctgcaggt

SEQ ID NO: 507 - DNA, 21 bp, synthetic construct
  source 1..21: mol_type = other DNA; note = H44A(-06+15)
gatctgtcaaatcgcctgcag

SEQ ID NO: 508 - DNA, 25 bp, synthetic construct
  source 1..25: mol_type = other DNA; note = H44A(-08+17)
cagatctgtcaaatcgcctgcaggt

SEQ ID NO: 509 - DNA, 23 bp, synthetic construct
  source 1..23: mol_type = other DNA; note = H44A(-06+17)
cagatctgtcaaatcgcctgcag

SEQ ID NO: 510 - DNA, 22 bp, synthetic construct
  source 1..22: mol_type = other DNA; note = H50D(+04-18)
gggatccagtatacttacaggc

SEQ ID NO: 511 - DNA, 25 bp, synthetic construct
  source 1..25: mol_type = other DNA; note = H50D(+07-18)
gggatccagtatacttacaggctcc

SEQ ID NO: 512 - DNA, 23 bp, synthetic construct
  source 1..23: mol_type = other DNA; note = H50D(+07-16)
gatccagtatacttacaggctcc

SEQ ID NO: 513 - DNA, 24 bp, synthetic construct
  source 1..24: mol_type = other DNA; note = H50D(+07-17)
ggatccagtatacttacaggctcc

SEQ ID NO: 514 - DNA, 26 bp, synthetic construct
  source 1..26: mol_type = other DNA; note = H50A(-19+07)
acttcctctttaacagaaaagcatac

SEQ ID NO: 515 - DNA, 22 bp, synthetic construct
  source 1..22: mol_type = other DNA; note = H50D(+07-15)
atccagtatacttacaggctcc

SEQ ID NO: 516 - DNA, 25 bp, synthetic construct
  source 1..25: mol_type = other DNA; note = H50A(-02+23)
gagctcagatcttctaacttcctct

SEQ ID NO: 517 - DNA, 24 bp, synthetic construct
  source 1..24: mol_type = other DNA; note = H50D(+06-18)
gggatccagtatacttacaggctc

SEQ ID NO: 518 - DNA, 27 bp, synthetic construct
  source 1..27: mol_type = other DNA; note = H50D(+07-20)
atgggatccagtatacttacaggctcc

SEQ ID NO: 519 - DNA, 25 bp, synthetic construct
  source 1..25: mol_type = other DNA; note = H52A(-01+24)
ctgttccaaatcctgcattgttgcc

SEQ ID NO: 520 - DNA, 23 bp, synthetic construct
  source 1..23: mol_type = other DNA; note = DYS-d52-F006
aaagcagcctgacctagctcctg

SEQ ID NO: 521 - DNA, 25 bp, synthetic construct
  source 1..25: mol_type = other DNA; note = DYS-d52-F006
tcagcttcttccttagcttccagcc

SEQ ID NO: 522 - DNA, 36 bp, synthetic construct
  source 1..36: mol_type = other DNA; note = DYS-WT-exon51-P001
acacaacctgtggttactaaggaaactgccatctcc

SEQ ID NO: 523 - DNA, 39 bp, synthetic construct
  source 1..39: mol_type = other DNA; note = DYS-d525KP51-P003
tgaccactattggagcctttgaaagaattcagaatcagt